SEQUENCE LISTING (1400 sequences)

SEQ ID NO: 1 - DNA, 45 bp, Artificial Sequence
  Target Specific Oligonucleotide
actactagagaccggtagaaatgagtaacgattaccgacttatcc

SEQ ID NO: 2 - DNA, 42 bp, Artificial Sequence
  Target Specific Oligonucleotide
tggcggggaacgaagtatatagtaacgattaccgacttatcc

SEQ ID NO: 3 - DNA, 40 bp, Artificial Sequence
  Target Specific Oligonucleotide
tggcgtcaatggttgcggtgtaacgattaccgacttatcc

SEQ ID NO: 4 - DNA, 41 bp, Artificial Sequence
  Target Specific Oligonucleotide
tcggtcactcgaataacccggtaacgattaccgacttatcc

SEQ ID NO: 5 - DNA, 41 bp, Artificial Sequence
  Target Specific Oligonucleotide
ctccaaatcgaggtgcaccagtaacgattaccgacttatcc

SEQ ID NO: 6 - DNA, 40 bp, Artificial Sequence
  Target Specific Oligonucleotide
ggccagttcccgagttggtgtaacgattaccgacttatcc

SEQ ID NO: 7 - DNA, 42 bp, Artificial Sequence
  Target Specific Oligonucleotide
agacttcaagcgacatgctctgtaacgattaccgacttatcc

SEQ ID NO: 8 - DNA, 42 bp, Artificial Sequence
  Target Specific Oligonucleotide
ggagcaccaccgtagatacaagtaacgattaccgacttatcc

SEQ ID NO: 9 - DNA, 44 bp, Artificial Sequence
  Target Specific Oligonucleotide
ctctccaaatcacgtatttgtgggtaacgattaccgacttatcc

SEQ ID NO: 10 - DNA, 45 bp, Artificial Sequence
  Target Specific Oligonucleotide
agagtttgcctatcaggtcttattgtaacgattaccgacttatcc

SEQ ID NO: 11 - DNA, 40 bp, Artificial Sequence
  Target Specific Oligonucleotide
ggccaagtcgcactccacagtaacgattaccgacttatcc

SEQ ID NO: 12 - DNA, 43 bp, Artificial Sequence
  Target Specific Oligonucleotide
tccttttctacgtcatgacacagtaacgattaccgacttatcc

SEQ ID NO: 13 - DNA, 46 bp, Artificial Sequence
  Target Specific Oligonucleotide
ttccattagacgaataagtttttcagtaacgattaccgacttatcc

SEQ ID NO: 14 - DNA, 44 bp, Artificial Sequence
  Target Specific Oligonucleotide
tttctggtaagctcatcacatctgtaacgattaccgacttatcc

SEQ ID NO: 15 - DNA, 46 bp, Artificial Sequence
  Target Specific Oligonucleotide
gagttagggtagtcataattgatgagtaacgattaccgacttatcc

SEQ ID NO: 16 - DNA, 47 bp, Artificial Sequence
  Target Specific Oligonucleotide
ttcttccaattcgatgaatataatccgtaacgattaccgacttatcc

SEQ ID NO: 17 - DNA, 48 bp, Artificial Sequence
  Target Specific Oligonucleotide
tgttattaggtgtaaagaaagtgtatggtaacgattaccgacttatcc

SEQ ID NO: 18 - DNA, 42 bp, Artificial Sequence
  Target Specific Oligonucleotide
gagataaggtcgctcacttgcgtaacgattaccgacttatcc

SEQ ID NO: 19 - DNA, 42 bp, Artificial Sequence
  Target Specific Oligonucleotide
acctctgtcttcgaccaactggtaacgattaccgacttatcc

SEQ ID NO: 20 - DNA, 41 bp, Artificial Sequence
  Target Specific Oligonucleotide
acccctggaacgacctgaacgtaacgattaccgacttatcc

SEQ ID NO: 21 - DNA, 41 bp, Artificial Sequence
  Target Specific Oligonucleotide
agtatctgtcccgacggtcagtaacgattaccgacttatcc

SEQ ID NO: 22 - DNA, 40 bp, Artificial Sequence
  Target Specific Oligonucleotide
accccttttgcccgcagaggtaacgattaccgacttatcc

SEQ ID NO: 23 - DNA, 44 bp, Artificial Sequence
  Target Specific Oligonucleotide
tttaagcaggctagagtaacctcgtaacgattaccgacttatcc

SEQ ID NO: 24 - DNA, 45 bp, Artificial Sequence
  Target Specific Oligonucleotide
gcattggataaccaatcataggtggtaacgattaccgacttatcc

SEQ ID NO: 25 - DNA, 41 bp, Artificial Sequence
  Target Specific Oligonucleotide
tcccctagtccgagttgctcgtaaccctaattatgggaatc

SEQ ID NO: 26 - DNA, 43 bp, Artificial Sequence
  Target Specific Oligonucleotide
ccaatttgcacttggatgtgtagtaaccctaattatgggaatc

SEQ ID NO: 27 - DNA, 43 bp, Artificial Sequence
  Target Specific Oligonucleotide
ttttgccagtagaacaagcatggtaaccctaattatgggaatc

SEQ ID NO: 28 - DNA, 40 bp, Artificial Sequence
  Target Specific Oligonucleotide
ggaggcaaggttccgcaacgtaaccctaattatgggaatc

SEQ ID NO: 29 - DNA, 44 bp, Artificial Sequence
  Target Specific Oligonucleotide
agaattccacgtaaatcacatgcgtaaccctaattatgggaatc

SEQ ID NO: 30 - DNA, 44 bp, Artificial Sequence
  Target Specific Oligonucleotide
tatgcatgtacagggaagtatccgtaaccctaattatgggaatc

SEQ ID NO: 31 - DNA, 43 bp, Artificial Sequence
  Target Specific Oligonucleotide
gtgtgcatgccttagaaaaagcgtaaccctaattatgggaatc

SEQ ID NO: 32 - DNA, 43 bp, Artificial Sequence
  Target Specific Oligonucleotide
agcttttgtgcccttatcatgagtaaccctaattatgggaatc

SEQ ID NO: 33 - DNA, 46 bp, Artificial Sequence
  Target Specific Oligonucleotide
cttcattagtatcttgtcacttggggtaaccctaattatgggaatc

SEQ ID NO: 34 - DNA, 45 bp, Artificial Sequence
  Target Specific Oligonucleotide
agatgatgaccctaccaaatttgggtaaccctaattatgggaatc

SEQ ID NO: 35 - DNA, 44 bp, Artificial Sequence
  Target Specific Oligonucleotide
aggcaagagatagatatgtgggcgtaaccctaattatgggaatc

SEQ ID NO: 36 - DNA, 44 bp, Artificial Sequence
  Target Specific Oligonucleotide
cagctgccactataatcatgtttgtaaccctaattatgggaatc

SEQ ID NO: 37 - DNA, 45 bp, Artificial Sequence
  Target Specific Oligonucleotide
ggaacatgctaatttaaggtgagtgtaaccctaattatgggaatc

SEQ ID NO: 38 - DNA, 46 bp, Artificial Sequence
  Target Specific Oligonucleotide
aggactttgctatatcaagtagttcgtaaccctaattatgggaatc

SEQ ID NO: 39 - DNA, 46 bp, Artificial Sequence
  Target Specific Oligonucleotide
aaaggagatagtaacaatggttttcgtaaccctaattatgggaatc

SEQ ID NO: 40 - DNA, 43 bp, Artificial Sequence
  Target Specific Oligonucleotide
aaatcagtggttcaccctgttcgtaaccctaattatgggaatc

SEQ ID NO: 41 - DNA, 48 bp, Artificial Sequence
  Target Specific Oligonucleotide
cttgtcataagataattaggcaaattagtaaccctaattatgggaatc

SEQ ID NO: 42 - DNA, 47 bp, Artificial Sequence
  Target Specific Oligonucleotide
tcattaagttaatgctaaggatctttgtaaccctaattatgggaatc

SEQ ID NO: 43 - DNA, 43 bp, Artificial Sequence
  Target Specific Oligonucleotide
tgtcactggtacaagtggacttgtaaccctaattatgggaatc

SEQ ID NO: 44 - DNA, 45 bp, Artificial Sequence
  Target Specific Oligonucleotide
ttggtcactatacaagtgacttctgtaaccctaattatgggaatc

SEQ ID NO: 45 - DNA, 43 bp, Artificial Sequence
  Target Specific Oligonucleotide
aggaaaatgggcgtaaaggagggtaaccctaattatgggaatc

SEQ ID NO: 46 - DNA, 46 bp, Artificial Sequence
  Target Specific Oligonucleotide
ttcatctacaatagggacaacaaacgtaaccctaattatgggaatc

SEQ ID NO: 47 - DNA, 44 bp, Artificial Sequence
  Target Specific Oligonucleotide
gataagtctacgtggaaaagcatgtaaccctaattatgggaatc

SEQ ID NO: 48 - DNA, 45 bp, Artificial Sequence
  Target Specific Oligonucleotide
caggactacagttaagcatttactgtaaccctaattatgggaatc

SEQ ID NO: 49 - DNA, 43 bp, Artificial Sequence
  Target Specific Oligonucleotide
gtctaggaaattgccgtggttggtaaccctaattatgggaatc

SEQ ID NO: 50 - DNA, 42 bp, Artificial Sequence
  Target Specific Oligonucleotide
aatcatcgtctcgaaagcggtgtaaccctaattatgggaatc

SEQ ID NO: 51 - DNA, 43 bp, Artificial Sequence
  Target Specific Oligonucleotide
cttgaccaaattcgaaggtccagtaaccctaattatgggaatc

SEQ ID NO: 52 - DNA, 43 bp, Artificial Sequence
  Target Specific Oligonucleotide
gatgactctgttcgcatcctcggtaaccctaattatgggaatc

SEQ ID NO: 53 - DNA, 42 bp, Artificial Sequence
  Target Specific Oligonucleotide
gatgctagtgtcaaacctgccgtaaccctaattatgggaatc

SEQ ID NO: 54 - DNA, 44 bp, Artificial Sequence
  Target Specific Oligonucleotide
tgatacagggctcgtacttatccgtaaccctaattatgggaatc

SEQ ID NO: 55 - DNA, 43 bp, Artificial Sequence
  Target Specific Oligonucleotide
aggagtcacacgagttgaaaaggtaaccctaattatgggaatc

SEQ ID NO: 56 - DNA, 44 bp, Artificial Sequence
  Target Specific Oligonucleotide
ctcattacaccgaggtatgaagggtaaccctaattatgggaatc

SEQ ID NO: 57 - DNA, 40 bp, Artificial Sequence
  Target Specific Oligonucleotide
ctctgtcacggctcgggtggtaaccctaattatgggaatc

SEQ ID NO: 58 - DNA, 43 bp, Artificial Sequence
  Target Specific Oligonucleotide
ctaccatatgtacccgacctcagtaaccctaattatgggaatc

SEQ ID NO: 59 - DNA, 44 bp, Artificial Sequence
  Target Specific Oligonucleotide
aaccttaggtcgggtatagagaggtaaccctaattatgggaatc

SEQ ID NO: 60 - DNA, 44 bp, Artificial Sequence
  Target Specific Oligonucleotide
gaagcacttagacactgtaaggcgtaaccctaattatgggaatc

SEQ ID NO: 61 - DNA, 42 bp, Artificial Sequence
  Target Specific Oligonucleotide
aataatcaccggcagtaacgggtaaccctaattatgggaatc

SEQ ID NO: 62 - DNA, 43 bp, Artificial Sequence
  Target Specific Oligonucleotide
aggaagaaacctatggcagacagtaaccctaattatgggaatc

SEQ ID NO: 63 - DNA, 46 bp, Artificial Sequence
  Target Specific Oligonucleotide
catgtgttattacgatgttctttgtgtaaccctaattatgggaatc

SEQ ID NO: 64 - DNA, 45 bp, Artificial Sequence
  Target Specific Oligonucleotide
actactgtttggtacttgtatctggtaaccctaattatgggaatc

SEQ ID NO: 65 - DNA, 42 bp, Artificial Sequence
  Target Specific Oligonucleotide
aaatcagtggcggacagtagcgtaaccctaattatgggaatc

SEQ ID NO: 66 - DNA, 41 bp, Artificial Sequence
  Target Specific Oligonucleotide
caggcaatgccgactggattgtaaccctaattatgggaatc

SEQ ID NO: 67 - DNA, 48 bp, Artificial Sequence
  Target Specific Oligonucleotide
tcttatctaagaccaactataggtatggtaaccctaattatgggaatc

SEQ ID NO: 68 - DNA, 43 bp, Artificial Sequence
  Target Specific Oligonucleotide
gggaactgtctattgagcactcgtaaccctaattatgggaatc

SEQ ID NO: 69 - DNA, 47 bp, Artificial Sequence
  Target Specific Oligonucleotide
attacaatctttagtactcatggaaagtaaccctaattatgggaatc

SEQ ID NO: 70 - DNA, 46 bp, Artificial Sequence
  Target Specific Oligonucleotide
aaggtattctcatgcctagaatattgtaaccctaattatgggaatc

SEQ ID NO: 71 - DNA, 43 bp, Artificial Sequence
  Target Specific Oligonucleotide
tggacagacacgttgtcatttggtaaccctaattatgggaatc

SEQ ID NO: 72 - DNA, 48 bp, Artificial Sequence
  Target Specific Oligonucleotide
ctctcattacaatgctatacatttaacgtaaccctaattatgggaatc

SEQ ID NO: 73 - DNA, 41 bp, Artificial Sequence
  Target Specific Oligonucleotide
tgcagctcgcaacggaacaagtaacggattttacaacttta

SEQ ID NO: 74 - DNA, 43 bp, Artificial Sequence
  Target Specific Oligonucleotide
gagtccatccggacattgacctgtaacggattttacaacttta

SEQ ID NO: 75 - DNA, 41 bp, Artificial Sequence
  Target Specific Oligonucleotide
cttgaacggcctcgacgagggtaacggattttacaacttta

SEQ ID NO: 76 - DNA, 43 bp, Artificial Sequence
  Target Specific Oligonucleotide
cctcctcggttatattggccccgtaacggattttacaacttta

SEQ ID NO: 77 - DNA, 44 bp, Artificial Sequence
  Target Specific Oligonucleotide
accgagacttcgaaaatgaacgagtaacggattttacaacttta

SEQ ID NO: 78 - DNA, 43 bp, Artificial Sequence
  Target Specific Oligonucleotide
cctctggctcgaaactgaaagggtaacggattttacaacttta

SEQ ID NO: 79 - DNA, 44 bp, Artificial Sequence
  Target Specific Oligonucleotide
tgctatggactagatctcggcaagtaacggattttacaacttta

SEQ ID NO: 80 - DNA, 42 bp, Artificial Sequence
  Target Specific Oligonucleotide
gccttgaccggtcagaagacggtaacggattttacaacttta

SEQ ID NO: 81 - DNA, 42 bp, Artificial Sequence
  Target Specific Oligonucleotide
taccacagagatcgcagctgcgtaacggattttacaacttta

SEQ ID NO: 82 - DNA, 40 bp, Artificial Sequence
  Target Specific Oligonucleotide
accagcccgaacctcgcccgtaacggattttacaacttta

SEQ ID NO: 83 - DNA, 44 bp, Artificial Sequence
  Target Specific Oligonucleotide
actgtatgcttgccggtaattctgtaacggattttacaacttta

SEQ ID NO: 84 - DNA, 41 bp, Artificial Sequence
  Target Specific Oligonucleotide
gaacatgaaccggccccgacgtaacggattttacaacttta

SEQ ID NO: 85 - DNA, 42 bp, Artificial Sequence
  Target Specific Oligonucleotide
caggaatcccgtcaagggtgggtaacggattttacaacttta

SEQ ID NO: 86 - DNA, 47 bp, Artificial Sequence
  Target Specific Oligonucleotide
tctcgatttacctcgtttaagatctcgtaacggattttacaacttta

SEQ ID NO: 87 - DNA, 43 bp, Artificial Sequence
  Target Specific Oligonucleotide
agaaccatggtcgaactgacctgtaacggattttacaacttta

SEQ ID NO: 88 - DNA, 41 bp, Artificial Sequence
  Target Specific Oligonucleotide
tctgaggggctacgctgagagtaacggattttacaacttta

SEQ ID NO: 89 - DNA, 41 bp, Artificial Sequence
  Target Specific Oligonucleotide
aagcctctgttacgcccacggtaacggattttacaacttta

SEQ ID NO: 90 - DNA, 46 bp, Artificial Sequence
  Target Specific Oligonucleotide
aatgattcaactcgtactggatcccgtaacggattttacaacttta

SEQ ID NO: 91 - DNA, 44 bp, Artificial Sequence
  Target Specific Oligonucleotide
cctcggaaatcccgattctgatagtaacggattttacaacttta

SEQ ID NO: 92 - DNA, 41 bp, Artificial Sequence
  Target Specific Oligonucleotide
ggcccctactcggttgtggggtaacggattttacaacttta

SEQ ID NO: 93 - DNA, 45 bp, Artificial Sequence
  Target Specific Oligonucleotide
ggaaaatggaacgaaactcctgttgtaacggattttacaacttta

SEQ ID NO: 94 - DNA, 40 bp, Artificial Sequence
  Target Specific Oligonucleotide
ctgggtcgtgcgaggtcctgtaacggattttacaacttta

SEQ ID NO: 95 - DNA, 40 bp, Artificial Sequence
  Target Specific Oligonucleotide
cacggggctaggacggggtgtaacggattttacaacttta

SEQ ID NO: 96 - DNA, 39 bp, Artificial Sequence
  Target Specific Oligonucleotide
ccgatggcgtactcgtcgggtaacccttattcggtacta

SEQ ID NO: 97 - DNA, 42 bp, Artificial Sequence
  Target Specific Oligonucleotide
gatcagatcagaatccgaggtggtaacccttattcggtacta

SEQ ID NO: 98 - DNA, 40 bp, Artificial Sequence
  Target Specific Oligonucleotide
ggttgtactcgcgacagttggtaacccttattcggtacta

SEQ ID NO: 99 - DNA, 40 bp, Artificial Sequence
  Target Specific Oligonucleotide
gacctgctcattcgaggtgagtaacccttattcggtacta

SEQ ID NO: 100 - DNA, 39 bp, Artificial Sequence
  Target Specific Oligonucleotide
tcacgtagtagcggcttcggtaacccttattcggtacta

SEQ ID NO: 101 - DNA, 42 bp, Artificial Sequence
  Target Specific Oligonucleotide
ccaaagatgggcctacttgtcagtaacccttattcggtacta

SEQ ID NO: 102 - DNA, 39 bp, Artificial Sequence
  Target Specific Oligonucleotide
gacagagctgcacgcttgggtaacccttattcggtacta

SEQ ID NO: 103 - DNA, 39 bp, Artificial Sequence
  Target Specific Oligonucleotide
ctgggcctgtttgcgctcagtaacccttattcggtacta

SEQ ID NO: 104 - DNA, 38 bp, Artificial Sequence
  Target Specific Oligonucleotide
tccttaggcatgcgcggcgtaacccttattcggtacta

SEQ ID NO: 105 - DNA, 39 bp, Artificial Sequence
  Target Specific Oligonucleotide
cagcggcagtactcgctgtgtaacccttattcggtacta

SEQ ID NO: 106 - DNA, 38 bp, Artificial Sequence
  Target Specific Oligonucleotide
atgccaccgatgacccgcgtaacccttattcggtacta

SEQ ID NO: 107 - DNA, 38 bp, Artificial Sequence
  Target Specific Oligonucleotide
atcagcgtcgctccctcggtaacccttattcggtacta

SEQ ID NO: 108 - DNA, 41 bp, Artificial Sequence
  Target Specific Oligonucleotide
ctttgagagctcgaacatcgtgtaacccttattcggtacta

SEQ ID NO: 109 - DNA, 42 bp, Artificial Sequence
  Target Specific Oligonucleotide
ttccagtagttaaggcagagcagtaacccttattcggtacta

SEQ ID NO: 110 - DNA, 39 bp, Artificial Sequence
  Target Specific Oligonucleotide
gagctgcaggatcgggtccgtaacccttattcggtacta

SEQ ID NO: 111 - DNA, 41 bp, Artificial Sequence
  Target Specific Oligonucleotide
acatgagtggtttcgtagcgggtaacccttattcggtacta

SEQ ID NO: 112 - DNA, 40 bp, Artificial Sequence
  Target Specific Oligonucleotide
tgccgatgacatggaactcggtaacccttattcggtacta

SEQ ID NO: 113 - DNA, 43 bp, Artificial Sequence
  Target Specific Oligonucleotide
tggtgtaattgaccttgtaggtagtaacccttattcggtacta

SEQ ID NO: 114 - DNA, 39 bp, Artificial Sequence
  Target Specific Oligonucleotide
cattggccgaaagcaggctgtaacccttattcggtacta

SEQ ID NO: 115 - DNA, 40 bp, Artificial Sequence
  Target Specific Oligonucleotide
gagacctctgccgaaactgggtaacccttattcggtacta

SEQ ID NO: 116 - DNA, 41 bp, Artificial Sequence
  Target Specific Oligonucleotide
agtcttgtgcttcgggtcaaagtaacccttattcggtacta

SEQ ID NO: 117 - DNA, 41 bp, Artificial Sequence
  Target Specific Oligonucleotide
cctcttctcgcctggcatagggtaacccttattcggtacta

SEQ ID NO: 118 - DNA, 41 bp, Artificial Sequence
  Target Specific Oligonucleotide
gtaacggtgaaaatggaccgggtaacccttattcggtacta

SEQ ID NO: 119 - DNA, 42 bp, Artificial Sequence
  Target Specific Oligonucleotide
tccctctaagggattaatgccagtaacccttattcggtacta

SEQ ID NO: 120 - DNA, 43 bp, Artificial Sequence
  Target Specific Oligonucleotide
agttcaagtatcccgcgactagtaacaattagagtgaaatacc

SEQ ID NO: 121 - DNA, 43 bp, Artificial Sequence
  Target Specific Oligonucleotide
gaacgcaggctgtttactgttgtaacaattagagtgaaatacc

SEQ ID NO: 122 - DNA, 44 bp, Artificial Sequence
  Target Specific Oligonucleotide
ggtccaggtaaactaatggctggtaacaattagagtgaaatacc

SEQ ID NO: 123 - DNA, 44 bp, Artificial Sequence
  Target Specific Oligonucleotide
ctctgttgagtttacctcgcaagtaacaattagagtgaaatacc

SEQ ID NO: 124 - DNA, 44 bp, Artificial Sequence
  Target Specific Oligonucleotide
tacttcaactaaccagtccacggtaacaattagagtgaaatacc

SEQ ID NO: 125 - DNA, 44 bp, Artificial Sequence
  Target Specific Oligonucleotide
ccatgagacaaggcttaagactgtaacaattagagtgaaatacc

SEQ ID NO: 126 - DNA, 43 bp, Artificial Sequence
  Target Specific Oligonucleotide
cggccaaagaatagtcgtagcgtaacaattagagtgaaatacc

SEQ ID NO: 127 - DNA, 45 bp, Artificial Sequence
  Target Specific Oligonucleotide
ccatttccctaaggtatgtgtgagtaacaattagagtgaaatacc

SEQ ID NO: 128 - DNA, 46 bp, Artificial Sequence
  Target Specific Oligonucleotide
ctctcatactgttagtgatgtctggtaacaattagagtgaaatacc

SEQ ID NO: 129 - DNA, 42 bp, Artificial Sequence
  Target Specific Oligonucleotide
cagctttgtcccgtgactgtgtaacaattagagtgaaatacc

SEQ ID NO: 130 - DNA, 41 bp, Artificial Sequence
  Target Specific Oligonucleotide
tgctgcaatgctagcagcggtaacaattagagtgaaatacc

SEQ ID NO: 131 - DNA, 46 bp, Artificial Sequence
  Target Specific Oligonucleotide
aagacaggaacctatcaatgtagtgtaacaattagagtgaaatacc

SEQ ID NO: 132 - DNA, 46 bp, Artificial Sequence
  Target Specific Oligonucleotide
tagattagattatgcccaagtcaggtaacaattagagtgaaatacc

SEQ ID NO: 133 - DNA, 46 bp, Artificial Sequence
  Target Specific Oligonucleotide
catgtaacccactttaggtttacagtaacaattagagtgaaatacc

SEQ ID NO: 134 - DNA, 47 bp, Artificial Sequence
  Target Specific Oligonucleotide
ccagtctttcgtattaatgattcaggtaacaattagagtgaaatacc

SEQ ID NO: 135 - DNA, 42 bp, Artificial Sequence
  Target Specific Oligonucleotide
tgcaacccgtctcgtcttcggtaacaattagagtgaaatacc

SEQ ID NO: 136 - DNA, 47 bp, Artificial Sequence
  Target Specific Oligonucleotide
gtgtaggtatcatctgtaatgtacagtaacaattagagtgaaatacc

SEQ ID NO: 137 - DNA, 41 bp, Artificial Sequence
  Target Specific Oligonucleotide
tcccggacttcagtaccgcgtaacaattagagtgaaatacc

SEQ ID NO: 138 - DNA, 41 bp, Artificial Sequence
  Target Specific Oligonucleotide
cgaggaggttgcgaaaggcgtaacaattagagtgaaatacc

SEQ ID NO: 139 - DNA, 49 bp, Artificial Sequence
  Target Specific Oligonucleotide
tatacatatactcaacacttatagagggtaacaattagagtgaaatacc

SEQ ID NO: 140 - DNA, 49 bp, Artificial Sequence
  Target Specific Oligonucleotide
gtaatagataccataatttgtacttgggtaacaattagagtgaaatacc

SEQ ID NO: 141 - DNA, 40 bp, Artificial Sequence
  Target Specific Oligonucleotide
tgctgctgcgctagacccgtaacaattagagtgaaatacc

SEQ ID NO: 142 - DNA, 40 bp, Artificial Sequence
  Target Specific Oligonucleotide
gcgtggcgagccaaagacgtaacaattagagtgaaatacc

SEQ ID NO: 143 - DNA, 40 bp, Artificial Sequence
  Target Specific Oligonucleotide
tccgcggttgttggacgggtaacaattagagtgaaatacc

SEQ ID NO: 144 - DNA, 41 bp, Artificial Sequence
  Target Specific Oligonucleotide
ctgtcacatacgcaaactggtgtaacatcgttatagctaga

SEQ ID NO: 145 - DNA, 41 bp, Artificial Sequence
  Target Specific Oligonucleotide
tcgccatataccggtcaaagagtaacatcgttatagctaga

SEQ ID NO: 146 - DNA, 42 bp, Artificial Sequence
  Target Specific Oligonucleotide
tggatggtgcaataatccgagggtaacatcgttatagctaga

SEQ ID NO: 147 - DNA, 43 bp, Artificial Sequence
  Target Specific Oligonucleotide
agtgctgatcccttaagtatgtcgtaacatcgttatagctaga

SEQ ID NO: 148 - DNA, 43 bp, Artificial Sequence
  Target Specific Oligonucleotide
acctccattaaccaatccagaaggtaacatcgttatagctaga

SEQ ID NO: 149 - DNA, 43 bp, Artificial Sequence
  Target Specific Oligonucleotide
tggtgtacttgacccacttatttgtaacatcgttatagctaga

SEQ ID NO: 150 - DNA, 41 bp, Artificial Sequence
  Target Specific Oligonucleotide
cagaagagaacgtggagcagggtaacatcgttatagctaga

SEQ ID NO: 151 - DNA, 44 bp, Artificial Sequence
  Target Specific Oligonucleotide
tccctgtgaagtttatagacttcagtaacatcgttatagctaga

SEQ ID NO: 152 - DNA, 40 bp, Artificial Sequence
  Target Specific Oligonucleotide
tgtaaaagcaaacgcacgccgtaacatcgttatagctaga

SEQ ID NO: 153 - DNA, 44 bp, Artificial Sequence
  Target Specific Oligonucleotide
attcagatgacgagaaatgatacagtaacatcgttatagctaga

SEQ ID NO: 154 - DNA, 39 bp, Artificial Sequence
  Target Specific Oligonucleotide
tggtcacgccatttccggcgtaacatcgttatagctaga

SEQ ID NO: 155 - DNA, 45 bp, Artificial Sequence
  Target Specific Oligonucleotide
agcaagtataccataaggaaattcagtaacatcgttatagctaga

SEQ ID NO: 156 - DNA, 40 bp, Artificial Sequence
  Target Specific Oligonucleotide
ctcgccgtcctgtcgattttgtaacatcgttatagctaga

SEQ ID NO: 157 - DNA, 38 bp, Artificial Sequence
  Target Specific Oligonucleotide
agcgagccgagaactccggtaacatcgttatagctaga

SEQ ID NO: 158 - DNA, 43 bp, Artificial Sequence
  Target Specific Oligonucleotide
tattttgttcagacaacatggctgtaacatcgttatagctaga

SEQ ID NO: 159 - DNA, 40 bp, Artificial Sequence
  Target Specific Oligonucleotide
ccacttggtacaacggagccgtaacatcgttatagctaga

SEQ ID NO: 160 - DNA, 40 bp, Artificial Sequence
  Target Specific Oligonucleotide
cagaacacctgcgaggagaggtaacatcgttatagctaga

SEQ ID NO: 161 - DNA, 39 bp, Artificial Sequence
  Target Specific Oligonucleotide
ttcatcagcgacgcccctggtaacatcgttatagctaga

SEQ ID NO: 162 - DNA, 41 bp, Artificial Sequence
  Target Specific Oligonucleotide
cctgcaaaaaaacggtcacgtgtaacatcgttatagctaga

SEQ ID NO: 163 - DNA, 41 bp, Artificial Sequence
  Target Specific Oligonucleotide
tgtgggagtcccttaggtcaagtaacatcgttatagctaga

SEQ ID NO: 164 - DNA, 38 bp, Artificial Sequence
  Target Specific Oligonucleotide
gcatcgggagcacgcactgtaacatcgttatagctaga

SEQ ID NO: 165 - DNA, 39 bp, Artificial Sequence
  Target Specific Oligonucleotide
ggctctgcacaacgcttgcgtaacatcgttatagctaga

SEQ ID NO: 166 - DNA, 41 bp, Artificial Sequence
  Target Specific Oligonucleotide
agccaggacacaatagtcagggtaacatcgttatagctaga

SEQ ID NO: 167 - DNA, 41 bp, Artificial Sequence; identical to SEQ ID NO: 166
  Target Specific Oligonucleotide

SEQ ID NO: 168 - DNA, 42 bp, Artificial Sequence
  Target Specific Oligonucleotide
ttgcacatcctacggtcttctgtaacttactacggagttaac

SEQ ID NO: 169 - DNA, 43 bp, Artificial Sequence
  Target Specific Oligonucleotide
gtagctcattcgcaaatcttgggtaacttactacggagttaac

SEQ ID NO: 170 - DNA, 41 bp, Artificial Sequence
  Target Specific Oligonucleotide
cctttaagcgcgtcgtgtccgtaacttactacggagttaac

SEQ ID NO: 171 - DNA, 39 bp, Artificial Sequence
  Target Specific Oligonucleotide
cctcgacgcatgatgccggtaacttactacggagttaac

SEQ ID NO: 172 - DNA, 40 bp, Artificial Sequence
  Target Specific Oligonucleotide
gccccatggctcgtgtagggtaacttactacggagttaac

SEQ ID NO: 173 - DNA, 42 bp, Artificial Sequence
  Target Specific Oligonucleotide
ccaattgtgttccggcaagttgtaacttactacggagttaac

SEQ ID NO: 174 - DNA, 43 bp, Artificial Sequence
  Target Specific Oligonucleotide
gataggatttggtcggaaacctgtaacttactacggagttaac

SEQ ID NO: 175 - DNA, 41 bp, Artificial Sequence
  Target Specific Oligonucleotide
tagttgccaacggtgttgtagtaacttactacggagttaac

SEQ ID NO: 176 - DNA, 42 bp, Artificial Sequence
  Target Specific Oligonucleotide
cttggcctatgcggaagtaacgtaacttactacggagttaac

SEQ ID NO: 177 - DNA, 41 bp, Artificial Sequence
  Target Specific Oligonucleotide
ctcgcagcgataggaaccatgtaacttactacggagttaac

SEQ ID NO: 178 - DNA, 44 bp, Artificial Sequence
  Target Specific Oligonucleotide
tttgcattcgtccatatcaactggtaacttactacggagttaac

SEQ ID NO: 179 - DNA, 43 bp, Artificial Sequence
  Target Specific Oligonucleotide
tcaatatcaacgggtaaaccgggtaacttactacggagttaac

SEQ ID NO: 180 - DNA, 45 bp, Artificial Sequence
  Target Specific Oligonucleotide
ttgttactgacgtgggaaatattggtaacttactacggagttaac

SEQ ID NO: 181 - DNA, 40 bp, Artificial Sequence
  Target Specific Oligonucleotide
tctccgctgatacccgggtgtaacttactacggagttaac

SEQ ID NO: 182 - DNA, 42 bp, Artificial Sequence
  Target Specific Oligonucleotide
ggccagtcaccgaaatttcatgtaacttactacggagttaac

SEQ ID NO: 183 - DNA, 43 bp, Artificial Sequence
  Target Specific Oligonucleotide
agttcgtagcctatctcacactgtaacttactacggagttaac

SEQ ID NO: 184 - DNA, 42 bp, Artificial Sequence
  Target Specific Oligonucleotide
cacattcccgtacgtttgctggtaacttactacggagttaac

SEQ ID NO: 185 - DNA, 45 bp, Artificial Sequence
  Target Specific Oligonucleotide
agtgagtccctatgtatcctttctgtaacttactacggagttaac

SEQ ID NO: 186 - DNA, 44 bp, Artificial Sequence
  Target Specific Oligonucleotide
aagcattataacgtgatccacaggtaacttactacggagttaac

SEQ ID NO: 187 - DNA, 38 bp, Artificial Sequence
  Target Specific Oligonucleotide
tgcgccctgaagcgcacgtaacttactacggagttaac

SEQ ID NO: 188 - DNA, 47 bp, Artificial Sequence
  Target Specific Oligonucleotide
taaaaaatagcacgattacagtatacgtaacttactacggagttaac

SEQ ID NO: 189 - DNA, 44 bp, Artificial Sequence
  Target Specific Oligonucleotide
catatcgacggattgagcctaacgtaacttactacggagttaac

SEQ ID NO: 190 - DNA, 42 bp, Artificial Sequence
  Target Specific Oligonucleotide
cggtttctcgcgagagaaatagtaacttactacggagttaac

SEQ ID NO: 191 - DNA, 43 bp, Artificial Sequence
  Target Specific Oligonucleotide
cttcccatctcgtgtaacatgagtaacttactacggagttaac

SEQ ID NO: 192 - DNA, 41 bp, Artificial Sequence
  Target Specific Oligonucleotide
gcatttcacatcggactgtaccgtaacttcgaaacggaaac

SEQ ID NO: 193 - DNA, 38 bp, Artificial Sequence
  Target Specific Oligonucleotide
tggaattctcggcggaccagtaacttcgaaacggaaac

SEQ ID NO: 194 - DNA, 41 bp, Artificial Sequence
  Target Specific Oligonucleotide
gaagtggaaatacgacctttgcgtaacttcgaaacggaaac

SEQ ID NO: 195 - DNA, 39 bp, Artificial Sequence
  Target Specific Oligonucleotide
gctggtcccttgcgtaacatgtaacttcgaaacggaaac

SEQ ID NO: 196 - DNA, 43 bp, Artificial Sequence
  Target Specific Oligonucleotide
ttttcatctattcgagatgctcccgtaacttcgaaacggaaac

SEQ ID NO: 197 - DNA, 40 bp, Artificial Sequence
  Target Specific Oligonucleotide
tattcatcccgtggggtagtagtaacttcgaaacggaaac

SEQ ID NO: 198 - DNA, 42 bp, Artificial Sequence
  Target Specific Oligonucleotide
taaaaataggcccggatttgtctgtaacttcgaaacggaaac

SEQ ID NO: 199 - DNA, 42 bp, Artificial Sequence
  Target Specific Oligonucleotide
tccaaccttgacgacaagatagggtaacttcgaaacggaaac

SEQ ID NO: 200 - DNA, 40 bp, Artificial Sequence
  Target Specific Oligonucleotide
aggcaacagtcgaaccattctgtaacttcgaaacggaaac

SEQ ID NO: 201 - DNA, 40 bp, Artificial Sequence
  Target Specific Oligonucleotide
gcttcatttgcgactgctcttgtaacttcgaaacggaaac

SEQ ID NO: 202 - DNA, 41 bp, Artificial Sequence
  Target Specific Oligonucleotide
catcaggacgcttgtattggtggtaacttcgaaacggaaac

SEQ ID NO: 203 - DNA, 41 bp, Artificial Sequence
  Target Specific Oligonucleotide
atcttcaatccgagaatccagcgtaacttcgaaacggaaac

SEQ ID NO: 204 - DNA, 42 bp, Artificial Sequence
  Target Specific Oligonucleotide
ctgtagtgtggtagtaaggaagagtaacttcgaaacggaaac

SEQ ID NO: 205 - DNA, 42 bp, Artificial Sequence
  Target Specific Oligonucleotide
gaacaccttacttacaacacctggtaacttcgaaacggaaac

SEQ ID NO: 206 - DNA, 43 bp, Artificial Sequence
  Target Specific Oligonucleotide
gtacattgtacaccgttacaatgggtaacttcgaaacggaaac

SEQ ID NO: 207 - DNA, 42 bp, Artificial Sequence
  Target Specific Oligonucleotide
tttcaagtcgtctatgttagctggtaacttcgaaacggaaac

SEQ ID NO: 208 - DNA, 44 bp, Artificial Sequence
  Target Specific Oligonucleotide
caaagcaactatatgaagcttcattgtaacttcgaaacggaaac

SEQ ID NO: 209 - DNA, 42 bp, Artificial Sequence
  Target Specific Oligonucleotide
catacttctatcgagagctcagggtaacttcgaaacggaaac

SEQ ID NO: 210 - DNA, 43 bp, Artificial Sequence
  Target Specific Oligonucleotide
acgaagaaatcgagtaggtctagggtaacttcgaaacggaaac

SEQ ID NO: 211 - DNA, 41 bp, Artificial Sequence
  Target Specific Oligonucleotide
gaatcattgagtcgacccttcagtaacttcgaaacggaaac

SEQ ID NO: 212 - DNA, 41 bp, Artificial Sequence
  Target Specific Oligonucleotide
tgatgtgcttaagtagtgcagcgtaacttcgaaacggaaac

SEQ ID NO: 213 - DNA, 41 bp, Artificial Sequence
  Target Specific Oligonucleotide
tccacggcatgatacatacaacgtaacttcgaaacggaaac

SEQ ID NO: 214 - DNA, 40 bp, Artificial Sequence
  Target Specific Oligonucleotide
tctcagagcatcccgaatccagtaacttcgaaacggaaac

SEQ ID NO: 215 - DNA, 42 bp, Artificial Sequence
  Target Specific Oligonucleotide
gtggaacgaagagtaggtagtttgtaacttcgaaacggaaac

SEQ ID NO: 216 - DNA, 39 bp, Artificial Sequence
  Target Specific Oligonucleotide
gatcaatactggacggagtcaggtaacctcgtcggatca

SEQ ID NO: 217 - DNA, 38 bp, Artificial Sequence
  Target Specific Oligonucleotide
gagcttgttactcgtgccttggtaacctcgtcggatca

SEQ ID NO: 218 - DNA, 37 bp, Artificial Sequence
  Target Specific Oligonucleotide
cttcttacacttgcggacgcgtaacctcgtcggatca

SEQ ID NO: 219 - DNA, 41 bp, Artificial Sequence
  Target Specific Oligonucleotide
caatacctattccgttacacacttgtaacctcgtcggatca

SEQ ID NO: 220 - DNA, 39 bp, Artificial Sequence
  Target Specific Oligonucleotide
gctccttcagtccggttttattgtaacctcgtcggatca

SEQ ID NO: 221 - DNA, 36 bp, Artificial Sequence
  Target Specific Oligonucleotide
gacgcacgagccgtgatctgtaacctcgtcggatca

SEQ ID NO: 222 - DNA, 41 bp, Artificial Sequence
  Target Specific Oligonucleotide
gactgctaaggcataggaattttcgtaacctcgtcggatca

SEQ ID NO: 223 - DNA, 39 bp, Artificial Sequence
  Target Specific Oligonucleotide
caccacataattacggggacacgtaacctcgtcggatca

SEQ ID NO: 224 - DNA, 34 bp, Artificial Sequence
  Target Specific Oligonucleotide
cggcaaggcccttcgcagtaacctcgtcggatca

SEQ ID NO: 225 - DNA, 37 bp, Artificial Sequence
  Target Specific Oligonucleotide
tccatctggtacgtggtggggtaacctcgtcggatca

SEQ ID NO: 226 - DNA, 38 bp, Artificial Sequence
  Target Specific Oligonucleotide
gtcctgccgcgtatgatttctgtaacctcgtcggatca

SEQ ID NO: 227 - DNA, 37 bp, Artificial Sequence
  Target Specific Oligonucleotide
tgttcaggctgacgactgcagtaacctcgtcggatca

SEQ ID NO: 228 - DNA, 36 bp, Artificial Sequence
  Target Specific Oligonucleotide
gcatggaggtccgtcctgtgtaacctcgtcggatca

SEQ ID NO: 229 - DNA, 38 bp, Artificial Sequence
  Target Specific Oligonucleotide
ttgagggagcgtaatcccaaggtaacctcgtcggatca

SEQ ID NO: 230 - DNA, 36 bp, Artificial Sequence
  Target Specific Oligonucleotide
ccggcgtctgcgtacttccgtaacctcgtcggatca

SEQ ID NO: 231 - DNA, 39 bp, Artificial Sequence
  Target Specific Oligonucleotide
cgactatctgcgtctatcatccgtaacctcgtcggatca

SEQ ID NO: 232 - DNA, 40 bp, Artificial Sequence
  Target Specific Oligonucleotide
gagaattcgatgatcaactcacggtaacctcgtcggatca

SEQ ID NO: 233 - DNA, 35 bp, Artificial Sequence
  Target Specific Oligonucleotide
tggcctgtcgtccggtctgtaacctcgtcggatca

SEQ ID NO: 234 - DNA, 38 bp, Artificial Sequence
  Target Specific Oligonucleotide
cagggattccgtcatatggctgtaacctcgtcggatca

SEQ ID NO: 235 - DNA, 40 bp, Artificial Sequence
  Target Specific Oligonucleotide
agacatcgatggtacatatgggtgtaacctcgtcggatca

SEQ ID NO: 236 - DNA, 37 bp, Artificial Sequence
  Target Specific Oligonucleotide
tctgagctgtatcgctgcaagtaacctcgtcggatca

SEQ ID NO: 237 - DNA, 37 bp, Artificial Sequence
  Target Specific Oligonucleotide
tcttaggcccattcgttggagtaacctcgtcggatca

SEQ ID NO: 238 - DNA, 37 bp, Artificial Sequence
  Target Specific Oligonucleotide
ttatacaccgtgccgaacgcgtaacctcgtcggatca

SEQ ID NO: 239 - DNA, 37 bp, Artificial Sequence
  Target Specific Oligonucleotide
atgccctttgcgatctgcacgtaacctcgtcggatca

SEQ ID NO: 240 - DNA, 42 bp, Artificial Sequence
  Target Specific Oligonucleotide
ggcagaattccgaagttcagcgtaacataatcgtagtttcgg

SEQ ID NO: 241 - DNA, 42 bp, Artificial Sequence
  Target Specific Oligonucleotide
gtttccttcacgacaggtgtggtaacataatcgtagtttcgg

SEQ ID NO: 242 - DNA, 44 bp, Artificial Sequence
  Target Specific Oligonucleotide
ctggtagaaatgcgactaaagacgtaacataatcgtagtttcgg

SEQ ID NO: 243 - DNA, 44 bp, Artificial Sequence
  Target Specific Oligonucleotide
tgaccagcatcgtttcatctaatgtaacataatcgtagtttcgg

SEQ ID NO: 244 - DNA, 42 bp, Artificial Sequence
  Target Specific Oligonucleotide
ttcgaagttcaaccgagtgacgtaacataatcgtagtttcgg

SEQ ID NO: 245 - DNA, 42 bp, Artificial Sequence
  Target Specific Oligonucleotide
ccaccaatccaatgcggaattgtaacataatcgtagtttcgg

SEQ ID NO: 246 - DNA, 42 bp, Artificial Sequence
  Target Specific Oligonucleotide
agactcggtgccattcgtattgtaacataatcgtagtttcgg

SEQ ID NO: 247 - DNA, 42 bp, Artificial Sequence
  Target Specific Oligonucleotide
ataactgctaactgcgcaaccgtaacataatcgtagtttcgg

SEQ ID NO: 248 - DNA, 43 bp, Artificial Sequence
  Target Specific Oligonucleotide
gtagtgaggccgcttataaccagtaacataatcgtagtttcgg

SEQ ID NO: 249 - DNA, 43 bp, Artificial Sequence
  Target Specific Oligonucleotide
ggtgatgattcgatggagtgaagtaacataatcgtagtttcgg

SEQ ID NO: 250 - DNA, 42 bp, Artificial Sequence
  Target Specific Oligonucleotide
ctgctgctttacgtttggtgcgtaacataatcgtagtttcgg

SEQ ID NO: 251 - DNA, 43 bp, Artificial Sequence
  Target Specific Oligonucleotide
tcatcaaccacgtctttggatagtaacataatcgtagtttcgg

SEQ ID NO: 252 - DNA, 44 bp, Artificial Sequence
  Target Specific Oligonucleotide
aggcagatcttaaagtgttggttgtaacataatcgtagtttcgg

SEQ ID NO: 253 - DNA, 43 bp, Artificial Sequence
  Target Specific Oligonucleotide
tgagggcttatacgaaagcaaggtaacataatcgtagtttcgg

SEQ ID NO: 254 - DNA, 44 bp, Artificial Sequence
  Target Specific Oligonucleotide
gctcagtactgactttggtatgtgtaacataatcgtagtttcgg

SEQ ID NO: 255 - DNA, 45 bp, Artificial Sequence
  Target Specific Oligonucleotide
cagtcaatcattagatccacatctgtaacataatcgtagtttcgg

SEQ ID NO: 256 - DNA, 40 bp, Artificial Sequence
  Target Specific Oligonucleotide
ggaagctgctcgtcgaagcgtaacataatcgtagtttcgg

SEQ ID NO: 257 - DNA, 47 bp, Artificial Sequence
  Target Specific Oligonucleotide
gagaagatacttatagcttcttgtctgtaacataatcgtagtttcgg

SEQ ID NO: 258 - DNA, 45 bp, Artificial Sequence
  Target Specific Oligonucleotide
tcaatgctatctaactgatgaagagtaacataatcgtagtttcgg

SEQ ID NO: 259 - DNA, 46 bp, Artificial Sequence
  Target Specific Oligonucleotide
atcttcccaattgatgtaagtacttgtaacataatcgtagtttcgg

SEQ ID NO: 260 - DNA, 40 bp, Artificial Sequence
  Target Specific Oligonucleotide
catcacagtccgagacgccgtaacataatcgtagtttcgg

SEQ ID NO: 261 - DNA, 40 bp, Artificial Sequence
  Target Specific Oligonucleotide
cattccaccggcctgtgcggtaacataatcgtagtttcgg

SEQ ID NO: 262 - DNA, 44 bp, Artificial Sequence
  Target Specific Oligonucleotide
ggacagcactactctagagtaaggtaacataatcgtagtttcgg

SEQ ID NO: 263 - DNA, 43 bp, Artificial Sequence
  Target Specific Oligonucleotide
tgccaagtaacttagcacacccgtaacataatcgtagtttcgg

SEQ ID NO: 264 - DNA, 38 bp, Artificial Sequence
  Target Specific Oligonucleotide
gtgccacctttcaccgtgagtaaccggtagaattacgg

SEQ ID NO: 265 - DNA, 38 bp, Artificial Sequence
  Target Specific Oligonucleotide
ccgcaggtacgacttgcctgtaaccggtagaattacgg

SEQ ID NO: 266 - DNA, 40 bp, Artificial Sequence
  Target Specific Oligonucleotide
tgggcttcaatcagatggtcagtaaccggtagaattacgg

SEQ ID NO: 267 - DNA, 41 bp, Artificial Sequence
  Target Specific Oligonucleotide
agaacaccagggtacgcatagtgtaaccggtagaattacgg

SEQ ID NO: 268 - DNA, 38 bp, Artificial Sequence
  Target Specific Oligonucleotide
tctccgagagtgtcagcgggtaaccggtagaattacgg

SEQ ID NO: 269 - DNA, 38 bp, Artificial Sequence
  Target Specific Oligonucleotide
gtcttcccggccggtcttggtaaccggtagaattacgg

SEQ ID NO: 270 - DNA, 39 bp, Artificial Sequence
  Target Specific Oligonucleotide
catgttgcagattgtcgccagtaaccggtagaattacgg

SEQ ID NO: 271 - DNA, 39 bp, Artificial Sequence
  Target Specific Oligonucleotide
ggttcagtcgtttgcgaacagtaaccggtagaattacgg

SEQ ID NO: 272 - DNA, 41 bp, Artificial Sequence
  Target Specific Oligonucleotide
agctaccaattagacccactcggtaaccggtagaattacgg

SEQ ID NO: 273 - DNA, 40 bp, Artificial Sequence
  Target Specific Oligonucleotide
cgtagttctcgtctccgatcagtaaccggtagaattacgg

SEQ ID NO: 274 - DNA, 39 bp, Artificial Sequence
  Target Specific Oligonucleotide
ggagctgaacttgacgccaggtaaccggtagaattacgg

SEQ ID NO: 275 - DNA, 40 bp, Artificial Sequence
  Target Specific Oligonucleotide
taatggatgtactcgttgggcgtaaccggtagaattacgg

SEQ ID NO: 276 - DNA, 38 bp, Artificial Sequence
  Target Specific Oligonucleotide
tctgtgcatagccgtcccggtaaccggtagaattacgg

SEQ ID NO: 277 - DNA, 38 bp, Artificial Sequence
  Target Specific Oligonucleotide
tggctggacggatcggatcgtaaccggtagaattacgg

SEQ ID NO: 278 - DNA, 37 bp, Artificial Sequence
  Target Specific Oligonucleotide
ggtttgcgtcgttgcggcgtaaccggtagaattacgg

SEQ ID NO: 279 - DNA, 39 bp, Artificial Sequence
  Target Specific Oligonucleotide
tctctggggacgtgacaaaggtaaccggtagaattacgg

SEQ ID NO: 280 - DNA, 42 bp, Artificial Sequence
  Target Specific Oligonucleotide
gaattcatcagctagattggcaagtaaccggtagaattacgg

SEQ ID NO: 281 - DNA, 38 bp, Artificial Sequence
  Target Specific Oligonucleotide
tcactccgtgggctaagcggtaaccggtagaattacgg

SEQ ID NO: 282 - DNA, 40 bp, Artificial Sequence
  Target Specific Oligonucleotide
tgtaagggaacacggaagtgggtaaccggtagaattacgg

SEQ ID NO: 283 - DNA, 38 bp, Artificial Sequence
  Target Specific Oligonucleotide
ctcaatggtggcgcggatcgtaaccggtagaattacgg

SEQ ID NO: 284 - DNA, 40 bp, Artificial Sequence
  Target Specific Oligonucleotide
aaccactcaatctgcgtctcggtaaccggtagaattacgg

SEQ ID NO: 285 - DNA, 39 bp, Artificial Sequence
  Target Specific Oligonucleotide
gctgtagggcgccattttgtgtaaccggtagaattacgg

SEQ ID NO: 286 - DNA, 38 bp, Artificial Sequence
  Target Specific Oligonucleotide
tgagtgacggcattgcgcagtaaccggtagaattacgg

SEQ ID NO: 287 - DNA, 39 bp, Artificial Sequence
  Target Specific Oligonucleotide
caaagtggctcatcgccaccgtaaccggtagaattacgg

SEQ ID NO: 288 - DNA, 45 bp, Artificial Sequence
  Target Specific Oligonucleotide
tctatatgctaaatgtattgccatggtaacgcttctcataacact

SEQ ID NO: 289 - DNA, 43 bp, Artificial Sequence
  Target Specific Oligonucleotide
ctatgaaacttcgtggtcactccgtaacgcttctcataacact

SEQ ID NO: 290 - DNA, 43 bp, Artificial Sequence
  Target Specific Oligonucleotide
aactaactcatctgcagtaccatgtaacgcttctcataacact

SEQ ID NO: 291 - DNA, 43 bp, Artificial Sequence
  Target Specific Oligonucleotide
aaaacctgcttgatccacattctgtaacgcttctcataacact

SEQ ID NO: 292 - DNA, 45 bp, Artificial Sequence
  Target Specific Oligonucleotide
acattaacaaatcactcttgattcagtaacgcttctcataacact

SEQ ID NO: 293 - DNA, 43 bp, Artificial Sequence
  Target Specific Oligonucleotide
ttttgcttagctcatggtaaacagtaacgcttctcataacact

SEQ ID NO: 294 - DNA, 45 bp, Artificial Sequence
  Target Specific Oligonucleotide
gaggagctgttggataaataattttgtaacgcttctcataacact

SEQ ID NO: 295 - DNA, 43 bp, Artificial Sequence
  Target Specific Oligonucleotide
aagcaggcaggtattgtatgattgtaacgcttctcataacact

SEQ ID NO: 296 - DNA, 45 bp, Artificial Sequence
  Target Specific Oligonucleotide
gcaacttatatatcagtggtgaatggtaacgcttctcataacact

SEQ ID NO: 297 - DNA, 45 bp, Artificial Sequence
  Target Specific Oligonucleotide
agattatgtatgcatgagaaccaacgtaacgcttctcataacact

SEQ ID NO: 298 - DNA, 45 bp, Artificial Sequence
  Target Specific Oligonucleotide
gatcatgactctccttatggttaatgtaacgcttctcataacact

SEQ ID NO: 299 - DNA, 43 bp, Artificial Sequence
  Target Specific Oligonucleotide
tgtgaaaccggtttataaacctagtaacgcttctcataacact

SEQ ID NO: 300 - DNA, 46 bp, Artificial Sequence
  Target Specific Oligonucleotide
aataagattagctactgtctacagtggtaacgcttctcataacact

SEQ ID NO: 301 - DNA, 45 bp, Artificial Sequence
  Target Specific Oligonucleotide
taaacaaatagtgatacatccacacgtaacgcttctcataacact

SEQ ID NO: 302 - DNA, 45 bp, Artificial Sequence
  Target Specific Oligonucleotide
ttcagtcatctaacaatgtattcctgtaacgcttctcataacact

SEQ ID NO: 303 - DNA, 43 bp, Artificial Sequence
  Target Specific Oligonucleotide
agctgtgggaaagtctttaactcgtaacgcttctcataacact

SEQ ID NO: 304 - DNA, 46 bp, Artificial Sequence
  Target Specific Oligonucleotide
atagatgaactattgatgtacacaacgtaacgcttctcataacact

SEQ ID NO: 305 - DNA, 45 bp, Artificial Sequence
  Target Specific Oligonucleotide
tacagatgaaattgagacctaaaacgtaacgcttctcataacact

SEQ ID NO: 306 - DNA, 45 bp, Artificial Sequence
  Target Specific Oligonucleotide
agcaatttgggttaacagaaatagagtaacgcttctcataacact

SEQ ID NO: 307 - DNA, 46 bp, Artificial Sequence
  Target Specific Oligonucleotide
aatccaatgggataagtactattagtgtaacgcttctcataacact

SEQ ID NO: 308 - DNA, 45 bp, Artificial Sequence
  Target Specific Oligonucleotide
aatgttattccgtggaaaattacatgtaacgcttctcataacact

SEQ ID NO: 309 - DNA, 47 bp, Artificial Sequence
  Target Specific Oligonucleotide
ttacagaattgtgtctgaaaaattatggtaacgcttctcataacact

SEQ ID NO: 310 - DNA, 40 bp, Artificial Sequence
  Target Specific Oligonucleotide
atcagagaggcgctatgcctgtaacgcttctcataacact

SEQ ID NO: 311 - DNA, 38 bp, Artificial Sequence
  Target Specific Oligonucleotide
cggcgacacgatacagcggtaacgcttctcataacact

SEQ ID NO: 312 - DNA, 40 bp, Artificial Sequence
  Target Specific Oligonucleotide
cacttcaaatgcgcaacaagcgtaacgtcgctgaaaaatc

SEQ ID NO: 313 - DNA, 39 bp, Artificial Sequence
  Target Specific Oligonucleotide
gtaagcactgcgcaagacaagtaacgtcgctgaaaaatc

SEQ ID NO: 314 - DNA, 41 bp, Artificial Sequence
  Target Specific Oligonucleotide
gtgatgagctcgacaggatattgtaacgtcgctgaaaaatc

SEQ ID NO: 315 - DNA, 37 bp, Artificial Sequence
  Target Specific Oligonucleotide
atggcgtcgtcggcacacgtaacgtcgctgaaaaatc

SEQ ID NO: 316 - DNA, 40 bp, Artificial Sequence
  Target Specific Oligonucleotide
tttacaacagcgtggcaagtggtaacgtcgctgaaaaatc

SEQ ID NO: 317 - DNA, 40 bp, Artificial Sequence
  Target Specific Oligonucleotide
aggcatgaagtgagacaatgcgtaacgtcgctgaaaaatc

SEQ ID NO: 318 - DNA, 41 bp, Artificial Sequence
  Target Specific Oligonucleotide
cctggctagtggtatatgtcacgtaacgtcgctgaaaaatc

SEQ ID NO: 319 - DNA, 39 bp, Artificial Sequence
  Target Specific Oligonucleotide
gctgatgatgttcaagcgcagtaacgtcgctgaaaaatc

SEQ ID NO: 320 - DNA, 41 bp, Artificial Sequence
  Target Specific Oligonucleotide
ctgccagttagttaggcaagttgtaacgtcgctgaaaaatc

SEQ ID NO: 321 - DNA, 38 bp, Artificial Sequence
  Target Specific Oligonucleotide
gcagctccgggctacaagtgtaacgtcgctgaaaaatc

SEQ ID NO: 322 - DNA, 40 bp, Artificial Sequence
  Target Specific Oligonucleotide
tggcagctgtctaactggagcgtaacgtcgctgaaaaatc

SEQ ID NO: 323 - DNA, 38 bp, Artificial Sequence
  Target Specific Oligonucleotide
acgcgtgtgcgagtagatggtaacgtcgctgaaaaatc

SEQ ID NO: 324 - DNA, 38 bp, Artificial Sequence
  Target Specific Oligonucleotide
agctccacgaaggatgccagtaacgtcgctgaaaaatc

SEQ ID NO: 325 - DNA, 39 bp, Artificial Sequence
  Target Specific Oligonucleotide
tgaggacggcatcgagatccgtaacgtcgctgaaaaatc

SEQ ID NO: 326 - DNA, 39 bp, Artificial Sequence
  Target Specific Oligonucleotide
gtcttgagacccggtcttgggtaacgtcgctgaaaaatc

SEQ ID NO: 327 - DNA, 38 bp, Artificial Sequence
  Target Specific Oligonucleotide
agtggtctggatcggtgcggtaacgtcgctgaaaaatc

SEQ ID NO: 328 - DNA, 39 bp, Artificial Sequence
  Target Specific Oligonucleotide
tccattctgggtcgagtggagtaacgtcgctgaaaaatc

SEQ ID NO: 329 - DNA, 39 bp, Artificial Sequence
  Target Specific Oligonucleotide
ggaccccaaggtgttccaaggtaacgtcgctgaaaaatc

SEQ ID NO: 330 - DNA, 38 bp, Artificial Sequence
  Target Specific Oligonucleotide
agcagcttggctactccccgtaacgtcgctgaaaaatc

SEQ ID NO: 331 - DNA, 42 bp, Artificial Sequence
  Target Specific Oligonucleotide
tttgggtatgggtactgtgtagagtaacgtcgctgaaaaatc

SEQ ID NO: 332 - DNA, 39 bp, Artificial Sequence
  Target Specific Oligonucleotide
gccgcagggatagatccagggtaacgtcgctgaaaaatc

SEQ ID NO: 333 - DNA, 42 bp, Artificial Sequence
  Target Specific Oligonucleotide
tgaagacagtcctatggacttccgtaacgtcgctgaaaaatc

SEQ ID NO: 334 - DNA, 37 bp, Artificial Sequence
  Target Specific Oligonucleotide
aagctgaagcgcgggtcagtaacgtcgctgaaaaatc

SEQ ID NO: 335 - DNA, 37 bp, Artificial Sequence
  Target Specific Oligonucleotide
ccacgcagcccttcgagagtaacgtcgctgaaaaatc

SEQ ID NO: 336 - DNA, 45 bp, Artificial Sequence
  Target Specific Oligonucleotide
tagtaggtgtcgacaactagagcgtaactcttcaagattaatacc

SEQ ID NO: 337 - DNA, 44 bp, Artificial Sequence
  Target Specific Oligonucleotide
gatggtcctgatcgagaaaccagtaactcttcaagattaatacc

SEQ ID NO: 338 - DNA, 44 bp, Artificial Sequence
  Target Specific Oligonucleotide
aagaggacttcgctgaattgacgtaactcttcaagattaatacc

SEQ ID NO: 339 - DNA, 44 bp, Artificial Sequence
  Target Specific Oligonucleotide
gaacctccgactgtatgtcagcgtaactcttcaagattaatacc

SEQ ID NO: 340 - DNA, 45 bp, Artificial Sequence
  Target Specific Oligonucleotide
tctgaattagagcgatgttgacagtaactcttcaagattaatacc

SEQ ID NO: 341 - DNA, 41 bp, Artificial Sequence
  Target Specific Oligonucleotide
gcattcctccgatcgcacagtaactcttcaagattaatacc

SEQ ID NO: 342 - DNA, 46 bp, Artificial Sequence
  Target Specific Oligonucleotide
ctctatatttagctcgctgttcaagtaactcttcaagattaatacc

SEQ ID NO: 343 - DNA, 41 bp, Artificial Sequence
  Target Specific Oligonucleotide
tattcatcacggcgcgcttgtaactcttcaagattaatacc

SEQ ID NO: 344 - DNA, 43 bp, Artificial Sequence
  Target Specific Oligonucleotide
cattggggaccgtgcataaaagtaactcttcaagattaatacc

SEQ ID NO: 345 - DNA, 43 bp, Artificial Sequence
  Target Specific Oligonucleotide
caaggggtgcactatttgggagtaactcttcaagattaatacc

SEQ ID NO: 346 - DNA, 43 bp, Artificial Sequence
  Target Specific Oligonucleotide
ggaccgtatttcggcgaaatagtaactcttcaagattaatacc

SEQ ID NO: 347 - DNA, 43 bp, Artificial Sequence
  Target Specific Oligonucleotide
atactgcacttgtcggcatgagtaactcttcaagattaatacc

SEQ ID NO: 348 - DNA, 43 bp, Artificial Sequence
  Target Specific Oligonucleotide
atggcagatcgatccattggtgtaactcttcaagattaatacc

SEQ ID NO: 349 - DNA, 43 bp, Artificial Sequence
  Target Specific Oligonucleotide
tccctttggaccgtcaagaaggtaactcttcaagattaatacc

SEQ ID NO: 350 - DNA, 42 bp, Artificial Sequence
  Target Specific Oligonucleotide
aggcttgctgacatacgcaggtaactcttcaagattaatacc

SEQ ID NO: 351 - DNA, 42 bp, Artificial Sequence
  Target Specific Oligonucleotide
cttgctttgtgcgaacacccgtaactcttcaagattaatacc

SEQ ID NO: 352 - DNA, 45 bp, Artificial Sequence
  Target Specific Oligonucleotide
ctgatatcgaatgcaatggatgagtaactcttcaagattaatacc

SEQ ID NO: 353 - DNA, 43 bp, Artificial Sequence
  Target Specific Oligonucleotide
taggactggtccgtcaaaaacgtaactcttcaagattaatacc

SEQ ID NO: 354 - DNA, 44 bp, Artificial Sequence
  Target Specific Oligonucleotide
tgaccattctcgggacactaacgtaactcttcaagattaatacc

SEQ ID NO: 355 - DNA, 44 bp, Artificial Sequence
  Target Specific Oligonucleotide
atgattgggtccgtaaaaatgcgtaactcttcaagattaatacc

SEQ ID NO: 356 - DNA, 45 bp, Artificial Sequence
  Target Specific Oligonucleotide
gtttcatgtatggtaggaccaccgtaactcttcaagattaatacc

SEQ ID NO: 357 - DNA, 43 bp, Artificial Sequence
  Target Specific Oligonucleotide
ctcttgcatcgtagcgaactagtaactcttcaagattaatacc

SEQ ID NO: 358 - DNA, 43 bp, Artificial Sequence
  Target Specific Oligonucleotide
atgatgattccctcggtcagagtaactcttcaagattaatacc

SEQ ID NO: 359 - DNA, 46 bp, Artificial Sequence
  Target Specific Oligonucleotide
agtagagaagatcgctgatatccggtaactcttcaagattaatacc

SEQ ID NO: 360 - DNA, 42 bp, Artificial Sequence
  Target Specific Oligonucleotide
aacccttgacatcgccagtttgtaacgtaccgtttgtatatg

SEQ ID NO: 361 - DNA, 45 bp, Artificial Sequence
  Target Specific Oligonucleotide
gaaggttaaacgagatttccaaaggtaacgtaccgtttgtatatg

SEQ ID NO: 362 - DNA, 41 bp, Artificial Sequence
  Target Specific Oligonucleotide
gccactcgacatttctgccggtaacgtaccgtttgtatatg

SEQ ID NO: 363 - DNA, 42 bp, Artificial Sequence
  Target Specific Oligonucleotide
tcatactgtccgcaacatccggtaacgtaccgtttgtatatg

SEQ ID NO: 364 - DNA, 41 bp, Artificial Sequence; identical to SEQ ID NO: 362
  Target Specific Oligonucleotide

SEQ ID NO: 365 - DNA, 42 bp, Artificial Sequence; identical to SEQ ID NO: 363
  Target Specific Oligonucleotide

SEQ ID NO: 366 - DNA, 43 bp, Artificial Sequence
  Target Specific Oligonucleotide
tgaattttgcccgaacttcactgtaacgtaccgtttgtatatg

SEQ ID NO: 367 - DNA, 38 bp, Artificial Sequence
  Target Specific Oligonucleotide
acacactcggagcgcgcgtaacgtaccgtttgtatatg

SEQ ID NO: 368 - DNA, 43 bp, Artificial Sequence
  Target Specific Oligonucleotide
caaacctatatgcccgttgactgtaacgtaccgtttgtatatg

SEQ ID NO: 369 - DNA, 44 bp, Artificial Sequence
  Target Specific Oligonucleotide
caggaacctttaccatgttcatggtaacgtaccgtttgtatatg

SEQ ID NO: 370 - DNA, 46 bp, Artificial Sequence
  Target Specific Oligonucleotide
gaaaatctctacggatgaatttcttgtaacgtaccgtttgtatatg

SEQ ID NO: 371 - DNA, 43 bp, Artificial Sequence
  Target Specific Oligonucleotide
gctgctaagatagccttgtgaggtaacgtaccgtttgtatatg

SEQ ID NO: 372 - DNA, 42 bp, Artificial Sequence
  Target Specific Oligonucleotide
tggcactaaaaaccggagaacgtaacgtaccgtttgtatatg

SEQ ID NO: 373 - DNA, 39 bp, Artificial Sequence
  Target Specific Oligonucleotide
tgcgggaccgccgatacagtaacgtaccgtttgtatatg

SEQ ID NO: 374 - DNA, 45 bp, Artificial Sequence
  Target Specific Oligonucleotide
ggaaattaaacggagtcttacaacgtaacgtaccgtttgtatatg

SEQ ID NO: 375 - DNA, 40 bp, Artificial Sequence
  Target Specific Oligonucleotide
agccagagagcggtatgccgtaacgtaccgtttgtatatg

SEQ ID NO: 376 - DNA, 41 bp, Artificial Sequence
  Target Specific Oligonucleotide
ccaccaagtggggatgtgacgtaacgtaccgtttgtatatg

SEQ ID NO: 377 - DNA, 42 bp, Artificial Sequence
  Target Specific Oligonucleotide
cacacatgacctttaagcgctgtaacgtaccgtttgtatatg

SEQ ID NO: 378 - DNA, 41 bp, Artificial Sequence
  Target Specific Oligonucleotide
tccacagattgcgctgtctagtaacgtaccgtttgtatatg

SEQ ID NO: 379 - DNA, 41 bp, Artificial Sequence
  Target Specific Oligonucleotide
ctccacggacaggttactgcgtaacgtaccgtttgtatatg

SEQ ID NO: 380 - DNA, 39 bp, Artificial Sequence
  Target Specific Oligonucleotide
gcgtcatggcgtcagcacgtaacgtaccgtttgtatatg

SEQ ID NO: 381 - DNA, 39 bp, Artificial Sequence
  Target Specific Oligonucleotide
tcctggccggcaacacacgtaacgtaccgtttgtatatg

SEQ ID NO: 382 - DNA, 42 bp, Artificial Sequence
  Target Specific Oligonucleotide
agccattttgtcgaggtttgggtaacgtaccgtttgtatatg

SEQ ID NO: 383 - DNA, 44 bp, Artificial Sequence
  Target Specific Oligonucleotide
catccgaagcatgatagttgatggtaacgtaccgtttgtatatg

SEQ ID NO: 384 - DNA, 43 bp, Artificial Sequence
  Target Specific Oligonucleotide
tagtgtgttgcgtatcaagtatctagacgatgcgaattaacac

SEQ ID NO: 385 - DNA, 45 bp, Artificial Sequence
  Target Specific Oligonucleotide
ttattgtgaactctagacatgagagtagacgatgcgaattaacac

SEQ ID NO: 386 - DNA, 46 bp, Artificial Sequence
  Target Specific Oligonucleotide
acagtgaatactaagactgtaaaaactagacgatgcgaattaacac

SEQ ID NO: 387 - DNA, 41 bp, Artificial Sequence
  Target Specific Oligonucleotide
acacaaggcacgtagaaacagtagacgatgcgaattaacac

SEQ ID NO: 388 - DNA, 41 bp, Artificial Sequence
  Target Specific Oligonucleotide
caatggacagcggttgtgaaatagacgatgcgaattaacac

SEQ ID NO: 389 - DNA, 42 bp, Artificial Sequence
  Target Specific Oligonucleotide
caccatcacctatcgacagagttagacgatgcgaattaacac

SEQ ID NO: 390 - DNA, 42 bp, Artificial Sequence
  Target Specific Oligonucleotide
cattgcattacgttccacatggtagacgatgcgaattaacac

SEQ ID NO: 391 - DNA, 41 bp, Artificial Sequence
  Target Specific Oligonucleotide
acacagagtgtccgatacccatagacgatgcgaattaacac

SEQ ID NO: 392 - DNA, 43 bp, Artificial Sequence
  Target Specific Oligonucleotide
ggtgattacattgagtgctaggatagacgatgcgaattaacac

SEQ ID NO: 393 - DNA, 41 bp, Artificial Sequence
  Target Specific Oligonucleotide
tccacataagcgttcaaggtatagacgatgcgaattaacac

SEQ ID NO: 394 - DNA, 44 bp, Artificial Sequence
  Target Specific Oligonucleotide
cagaataatcgctattcctagctgtagacgatgcgaattaacac

SEQ ID NO: 395 - DNA, 40 bp, Artificial Sequence
  Target Specific Oligonucleotide
ttacactcatacgtcgccggtagacgatgcgaattaacac

SEQ ID NO: 396 - DNA, 43 bp, Artificial Sequence
  Target Specific Oligonucleotide
tggccataaaggctacttacaattagacgatgcgaattaacac

SEQ ID NO: 397 - DNA, 45 bp, Artificial Sequence
  Target Specific Oligonucleotide
tttcaagttattcgatctgctaacctagacgatgcgaattaacac

SEQ ID NO: 398 - DNA, 45 bp, Artificial Sequence
  Target Specific Oligonucleotide
agttagtcaaggactttactaaggttagacgatgcgaattaacac

SEQ ID NO: 399 - DNA, 42 bp, Artificial Sequence
  Target Specific Oligonucleotide
ttacctcaaatacgggctaccatagacgatgcgaattaacac

SEQ ID NO: 400 - DNA, 47 bp, Artificial Sequence
  Target Specific Oligonucleotide
gagttatattactctaactaaagccagtagacgatgcgaattaacac

SEQ ID NO: 401 - DNA, 43 bp, Artificial Sequence
  Target Specific Oligonucleotide
ttgtcaacacgcataaaatctgctagacgatgcgaattaacac

SEQ ID NO: 402 - DNA, 43 bp, Artificial Sequence
  Target Specific Oligonucleotide
tcatatacaatcggggatctgagtagacgatgcgaattaacac

SEQ ID NO: 403 - DNA, 43 bp, Artificial Sequence
  Target Specific Oligonucleotide
cccacaaaaatacccgtaagttatagacgatgcgaattaacac

SEQ ID NO: 404 - DNA, 47 bp, Artificial Sequence
  Target Specific Oligonucleotide
cactaaattattacgaattttgcaaagtagacgatgcgaattaacac

SEQ ID NO: 405 - DNA, 45 bp, Artificial Sequence
  Target Specific Oligonucleotide
tcaaataaaaaccgtcaaaagtttatagacgatgcgaattaacac

SEQ ID NO: 406 - DNA, 42 bp, Artificial Sequence
  Target Specific Oligonucleotide
agtgcaatggtatcacgtactgtagacgatgcgaattaacac

SEQ ID NO: 407 - DNA, 43 bp, Artificial Sequence
  Target Specific Oligonucleotide
atgtgtcttgcaattggattccctagacgatgcgaattaacac

SEQ ID NO: 408 - DNA, 38 bp, Artificial Sequence
  Target Specific Oligonucleotide
ggtgtgcgcgtcgtacactagacccgttataagtgttg

SEQ ID NO: 409 - DNA, 40 bp, Artificial Sequence
  Target Specific Oligonucleotide
gtgtttagggtcgcggttgatagacccgttataagtgttg

SEQ ID NO: 410 - DNA, 42 bp, Artificial Sequence
  Target Specific Oligonucleotide
gagaatggcgaagtaaatgccctagacccgttataagtgttg

SEQ ID NO: 411 - DNA, 42 bp, Artificial Sequence
  Target Specific Oligonucleotide
gtagatggaatagacacggctgtagacccgttataagtgttg

SEQ ID NO: 412 - DNA, 43 bp, Artificial Sequence
  Target Specific Oligonucleotide
gctcttaatgcatggtacaactgtagacccgttataagtgttg

SEQ ID NO: 413 - DNA, 43 bp, Artificial Sequence
  Target Specific Oligonucleotide
aaaccagtatttcgtcacagtgatagacccgttataagtgttg

SEQ ID NO: 414 - DNA, 42 bp, Artificial Sequence
  Target Specific Oligonucleotide
gatgggaacggtgtagagatgttagacccgttataagtgttg

SEQ ID NO: 415 - DNA, 42 bp, Artificial Sequence
  Target Specific Oligonucleotide
ttccaaatgccgtcaaaactgttagacccgttataagtgttg

SEQ ID NO: 416 - DNA, 39 bp, Artificial Sequence
  Target Specific Oligonucleotide
tgggtcacagacggtgtggtagacccgttataagtgttg

SEQ ID NO: 417 - DNA, 46 bp, Artificial Sequence
  Target Specific Oligonucleotide
aatcaggtatacttctatccttgaaatagacccgttataagtgttg

SEQ ID NO: 418 - DNA, 43 bp, Artificial Sequence
  Target Specific Oligonucleotide
tgctgtattagcaacttggaacttagacccgttataagtgttg

SEQ ID NO: 419 - DNA, 42 bp, Artificial Sequence
  Target Specific Oligonucleotide
gccaataaagcgatggttgatctagacccgttataagtgttg

SEQ ID NO: 420 - DNA, 43 bp, Artificial Sequence
  Target Specific Oligonucleotide
gtaaggatcactggatcctactgtagacccgttataagtgttg

SEQ ID NO: 421 - DNA, 43 bp, Artificial Sequence
  Target Specific Oligonucleotide
gacctcattcagttgatgagaggtagacccgttataagtgttg

SEQ ID NO: 422 - DNA, 41 bp, Artificial Sequence
  Target Specific Oligonucleotide
gcataacagacggttgcaagttagacccgttataagtgttg

SEQ ID NO: 423 - DNA, 41 bp, Artificial Sequence
  Target Specific Oligonucleotide
acctaaagcgagttgctgagttagacccgttataagtgttg

SEQ ID NO: 424 - DNA, 41 bp, Artificial Sequence
  Target Specific Oligonucleotide
gagtcagaaacacgcatggaatagacccgttataagtgttg

SEQ ID NO: 425 - DNA, 42 bp, Artificial Sequence
  Target Specific Oligonucleotide
agcaactctgataggctcacactagacccgttataagtgttg

SEQ ID NO: 426 - DNA, 44 bp, Artificial Sequence
  Target Specific Oligonucleotide
ccatacccactacggataaagatgtagacccgttataagtgttg

SEQ ID NO: 427 - DNA, 42 bp, Artificial Sequence
  Target Specific Oligonucleotide
gctttctgtacgactcaggttttagacccgttataagtgttg

SEQ ID NO: 428 - DNA, 42 bp, Artificial Sequence
  Target Specific Oligonucleotide
caatgttgagccactaaaccactagacccgttataagtgttg

SEQ ID NO: 429 - DNA, 42 bp, Artificial Sequence
  Target Specific Oligonucleotide
atgcttggattaggtccaaagctagacccgttataagtgttg

SEQ ID NO: 430 - DNA, 41 bp, Artificial Sequence
  Target Specific Oligonucleotide
cccggtgatggattagtttggtagacccgttataagtgttg

SEQ ID NO: 431 - DNA, 41 bp, Artificial Sequence
  Target Specific Oligonucleotide
ccatggccataccctggaatttagacccgttataagtgttg

SEQ ID NO: 432 - DNA, 41 bp, Artificial Sequence
  Target Specific Oligonucleotide
attatactgtccagcgtaggtggtagacaaccgtcgttaag

SEQ ID NO: 433 - DNA, 41 bp, Artificial Sequence
  Target Specific Oligonucleotide
tcctggtaaggataggtaccatgtagacaaccgtcgttaag

SEQ ID NO: 434 - DNA, 41 bp, Artificial Sequence
  Target Specific Oligonucleotide
cactgaaataggacggaatctgctagacaaccgtcgttaag

SEQ ID NO: 435 - DNA, 39 bp, Artificial Sequence
  Target Specific Oligonucleotide
aaagtttattggcgcttgccgtagacaaccgtcgttaag

SEQ ID NO: 436 - DNA, 39 bp, Artificial Sequence
  Target Specific Oligonucleotide
gcaatgccattagcgatacgatagacaaccgtcgttaag

SEQ ID NO: 437 - DNA, 38 bp, Artificial Sequence
  Target Specific Oligonucleotide
tgattgaacaccacgcgacatagacaaccgtcgttaag

SEQ ID NO: 438 - DNA, 40 bp, Artificial Sequence
  Target Specific Oligonucleotide
agtcttcgaagcactattgccatagacaaccgtcgttaag

SEQ ID NO: 439 - DNA, 39 bp, Artificial Sequence
  Target Specific Oligonucleotide
tcaatggttgatcggcctctctagacaaccgtcgttaag

SEQ ID NO: 440 - DNA, 41 bp, Artificial Sequence
  Target Specific Oligonucleotide
tggcatgatgtctaataggagtctagacaaccgtcgttaag

SEQ ID NO: 441 - DNA, 38 bp, Artificial Sequence
  Target Specific Oligonucleotide
ctgcggtttagccttgacgttagacaaccgtcgttaag

SEQ ID NO: 442 - DNA, 38 bp, Artificial Sequence
  Target Specific Oligonucleotide
cccagaggacgccatcattttagacaaccgtcgttaag

SEQ ID NO: 443 - DNA, 40 bp, Artificial Sequence
  Target Specific Oligonucleotide
tttcttgaaagacgacagcagttagacaaccgtcgttaag

SEQ ID NO: 444 - DNA, 41 bp, Artificial Sequence
  Target Specific Oligonucleotide
attaaatgttcggaaggatgacctagacaaccgtcgttaag

SEQ ID NO: 445 - DNA, 38 bp, Artificial Sequence
  Target Specific Oligonucleotide
tcttttgtcagcacggttgctagacaaccgtcgttaag

SEQ ID NO: 446 - DNA, 38 bp, Artificial Sequence
  Target Specific Oligonucleotide
ctggatcagctcgaccaggatagacaaccgtcgttaag

SEQ ID NO: 447 - DNA, 38 bp, Artificial Sequence
  Target Specific Oligonucleotide
accatggtccgacttctgcctagacaaccgtcgttaag

SEQ ID NO: 448 - DNA, 40 bp, Artificial Sequence
  Target Specific Oligonucleotide
ctggatattcggttatctgggctagacaaccgtcgttaag

SEQ ID NO: 449 - DNA, 41 bp, Artificial Sequence
  Target Specific Oligonucleotide
atcccctttacgatactcttcagtagacaaccgtcgttaag

SEQ ID NO: 450 - DNA, 38 bp, Artificial Sequence
  Target Specific Oligonucleotide
tccagagcgggaacagtatgtagacaaccgtcgttaag

SEQ ID NO: 451 - DNA, 38 bp, Artificial Sequence
  Target Specific Oligonucleotide
gttgcctgcataagatgggctagacaaccgtcgttaag

SEQ ID NO: 452 - DNA, 38 bp, Artificial Sequence
  Target Specific Oligonucleotide
ccttttgtgccgatgacccgtagacaaccgtcgttaag

SEQ ID NO: 453 - DNA, 37 bp, Artificial Sequence
  Target Specific Oligonucleotide
gcttccgtggactgggacgtagacaaccgtcgttaag

SEQ ID NO: 454 - DNA, 41 bp, Artificial Sequence
  Target Specific Oligonucleotide
ccatagaaggacgaggtatttcctagacaaccgtcgttaag

SEQ ID NO: 455 - DNA, 39 bp, Artificial Sequence
  Target Specific Oligonucleotide
agaatgagtcgctgctcgatatagacaaccgtcgttaag

SEQ ID NO: 456 - DNA, 41 bp, Artificial Sequence
  Target Specific Oligonucleotide
gcttcgacaaacgcaaagcgtagactcaatgatgataaaga

SEQ ID NO: 457 - DNA, 44 bp, Artificial Sequence
  Target Specific Oligonucleotide
gctagttcacagaaagaccattttagactcaatgatgataaaga

SEQ ID NO: 458 - DNA, 41 bp, Artificial Sequence
  Target Specific Oligonucleotide
cagctcatcgaaagcgaccctagactcaatgatgataaaga

SEQ ID NO: 459 - DNA, 40 bp, Artificial Sequence
  Target Specific Oligonucleotide
cggctgctacaaacctcggtagactcaatgatgataaaga

SEQ ID NO: 460 - DNA, 42 bp, Artificial Sequence
  Target Specific Oligonucleotide
aacaatttgaaggccccacaatagactcaatgatgataaaga

SEQ ID NO: 461 - DNA, 39 bp, Artificial Sequence
  Target Specific Oligonucleotide
gcgtctgcaccggagacttagactcaatgatgataaaga

SEQ ID NO: 462 - DNA, 39 bp, Artificial Sequence
  Target Specific Oligonucleotide
gcgctgctcaggcattggtagactcaatgatgataaaga

SEQ ID NO: 463 - DNA, 47 bp, Artificial Sequence
  Target Specific Oligonucleotide
ttgttgtgtaacaacataatttcaagtagactcaatgatgataaaga

SEQ ID NO: 464 - DNA, 38 bp, Artificial Sequence
  Target Specific Oligonucleotide
ctccacgtcggcgtgcatagactcaatgatgataaaga

SEQ ID NO: 465 - DNA, 40 bp, Artificial Sequence
  Target Specific Oligonucleotide
ctgcggggattgtagccggtagactcaatgatgataaaga

SEQ ID NO: 466 - DNA, 42 bp, Artificial Sequence
  Target Specific Oligonucleotide
atcttagctactgaccggctatagactcaatgatgataaaga

SEQ ID NO: 467 - DNA, 43 bp, Artificial Sequence
  Target Specific Oligonucleotide
aagagattatacgcctcacggatagactcaatgatgataaaga

SEQ ID NO: 468 - DNA, 44 bp, Artificial Sequence
  Target Specific Oligonucleotide
tttcgagtaacgaaattagctcctagactcaatgatgataaaga

SEQ ID NO: 469 - DNA, 39 bp, Artificial Sequence
  Target Specific Oligonucleotide
gacccgcagattggcacgtagactcaatgatgataaaga

SEQ ID NO: 470 - DNA, 41 bp, Artificial Sequence
  Target Specific Oligonucleotide
atggccccaatctcgtttggtagactcaatgatgataaaga

SEQ ID NO: 471 - DNA, 44 bp, Artificial Sequence
  Target Specific Oligonucleotide
ccagtagctataacgaagtcctctagactcaatgatgataaaga

SEQ ID NO: 472 - DNA, 43 bp, Artificial Sequence
  Target Specific Oligonucleotide
acaaattcccggacactatggatagactcaatgatgataaaga

SEQ ID NO: 473 - DNA, 43 bp, Artificial Sequence
  Target Specific Oligonucleotide
ttcacagagttgataaggccactagactcaatgatgataaaga

SEQ ID NO: 474 - DNA, 43 bp, Artificial Sequence
  Target Specific Oligonucleotide
aactttgccggtctctttacattagactcaatgatgataaaga

SEQ ID NO: 475 - DNA, 45 bp, Artificial Sequence
  Target Specific Oligonucleotide
ttccaatgtgcaagaatgatttcttagactcaatgatgataaaga

SEQ ID NO: 476 - DNA, 43 bp, Artificial Sequence
  Target Specific Oligonucleotide
ctccttctggggtatttcctgctagactcaatgatgataaaga

SEQ ID NO: 477 - DNA, 40 bp, Artificial Sequence
  Target Specific Oligonucleotide
ccacttcagttggccggtatagactcaatgatgataaaga

SEQ ID NO: 478 - DNA, 39 bp, Artificial Sequence
  Target Specific Oligonucleotide
tccgtcaacgtccgcagttagactcaatgatgataaaga

SEQ ID NO: 479 - DNA, 42 bp, Artificial Sequence
  Target Specific Oligonucleotide
agtgagatcgccatagtgcaatagactcaatgatgataaaga

SEQ ID NO: 480 - DNA, 43 bp, Artificial Sequence
  Target Specific Oligonucleotide
gaaggtgaccctataaggagtcatagactacaacaaaagatcg

SEQ ID NO: 481 - DNA, 41 bp, Artificial Sequence
  Target Specific Oligonucleotide
aacaccgcttgcatagttgtgtagactacaacaaaagatcg

SEQ ID NO: 482 - DNA, 41 bp, Artificial Sequence
  Target Specific Oligonucleotide
tacagacgcggaatcattctctagactacaacaaaagatcg

SEQ ID NO: 483 - DNA, 41 bp, Artificial Sequence
  Target Specific Oligonucleotide
tggaggaaatgagcatgaccttagactacaacaaaagatcg

SEQ ID NO: 484 - DNA, 41 bp, Artificial Sequence
  Target Specific Oligonucleotide
atggtgtacactcgaggctgatagactacaacaaaagatcg

SEQ ID NO: 485 - DNA, 41 bp, Artificial Sequence
  Target Specific Oligonucleotide
ccttgtggtagacgttcagcttagactacaacaaaagatcg

SEQ ID NO: 486 - DNA, 44 bp, Artificial Sequence
  Target Specific Oligonucleotide
ggatcaggtttaatggtcactatgtagactacaacaaaagatcg

SEQ ID NO: 487 - DNA, 40 bp, Artificial Sequence
  Target Specific Oligonucleotide
cacacggtcaacgctgtacatagactacaacaaaagatcg

SEQ ID NO: 488 - DNA, 40 bp, Artificial Sequence
  Target Specific Oligonucleotide
caggtgtttgcggaagttcctagactacaacaaaagatcg

SEQ ID NO: 489 - DNA, 40 bp, Artificial Sequence
  Target Specific Oligonucleotide
gtgctcatggtcgtagaggatagactacaacaaaagatcg

SEQ ID NO: 490 - DNA, 43 bp, Artificial Sequence
  Target Specific Oligonucleotide
aagtcaaagtacgtctcgatcattagactacaacaaaagatcg

SEQ ID NO: 491 - DNA, 39 bp, Artificial Sequence
  Target Specific Oligonucleotide
tcgtagcagccgttggagatagactacaacaaaagatcg

SEQ ID NO: 492 - DNA, 39 bp, Artificial Sequence
  Target Specific Oligonucleotide
tcgtggccagagtaggcattagactacaacaaaagatcg

SEQ ID NO: 493 - DNA, 40 bp, Artificial Sequence
  Target Specific Oligonucleotide
ggtactctccaaacgctcggtagactacaacaaaagatcg

SEQ ID NO: 494 - DNA, 42 bp, Artificial Sequence
  Target Specific Oligonucleotide
aggtacgaatagggatgtcgtctagactacaacaaaagatcg

SEQ ID NO: 495 - DNA, 42 bp, Artificial Sequence
  Target Specific Oligonucleotide
agggtatcgtacatcgttccaatagactacaacaaaagatcg

SEQ ID NO: 496 - DNA, 39 bp, Artificial Sequence
  Target Specific Oligonucleotide
caggtgatctgcgccgttgtagactacaacaaaagatcg

SEQ ID NO: 497 - DNA, 39 bp, Artificial Sequence
  Target Specific Oligonucleotide
ccacaggcgatacaaccggtagactacaacaaaagatcg

SEQ ID NO: 498 - DNA, 39 bp, Artificial Sequence
  Target Specific Oligonucleotide
cagcagcttcgagtgctggtagactacaacaaaagatcg

SEQ ID NO: 499 - DNA, 39 bp, Artificial Sequence
  Target Specific Oligonucleotide
ccctcacaggacgtcgtcatagactacaacaaaagatcg

SEQ ID NO: 500 - DNA, 39 bp, Artificial Sequence
  Target Specific Oligonucleotide
gcacgaagccttcggtgtctagactacaacaaaagatcg

SEQ ID NO: 501 - DNA, 41 bp, Artificial Sequence
  Target Specific Oligonucleotide
tgggaagtgtcggctttcatgtagactacaacaaaagatcg

SEQ ID NO: 502 - DNA, 41 bp, Artificial Sequence; identical to SEQ ID NO: 501
  Target Specific Oligonucleotide

SEQ ID NO: 503 - DNA, 38 bp, Artificial Sequence
  Target Specific Oligonucleotide
tgctcgcactttggccgctagactacaacaaaagatcg

SEQ ID NO: 504 - DNA, 40 bp, Artificial Sequence
  Target Specific Oligonucleotide
gtcttttggcacggtttctgttagactgtattcaacgtcc

SEQ ID NO: 505 - DNA, 40 bp, Artificial Sequence
  Target Specific Oligonucleotide
tacttcttcaacgcgaagagctagactgtattcaacgtcc

SEQ ID NO: 506 - DNA, 40 bp, Artificial Sequence
  Target Specific Oligonucleotide
tttgaggttgtatccgctgcttagactgtattcaacgtcc

SEQ ID NO: 507 - DNA, 44 bp, Artificial Sequence
  Target Specific Oligonucleotide
caagttgactaaatctcgtactttctagactgtattcaacgtcc

SEQ ID NO: 508 - DNA, 43 bp, Artificial Sequence
  Target Specific Oligonucleotide
gccttattaacggtatcttcagaatagactgtattcaacgtcc

SEQ ID NO: 509 - DNA, 40 bp, Artificial Sequence
  Target Specific Oligonucleotide
tgttctggatttcgcaggtcctagactgtattcaacgtcc

SEQ ID NO: 510 - DNA, 42 bp, Artificial Sequence
  Target Specific Oligonucleotide
gctttatcaggttatgttgcatgtagactgtattcaacgtcc

SEQ ID NO: 511 - DNA, 43 bp, Artificial Sequence
  Target Specific Oligonucleotide
gattctggcttatagggtattcactagactgtattcaacgtcc

SEQ ID NO: 512 - DNA, 41 bp, Artificial Sequence
  Target Specific Oligonucleotide
ttttcctcccgcaattcctagatagactgtattcaacgtcc

SEQ ID NO: 513 - DNA, 40 bp, Artificial Sequence
  Target Specific Oligonucleotide
tgtttccgtcaaatcgtgtggtagactgtattcaacgtcc

SEQ ID NO: 514 - DNA, 41 bp, Artificial Sequence
  Target Specific Oligonucleotide
ggttccctctagatcttgcctttagactgtattcaacgtcc

SEQ ID NO: 515 - DNA, 43 bp, Artificial Sequence
  Target Specific Oligonucleotide
gcatgtaccacctatcatctaatgtagactgtattcaacgtcc

SEQ ID NO: 516 - DNA, 39 bp, Artificial Sequence
  Target Specific Oligonucleotide
gttgccaacacgagctgacttagactgtattcaacgtcc

SEQ ID NO: 517 - DNA, 41 bp, Artificial Sequence
  Target Specific Oligonucleotide
actgactactagttcaagcgcatagactgtattcaacgtcc

SEQ ID NO: 518 - DNA, 39 bp, Artificial Sequence
  Target Specific Oligonucleotide
tctcttgctcgctttggacctagactgtattcaacgtcc

SEQ ID NO: 519 - DNA, 44 bp, Artificial Sequence
  Target Specific Oligonucleotide
cactgctagaacaactatcaatttgtagactgtattcaacgtcc

SEQ ID NO: 520 - DNA, 41 bp, Artificial Sequence
  Target Specific Oligonucleotide
ttacatggcttaagttggggagtagactgtattcaacgtcc

SEQ ID NO: 521 - DNA, 40 bp, Artificial Sequence
  Target Specific Oligonucleotide
gtgaggggacgctcttgtatttagactgtattcaacgtcc

SEQ ID NO: 522 - DNA, 40 bp, Artificial Sequence
  Target Specific Oligonucleotide
aacggtgctatgcctagtagatagactgtattcaacgtcc

SEQ ID NO: 523 - DNA, 40 bp, Artificial Sequence
  Target Specific Oligonucleotide
tttgccttccctagagtgctatagactgtattcaacgtcc

SEQ ID NO: 524 - DNA, 43 bp, Artificial Sequence
  Target Specific Oligonucleotide
atgtcccaatggatacttaaagcctagactgtattcaacgtcc

SEQ ID NO: 525 - DNA, 43 bp, Artificial Sequence
  Target Specific Oligonucleotide
cttccagtaacgagatactttccttagactgtattcaacgtcc

SEQ ID NO: 526 - DNA, 42 bp, Artificial Sequence
  Target Specific Oligonucleotide
caaccatgaattagtcccttgggtagactgtattcaacgtcc

SEQ ID NO: 527 - DNA, 44 bp, Artificial Sequence
  Target Specific Oligonucleotide
cagcattattagacactttaactgttagactgtattcaacgtcc

SEQ ID NO: 528 - DNA, 43 bp, Artificial Sequence
  Target Specific Oligonucleotide
tgcacatatgattgacgctcagtagactcttatattgagtggt

SEQ ID NO: 529 - DNA, 40 bp, Artificial Sequence
  Target Specific Oligonucleotide
ttcaccgcctgcaacaagatagactcttatattgagtggt

SEQ ID NO: 530 - DNA, 44 bp, Artificial Sequence
  Target Specific Oligonucleotide
ttcacttacaggtaacaccaagttagactcttatattgagtggt

SEQ ID NO: 531 - DNA, 45 bp, Artificial Sequence
  Target Specific Oligonucleotide
aagcaatagatcgtccataagttatagactcttatattgagtggt

SEQ ID NO: 532 - DNA, 48 bp, Artificial Sequence
  Target Specific Oligonucleotide
tacaagaaccctaattgtaataatagatagactcttatattgagtggt

SEQ ID NO: 533 - DNA, 44 bp, Artificial Sequence
  Target Specific Oligonucleotide
gaataggaccaaagtgtcccttgtagactcttatattgagtggt

SEQ ID NO: 534 - DNA, 44 bp, Artificial Sequence
  Target Specific Oligonucleotide
tccatcaggactaaatctcacactagactcttatattgagtggt

SEQ ID NO: 535 - DNA, 44 bp, Artificial Sequence
  Target Specific Oligonucleotide
aaaggccatacgtttttcctacctagactcttatattgagtggt

SEQ ID NO: 536 - DNA, 44 bp, Artificial Sequence
  Target Specific Oligonucleotide
gcagtgtacaatgaacgagaaattagactcttatattgagtggt

SEQ ID NO: 537 - DNA, 44 bp, Artificial Sequence
  Target Specific Oligonucleotide
ggcattctgctttaagatcagaatagactcttatattgagtggt

SEQ ID NO: 538 - DNA, 40 bp, Artificial Sequence
  Target Specific Oligonucleotide
ggccagccacgattcacagtagactcttatattgagtggt

SEQ ID NO: 539 - DNA, 39 bp, Artificial Sequence
  Target Specific Oligonucleotide
gaagccagcgaccggacttagactcttatattgagtggt

SEQ ID NO: 540 - DNA, 41 bp, Artificial Sequence
  Target Specific Oligonucleotide
ctgagggtcgtcgttgtctttagactcttatattgagtggt

SEQ ID NO: 541 - DNA, 43 bp, Artificial Sequence
  Target Specific Oligonucleotide
ctgaaccactggcatagagttctagactcttatattgagtggt

SEQ ID NO: 542 - DNA, 40 bp, Artificial Sequence
  Target Specific Oligonucleotide
aaatcaaccacgggtcgcgtagactcttatattgagtggt

SEQ ID NO: 543 - DNA, 40 bp, Artificial Sequence
  Target Specific Oligonucleotide
agcaacagtcgacgagggatagactcttatattgagtggt

SEQ ID NO: 544 - DNA, 48 bp, Artificial Sequence
  Target Specific Oligonucleotide
atatattccatactactaacagacatatagactcttatattgagtggt

SEQ ID NO: 545 - DNA, 38 bp, Artificial Sequence
  Target Specific Oligonucleotide
tccacaccggcatcggctagactcttatattgagtggt

SEQ ID NO: 546 - DNA, 40 bp, Artificial Sequence
  Target Specific Oligonucleotide
agaagggcaatccggtggctagactcttatattgagtggt

SEQ ID NO: 547 - DNA, 40 bp, Artificial Sequence
  Target Specific Oligonucleotide
tcagggagtctgcgaccagtagactcttatattgagtggt

SEQ ID NO: 548 - DNA, 40 bp, Artificial Sequence
  Target Specific Oligonucleotide
gctttgccagctcaccacttagactcttatattgagtggt

SEQ ID NO: 549 - DNA, 43 bp, Artificial Sequence
  Target Specific Oligonucleotide
aaatacccataaggcgtgatgctagactcttatattgagtggt

SEQ ID NO: 550 - DNA, 44 bp, Artificial Sequence
  Target Specific Oligonucleotide
attactatcctgcgtgaaatccatagactcttatattgagtggt

SEQ ID NO: 551 - DNA, 43 bp, Artificial Sequence
  Target Specific Oligonucleotide
cccaaagtcgaacagttttgtctagactcttatattgagtggt

SEQ ID NO: 552 - DNA, 41 bp, Artificial Sequence
  Target Specific Oligonucleotide
tcctcaaatgtccgaggatgatagacctattctatgcttcg

SEQ ID NO: 553 - DNA, 42 bp, Artificial Sequence
  Target Specific Oligonucleotide
gtgaacacaacgtaagaacaggtagacctattctatgcttcg

SEQ ID NO: 554 - DNA, 42 bp, Artificial Sequence
  Target Specific Oligonucleotide
actcctctggttacgcttcatttagacctattctatgcttcg

SEQ ID NO: 555 - DNA, 41 bp, Artificial Sequence
  Target Specific Oligonucleotide
acctaacataaccgtgctgcctagacctattctatgcttcg

SEQ ID NO: 556 - DNA, 41 bp, Artificial Sequence
  Target Specific Oligonucleotide
ttcagttgaccgtctggacactagacctattctatgcttcg

SEQ ID NO: 557 - DNA, 43 bp, Artificial Sequence
  Target Specific Oligonucleotide
ctctgacacacgtaacaataacatagacctattctatgcttcg

SEQ ID NO: 558 - DNA, 43 bp, Artificial Sequence
  Target Specific Oligonucleotide
agtaatgtgaggtacaactgcattagacctattctatgcttcg

SEQ ID NO: 559 - DNA, 41 bp, Artificial Sequence
  Target Specific Oligonucleotide
gcttctttcccgcccaaaatatagacctattctatgcttcg

SEQ ID NO: 560 - DNA, 44 bp, Artificial Sequence
  Target Specific Oligonucleotide
aaagaaaagcggtctactagattatagacctattctatgcttcg

SEQ ID NO: 561 - DNA, 41 bp, Artificial Sequence
  Target Specific Oligonucleotide
tgacgtgcggcgaaatttatgtagacctattctatgcttcg

SEQ ID NO: 562 - DNA, 41 bp, Artificial Sequence
  Target Specific Oligonucleotide
gcttattgcagcgatggatgatagacctattctatgcttcg

SEQ ID NO: 563 - DNA, 42 bp, Artificial Sequence
  Target Specific Oligonucleotide
tgtcagtagacgatagaggaggtagacctattctatgcttcg

SEQ ID NO: 564 - DNA, 41 bp, Artificial Sequence
  Target Specific Oligonucleotide
ttggttccgactacccaacagtagacctattctatgcttcg

SEQ ID NO: 565 - DNA, 45 bp, Artificial Sequence
  Target Specific Oligonucleotide
ctcttaagtacgcaataattctctctagacctattctatgcttcg

SEQ ID NO: 566 - DNA, 43 bp, Artificial Sequence
  Target Specific Oligonucleotide
gttccgtatatgtcggatctctctagacctattctatgcttcg

SEQ ID NO: 567 - DNA, 41 bp, Artificial Sequence
  Target Specific Oligonucleotide
aaattcaccggacggagtgtttagacctattctatgcttcg

SEQ ID NO: 568 - DNA, 42 bp, Artificial Sequence
  Target Specific Oligonucleotide
tttgtgaaaccgtagtggctcttagacctattctatgcttcg

SEQ ID NO: 569 - DNA, 42 bp, Artificial Sequence
  Target Specific Oligonucleotide
acctctttaacgaaggtgtcagtagacctattctatgcttcg

SEQ ID NO: 570 - DNA, 41 bp, Artificial Sequence
  Target Specific Oligonucleotide
ggactccggttctaacttggttagacctattctatgcttcg

SEQ ID NO: 571 - DNA, 43 bp, Artificial Sequence
  Target Specific Oligonucleotide
atctctatatgacgtgctgttggtagacctattctatgcttcg

SEQ ID NO: 572 - DNA, 43 bp, Artificial Sequence
  Target Specific Oligonucleotide
gttggaattattcggagactgagtagacctattctatgcttcg

SEQ ID NO: 573 - DNA, 40 bp, Artificial Sequence
  Target Specific Oligonucleotide
gcacatttgacgacggcttctagacctattctatgcttcg

SEQ ID NO: 574 - DNA, 40 bp, Artificial Sequence
  Target Specific Oligonucleotide
actccgtgtaaacgcagtggtagacctattctatgcttcg

SEQ ID NO: 575 - DNA, 43 bp, Artificial Sequence
  Target Specific Oligonucleotide
acacataactcgttaacacgttctagacctattctatgcttcg

SEQ ID NO: 576 - DNA, 42 bp, Artificial Sequence
  Target Specific Oligonucleotide
cagatctgttgagctaacagggtagacatgttataccatgcg

SEQ ID NO: 577 - DNA, 44 bp, Artificial Sequence
  Target Specific Oligonucleotide
ttctagactatgcaaccctctaggtagacatgttataccatgcg

SEQ ID NO: 578 - DNA, 43 bp, Artificial Sequence
  Target Specific Oligonucleotide
cacccatgtgagtaatacactgctagacatgttataccatgcg

SEQ ID NO: 579 - DNA, 42 bp, Artificial Sequence
  Target Specific Oligonucleotide
agtcccatattgccgttcatgctagacatgttataccatgcg

SEQ ID NO: 580 - DNA, 42 bp, Artificial Sequence
  Target Specific Oligonucleotide
atttcctcataacggtcatggctagacatgttataccatgcg

SEQ ID NO: 581 - DNA, 42 bp, Artificial Sequence
  Target Specific Oligonucleotide
ttactgcctagctagcaggttatagacatgttataccatgcg

SEQ ID NO: 582 - DNA, 41 bp, Artificial Sequence
  Target Specific Oligonucleotide
acgtgcccttggtactatgactagacatgttataccatgcg

SEQ ID NO: 583 - DNA, 41 bp, Artificial Sequence
  Target Specific Oligonucleotide
cttgccaaaccctagcttggatagacatgttataccatgcg

SEQ ID NO: 584 - DNA, 45 bp, Artificial Sequence
  Target Specific Oligonucleotide
attatcacttaacgaaggtcctttgtagacatgttataccatgcg

SEQ ID NO: 585 - DNA, 45 bp, Artificial Sequence
  Target Specific Oligonucleotide
atatacagcctaagcaccaattatgtagacatgttataccatgcg

SEQ ID NO: 586 - DNA, 40 bp, Artificial Sequence
  Target Specific Oligonucleotide
ggcaacaaaacgtcatggcatagacatgttataccatgcg

SEQ ID NO: 587 - DNA, 43 bp, Artificial Sequence
  Target Specific Oligonucleotide
gtctgaggactaaccctaaagggtagacatgttataccatgcg

SEQ ID NO: 588 - DNA, 46 bp, Artificial Sequence
  Target Specific Oligonucleotide
cttaaaatagatcgaaactctgtctctagacatgttataccatgcg

SEQ ID NO: 589 - DNA, 44 bp, Artificial Sequence
  Target Specific Oligonucleotide
tgatctgtacaagctacgattttatagacatgttataccatgcg

SEQ ID NO: 590 - DNA, 43 bp, Artificial Sequence
  Target Specific Oligonucleotide
aaatcagttataccaaggggagatagacatgttataccatgcg

SEQ ID NO: 591 - DNA, 45 bp, Artificial Sequence
  Target Specific Oligonucleotide
taggaaaacatacgtatactgaatatagacatgttataccatgcg

SEQ ID NO: 592 - DNA, 40 bp, Artificial Sequence
  Target Specific Oligonucleotide
agttggcctcacgttgcatttagacatgttataccatgcg

SEQ ID NO: 593 - DNA, 40 bp, Artificial Sequence
  Target Specific Oligonucleotide
tttggtctcggcttgcgaattagacatgttataccatgcg

SEQ ID NO: 594 - DNA, 41 bp, Artificial Sequence
  Target Specific Oligonucleotide
cagtagtcaacgcagcactcatagacatgttataccatgcg

SEQ ID NO: 595 - DNA, 44 bp, Artificial Sequence
  Target Specific Oligonucleotide
caacaggatcacctaatattcccatagacatgttataccatgcg

SEQ ID NO: 596 - DNA, 43 bp, Artificial Sequence
  Target Specific Oligonucleotide
tttggacagatatctcctcgaaatagacatgttataccatgcg

SEQ ID NO: 597 - DNA, 43 bp, Artificial Sequence
  Target Specific Oligonucleotide
aaaggctatctacactttggcaatagacatgttataccatgcg

SEQ ID NO: 598 - DNA, 40 bp, Artificial Sequence
  Target Specific Oligonucleotide
tgcctggatcctatcccgactagacatgttataccatgcg

SEQ ID NO: 599 - DNA, 40 bp, Artificial Sequence
  Target Specific Oligonucleotide
tgtggtcacctacacgctgctagacatgttataccatgcg

SEQ ID NO: 600 - DNA, 43 bp, Artificial Sequence
  Target Specific Oligonucleotide
ggtgagctggtaatctgaccttagacaatctatttaccctacg

SEQ ID NO: 601 - DNA, 41 bp, Artificial Sequence
  Target Specific Oligonucleotide
gttgcaggcatgacgcaagtagacaatctatttaccctacg

SEQ ID NO: 602 - DNA, 43 bp, Artificial Sequence
  Target Specific Oligonucleotide
aaaacgcctacgcatcatgtctagacaatctatttaccctacg

SEQ ID NO: 603 - DNA, 40 bp, Artificial Sequence
  Target Specific Oligonucleotide
tggtcctgcgcggatgtctagacaatctatttaccctacg

SEQ ID NO: 604 - DNA, 41 bp, Artificial Sequence
  Target Specific Oligonucleotide
ggtgagcttctccgtggcgtagacaatctatttaccctacg

SEQ ID NO: 605 - DNA, 41 bp, Artificial Sequence
  Target Specific Oligonucleotide
agcgcagggccaattgacttagacaatctatttaccctacg

SEQ ID NO: 606 - DNA, 43 bp, Artificial Sequence
  Target Specific Oligonucleotide
ccgggtacacggttttgatcttagacaatctatttaccctacg

SEQ ID NO: 607 - DNA, 40 bp, Artificial Sequence
  Target Specific Oligonucleotide
ccggctcgatgtcgggtatagacaatctatttaccctacg

SEQ ID NO: 608 - DNA, 40 bp, Artificial Sequence
  Target Specific Oligonucleotide
ggcgccttctcgccacattagacaatctatttaccctacg

SEQ ID NO: 609 - DNA, 40 bp, Artificial Sequence
  Target Specific Oligonucleotide
cacgttgaagcgcgggtgtagacaatctatttaccctacg

SEQ ID NO: 610 - DNA, 44 bp, Artificial Sequence
  Target Specific Oligonucleotide
gcccacagaaaccggtttaatgtagacaatctatttaccctacg

SEQ ID NO: 611 - DNA, 43 bp, Artificial Sequence
  Target Specific Oligonucleotide
tcagaatgagcgcaatccaggtagacaatctatttaccctacg

SEQ ID NO: 612 - DNA, 42 bp, Artificial Sequence
  Target Specific Oligonucleotide
ttcggacacaaagacgctcctagacaatctatttaccctacg

SEQ ID NO: 613 - DNA, 42 bp, Artificial Sequence
  Target Specific Oligonucleotide
agcttgacgtaggtgtcggttagacaatctatttaccctacg

SEQ ID NO: 614 - DNA, 42 bp, Artificial Sequence
  Target Specific Oligonucleotide
ggttcccgccaaatttcccgtagacaatctatttaccctacg

SEQ ID NO: 615 - DNA, 45 bp, Artificial Sequence
  Target Specific Oligonucleotide
cagggctcatgatagtacaacagtagacaatctatttaccctacg

SEQ ID NO: 616 - DNA, 41 bp, Artificial Sequence
  Target Specific Oligonucleotide
ggactttagcgcggtctcctagacaatctatttaccctacg

SEQ ID NO: 617 - DNA, 41 bp, Artificial Sequence
  Target Specific Oligonucleotide
gcagccgttctaagcgctgtagacaatctatttaccctacg

SEQ ID NO: 618 - DNA, 41 bp, Artificial Sequence
  Target Specific Oligonucleotide
gctggtaaaggcgcttccctagacaatctatttaccctacg

SEQ ID NO: 619 - DNA, 41 bp, Artificial Sequence
  Target Specific Oligonucleotide
gaagaagtcggtgacgcggtagacaatctatttaccctacg

SEQ ID NO: 620 - DNA, 41 bp, Artificial Sequence
  Target Specific Oligonucleotide
ggctggaaacctcgtccactagacaatctatttaccctacg

SEQ ID NO: 621 - DNA, 48 bp, Artificial Sequence
  Target Specific Oligonucleotide
tttattagagtttaggaggtcagaaatagacaatctatttaccctacg

SEQ ID NO: 622 - DNA, 44 bp, Artificial Sequence
  Target Specific Oligonucleotide
tgggaggtcccatattcagaggtagacaatctatttaccctacg

SEQ ID NO: 623 - DNA, 44 bp, Artificial Sequence
  Target Specific Oligonucleotide
tccagggatgtcctacttgtcctagacaatctatttaccctacg

SEQ ID NO: 624 - DNA, 40 bp, Artificial Sequence
  Target Specific Oligonucleotide
ccaggtttccggcacagtgggtaactgaattgtcacttta

SEQ ID NO: 625 - DNA, 46 bp, Artificial Sequence
  Target Specific Oligonucleotide
tgttatactttgtcgctcttagtagagtaactgaattgtcacttta

SEQ ID NO: 626 - DNA, 44 bp, Artificial Sequence
  Target Specific Oligonucleotide
agcttttaaccaggtttcgacttcgtaactgaattgtcacttta

SEQ ID NO: 627 - DNA, 45 bp, Artificial Sequence
  Target Specific Oligonucleotide
gtatgagaatcccgataaaactggtgtaactgaattgtcacttta

SEQ ID NO: 628 - DNA, 43 bp, Artificial Sequence
  Target Specific Oligonucleotide
aagaggacatcgaccaatcctcagtaactgaattgtcacttta

SEQ ID NO: 629 - DNA, 42 bp, Artificial Sequence
  Target Specific Oligonucleotide
ggtttgtgcaagggtcgaaaacgtaactgaattgtcacttta

SEQ ID NO: 630 - DNA, 44 bp, Artificial Sequence
  Target Specific Oligonucleotide
acacctctcgtagctatagatgtggtaactgaattgtcacttta

SEQ ID NO: 631 - DNA, 41 bp, Artificial Sequence
  Target Specific Oligonucleotide
atgagagggcaaccgaggtgagtaactgaattgtcacttta

SEQ ID NO: 632 - DNA, 43 bp, Artificial Sequence
  Target Specific Oligonucleotide
ccgctgtcagggtctatcattccgtaactgaattgtcacttta

SEQ ID NO: 633 - DNA, 41 bp, Artificial Sequence
  Target Specific Oligonucleotide
gcaatgagcatatcctcgggcgtaactgaattgtcacttta

SEQ ID NO: 634 - DNA, 41 bp, Artificial Sequence
  Target Specific Oligonucleotide
ggcttgagtgggttcacccaggtaactgaattgtcacttta

SEQ ID NO: 635 - DNA, 43 bp, Artificial Sequence
  Target Specific Oligonucleotide
gcaaggtatggcaatagctgagtgtaactgaattgtcacttta

SEQ ID NO: 636 - DNA, 45 bp, Artificial Sequence
  Target Specific Oligonucleotide
cagtaatagtagagacgccactacagtaactgaattgtcacttta

SEQ ID NO: 637 - DNA, 43 bp, Artificial Sequence
  Target Specific Oligonucleotide
cttccaaagtgtcgcttcagagggtaactgaattgtcacttta

SEQ ID NO: 638 - DNA, 46 bp, Artificial Sequence
  Target Specific Oligonucleotide
aggcaattttcccgatactttttattgtaactgaattgtcacttta

SEQ ID NO: 639 - DNA, 46 bp, Artificial Sequence
  Target Specific Oligonucleotide
tgttagcttctatagtcactattcgagtaactgaattgtcacttta

SEQ ID NO: 640 - DNA, 45 bp, Artificial Sequence
  Target Specific Oligonucleotide
tcaacaagtaatgtatcccgttcatgtaactgaattgtcacttta

SEQ ID NO: 641 - DNA, 44 bp, Artificial Sequence
  Target Specific Oligonucleotide
tttccttggccgtaagttgttttcgtaactgaattgtcacttta

SEQ ID NO: 642 - DNA, 42 bp, Artificial Sequence
  Target Specific Oligonucleotide
gggtcaacccagtctgttacctgtaactgaattgtcacttta

SEQ ID NO: 643 - DNA, 46 bp, Artificial Sequence
  Target Specific Oligonucleotide
tctgagactgatagggaaacatatgggtaactgaattgtcacttta

SEQ ID NO: 644 - DNA, 42 bp, Artificial Sequence
  Target Specific Oligonucleotide
tctttgccaccaatagcttggagtaactgaattgtcacttta

SEQ ID NO: 645 - DNA, 45 bp, Artificial Sequence
  Target Specific Oligonucleotide
ctcattggaatggttagtagagcaagtaactgaattgtcacttta

SEQ ID NO: 646 - DNA, 44 bp, Artificial Sequence
  Target Specific Oligonucleotide
ctgagtaaagagcgtgtattccaggtaactgaattgtcacttta

SEQ ID NO: 647 - DNA, 42 bp, Artificial Sequence
  Target Specific Oligonucleotide
tggaggcataaagctggtaggcgtaactgaattgtcacttta

SEQ ID NO: 648 - DNA, 42 bp, Artificial Sequence
  Target Specific Oligonucleotide
ggatctcaagcgttgcaggcgtaacgatagatttatacgacg

SEQ ID NO: 649 - DNA, 39 bp, Artificial Sequence
  Target Specific Oligonucleotide
gcaccgcgagcggactagtaacgatagatttatacgacg

SEQ ID NO: 650 - DNA, 41 bp, Artificial Sequence
  Target Specific Oligonucleotide
tgctgaggtcgctcacgaagtaacgatagatttatacgacg

SEQ ID NO: 651 - DNA, 44 bp, Artificial Sequence
  Target Specific Oligonucleotide
ccggaccacgtagttaaatcttgtaacgatagatttatacgacg

SEQ ID NO: 652 - DNA, 43 bp, Artificial Sequence
  Target Specific Oligonucleotide
aagttcttttgggcgatgccagtaacgatagatttatacgacg

SEQ ID NO: 653 - DNA, 48 bp, Artificial Sequence
  Target Specific Oligonucleotide
gttcccttattagtctaatgttttgcgtaacgatagatttatacgacg

SEQ ID NO: 654 - DNA, 45 bp, Artificial Sequence
  Target Specific Oligonucleotide
ttggaccagtgtacataaggatggtaacgatagatttatacgacg

SEQ ID NO: 655 - DNA, 41 bp, Artificial Sequence
  Target Specific Oligonucleotide
tggcagtgcacggatggtcgtaacgatagatttatacgacg

SEQ ID NO: 656 - DNA, 47 bp, Artificial Sequence
  Target Specific Oligonucleotide
ataccaattctagtgagaagttcacgtaacgatagatttatacgacg

SEQ ID NO: 657 - DNA, 44 bp, Artificial Sequence
  Target Specific Oligonucleotide
tctctgtagggtaaggctctgtgtaacgatagatttatacgacg

SEQ ID NO: 658 - DNA, 45 bp, Artificial Sequence
  Target Specific Oligonucleotide
tttagaggtagaccattgtgtgggtaacgatagatttatacgacg

SEQ ID NO: 659 - DNA, 42 bp, Artificial Sequence
  Target Specific Oligonucleotide
agagagggcactctacgcttgtaacgatagatttatacgacg

SEQ ID NO: 660 - DNA, 44 bp, Artificial Sequence
  Target Specific Oligonucleotide
caaaaagccctagctcagatgggtaacgatagatttatacgacg

SEQ ID NO: 661 - DNA, 43 bp, Artificial Sequence
  Target Specific Oligonucleotide
cctctcctccctatttggctggtaacgatagatttatacgacg

SEQ ID NO: 662 - DNA, 46 bp, Artificial Sequence
  Target Specific Oligonucleotide
ttttacaacgaactcattaggtccgtaacgatagatttatacgacg

SEQ ID NO: 663 - DNA, 45 bp, Artificial Sequence
  Target Specific Oligonucleotide
ggagtagatcccccatctattccgtaacgatagatttatacgacg

SEQ ID NO: 664 - DNA, 44 bp, Artificial Sequence
  Target Specific Oligonucleotide
gtctcttgcctcgtttctctccgtaacgatagatttatacgacg

SEQ ID NO: 665 - DNA, 43 bp, Artificial Sequence
  Target Specific Oligonucleotide
aattctggcgtagcacaaacagtaacgatagatttatacgacg

SEQ ID NO: 666 - DNA, 45 bp, Artificial Sequence
  Target Specific Oligonucleotide
ggccatgaacccctaaatctaaagtaacgatagatttatacgacg

SEQ ID NO: 667 - DNA, 42 bp, Artificial Sequence
  Target Specific Oligonucleotide
gcccactggtaccatcttgggtaacgatagatttatacgacg

SEQ ID NO: 668 - DNA, 42 bp, Artificial Sequence
  Target Specific Oligonucleotide
taccacgtggcactaacacagtaacgatagatttatacgacg

SEQ ID NO: 669 - DNA, 45 bp, Artificial Sequence
  Target Specific Oligonucleotide
ttctagcttttagaccagcagttgtaacgatagatttatacgacg

SEQ ID NO: 670 - DNA, 44 bp, Artificial Sequence
  Target Specific Oligonucleotide
cccaagtgctagtagaacctgcgtaacgatagatttatacgacg

SEQ ID NO: 671 - DNA, 47 bp, Artificial Sequence
  Target Specific Oligonucleotide
aaacacatctatactcaaagacagagtaacgatagatttatacgacg

SEQ ID NO: 672 - DNA, 43 bp, Artificial Sequence
  Target Specific Oligonucleotide
cacagacttgccgactctttggtaacgatagatttatacgacg

SEQ ID NO: 673 - DNA, 44 bp, Artificial Sequence
  Target Specific Oligonucleotide
cacatgttaatgttcccgaccagtaacgatagatttatacgacg

SEQ ID NO: 674 - DNA, 44 bp, Artificial Sequence
  Target Specific Oligonucleotide
attaatcgtttgcggatctgccgtaacgatagatttatacgacg

SEQ ID NO: 675 - DNA, 44 bp, Artificial Sequence
  Target Specific Oligonucleotide
tgggtaacatcggacaagtttggtaacgatagatttatacgacg

SEQ ID NO: 676 - DNA, 44 bp, Artificial Sequence
  Target Specific Oligonucleotide
tctgaagtttcgagccaatgttgtaacgatagatttatacgacg

SEQ ID NO: 677 - DNA, 44 bp, Artificial Sequence
  Target Specific Oligonucleotide
ccattttcaccgctgaaatagagtaacgatagatttatacgacg

SEQ ID NO: 678 - DNA, 43 bp, Artificial Sequence
  Target Specific Oligonucleotide
ctggatcacatgcgttgtcaggtaacgatagatttatacgacg

SEQ ID NO: 679 - DNA, 45 bp, Artificial Sequence
  Target Specific Oligonucleotide
atatttgcctcgtaagatcccctgtaacgatagatttatacgacg

SEQ ID NO: 680 - DNA, 45 bp, Artificial Sequence
  Target Specific Oligonucleotide
ctgaagcggtgttgtatcatagcgtaacgatagatttatacgacg

SEQ ID NO: 681 - DNA, 45 bp, Artificial Sequence
  Target Specific Oligonucleotide
tagaataatggttcgtgaccttcgtaacgatagatttatacgacg

SEQ ID NO: 682 - DNA, 41 bp, Artificial Sequence
  Target Specific Oligonucleotide
acctgaaggcgacgaggaggtaacgatagatttatacgacg

SEQ ID NO: 683 - DNA, 44 bp, Artificial Sequence
  Target Specific Oligonucleotide
gcctggagtctatcgtacaatcgtaacgatagatttatacgacg

SEQ ID NO: 684 - DNA, 47 bp, Artificial Sequence
  Target Specific Oligonucleotide
acatttaattagaccgtaacccttcgtaacgatagatttatacgacg

SEQ ID NO: 685 - DNA, 45 bp, Artificial Sequence
  Target Specific Oligonucleotide
gctctggccatacttaacagatagtaacgatagatttatacgacg

SEQ ID NO: 686 - DNA, 46 bp, Artificial Sequence
  Target Specific Oligonucleotide
cagcattgagtgataatgcaatctgtaacgatagatttatacgacg

SEQ ID NO: 687 - DNA, 44 bp, Artificial Sequence
  Target Specific Oligonucleotide
atacacacccgctaccttactggtaacgatagatttatacgacg

SEQ ID NO: 688 - DNA, 44 bp, Artificial Sequence
  Target Specific Oligonucleotide
acaaagacgagaactatggcaagtaacgatagatttatacgacg

SEQ ID NO: 689 - DNA, 44 bp, Artificial Sequence
  Target Specific Oligonucleotide
aggcagacgctctaagtctaaagtaacgatagatttatacgacg

SEQ ID NO: 690 - DNA, 44 bp, Artificial Sequence
  Target Specific Oligonucleotide
tctcttactcatcgtgaaaggcgtaacgatagatttatacgacg

SEQ ID NO: 691 - DNA, 42 bp, Artificial Sequence
  Target Specific Oligonucleotide
gcctcaagttcgcattcagcgtaacgatagatttatacgacg

SEQ ID NO: 692 - DNA, 44 bp, Artificial Sequence
  Target Specific Oligonucleotide
gaaatggaaacgttaacagccagtaacgatagatttatacgacg

SEQ ID NO: 693 - DNA, 43 bp, Artificial Sequence
  Target Specific Oligonucleotide
cacctgcttcggaatctcagtgtaacgatagatttatacgacg

SEQ ID NO: 694 - DNA, 46 bp, Artificial Sequence
  Target Specific Oligonucleotide
gacttgtgcttcgttaattaaacagtaacgatagatttatacgacg

SEQ ID NO: 695 - DNA, 46 bp, Artificial Sequence
  Target Specific Oligonucleotide
gctgtaatatacgcccactttagcgtaacgatagatttatacgacg

SEQ ID NO: 696 - DNA, 44 bp, Artificial Sequence
  Target Specific Oligonucleotide
ttccattgtgcggttgggattgtaacttataaaggatagacatc

SEQ ID NO: 697 - DNA, 45 bp, Artificial Sequence
  Target Specific Oligonucleotide
ctccagtaatcgcttatggtccgtaacttataaaggatagacatc

SEQ ID NO: 698 - DNA, 45 bp, Artificial Sequence
  Target Specific Oligonucleotide
cctcttctccgtcaagaagttcgtaacttataaaggatagacatc

SEQ ID NO: 699 - DNA, 45 bp, Artificial Sequence
  Target Specific Oligonucleotide
tgattgagatggcggtatttgagtaacttataaaggatagacatc

SEQ ID NO: 700 - DNA, 45 bp, Artificial Sequence
  Target Specific Oligonucleotide
gccaattataagtactcgggctgtaacttataaaggatagacatc

SEQ ID NO: 701 - DNA, 42 bp, Artificial Sequence
  Target Specific Oligonucleotide
ccaggccagcgtatgttccgtaacttataaaggatagacatc

SEQ ID NO: 702 - DNA, 43 bp, Artificial Sequence
  Target Specific Oligonucleotide
ctggggcaaccaacacttgtgtaacttataaaggatagacatc

SEQ ID NO: 703 - DNA, 43 bp, Artificial Sequence
  Target Specific Oligonucleotide
ggctggacaataggctcccagtaacttataaaggatagacatc

SEQ ID NO: 704 - DNA, 44 bp, Artificial Sequence
  Target Specific Oligonucleotide
aaagctatggcacgaaagccggtaacttataaaggatagacatc

SEQ ID NO: 705 - DNA, 44 bp, Artificial Sequence
  Target Specific Oligonucleotide
gcagtccatctaaggtggactgtaacttataaaggatagacatc

SEQ ID NO: 706 - DNA, 46 bp, Artificial Sequence
  Target Specific Oligonucleotide
tgtatatggagtaccctcttgctgtaacttataaaggatagacatc

SEQ ID NO: 707 - DNA, 44 bp, Artificial Sequence
  Target Specific Oligonucleotide
ctggatcaccaaaacggcaatgtaacttataaaggatagacatc

SEQ ID NO: 708 - DNA, 45 bp, Artificial Sequence
  Target Specific Oligonucleotide
ccaggataacctttacttgccagtaacttataaaggatagacatc

SEQ ID NO: 709 - DNA, 46 bp, Artificial Sequence
  Target Specific Oligonucleotide
atttccagctgcgatatctactcgtaacttataaaggatagacatc

SEQ ID NO: 710 - DNA, 47 bp, Artificial Sequence
  Target Specific Oligonucleotide
tttatagcagctagtcctttcttggtaacttataaaggatagacatc

SEQ ID NO: 711 - DNA, 46 bp, Artificial Sequence
  Target Specific Oligonucleotide
tgatatgagattttcccggatgggtaacttataaaggatagacatc

SEQ ID NO: 712 - DNA, 43 bp, Artificial Sequence
  Target Specific Oligonucleotide
tgacaacagttagggcggtggtaacttataaaggatagacatc

SEQ ID NO: 713 - DNA, 44 bp, Artificial Sequence
  Target Specific Oligonucleotide
ttggttcccgagtactgtctagtaacttataaaggatagacatc

SEQ ID NO: 714 - DNA, 45 bp, Artificial Sequence
  Target Specific Oligonucleotide
gtttagttccaacgccatctgtgtaacttataaaggatagacatc

SEQ ID NO: 715 - DNA, 45 bp, Artificial Sequence
  Target Specific Oligonucleotide
aagagttgcgacagtatcacccgtaacttataaaggatagacatc

SEQ ID NO: 716 - DNA, 44 bp, Artificial Sequence
  Target Specific Oligonucleotide
gctgctggatatccctcagaagtaacttataaaggatagacatc

SEQ ID NO: 717 - DNA, 46 bp, Artificial Sequence
  Target Specific Oligonucleotide
tgcaagacagactatgtctatgggtaacttataaaggatagacatc

SEQ ID NO: 718 - DNA, 46 bp, Artificial Sequence
  Target Specific Oligonucleotide
cttcaaggactaggtcaattgcagtaacttataaaggatagacatc

SEQ ID NO: 719 - DNA, 47 bp, Artificial Sequence
  Target Specific Oligonucleotide
gatatgctgcactaactagtcttggtaacttataaaggatagacatc

SEQ ID NO: 720 - DNA, 40 bp, Artificial Sequence
  Target Specific Oligonucleotide
atctcgagcaagacgttcagtgtaacggttatcgtggaaa

SEQ ID NO: 721 - DNA, 43 bp, Artificial Sequence
  Target Specific Oligonucleotide
ctgtccataattagtccatgaggagtaacggttatcgtggaaa

SEQ ID NO: 722 - DNA, 43 bp, Artificial Sequence
  Target Specific Oligonucleotide
atctttggattatactgcctgaccgtaacggttatcgtggaaa

SEQ ID NO: 723 - DNA, 39 bp, Artificial Sequence
  Target Specific Oligonucleotide
cagcaagcttgcgaccttgagtaacggttatcgtggaaa

SEQ ID NO: 724 - DNA, 44 bp, Artificial Sequence
  Target Specific Oligonucleotide
agttaaagttgagagatcatctccagtaacggttatcgtggaaa

SEQ ID NO: 725 - DNA, 44 bp, Artificial Sequence
  Target Specific Oligonucleotide
aggaatggatctatcactatttctagtaacggttatcgtggaaa

SEQ ID NO: 726 - DNA, 44 bp, Artificial Sequence
  Target Specific Oligonucleotide
cagcataatgattaggtatgcaaaagtaacggttatcgtggaaa

SEQ ID NO: 727 - DNA, 44 bp, Artificial Sequence
  Target Specific Oligonucleotide
ttcagtctgataaaatctacagtcagtaacggttatcgtggaaa

SEQ ID NO: 728 - DNA, 39 bp, Artificial Sequence
  Target Specific Oligonucleotide
tccaacacttcgtggggtccgtaacggttatcgtggaaa

SEQ ID NO: 729 - DNA, 39 bp, Artificial Sequence
  Target Specific Oligonucleotide
agggctacaatgtgatggccgtaacggttatcgtggaaa

SEQ ID NO: 730 - DNA, 42 bp, Artificial Sequence
  Target Specific Oligonucleotide
tcaaggtcataacctggttcatcgtaacggttatcgtggaaa

SEQ ID NO: 731 - DNA, 42 bp, Artificial Sequence
  Target Specific Oligonucleotide
cctacaacaaacttgtctggaatgtaacggttatcgtggaaa

SEQ ID NO: 732 - DNA, 40 bp, Artificial Sequence
  Target Specific Oligonucleotide
aaacaacaatccgcccaaagggtaacggttatcgtggaaa

SEQ ID NO: 733 - DNA, 41 bp, Artificial Sequence
  Target Specific Oligonucleotide
aaagctctactaagcagatggcgtaacggttatcgtggaaa

SEQ ID NO: 734 - DNA, 41 bp, Artificial Sequence
  Target Specific Oligonucleotide
actgctgacaaagattcactgggtaacggttatcgtggaaa

SEQ ID NO: 735 - DNA, 44 bp, Artificial Sequence
  Target Specific Oligonucleotide
ctgatagtctataggctcatagtgcgtaacggttatcgtggaaa

SEQ ID NO: 736 - DNA, 40 bp, Artificial Sequence
  Target Specific Oligonucleotide
atcactaatcacgacgccagggtaacggttatcgtggaaa

SEQ ID NO: 737 - DNA, 40 bp, Artificial Sequence
  Target Specific Oligonucleotide
tggcgatgtcaataggactccgtaacggttatcgtggaaa

SEQ ID NO: 738 - DNA, 42 bp, Artificial Sequence
  Target Specific Oligonucleotide
gaactctcatcttaggctttgtagtaacggttatcgtggaaa

SEQ ID NO: 739 - DNA, 44 bp, Artificial Sequence
  Target Specific Oligonucleotide
tcaacattactgaaacactactaaagtaacggttatcgtggaaa

SEQ ID NO: 740 - DNA, 38 bp, Artificial Sequence
  Target Specific Oligonucleotide
ggtcgccataacggagccggtaacggttatcgtggaaa

SEQ ID NO: 741 - DNA, 45 bp, Artificial Sequence
  Target Specific Oligonucleotide
ttcttacagatatgagttcaatgtttgtaacggttatcgtggaaa

SEQ ID NO: 742 - DNA, 44 bp, Artificial Sequence
  Target Specific Oligonucleotide
aggacagaacaaaacttcttagatggtaacggttatcgtggaaa

SEQ ID NO: 743 - DNA, 36 bp, Artificial Sequence
  Target Specific Oligonucleotide
tcgcagccgcctgaagcgtaacggttatcgtggaaa

SEQ ID NO: 744 - DNA, 41 bp, Artificial Sequence
  Target Specific Oligonucleotide
ggaatttggaaccggcttgcgtaacggtactttatctagct

SEQ ID NO: 745 - DNA, 46 bp, Artificial Sequence
  Target Specific Oligonucleotide
agagtcttgagtataatcttggtaggtaacggtactttatctagct

SEQ ID NO: 746 - DNA, 46 bp, Artificial Sequence
  Target Specific Oligonucleotide
attcaggaagcatacactaattcttgtaacggtactttatctagct

SEQ ID NO: 747 - DNA, 44 bp, Artificial Sequence
  Target Specific Oligonucleotide
tccagaaataagcgaaaatagcagtaacggtactttatctagct

SEQ ID NO: 748 - DNA, 47 bp, Artificial Sequence
  Target Specific Oligonucleotide
atcttttgaactactagctacaaaatgtaacggtactttatctagct

SEQ ID NO: 749 - DNA, 42 bp, Artificial Sequence
  Target Specific Oligonucleotide
taacaagctcgtaatggcccagtaacggtactttatctagct

SEQ ID NO: 750 - DNA, 42 bp, Artificial Sequence
  Target Specific Oligonucleotide
catccaaaccgtgtgaaagctgtaacggtactttatctagct

SEQ ID NO: 751 - DNA, 41 bp, Artificial Sequence
  Target Specific Oligonucleotide
acaacgccatgtgcgatttggtaacggtactttatctagct

SEQ ID NO: 752 - DNA, 45 bp, Artificial Sequence
  Target Specific Oligonucleotide
ctacctaccactaggtacctatcagtaacggtactttatctagct

SEQ ID NO: 753 - DNA, 43 bp, Artificial Sequence
  Target Specific Oligonucleotide
aggacatttacgtgtacagcacgtaacggtactttatctagct

SEQ ID NO: 754 - DNA, 43 bp, Artificial Sequence
  Target Specific Oligonucleotide
cgcctgaatacaacgagttaacgtaacggtactttatctagct

SEQ ID NO: 755 - DNA, 44 bp, Artificial Sequence
  Target Specific Oligonucleotide
cttccagattgtcgccgttaattgtaacggtactttatctagct

SEQ ID NO: 756 - DNA, 44 bp, Artificial Sequence
  Target Specific Oligonucleotide
cagatttacctaaaccaccaccagtaacggtactttatctagct

SEQ ID NO: 757 - DNA, 45 bp, Artificial Sequence
  Target Specific Oligonucleotide
tttgagctgatctagtaggtcttcgtaacggtactttatctagct

SEQ ID NO: 758 - DNA, 41 bp, Artificial Sequence
  Target Specific Oligonucleotide
tctgtagctcgatccctggagtaacggtactttatctagct

SEQ ID NO: 759 - DNA, 44 bp, Artificial Sequence
  Target Specific Oligonucleotide
caactgtcattcggttaaactgagtaacggtactttatctagct

SEQ ID NO: 760 - DNA, 44 bp, Artificial Sequence
  Target Specific Oligonucleotide
tttgcttccgttcatcattagtcgtaacggtactttatctagct

SEQ ID NO: 761 - DNA, 45 bp, Artificial Sequence
  Target Specific Oligonucleotide
acttccaatagtctggaaatcacagtaacggtactttatctagct

SEQ ID NO: 762 - DNA, 45 bp, Artificial Sequence
  Target Specific Oligonucleotide
tcatctgctggattaagctaatacgtaacggtactttatctagct

SEQ ID NO: 763 - DNA, 41 bp, Artificial Sequence
  Target Specific Oligonucleotide
agataggctcccgtatgcccgtaacggtactttatctagct

SEQ ID NO: 764 - DNA, 41 bp, Artificial Sequence
  Target Specific Oligonucleotide
gcttctcggctaccacagacgtaacggtactttatctagct

SEQ ID NO: 765 - DNA, 46 bp, Artificial Sequence
  Target Specific Oligonucleotide
agacttactcttaagaccatacttcgtaacggtactttatctagct

SEQ ID NO: 766 - DNA, 42 bp, Artificial Sequence
  Target Specific Oligonucleotide
gttgagaggtggcgtttttgagtaacggtactttatctagct

SEQ ID NO: 767 - DNA, 44 bp, Artificial Sequence
  Target Specific Oligonucleotide
tcaatacgtttctgcaagagtttgtaacggtactttatctagct

SEQ ID NO: 768 - DNA, 41 bp, Artificial Sequence
  Target Specific Oligonucleotide
ttatggcctctatagcatctcccgtaactccatctagaccg

SEQ ID NO: 769 - DNA, 40 bp, Artificial Sequence
  Target Specific Oligonucleotide
cagtccgggcaactatcttattgtaactccatctagaccg

SEQ ID NO: 770 - DNA, 42 bp, Artificial Sequence
  Target Specific Oligonucleotide
ttcaagatacgaataatcctccgagtaactccatctagaccg

SEQ ID NO: 771 - DNA, 40 bp, Artificial Sequence
  Target Specific Oligonucleotide
gtaggcgcttagcttgattttcgtaactccatctagaccg

SEQ ID NO: 772 - DNA, 38 bp, Artificial Sequence
  Target Specific Oligonucleotide
cttcaagtcgggctcttgcagtaactccatctagaccg

SEQ ID NO: 773 - DNA, 38 bp, Artificial Sequence
  Target Specific Oligonucleotide
agaatgaggcacgcaaagctgtaactccatctagaccg

SEQ ID NO: 774 - DNA, 40 bp, Artificial Sequence
  Target Specific Oligonucleotide
ctcagcatccgataggactttcgtaactccatctagaccg

SEQ ID NO: 775 - DNA, 40 bp, Artificial Sequence
  Target Specific Oligonucleotide
aagtcactataacacccgtcctgtaactccatctagaccg

SEQ ID NO: 776 - DNA, 39 bp, Artificial Sequence
  Target Specific Oligonucleotide
ttggcatggtagatcgtccaggtaactccatctagaccg

SEQ ID NO: 777 - DNA, 39 bp, Artificial Sequence
  Target Specific Oligonucleotide
caccttccaacgactcaatccgtaactccatctagaccg

SEQ ID NO: 778 - DNA, 42 bp, Artificial Sequence
  Target Specific Oligonucleotide
acaacctgtaagtgtagttctctggtaactccatctagaccg

SEQ ID NO: 779 - DNA, 43 bp, Artificial Sequence
  Target Specific Oligonucleotide
aaatcagatactcgtctacagaatggtaactccatctagaccg

SEQ ID NO: 780 - DNA, 38 bp, Artificial Sequence
  Target Specific Oligonucleotide
agcagcaagtcgcattttccgtaactccatctagaccg

SEQ ID NO: 781 - DNA, 39 bp, Artificial Sequence
  Target Specific Oligonucleotide
actggcatggcggataaaaaggtaactccatctagaccg

SEQ ID NO: 782 - DNA, 38 bp, Artificial Sequence
  Target Specific Oligonucleotide
cgggtgctctcgtgaacaaagtaactccatctagaccg

SEQ ID NO: 783 - DNA, 41 bp, Artificial Sequence
  Target Specific Oligonucleotide
gatcgaagagtcgagataaggatgtaactccatctagaccg

SEQ ID NO: 784 - DNA, 41 bp, Artificial Sequence
  Target Specific Oligonucleotide
ttgttgaagacgcttccataagtgtaactccatctagaccg

SEQ ID NO: 785 - DNA, 37 bp, Artificial Sequence
  Target Specific Oligonucleotide
gtggtgacgcagaatcccggtaactccatctagaccg

SEQ ID NO: 786 - DNA, 45 bp, Artificial Sequence
  Target Specific Oligonucleotide
cattataaagacgtaataatttagggtgtaactccatctagaccg

SEQ ID NO: 787 - DNA, 39 bp, Artificial Sequence
  Target Specific Oligonucleotide
gctcccactcctagacatgttgtaactccatctagaccg

SEQ ID NO: 788 - DNA, 40 bp, Artificial Sequence
  Target Specific Oligonucleotide
tttgttacacggttaaacagccgtaactccatctagaccg

SEQ ID NO: 789 - DNA, 42 bp, Artificial Sequence
  Target Specific Oligonucleotide
gaggtaaataaacgtcgataggaagtaactccatctagaccg

SEQ ID NO: 790 - DNA, 40 bp, Artificial Sequence
  Target Specific Oligonucleotide
ttaatgtggagtgcaagttggggtaactccatctagaccg

SEQ ID NO: 791 - DNA, 41 bp, Artificial Sequence
  Target Specific Oligonucleotide
ccttgtatcttagcttgggatgtgtaactccatctagaccg

SEQ ID NO: 792 - DNA, 43 bp, Artificial Sequence
  Target Specific Oligonucleotide
tcaatgatcagcggatacctattgtaactcagtagatatagcg

SEQ ID NO: 793 - DNA, 43 bp, Artificial Sequence
  Target Specific Oligonucleotide
cgtgtgatcttacgatccttatagtaactcagtagatatagcg

SEQ ID NO: 794 - DNA, 43 bp, Artificial Sequence
  Target Specific Oligonucleotide
tcatcctctacgagtctatcttggtaactcagtagatatagcg

SEQ ID NO: 795 - DNA, 40 bp, Artificial Sequence
  Target Specific Oligonucleotide
tcaaagtaacccgcgtaggcgtaactcagtagatatagcg

SEQ ID NO: 796 - DNA, 41 bp, Artificial Sequence
  Target Specific Oligonucleotide
ataggacgcatactgtcgcaagtaactcagtagatatagcg

SEQ ID NO: 797 - DNA, 41 bp, Artificial Sequence
  Target Specific Oligonucleotide
ttatgtccgtcgaccttgcatgtaactcagtagatatagcg

SEQ ID NO: 798 - DNA, 42 bp, Artificial Sequence
  Target Specific Oligonucleotide
ttccaaggaacggacttctagggtaactcagtagatatagcg

SEQ ID NO: 799 - DNA, 42 bp, Artificial Sequence
  Target Specific Oligonucleotide
ataggtacgaacgctctcgatcgtaactcagtagatatagcg

SEQ ID NO: 800 - DNA, 42 bp, Artificial Sequence
  Target Specific Oligonucleotide
tcttggatgatcgcatgaaaccgtaactcagtagatatagcg

SEQ ID NO: 801 - DNA, 42 bp, Artificial Sequence
  Target Specific Oligonucleotide
acaacccaattaacgtgaagccgtaactcagtagatatagcg

SEQ ID NO: 802 - DNA, 41 bp, Artificial Sequence
  Target Specific Oligonucleotide
atgcggtataacgcacgttccgtaactcagtagatatagcg

SEQ ID NO: 803 - DNA, 41 bp, Artificial Sequence
  Target Specific Oligonucleotide
ctcaagtcacgacgcagttctgtaactcagtagatatagcg

SEQ ID NO: 804 - DNA, 40 bp, Artificial Sequence
  Target Specific Oligonucleotide
gcaagtcacgaacgtgactcgtaactcagtagatatagcg

SEQ ID NO: 805 - DNA, 40 bp, Artificial Sequence
  Target Specific Oligonucleotide
ttggcgttatactgcgccacgtaactcagtagatatagcg

SEQ ID NO: 806 - DNA, 40 bp, Artificial Sequence
  Target Specific Oligonucleotide
agtggtgcgtatcgtaagcggtaactcagtagatatagcg

SEQ ID NO: 807 - DNA, 40 bp, Artificial Sequence
  Target Specific Oligonucleotide
atccaggcgacgaaacgagagtaactcagtagatatagcg

SEQ ID NO: 808 - DNA, 40 bp, Artificial Sequence
  Target Specific Oligonucleotide
ttggatgaatcggcaggctggtaactcagtagatatagcg

SEQ ID NO: 809 - DNA, 43 bp, Artificial Sequence
  Target Specific Oligonucleotide
tcagtcaattcgtaatgtacaccgtaactcagtagatatagcg

SEQ ID NO: 810 - DNA, 41 bp, Artificial Sequence
  Target Specific Oligonucleotide
catcgaagacgatccactggcgtaactcagtagatatagcg

SEQ ID NO: 811 - DNA, 39 bp, Artificial Sequence
  Target Specific Oligonucleotide
cacggatcaagcggcacttgtaactcagtagatatagcg

SEQ ID NO: 812 - DNA, 39 bp, Artificial Sequence
  Target Specific Oligonucleotide
tccaccgtatcgcaagctggtaactcagtagatatagcg

SEQ ID NO: 813 - DNA, 41 bp, Artificial Sequence
  Target Specific Oligonucleotide
atgcaaagtttcgtagggcgagtaactcagtagatatagcg

SEQ ID NO: 814 - DNA, 38 bp, Artificial Sequence
  Target Specific Oligonucleotide
gcgttggcacgacgacatgtaactcagtagatatagcg

SEQ ID NO: 815 - DNA, 38 bp, Artificial Sequence
  Target Specific Oligonucleotide
ttgcagacggacgcccaagtaactcagtagatatagcg

SEQ ID NO: 816 - DNA, 40 bp, Artificial Sequence
  Target Specific Oligonucleotide
agctgtatcgtcaaggcactcgtaaccttttgaaacgcta

SEQ ID NO: 817 - DNA, 39 bp, Artificial Sequence
  Target Specific Oligonucleotide
ctcgctcccagtccgaaatggtaaccttttgaaacgcta

SEQ ID NO: 818 - DNA, 44 bp, Artificial Sequence
  Target Specific Oligonucleotide
acttgtactagtatgccttaagaaagtaaccttttgaaacgcta

SEQ ID NO: 819 - DNA, 42 bp, Artificial Sequence
  Target Specific Oligonucleotide
gtgaccatgactaatagcagtgggtaaccttttgaaacgcta

SEQ ID NO: 820 - DNA, 41 bp, Artificial Sequence
  Target Specific Oligonucleotide
gctgtgtcgagaatatccaagagtaaccttttgaaacgcta

SEQ ID NO: 821 - DNA, 41 bp, Artificial Sequence
  Target Specific Oligonucleotide
cctactgtcgctaatggattgggtaaccttttgaaacgcta

SEQ ID NO: 822 - DNA, 41 bp, Artificial Sequence
  Target Specific Oligonucleotide
acactagcactacctaaggaccgtaaccttttgaaacgcta

SEQ ID NO: 823 - DNA, 41 bp, Artificial Sequence
  Target Specific Oligonucleotide
gtctgtgaactagttcaggcacgtaaccttttgaaacgcta

SEQ ID NO: 824 - DNA, 40 bp, Artificial Sequence
  Target Specific Oligonucleotide
ctaggtcagcgcaaccaaatggtaaccttttgaaacgcta

SEQ ID NO: 825 - DNA, 42 bp, Artificial Sequence
  Target Specific Oligonucleotide
cctaataagctataactggcccagtaaccttttgaaacgcta

SEQ ID NO: 826 - DNA, 43 bp, Artificial Sequence
  Target Specific Oligonucleotide
ctaaattcctatgcagtgtgactcgtaaccttttgaaacgcta

SEQ ID NO: 827 - DNA, 42 bp, Artificial Sequence
  Target Specific Oligonucleotide
atcttgttgagctatccaaactggtaaccttttgaaacgcta

SEQ ID NO: 828 - DNA, 42 bp, Artificial Sequence
  Target Specific Oligonucleotide
aaatacgcatcgtgttatctctggtaaccttttgaaacgcta

SEQ ID NO: 829 - DNA, 42 bp, Artificial Sequence
  Target Specific Oligonucleotide
tgttaagagaactagccaaacctgtaaccttttgaaacgcta

SEQ ID NO: 830 - DNA, 43 bp, Artificial Sequence
  Target Specific Oligonucleotide
cataaagtagcttgatcgaagagtgtaaccttttgaaacgcta

SEQ ID NO: 831 - DNA, 41 bp, Artificial Sequence
  Target Specific Oligonucleotide
taagcaaacacgcctttacatagtaaccttttgaaacgcta

SEQ ID NO: 832 - DNA, 44 bp, Artificial Sequence
  Target Specific Oligonucleotide
aaggccttagtaagatattacagacgtaaccttttgaaacgcta

SEQ ID NO: 833 - DNA, 45 bp, Artificial Sequence
  Target Specific Oligonucleotide
acaatatacgtctgctatattcttccgtaaccttttgaaacgcta

SEQ ID NO: 834 - DNA, 41 bp, Artificial Sequence
  Target Specific Oligonucleotide
tccactggatagggttctgtctgtaaccttttgaaacgcta

SEQ ID NO: 835 - DNA, 44 bp, Artificial Sequence
  Target Specific Oligonucleotide
aagttatactatgaaagagcagtctgtaaccttttgaaacgcta

SEQ ID NO: 836 - DNA, 44 bp, Artificial Sequence
  Target Specific Oligonucleotide
acatgttaatgcctaagtctatgtagtaaccttttgaaacgcta

SEQ ID NO: 837 - DNA, 40 bp, Artificial Sequence
  Target Specific Oligonucleotide
gcctagaatgcctacttgggagtaaccttttgaaacgcta

SEQ ID NO: 838 - DNA, 41 bp, Artificial Sequence
  Target Specific Oligonucleotide
tcagtggaatcgtagcaaaacagtaaccttttgaaacgcta

SEQ ID NO: 839 - DNA, 45 bp, Artificial Sequence
  Target Specific Oligonucleotide
acaataattaggagtagtacagttcagtaaccttttgaaacgcta

SEQ ID NO: 840 - DNA, 42 bp, Artificial Sequence
  Target Specific Oligonucleotide
ttccactcggataagatgctgagtaactcagacatgtagatt

SEQ ID NO: 841 - DNA, 42 bp, Artificial Sequence
  Target Specific Oligonucleotide
aatactccacacgcaaatttccgtaactcagacatgtagatt

SEQ ID NO: 842 - DNA, 40 bp, Artificial Sequence
  Target Specific Oligonucleotide
ggccagaccatcgctatctggtaactcagacatgtagatt

SEQ ID NO: 843 - DNA, 42 bp, Artificial Sequence
  Target Specific Oligonucleotide
caccacactatgtcgaaaagtggtaactcagacatgtagatt

SEQ ID NO: 844 - DNA, 42 bp, Artificial Sequence
  Target Specific Oligonucleotide
ccctttcttgcggagattctctgtaactcagacatgtagatt

SEQ ID NO: 845 - DNA, 40 bp, Artificial Sequence
  Target Specific Oligonucleotide
acaaacacgcacctcaaagcgtaactcagacatgtagatt

SEQ ID NO: 846 - DNA, 42 bp, Artificial Sequence
  Target Specific Oligonucleotide
cccagtagattaccactggagtgtaactcagacatgtagatt

SEQ ID NO: 847 - DNA, 39 bp, Artificial Sequence
  Target Specific Oligonucleotide
gcagtgctcgcttagtgctgtaactcagacatgtagatt

SEQ ID NO: 848 - DNA, 40 bp, Artificial Sequence
  Target Specific Oligonucleotide
ggctcgacgctaggatctgagtaactcagacatgtagatt

SEQ ID NO: 849 - DNA, 39 bp, Artificial Sequence
  Target Specific Oligonucleotide
aggatgggcctccggttcagtaactcagacatgtagatt

SEQ ID NO: 850 - DNA, 40 bp, Artificial Sequence
  Target Specific Oligonucleotide
ggaacatctcgaagcgctcagtaactcagacatgtagatt

SEQ ID NO: 851 - DNA, 40 bp, Artificial Sequence
  Target Specific Oligonucleotide
agacggaaaccgtagctgccgtaactcagacatgtagatt

SEQ ID NO: 852 - DNA, 41 bp, Artificial Sequence
  Target Specific Oligonucleotide
tgttcaatatcgtccggggacgtaactcagacatgtagatt

SEQ ID NO: 853 - DNA, 40 bp, Artificial Sequence
  Target Specific Oligonucleotide
cattgcttgggacggcaagggtaactcagacatgtagatt

SEQ ID NO: 854 - DNA, 42 bp, Artificial Sequence
  Target Specific Oligonucleotide
agtggagaatgtcagtctgagtgtaactcagacatgtagatt

SEQ ID NO: 855 - DNA, 43 bp, Artificial Sequence
  Target Specific Oligonucleotide
agttttttatggcgggaggtagagtaactcagacatgtagatt

SEQ ID NO: 856 - DNA, 41 bp, Artificial Sequence
  Target Specific Oligonucleotide
tagaaactaccaacccaccgagtaactcagacatgtagatt

SEQ ID NO: 857 - DNA, 42 bp, Artificial Sequence
  Target Specific Oligonucleotide
ttccctggttagtacggtgaaggtaactcagacatgtagatt

SEQ ID NO: 858 - DNA, 42 bp, Artificial Sequence
  Target Specific Oligonucleotide
taagctggtatgtcctactcccgtaactcagacatgtagatt

SEQ ID NO: 859 - DNA, 41 bp, Artificial Sequence
  Target Specific Oligonucleotide
ctgacgcacacctattgcaaggtaactcagacatgtagatt

SEQ ID NO: 860 - DNA, 39 bp, Artificial Sequence
  Target Specific Oligonucleotide
tcaccgtcgtggaaagcacgtaactcagacatgtagatt

SEQ ID NO: 861 - DNA, 44 bp, Artificial Sequence
  Target Specific Oligonucleotide
gtaaactaacccttaactgcaagagtaactcagacatgtagatt

SEQ ID NO: 862 - DNA, 44 bp, Artificial Sequence
  Target Specific Oligonucleotide
tttaggtactaaggttcaccaagagtaactcagacatgtagatt

SEQ ID NO: 863 - DNA, 46 bp, Artificial Sequence
  Target Specific Oligonucleotide
tcatcatatacaagagatgaaatcctgtaactcagacatgtagatt

SEQ ID NO: 864 - DNA, 41 bp, Artificial Sequence
  Target Specific Oligonucleotide
ccatggtgacgatcctcacggtaactaacataaattcgatg

SEQ ID NO: 865 - DNA, 42 bp, Artificial Sequence
  Target Specific Oligonucleotide
gtgaacgtcttgggttgcttcgtaactaacataaattcgatg

SEQ ID NO: 866 - DNA, 39 bp, Artificial Sequence
  Target Specific Oligonucleotide
ggcctggacggcgtaagagtaactaacataaattcgatg

SEQ ID NO: 867 - DNA, 40 bp, Artificial Sequence
  Target Specific Oligonucleotide
gaagctcatgcgggaagcggtaactaacataaattcgatg

SEQ ID NO: 868 - DNA, 43 bp, Artificial Sequence
  Target Specific Oligonucleotide
tgtcactgtacgagatgtttcggtaactaacataaattcgatg

SEQ ID NO: 869 - DNA, 43 bp, Artificial Sequence
  Target Specific Oligonucleotide
ttgaagcttggtatgtcaggaggtaactaacataaattcgatg

SEQ ID NO: 870 - DNA, 42 bp, Artificial Sequence
  Target Specific Oligonucleotide
ggttcccgatacatagctggagtaactaacataaattcgatg

SEQ ID NO: 871 - DNA, 44 bp, Artificial Sequence
  Target Specific Oligonucleotide
atctttgaagtcgaaagacaggcgtaactaacataaattcgatg

SEQ ID NO: 872 - DNA, 44 bp, Artificial Sequence
  Target Specific Oligonucleotide
gcttattaacacgaagctttgaggtaactaacataaattcgatg

SEQ ID NO: 873 - DNA, 42 bp, Artificial Sequence
  Target Specific Oligonucleotide
tttcattccacgggaaggagagtaactaacataaattcgatg

SEQ ID NO: 874 - DNA, 41 bp, Artificial Sequence
  Target Specific Oligonucleotide
tcagtgcttcgttggccattgtaactaacataaattcgatg

SEQ ID NO: 875 - DNA, 43 bp, Artificial Sequence
  Target Specific Oligonucleotide
ctctgcgtaccaagcagtaattgtaactaacataaattcgatg

SEQ ID NO: 876 - DNA, 43 bp, Artificial Sequence
  Target Specific Oligonucleotide
tttcaaggcctcgatttctgtcgtaactaacataaattcgatg

SEQ ID NO: 877 - DNA, 43 bp, Artificial Sequence
  Target Specific Oligonucleotide
tagttcacaccgtacatctccagtaactaacataaattcgatg

SEQ ID NO: 878 - DNA, 42 bp, Artificial Sequence
  Target Specific Oligonucleotide
cccacatttccggagtcatctgtaactaacataaattcgatg

SEQ ID NO: 879 - DNA, 45 bp, Artificial Sequence
  Target Specific Oligonucleotide
ggtcctgagctatcttcagatattgtaactaacataaattcgatg

SEQ ID NO: 880 - DNA, 39 bp, Artificial Sequence
  Target Specific Oligonucleotide
tcctccgtgcgaatcgctgtaactaacataaattcgatg

SEQ ID NO: 881 - DNA, 43 bp, Artificial Sequence
  Target Specific Oligonucleotide
gccctttttattccggattgcagtaactaacataaattcgatg

SEQ ID NO: 882 - DNA, 40 bp, Artificial Sequence
  Target Specific Oligonucleotide
gctgtctcctcagaccgcagtaactaacataaattcgatg

SEQ ID NO: 883 - DNA, 41 bp, Artificial Sequence
  Target Specific Oligonucleotide
cacccctgtcggagttctcagtaactaacataaattcgatg

SEQ ID NO: 884 - DNA, 46 bp, Artificial Sequence
  Target Specific Oligonucleotide
cagatttattaccctttttggaagcgtaactaacataaattcgatg

SEQ ID NO: 885 - DNA, 39 bp, Artificial Sequence
  Target Specific Oligonucleotide
tcatgggcgggtacgtgggtaactaacataaattcgatg

SEQ ID NO: 886 - DNA, 42 bp, Artificial Sequence
  Target Specific Oligonucleotide
caaaaatccccgcttgtgaacgtaactaacataaattcgatg

SEQ ID NO: 887 - DNA, 42 bp, Artificial Sequence
  Target Specific Oligonucleotide
ctggggtcgtagtcaccatacgtaactaacataaattcgatg

SEQ ID NO: 888 - DNA, 39 bp, Artificial Sequence
  Target Specific Oligonucleotide
cacacgataccggcaaagaaggtaacgtgacgattaccc

SEQ ID NO: 889 - DNA, 38 bp, Artificial Sequence
  Target Specific Oligonucleotide
ggtgtcagtctccgacgtgagtaacgtgacgattaccc

SEQ ID NO: 890 - DNA, 39 bp, Artificial Sequence
  Target Specific Oligonucleotide
gtgatggtgatcatctgggccgtaacgtgacgattaccc

SEQ ID NO: 891 - DNA, 36 bp, Artificial Sequence
  Target Specific Oligonucleotide
ttggcctcgggaccaagcgtaacgtgacgattaccc

SEQ ID NO: 892 - DNA, 39 bp, Artificial Sequence
  Target Specific Oligonucleotide
ctgtcatcttggtcaggtggtgtaacgtgacgattaccc

SEQ ID NO: 893 - DNA, 38 bp, Artificial Sequence
  Target Specific Oligonucleotide
atcaccttgccgaaagtgccgtaacgtgacgattaccc

SEQ ID NO: 894 - DNA, 37 bp, Artificial Sequence
  Target Specific Oligonucleotide
actgcacggccgtagtcatgtaacgtgacgattaccc

SEQ ID NO: 895 - DNA, 37 bp, Artificial Sequence
  Target Specific Oligonucleotide
aggtactcaggtgtgccgcgtaacgtgacgattaccc

SEQ ID NO: 896 - DNA, 38 bp, Artificial Sequence
  Target Specific Oligonucleotide
gaaggtgcgttcgatgacaggtaacgtgacgattaccc

SEQ ID NO: 897 - DNA, 39 bp, Artificial Sequence
  Target Specific Oligonucleotide
ctcctcaggagtctccacatggtaacgtgacgattaccc

SEQ ID NO: 898 - DNA, 40 bp, Artificial Sequence
  Target Specific Oligonucleotide
gtctggaaagagtacttcaggggtaacgtgacgattaccc

SEQ ID NO: 899 - DNA, 36 bp, Artificial Sequence
  Target Specific Oligonucleotide
caggacgcggttctcggtgtaacgtgacgattaccc

SEQ ID NO: 900 - DNA, 38 bp, Artificial Sequence
  Target Specific Oligonucleotide
atctcagcgccatagaagcggtaacgtgacgattaccc

SEQ ID NO: 901 - DNA, 37 bp, Artificial Sequence
  Target Specific Oligonucleotide
tcgttcatggtcacgcggtgtaacgtgacgattaccc

SEQ ID NO: 902 - DNA, 39 bp, Artificial Sequence
  Target Specific Oligonucleotide
cagcccgaagtctgtgatcttgtaacgtgacgattaccc

SEQ ID NO: 903 - DNA, 37 bp, Artificial Sequence
  Target Specific Oligonucleotide
tcatggtggcaccgtccttgtaacgtgacgattaccc

SEQ ID NO: 904 - DNA, 38 bp, Artificial Sequence
  Target Specific Oligonucleotide
ggtagtccagggctgacacagtaacgtgacgattaccc

SEQ ID NO: 905 - DNA, 41 bp, Artificial Sequence
  Target Specific Oligonucleotide
ccatcattcttgaggaggaagtagtaacgtgacgattaccc

SEQ ID NO: 906 - DNA, 37 bp, Artificial Sequence
  Target Specific Oligonucleotide
acaaagcagaggcggtcgtgtaacgtgacgattaccc

SEQ ID NO: 907 - DNA, 38 bp, Artificial Sequence
  Target Specific Oligonucleotide
cggtcctcggagaacacacggtaacgtgacgattaccc

SEQ ID NO: 908 - DNA, 37 bp, Artificial Sequence
  Target Specific Oligonucleotide
gagcctcacgttggtccacgtaacgtgacgattaccc

SEQ ID NO: 909 - DNA, 40 bp, Artificial Sequence
  Target Specific Oligonucleotide
ctccttgtagccaatgaaggtggtaacgtgacgattaccc

SEQ ID NO: 910 - DNA, 38 bp, Artificial Sequence
  Target Specific Oligonucleotide
ttcacaatagccacgtcgctgtaacgtgacgattaccc

SEQ ID NO: 911 - DNA, 37 bp, Artificial Sequence
  Target Specific Oligonucleotide
ccctcgtttgtgcagccaagtaacgtgacgattaccc

SEQ ID NO: 912 - DNA, 43 bp, Artificial Sequence
  Target Specific Oligonucleotide
agctatgtccgctatcttcatcggtaactctcttctattgcag

SEQ ID NO: 913 - DNA, 41 bp, Artificial Sequence
  Target Specific Oligonucleotide
ttggcacatcgagacatggttgtaactctcttctattgcag

SEQ ID NO: 914 - DNA, 42 bp, Artificial Sequence
  Target Specific Oligonucleotide
ccatcttggtcgtactttgtgagtaactctcttctattgcag

SEQ ID NO: 915 - DNA, 41 bp, Artificial Sequence
  Target Specific Oligonucleotide
ccaactgagcatacgctaggagtaactctcttctattgcag

SEQ ID NO: 916 - DNA, 41 bp, Artificial Sequence
  Target Specific Oligonucleotide
tcacgtactagccgttccatcgtaactctcttctattgcag

SEQ ID NO: 917 - DNA, 43 bp, Artificial Sequence
  Target Specific Oligonucleotide
tttctccaagtcgtctctcatctgtaactctcttctattgcag

SEQ ID NO: 918 - DNA, 42 bp, Artificial Sequence
  Target Specific Oligonucleotide
tcgtaagaaacgccactagaaagtaactctcttctattgcag

SEQ ID NO: 919 - DNA, 39 bp, Artificial Sequence
  Target Specific Oligonucleotide
cactggcgtgcctaaaccagtaactctcttctattgcag

SEQ ID NO: 920 - DNA, 45 bp, Artificial Sequence
  Target Specific Oligonucleotide
acatatcatacgtggacattagaacgtaactctcttctattgcag

SEQ ID NO: 921 - DNA, 40 bp, Artificial Sequence
  Target Specific Oligonucleotide
gccgatggaatgctccatccgtaactctcttctattgcag

SEQ ID NO: 922 - DNA, 43 bp, Artificial Sequence
  Target Specific Oligonucleotide
gattagcaatcggtcacaaagacgtaactctcttctattgcag

SEQ ID NO: 923 - DNA, 43 bp, Artificial Sequence
  Target Specific Oligonucleotide
ggtgtagtagtacacattggagcgtaactctcttctattgcag

SEQ ID NO: 924 - DNA, 42 bp, Artificial Sequence
  Target Specific Oligonucleotide
gatgaaacttcggttgtcagctgtaactctcttctattgcag

SEQ ID NO: 925 - DNA, 42 bp, Artificial Sequence
  Target Specific Oligonucleotide
ttcttgaggctagcaacttgtggtaactctcttctattgcag

SEQ ID NO: 926 - DNA, 43 bp, Artificial Sequence
  Target Specific Oligonucleotide
gtcccaaaactcgattagcttccgtaactctcttctattgcag

SEQ ID NO: 927 - DNA, 42 bp, Artificial Sequence
  Target Specific Oligonucleotide
tattcgtggaacccctaacagcgtaactctcttctattgcag

SEQ ID NO: 928 - DNA, 43 bp, Artificial Sequence
  Target Specific Oligonucleotide
caaagttgatatcgcccaaaatggtaactctcttctattgcag

SEQ ID NO: 929 - DNA, 42 bp, Artificial Sequence
  Target Specific Oligonucleotide
tagatccaagcggttccatttcgtaactctcttctattgcag

SEQ ID NO: 930 - DNA, 42 bp, Artificial Sequence
  Target Specific Oligonucleotide
tgaccagttcccgtaaaacactgtaactctcttctattgcag

SEQ ID NO: 931 - DNA, 41 bp, Artificial Sequence
  Target Specific Oligonucleotide
tggcacatggtcgatacacatgtaactctcttctattgcag

SEQ ID NO: 932 - DNA, 41 bp, Artificial Sequence
  Target Specific Oligonucleotide
tgacagcacaacgatcacaacgtaactctcttctattgcag

SEQ ID NO: 933 - DNA, 42 bp, Artificial Sequence
  Target Specific Oligonucleotide
aagctcatgaactatgccagaggtaactctcttctattgcag

SEQ ID NO: 934 - DNA, 41 bp, Artificial Sequence
  Target Specific Oligonucleotide
cgtcatacctgaccgagtactgtaactctcttctattgcag

SEQ ID NO: 935 - DNA, 43 bp, Artificial Sequence
  Target Specific Oligonucleotide
tcttgattgtcgagaaagacttggtaactctcttctattgcag

SEQ ID NO: 936 - DNA, 42 bp, Artificial Sequence
  Target Specific Oligonucleotide
tactgagccctatcggaaagtgtaactgatcttcatattacg

SEQ ID NO: 937 - DNA, 42 bp, Artificial Sequence
  Target Specific Oligonucleotide
ccaaatcccttcgcagcaattgtaactgatcttcatattacg

SEQ ID NO: 938 - DNA, 42 bp, Artificial Sequence
  Target Specific Oligonucleotide
gccatgatcaatcgcatcactgtaactgatcttcatattacg

SEQ ID NO: 939 - DNA, 43 bp, Artificial Sequence
  Target Specific Oligonucleotide
atcatccagattcgaaccgtccgtaactgatcttcatattacg

SEQ ID NO: 940 - DNA, 43 bp, Artificial Sequence
  Target Specific Oligonucleotide
ggactacattcgtgaagaagctgtaactgatcttcatattacg

SEQ ID NO: 941 - DNA, 42 bp, Artificial Sequence
  Target Specific Oligonucleotide
ggagaggatccgtccaagaaagtaactgatcttcatattacg

SEQ ID NO: 942 - DNA, 42 bp, Artificial Sequence
  Target Specific Oligonucleotide
gtcccagcattagaacctgtggtaactgatcttcatattacg

SEQ ID NO: 943 - DNA, 42 bp, Artificial Sequence
  Target Specific Oligonucleotide
actctcagccagacctttagcgtaactgatcttcatattacg

SEQ ID NO: 944 - DNA, 44 bp, Artificial Sequence
  Target Specific Oligonucleotide
catagtgtcatgtgcatgtagtcgtaactgatcttcatattacg

SEQ ID NO: 945 - DNA, 44 bp, Artificial Sequence
  Target Specific Oligonucleotide
ccctgaccagcatattatctacagtaactgatcttcatattacg

SEQ ID NO: 946 - DNA, 41 bp, Artificial Sequence
  Target Specific Oligonucleotide
cagcgcctgttacaagctctgtaactgatcttcatattacg

SEQ ID NO: 947 - DNA, 41 bp, Artificial Sequence
  Target Specific Oligonucleotide
cgccagtcttaactgccatggtaactgatcttcatattacg

SEQ ID NO: 948 - DNA, 43 bp, Artificial Sequence
  Target Specific Oligonucleotide
tcttcccaacaccgtaatctttgtaactgatcttcatattacg

SEQ ID NO: 949 - DNA, 43 bp, Artificial Sequence
  Target Specific Oligonucleotide
gtgacttatgttcgggtctctcgtaactgatcttcatattacg

SEQ ID NO: 950 - DNA, 43 bp, Artificial Sequence
  Target Specific Oligonucleotide
tgttaggttctagggccatagcgtaactgatcttcatattacg

SEQ ID NO: 951 - DNA, 42 bp, Artificial Sequence
  Target Specific Oligonucleotide
ctggatagctttgggcaactcgtaactgatcttcatattacg

SEQ ID NO: 952 - DNA, 45 bp, Artificial Sequence
  Target Specific Oligonucleotide
tgcagacagggtatgtatatttgggtaactgatcttcatattacg

SEQ ID NO: 953 - DNA, 44 bp, Artificial Sequence
  Target Specific Oligonucleotide
aagaaaatatagctgagctccctgtaactgatcttcatattacg

SEQ ID NO: 954 - DNA, 40 bp, Artificial Sequence
  Target Specific Oligonucleotide
gagaccggagaaacccggcgtaactgatcttcatattacg

SEQ ID NO: 955 - DNA, 40 bp, Artificial Sequence
  Target Specific Oligonucleotide
ccgcaggagaccgaggtctgtaactgatcttcatattacg

SEQ ID NO: 956 - DNA, 42 bp, Artificial Sequence
  Target Specific Oligonucleotide
aggctagggctcaagacaagggtaactgatcttcatattacg

SEQ ID NO: 957 - DNA, 38 bp, Artificial Sequence
  Target Specific Oligonucleotide
gcgtccggtgtcgccatgtaactgatcttcatattacg

SEQ ID NO: 958 - DNA, 41 bp, Artificial Sequence
  Target Specific Oligonucleotide
actctgggtcggcttgatccgtaactgatcttcatattacg

SEQ ID NO: 959 - DNA, 41 bp, Artificial Sequence
  Target Specific Oligonucleotide
catgggaggatcgagcctgggtaactgatcttcatattacg

SEQ ID NO: 960 - DNA, 39 bp, Artificial Sequence
  Target Specific Oligonucleotide
ggcactggaaacgattgacttgtaacggcaatacatcgt

SEQ ID NO: 961 - DNA, 38 bp, Artificial Sequence
  Target Specific Oligonucleotide
tcaggcattcgttcacctgtgtaacggcaatacatcgt

SEQ ID NO: 962 - DNA, 40 bp, Artificial Sequence
  Target Specific Oligonucleotide
actgtagcgattaatgccatccgtaacggcaatacatcgt

SEQ ID NO: 963 - DNA, 38 bp, Artificial Sequence
  Target Specific Oligonucleotide
atagcctccattgcggttgggtaacggcaatacatcgt

SEQ ID NO: 964 - DNA, 40 bp, Artificial Sequence
  Target Specific Oligonucleotide
cattcgggtgatcgatacacttgtaacggcaatacatcgt

SEQ ID NO: 965 - DNA, 39 bp, Artificial Sequence
  Target Specific Oligonucleotide
gcacactcatcgagttgctccgtaacggcaatacatcgt

SEQ ID NO: 966 - DNA, 37 bp, Artificial Sequence
  Target Specific Oligonucleotide
tcccgcttaatgcgcaggtgtaacggcaatacatcgt

SEQ ID NO: 967 - DNA, 37 bp, Artificial Sequence
  Target Specific Oligonucleotide
ggtcccctgtatggcgtgagtaacggcaatacatcgt

SEQ ID NO: 968 - DNA, 38 bp, Artificial Sequence
  Target Specific Oligonucleotide
aacacactggcggttgtcaagtaacggcaatacatcgt

SEQ ID NO: 969 - DNA, 39 bp, Artificial Sequence
  Target Specific Oligonucleotide
cacacatcgacgaaggtttcagtaacggcaatacatcgt

SEQ ID NO: 970 - DNA, 39 bp, Artificial Sequence
  Target Specific Oligonucleotide
ggctcttgcacgagttaacttgtaacggcaatacatcgt

SEQ ID NO: 971 - DNA, 39 bp, Artificial Sequence
  Target Specific Oligonucleotide
atgtcaatggtacaccgctgagtaacggcaatacatcgt

SEQ ID NO: 972 - DNA, 38 bp, Artificial Sequence
  Target Specific Oligonucleotide
gccatgcttacgctttcgttgtaacggcaatacatcgt

SEQ ID NO: 973 - DNA, 40 bp, Artificial Sequence
  Target Specific Oligonucleotide
ggtataccagtaacacggaccagtaacggcaatacatcgt

SEQ ID NO: 974 - DNA, 40 bp, Artificial Sequence
  Target Specific Oligonucleotide
gactctctcacgcataaacctggtaacggcaatacatcgt

SEQ ID NO: 975 - DNA, 40 bp, Artificial Sequence
  Target Specific Oligonucleotide
aggagatgcgtaggtcaattcagtaacggcaatacatcgt

SEQ ID NO: 976 - DNA, 39 bp, Artificial Sequence
  Target Specific Oligonucleotide
ccaacaggacgctagtgtagagtaacggcaatacatcgt

SEQ ID NO: 977 - DNA, 42 bp, Artificial Sequence
  Target Specific Oligonucleotide
cagtaccaattaggttagcttctggtaacggcaatacatcgt

SEQ ID NO: 978 - DNA, 38 bp, Artificial Sequence
  Target Specific Oligonucleotide
tccatggccgtcgatcaatggtaacggcaatacatcgt

SEQ ID NO: 979 - DNA, 40 bp, Artificial Sequence
  Target Specific Oligonucleotide
tagtcaggaatatgcggtcattgtaacggcaatacatcgt

SEQ ID NO: 980 - DNA, 41 bp, Artificial Sequence
  Target Specific Oligonucleotide
tatagcaactacttcgcatttccgtaacggcaatacatcgt

SEQ ID NO: 981 - DNA, 39 bp, Artificial Sequence
  Target Specific Oligonucleotide
cgatggtgtcctacggatgtcgtaacggcaatacatcgt

SEQ ID NO: 982 - DNA, 44 bp, Artificial Sequence
  Target Specific Oligonucleotide
gtaaccatagtaccacttattagtgggtaacggcaatacatcgt

SEQ ID NO: 983 - DNA, 37 bp, Artificial Sequence
  Target Specific Oligonucleotide
cagcatcagcccgtgagtagtaacggcaatacatcgt

SEQ ID NO: 984 - DNA, 39 bp, Artificial Sequence
  Target Specific Oligonucleotide
tcagatgctactggccgcttagacaagttaagatcggaa

SEQ ID NO: 985 - DNA, 41 bp, Artificial Sequence
  Target Specific Oligonucleotide
cttgatcaaggcgctgaacaatagacaagttaagatcggaa

SEQ ID NO: 986 - DNA, 41 bp, Artificial Sequence
  Target Specific Oligonucleotide
tcccttcgtatctcagcgagatagacaagttaagatcggaa

SEQ ID NO: 987 - DNA, 41 bp, Artificial Sequence
  Target Specific Oligonucleotide
agactgttgactggcgtgatgtagacaagttaagatcggaa

SEQ ID NO: 988 - DNA, 41 bp, Artificial Sequence
  Target Specific Oligonucleotide
ccaacatgctcgcatgagttctagacaagttaagatcggaa

SEQ ID NO: 989 - DNA, 41 bp, Artificial Sequence
  Target Specific Oligonucleotide
ttccttcaccgaactgaggagtagacaagttaagatcggaa

SEQ ID NO: 990 - DNA, 40 bp, Artificial Sequence
  Target Specific Oligonucleotide
agttccaacgagcggcttcatagacaagttaagatcggaa

SEQ ID NO: 991 - DNA, 40 bp, Artificial Sequence
  Target Specific Oligonucleotide
ccatggctgacgagatctgatagacaagttaagatcggaa

SEQ ID NO: 992 - DNA, 40 bp, Artificial Sequence
  Target Specific Oligonucleotide
tagcctaagacccggagctttagacaagttaagatcggaa

SEQ ID NO: 993 - DNA, 40 bp, Artificial Sequence
  Target Specific Oligonucleotide
gcagatactcagcggcattgtagacaagttaagatcggaa

SEQ ID NO: 994 - DNA, 40 bp, Artificial Sequence
  Target Specific Oligonucleotide
catagctcacgcagaacgtgtagacaagttaagatcggaa

SEQ ID NO: 995 - DNA, 39 bp, Artificial Sequence
  Target Specific Oligonucleotide
gtcggccaccgttgaatgatagacaagttaagatcggaa

SEQ ID NO: 996 - DNA, 39 bp, Artificial Sequence
  Target Specific Oligonucleotide
agaacggtcagcgagcgtatagacaagttaagatcggaa

SEQ ID NO: 997 - DNA, 44 bp, Artificial Sequence
  Target Specific Oligonucleotide
gttatgcttagagtgttatctccatagacaagttaagatcggaa

SEQ ID NO: 998 - DNA, 40 bp, Artificial Sequence
  Target Specific Oligonucleotide
tggtgatgtccgtgcgttcctagacaagttaagatcggaa

SEQ ID NO: 999 - DNA, 40 bp, Artificial Sequence
  Target Specific Oligonucleotide
ggtccagaggatcgctctcttagacaagttaagatcggaa

SEQ ID NO: 1000 - DNA, 39 bp, Artificial Sequence
  Target Specific Oligonucleotide
cgtggactgcaagtcccgatagacaagttaagatcggaa

SEQ ID NO: 1001 - DNA, 41 bp, Artificial Sequence
  Target Specific Oligonucleotide
acaggtcaattcccgggtaagtagacaagttaagatcggaa

SEQ ID NO: 1002 - DNA, 41 bp, Artificial Sequence
  Target Specific Oligonucleotide
ctgcaccaggttagggtgttttagacaagttaagatcggaa

SEQ ID NO: 1003 - DNA, 43 bp, Artificial Sequence
  Target Specific Oligonucleotide
ggaaatggcatatacgtgggatttagacaagttaagatcggaa

SEQ ID NO: 1004 - DNA, 40 bp, Artificial Sequence
  Target Specific Oligonucleotide
cttttgtctttgcgcccaggtagacaagttaagatcggaa

SEQ ID NO: 1005 - DNA, 38 bp, Artificial Sequence
  Target Specific Oligonucleotide
tcctgccggttgcactcctagacaagttaagatcggaa

SEQ ID NO: 1006 - DNA, 43 bp, Artificial Sequence
  Target Specific Oligonucleotide
atcatacagtgcaacgaaaaggttagacaagttaagatcggaa

SEQ ID NO: 1007 - DNA, 40 bp, Artificial Sequence
  Target Specific Oligonucleotide
tcccacttgtcgtagttgggtagacaagttaagatcggaa

SEQ ID NO: 1008 - DNA, 39 bp, Artificial Sequence
  Target Specific Oligonucleotide
aggtggtgttccgaacgagctagacagcccaattaaacg

SEQ ID NO: 1009 - DNA, 39 bp, Artificial Sequence
  Target Specific Oligonucleotide
agatccgtgagcggaatgtatagacagcccaattaaacg

SEQ ID NO: 1010 - DNA, 41 bp, Artificial Sequence
  Target Specific Oligonucleotide
ctcactcgctttagtggactcctagacagcccaattaaacg

SEQ ID NO: 1011 - DNA, 37 bp, Artificial Sequence
  Target Specific Oligonucleotide
gccaaagaccgtggcgagtagacagcccaattaaacg

SEQ ID NO: 1012 - DNA, 41 bp, Artificial Sequence
  Target Specific Oligonucleotide
agagttcttggtcgttggatcctagacagcccaattaaacg

SEQ ID NO: 1013 - DNA, 39 bp, Artificial Sequence
  Target Specific Oligonucleotide
ctcctttgcaaccgggtctgtagacagcccaattaaacg

SEQ ID NO: 1014 - DNA, 41 bp, Artificial Sequence
  Target Specific Oligonucleotide
cttgttattgacgtcgaaggcttagacagcccaattaaacg

SEQ ID NO: 1015 - DNA, 40 bp, Artificial Sequence
  Target Specific Oligonucleotide
caggaacgtgtaactcttgcctagacagcccaattaaacg

SEQ ID NO: 1016 - DNA, 40 bp, Artificial Sequence
  Target Specific Oligonucleotide
ggagtttcacacacgagttggtagacagcccaattaaacg

SEQ ID NO: 1017 - DNA, 39 bp, Artificial Sequence
  Target Specific Oligonucleotide
aggttcccgctctacggatgtagacagcccaattaaacg

SEQ ID NO: 1018 - DNA, 38 bp, Artificial Sequence
  Target Specific Oligonucleotide
accgtcatggactgccgtctagacagcccaattaaacg

SEQ ID NO: 1019 - DNA, 40 bp, Artificial Sequence
  Target Specific Oligonucleotide
tgggacctttagacttcggtgtagacagcccaattaaacg

SEQ ID NO: 1020 - DNA, 37 bp, Artificial Sequence
  Target Specific Oligonucleotide
gtccctgtagacgcgcgttagacagcccaattaaacg

SEQ ID NO: 1021 - DNA, 38 bp, Artificial Sequence
  Target Specific Oligonucleotide
ccagagagggcgcatcttgtagacagcccaattaaacg

SEQ ID NO: 1022 - DNA, 40 bp, Artificial Sequence
  Target Specific Oligonucleotide
tccactctgcacgctcatagttagacagcccaattaaacg

SEQ ID NO: 1023 - DNA, 40 bp, Artificial Sequence
  Target Specific Oligonucleotide
gtgaaactcgacgttcacgtatagacagcccaattaaacg

SEQ ID NO: 1024 - DNA, 40 bp, Artificial Sequence
  Target Specific Oligonucleotide
acacctctttgtcgttgaccttagacagcccaattaaacg

SEQ ID NO: 1025 - DNA, 39 bp, Artificial Sequence
  Target Specific Oligonucleotide
actgttgcgagttctgcgagtagacagcccaattaaacg

SEQ ID NO: 1026 - DNA, 39 bp, Artificial Sequence
  Target Specific Oligonucleotide
tggcaactccgtagttgtcctagacagcccaattaaacg

SEQ ID NO: 1027 - DNA, 38 bp, Artificial Sequence
  Target Specific Oligonucleotide
tcctcggacgctaagctcatagacagcccaattaaacg

SEQ ID NO: 1028 - DNA, 38 bp, Artificial Sequence
  Target Specific Oligonucleotide
ttacccggagaaccctcgctagacagcccaattaaacg

SEQ ID NO: 1029 - DNA, 38 bp, Artificial Sequence
  Target Specific Oligonucleotide
tcccggaacatgcggtaggtagacagcccaattaaacg

SEQ ID NO: 1030 - DNA, 38 bp, Artificial Sequence
  Target Specific Oligonucleotide
tgctcgatgtcgcccactgtagacagcccaattaaacg

SEQ ID NO: 1031 - DNA, 37 bp, Artificial Sequence
  Target Specific Oligonucleotide
tgccggtgccgcttatggtagacagcccaattaaacg

SEQ ID NO: 1032 - DNA, 45 bp, Artificial Sequence
  Target Specific Oligonucleotide
ccaaagtctgctagcttgatggtagacggtataatacttatttcc

SEQ ID NO: 1033 - DNA, 48 bp, Artificial Sequence
  Target Specific Oligonucleotide
tattaggatggttaagctccttaagtagacggtataatacttatttcc

SEQ ID NO: 1034 - DNA, 45 bp, Artificial Sequence
  Target Specific Oligonucleotide
catgggtgtaagtacgaacaggtagacggtataatacttatttcc

SEQ ID NO: 1035 - DNA, 43 bp, Artificial Sequence
  Target Specific Oligonucleotide
ggcagaaagctaggccctggtagacggtataatacttatttcc

SEQ ID NO: 1036 - DNA, 43 bp, Artificial Sequence
  Target Specific Oligonucleotide
gtacacaactccgtacgtgctagacggtataatacttatttcc

SEQ ID NO: 1037 - DNA, 45 bp, Artificial Sequence
  Target Specific Oligonucleotide
atggaacgcagtatacctctcgtagacggtataatacttatttcc

SEQ ID NO: 1038 - DNA, 46 bp, Artificial Sequence
  Target Specific Oligonucleotide
cttggcttgtaatcaggcatagatagacggtataatacttatttcc

SEQ ID NO: 1039 - DNA, 41 bp, Artificial Sequence
  Target Specific Oligonucleotide
ctccatgaagcgccagcgtagacggtataatacttatttcc

SEQ ID NO: 1040 - DNA, 43 bp, Artificial Sequence
  Target Specific Oligonucleotide
ccgcttgttagggtcgtagttagacggtataatacttatttcc

SEQ ID NO: 1041 - DNA, 42 bp, Artificial Sequence
  Target Specific Oligonucleotide
ctcggtggaggacccgatgtagacggtataatacttatttcc

SEQ ID NO: 1042 - DNA, 44 bp, Artificial Sequence
  Target Specific Oligonucleotide
ttactaaaatcttgccgggcctagacggtataatacttatttcc

SEQ ID NO: 1043 - DNA, 44 bp, Artificial Sequence
  Target Specific Oligonucleotide
gcagcatttgcgataacaagctagacggtataatacttatttcc

SEQ ID NO: 1044 - DNA, 45 bp, Artificial Sequence
  Target Specific Oligonucleotide
agaaggctatcagagtcgaagatagacggtataatacttatttcc

SEQ ID NO: 1045 - DNA, 43 bp, Artificial Sequence
  Target Specific Oligonucleotide
ttcaggagctcggtaccacatagacggtataatacttatttcc

SEQ ID NO: 1046 - DNA, 44 bp, Artificial Sequence
  Target Specific Oligonucleotide
ccccagagtccgaaagatccgtagacggtataatacttatttcc

SEQ ID NO: 1047 - DNA, 42 bp, Artificial Sequence
  Target Specific Oligonucleotide
acagggcccagtaccgtggtagacggtataatacttatttcc

SEQ ID NO: 1048 - DNA, 46 bp, Artificial Sequence
  Target Specific Oligonucleotide
tcagcaataactaagagaaggggtagacggtataatacttatttcc

SEQ ID NO: 1049 - DNA, 46 bp, Artificial Sequence
  Target Specific Oligonucleotide
gtacggcaaatctaacgtgtaggtagacggtataatacttatttcc

SEQ ID NO: 1050 - DNA, 44 bp, Artificial Sequence
  Target Specific Oligonucleotide
tgaaacaatgttgccgcctcctagacggtataatacttatttcc

SEQ ID NO: 1051 - DNA, 47 bp, Artificial Sequence
  Target Specific Oligonucleotide
taataattatggggcattcagagatagacggtataatacttatttcc

SEQ ID NO: 1052 - DNA, 43 bp, Artificial Sequence
  Target Specific Oligonucleotide
tctcacctgcctcataaggctagacggtataatacttatttcc

SEQ ID NO: 1053 - DNA, 47 bp, Artificial Sequence
  Target Specific Oligonucleotide
atggtgaagcaatagattggaatgtagacggtataatacttatttcc

SEQ ID NO: 1054 - DNA, 49 bp, Artificial Sequence
  Target Specific Oligonucleotide
cactaactagggtataaactgattggtagacggtataatacttatttcc

SEQ ID NO: 1055 - DNA, 44 bp, Artificial Sequence
  Target Specific Oligonucleotide
agaagggacaaacaaggggactagacggtataatacttatttcc

SEQ ID NO: 1056 - DNA, 43 bp, Artificial Sequence
  Target Specific Oligonucleotide
agtccgatactcaagcgcaaatagacaataaattctaacgagc

SEQ ID NO: 1057 - DNA, 42 bp, Artificial Sequence
  Target Specific Oligonucleotide
cttgttgcgctcacggtatgtagacaataaattctaacgagc

SEQ ID NO: 1058 - DNA, 43 bp, Artificial Sequence
  Target Specific Oligonucleotide
ccagagttgtagacgagcagctagacaataaattctaacgagc

SEQ ID NO: 1059 - DNA, 41 bp, Artificial Sequence
  Target Specific Oligonucleotide
ttagcatggcaacacggcgtagacaataaattctaacgagc

SEQ ID NO: 1060 - DNA, 41 bp, Artificial Sequence
  Target Specific Oligonucleotide
atgatgcgcacttcacgggtagacaataaattctaacgagc

SEQ ID NO: 1061 - DNA, 42 bp, Artificial Sequence
  Target Specific Oligonucleotide
cccaccgctaggagggtagctagacaataaattctaacgagc

SEQ ID NO: 1062 - DNA, 42 bp, Artificial Sequence
  Target Specific Oligonucleotide
gcagtgtggttaggccgatgtagacaataaattctaacgagc

SEQ ID NO: 1063 - DNA, 41 bp, Artificial Sequence
  Target Specific Oligonucleotide
gacacggcggtgcactatctagacaataaattctaacgagc

SEQ ID NO: 1064 - DNA, 40 bp, Artificial Sequence
  Target Specific Oligonucleotide
gcccaaagcggcttgcagtagacaataaattctaacgagc

SEQ ID NO: 1065 - DNA, 44 bp, Artificial Sequence
  Target Specific Oligonucleotide
atacacacacggctaatactgctagacaataaattctaacgagc

SEQ ID NO: 1066 - DNA, 43 bp, Artificial Sequence
  Target Specific Oligonucleotide
gtgaccgtgtacaccaacaactagacaataaattctaacgagc

SEQ ID NO: 1067 - DNA, 46 bp, Artificial Sequence
  Target Specific Oligonucleotide
ctcttcatactagcatgactgtcatagacaataaattctaacgagc

SEQ ID NO: 1068 - DNA, 42 bp, Artificial Sequence
  Target Specific Oligonucleotide
agaaggctttcgtcgttccctagacaataaattctaacgagc

SEQ ID NO: 1069 - DNA, 42 bp, Artificial Sequence
  Target Specific Oligonucleotide
tttgctccgtgcgttcaaggtagacaataaattctaacgagc

SEQ ID NO: 1070 - DNA, 42 bp, Artificial Sequence
  Target Specific Oligonucleotide
tccagtgcatccgtcacatctagacaataaattctaacgagc

SEQ ID NO: 1071 - DNA, 41 bp, Artificial Sequence
  Target Specific Oligonucleotide
gatgaacagcggtgcgttgtagacaataaattctaacgagc

SEQ ID NO: 1072 - DNA, 42 bp, Artificial Sequence
  Target Specific Oligonucleotide
cactgataccggctctccgttagacaataaattctaacgagc

SEQ ID NO: 1073 - DNA, 42 bp, Artificial Sequence
  Target Specific Oligonucleotide
gaacgcttgcgttccctagctagacaataaattctaacgagc

SEQ ID NO: 1074 - DNA, 45 bp, Artificial Sequence
  Target Specific Oligonucleotide
cagatagatttccgggatacagctagacaataaattctaacgagc

SEQ ID NO: 1075 - DNA, 40 bp, Artificial Sequence
  Target Specific Oligonucleotide
tctgctgcggcgattgtgtagacaataaattctaacgagc

SEQ ID NO: 1076 - DNA, 41 bp, Artificial Sequence
  Target Specific Oligonucleotide
ctcggtgatcatgcgtccctagacaataaattctaacgagc

SEQ ID NO: 1077 - DNA, 43 bp, Artificial Sequence
  Target Specific Oligonucleotide
tccaatttcagggcgttctcgtagacaataaattctaacgagc

SEQ ID NO: 1078 - DNA, 40 bp, Artificial Sequence
  Target Specific Oligonucleotide
tgcgcaggtcgacgtcagtagacaataaattctaacgagc

SEQ ID NO: 1079 - DNA, 43 bp, Artificial Sequence
  Target Specific Oligonucleotide
gtccaggtagtatccggagggtagacaataaattctaacgagc

SEQ ID NO: 1080 - DNA, 40 bp, Artificial Sequence
  Target Specific Oligonucleotide
attcccgacagactcatcgctagacgataaattgcgaacg

SEQ ID NO: 1081 - DNA, 40 bp, Artificial Sequence
  Target Specific Oligonucleotide
cagttacatgttcgcgcacgtagacgataaattgcgaacg

SEQ ID NO: 1082 - DNA, 39 bp, Artificial Sequence
  Target Specific Oligonucleotide
acgagcactagggtcgtcgtagacgataaattgcgaacg

SEQ ID NO: 1083 - DNA, 46 bp, Artificial Sequence
  Target Specific Oligonucleotide
ttttgataatcgaatgaaaaagtcactagacgataaattgcgaacg

SEQ ID NO: 1084 - DNA, 43 bp, Artificial Sequence
  Target Specific Oligonucleotide
ctgaatactgctagacgagaagctagacgataaattgcgaacg

SEQ ID NO: 1085 - DNA, 43 bp, Artificial Sequence
  Target Specific Oligonucleotide
ctccttggaacgaactaatcctgtagacgataaattgcgaacg

SEQ ID NO: 1086 - DNA, 42 bp, Artificial Sequence
  Target Specific Oligonucleotide
ttcaggcggttaattggtaccctagacgataaattgcgaacg

SEQ ID NO: 1087 - DNA, 42 bp, Artificial Sequence
  Target Specific Oligonucleotide
gttaggaatggcgaacagacagtagacgataaattgcgaacg

SEQ ID NO: 1088 - DNA, 42 bp, Artificial Sequence
  Target Specific Oligonucleotide
gatggtcttaaagcgtgaagcctagacgataaattgcgaacg

SEQ ID NO: 1089 - DNA, 42 bp, Artificial Sequence
  Target Specific Oligonucleotide
ctgtgcctagccctaacttatatagacgataaattgcgaacg

SEQ ID NO: 1090 - DNA, 42 bp, Artificial Sequence
  Target Specific Oligonucleotide
agagaccacccataacaagagttagacgataaattgcgaacg

SEQ ID NO: 1091 - DNA, 43 bp, Artificial Sequence
  Target Specific Oligonucleotide
ttggagcaccgatatagatatggtagacgataaattgcgaacg

SEQ ID NO: 1092 - DNA, 41 bp, Artificial Sequence
  Target Specific Oligonucleotide
tcaaagacgcaaacgtccaaatagacgataaattgcgaacg

SEQ ID NO: 1093 - DNA, 42 bp, Artificial Sequence
  Target Specific Oligonucleotide
gagagctgattaggtgctcaggtagacgataaattgcgaacg

SEQ ID NO: 1094 - DNA, 40 bp, Artificial Sequence
  Target Specific Oligonucleotide
tgcttgtacgcggagaaggttagacgataaattgcgaacg

SEQ ID NO: 1095 - DNA, 40 bp, Artificial Sequence
  Target Specific Oligonucleotide
ctgcagacacgtgtatgtgctagacgataaattgcgaacg

SEQ ID NO: 1096 - DNA, 40 bp, Artificial Sequence
  Target Specific Oligonucleotide
catccaatgcaagtccggtgtagacgataaattgcgaacg

SEQ ID NO: 1097 - DNA, 42 bp, Artificial Sequence
  Target Specific Oligonucleotide
accaagtgataacgtgttcagatagacgataaattgcgaacg

SEQ ID NO: 1098 - DNA, 42 bp, Artificial Sequence
  Target Specific Oligonucleotide
aatctgagctttgcgaaatggttagacgataaattgcgaacg

SEQ ID NO: 1099 - DNA, 44 bp, Artificial Sequence
  Target Specific Oligonucleotide
caaagcccacataacactatcttttagacgataaattgcgaacg

SEQ ID NO: 1100 - DNA, 44 bp, Artificial Sequence
  Target Specific Oligonucleotide
tgtactggactatagacaccaatttagacgataaattgcgaacg

SEQ ID NO: 1101 - DNA, 45 bp, Artificial Sequence
  Target Specific Oligonucleotide
atcaatataccacgttagtagtcactagacgataaattgcgaacg

SEQ ID NO: 1102 - DNA, 42 bp, Artificial Sequence
  Target Specific Oligonucleotide
tgtattttaacgccaagcaggctagacgataaattgcgaacg

SEQ ID NO: 1103 - DNA, 42 bp, Artificial Sequence
  Target Specific Oligonucleotide
cactgaaaacacggttatgtcctagacgataaattgcgaacg

SEQ ID NO: 1104 - DNA, 40 bp, Artificial Sequence
  Target Specific Oligonucleotide
cgagacagatcggagtagctgtagacgaccgaatttacgg

SEQ ID NO: 1105 - DNA, 41 bp, Artificial Sequence
  Target Specific Oligonucleotide
tctctcaatgacgggacaaagatagacgaccgaatttacgg

SEQ ID NO: 1106 - DNA, 41 bp, Artificial Sequence
  Target Specific Oligonucleotide
cagatgtggtgtatggtccacatagacgaccgaatttacgg

SEQ ID NO: 1107 - DNA, 40 bp, Artificial Sequence
  Target Specific Oligonucleotide
ttctcctcggttacttcgtgatagacgaccgaatttacgg

SEQ ID NO: 1108 - DNA, 39 bp, Artificial Sequence
  Target Specific Oligonucleotide
tgtgctaagccgaaggaggttagacgaccgaatttacgg

SEQ ID NO: 1109 - DNA, 44 bp, Artificial Sequence
  Target Specific Oligonucleotide
gtaaacatagaacgactcatagtcatagacgaccgaatttacgg

SEQ ID NO: 1110 - DNA, 43 bp, Artificial Sequence
  Target Specific Oligonucleotide
ttccacttcaattagctcttgaattagacgaccgaatttacgg

SEQ ID NO: 1111 - DNA, 40 bp, Artificial Sequence
  Target Specific Oligonucleotide
ctggggtagagatagctcgcttagacgaccgaatttacgg

SEQ ID NO: 1112 - DNA, 41 bp, Artificial Sequence
  Target Specific Oligonucleotide
gctataagctcgcctaccattctagacgaccgaatttacgg

SEQ ID NO: 1113 - DNA, 39 bp, Artificial Sequence
  Target Specific Oligonucleotide
ttcttggggcgggtagagtgtagacgaccgaatttacgg

SEQ ID NO: 1114 - DNA, 40 bp, Artificial Sequence
  Target Specific Oligonucleotide
aagacctcagaccgtcagaagtagacgaccgaatttacgg

SEQ ID NO: 1115 - DNA, 39 bp, Artificial Sequence
  Target Specific Oligonucleotide
tcagatcctgacgacctgcatagacgaccgaatttacgg

SEQ ID NO: 1116 - DNA, 40 bp, Artificial Sequence
  Target Specific Oligonucleotide
gcagaaatctcgagaggacgctagacgaccgaatttacgg

SEQ ID NO: 1117 - DNA, 39 bp, Artificial Sequence
  Target Specific Oligonucleotide
ctcgcagtctaggccgaagatagacgaccgaatttacgg

SEQ ID NO: 1118 - DNA, 39 bp, Artificial Sequence
  Target Specific Oligonucleotide
ccggtctccctcgagaatcatagacgaccgaatttacgg

SEQ ID NO: 1119 - DNA, 39 bp, Artificial Sequence
  Target Specific Oligonucleotide
tcctggtgagaacgtcctgctagacgaccgaatttacgg

SEQ ID NO: 1120 - DNA, 38 bp, Artificial Sequence
  Target Specific Oligonucleotide
tgccctttggtaaggcgcctagacgaccgaatttacgg

SEQ ID NO: 1121 - DNA, 38 bp, Artificial Sequence
  Target Specific Oligonucleotide
cacagacacagccgaggactagacgaccgaatttacgg

SEQ ID NO: 1122 - DNA, 39 bp, Artificial Sequence
  Target Specific Oligonucleotide
tcccaaaagctaccgctgagtagacgaccgaatttacgg

SEQ ID NO: 1123 - DNA, 39 bp, Artificial Sequence
  Target Specific Oligonucleotide
taaggtagagtcgggcgaagtagacgaccgaatttacgg

SEQ ID NO: 1124 - DNA, 38 bp, Artificial Sequence
  Target Specific Oligonucleotide
gagtggccggaaaaacccgtagacgaccgaatttacgg

SEQ ID NO: 1125 - DNA, 37 bp, Artificial Sequence
  Target Specific Oligonucleotide
atgcggacatggtcgccctagacgaccgaatttacgg

SEQ ID NO: 1126 - DNA, 37 bp, Artificial Sequence
  Target Specific Oligonucleotide
ggctcccaaacgccctgatagacgaccgaatttacgg

SEQ ID NO: 1127 - DNA, 40 bp, Artificial Sequence
  Target Specific Oligonucleotide
tccatttcaccgtcagaaaggtagacgaccgaatttacgg

SEQ ID NO: 1128 - DNA, 43 bp, Artificial Sequence
  Target Specific Oligonucleotide
ttgcctagacagcaccgtaatgtagacacgaatattccaattc

SEQ ID NO: 1129 - DNA, 40 bp, Artificial Sequence
  Target Specific Oligonucleotide
ttcccagagcgtggttccatagacacgaatattccaattc

SEQ ID NO: 1130 - DNA, 42 bp, Artificial Sequence
  Target Specific Oligonucleotide
taaggacatcgcttttccggttagacacgaatattccaattc

SEQ ID NO: 1131 - DNA, 40 bp, Artificial Sequence
  Target Specific Oligonucleotide
ttctccacgtcgcggacactagacacgaatattccaattc

SEQ ID NO: 1132 - DNA, 46 bp, Artificial Sequence
  Target Specific Oligonucleotide
gttctcatgatagaggttccttaagtagacacgaatattccaattc

SEQ ID NO: 1133 - DNA, 38 bp, Artificial Sequence
  Target Specific Oligonucleotide
ggccacgtcggtgcagatagacacgaatattccaattc

SEQ ID NO: 1134 - DNA, 41 bp, Artificial Sequence
  Target Specific Oligonucleotide
gttgtggacgatcaacggggtagacacgaatattccaattc

SEQ ID NO: 1135 - DNA, 42 bp, Artificial Sequence
  Target Specific Oligonucleotide
tgtccccttcggggtcatacctagacacgaatattccaattc

SEQ ID NO: 1136 - DNA, 44 bp, Artificial Sequence
  Target Specific Oligonucleotide
aatagaacctcatccggtagtggtagacacgaatattccaattc

SEQ ID NO: 1137 - DNA, 42 bp, Artificial Sequence
  Target Specific Oligonucleotide
cagtgcagcgtatgtggtatttagacacgaatattccaattc

SEQ ID NO: 1138 - DNA, 42 bp, Artificial Sequence
  Target Specific Oligonucleotide
aaagcttgtccgattggatggtagacacgaatattccaattc

SEQ ID NO: 1139 - DNA, 42 bp, Artificial Sequence
  Target Specific Oligonucleotide
aacccttgtagatcggtggtatagacacgaatattccaattc

SEQ ID NO: 1140 - DNA, 42 bp, Artificial Sequence
  Target Specific Oligonucleotide
agtagagaacgtttccaccgttagacacgaatattccaattc

SEQ ID NO: 1141 - DNA, 42 bp, Artificial Sequence
  Target Specific Oligonucleotide
actgctcattgtcgttggttctagacacgaatattccaattc

SEQ ID NO: 1142 - DNA, 41 bp, Artificial Sequence
  Target Specific Oligonucleotide
aacgtattgccgagaacccatagacacgaatattccaattc

SEQ ID NO: 1143 - DNA, 43 bp, Artificial Sequence
  Target Specific Oligonucleotide
acaaagtgcatatagagtggcctagacacgaatattccaattc

SEQ ID NO: 1144 - DNA, 42 bp, Artificial Sequence
  Target Specific Oligonucleotide
agccatccctagacactcgtttagacacgaatattccaattc

SEQ ID NO: 1145 - DNA, 42 bp, Artificial Sequence
  Target Specific Oligonucleotide
gcgatttggagcataccagagtagacacgaatattccaattc

SEQ ID NO: 1146 - DNA, 42 bp, Artificial Sequence
  Target Specific Oligonucleotide
aggatccgatcgaaactcagctagacacgaatattccaattc

SEQ ID NO: 1147 - DNA, 44 bp, Artificial Sequence
  Target Specific Oligonucleotide
ctggaatatatcgcttgtagctgtagacacgaatattccaattc

SEQ ID NO: 1148 - DNA, 41 bp, Artificial Sequence
  Target Specific Oligonucleotide
cttcaggtcatgcgtggacatagacacgaatattccaattc

SEQ ID NO: 1149 - DNA, 41 bp, Artificial Sequence
  Target Specific Oligonucleotide
agagaagcgcacttccgtgttagacacgaatattccaattc

SEQ ID NO: 1150 - DNA, 41 bp, Artificial Sequence
  Target Specific Oligonucleotide
tgatgttatccgtgcgcaggtagacacgaatattccaattc

SEQ ID NO: 1151 - DNA, 44 bp, Artificial Sequence
  Target Specific Oligonucleotide
aaagaccgtaatcgttcacattgtagacacgaatattccaattc

SEQ ID NO: 1152 - DNA, 42 bp, Artificial Sequence
  Target Specific Oligonucleotide
agagatttgtctacgtagagccgtagactcagaaatctcacg

SEQ ID NO: 1153 - DNA, 41 bp, Artificial Sequence
  Target Specific Oligonucleotide
tcctttgcatacggtctcttcgtagactcagaaatctcacg

SEQ ID NO: 1154 - DNA, 43 bp, Artificial Sequence
  Target Specific Oligonucleotide
aagtttgtttacatccgatgttactagactcagaaatctcacg

SEQ ID NO: 1155 - DNA, 39 bp, Artificial Sequence
  Target Specific Oligonucleotide
ttcccatacagaacgtggcctagactcagaaatctcacg

SEQ ID NO: 1156 - DNA, 40 bp, Artificial Sequence
  Target Specific Oligonucleotide
aacagatcaacggcaaaagcctagactcagaaatctcacg

SEQ ID NO: 1157 - DNA, 42 bp, Artificial Sequence
  Target Specific Oligonucleotide
catccttgggtagtaggatgaactagactcagaaatctcacg

SEQ ID NO: 1158 - DNA, 40 bp, Artificial Sequence
  Target Specific Oligonucleotide
gaatttgctagttgcagggcatagactcagaaatctcacg

SEQ ID NO: 1159 - DNA, 45 bp, Artificial Sequence
  Target Specific Oligonucleotide
tgatcttacaattgatactgtcaatgtagactcagaaatctcacg

SEQ ID NO: 1160 - DNA, 38 bp, Artificial Sequence
  Target Specific Oligonucleotide
tggatgtggaggcgaccgttagactcagaaatctcacg

SEQ ID NO: 1161 - DNA, 42 bp, Artificial Sequence
  Target Specific Oligonucleotide
acttctggacctatagtagcctgtagactcagaaatctcacg

SEQ ID NO: 1162 - DNA, 42 bp, Artificial Sequence
  Target Specific Oligonucleotide
caacacggatagcttatccaacctagactcagaaatctcacg

SEQ ID NO: 1163 - DNA, 40 bp, Artificial Sequence
  Target Specific Oligonucleotide
tgtcaaggtaacgtgagcacttagactcagaaatctcacg

SEQ ID NO: 1164 - DNA, 41 bp, Artificial Sequence
  Target Specific Oligonucleotide
tttacagaaatcggcatccacatagactcagaaatctcacg

SEQ ID NO: 1165 - DNA, 44 bp, Artificial Sequence
  Target Specific Oligonucleotide
cagctactagaggtctataattccttagactcagaaatctcacg

SEQ ID NO: 1166 - DNA, 40 bp, Artificial Sequence
  Target Specific Oligonucleotide
ctggcacctctatggaatccctagactcagaaatctcacg

SEQ ID NO: 1167 - DNA, 43 bp, Artificial Sequence
  Target Specific Oligonucleotide
attctgatatcggctattgtgagatagactcagaaatctcacg

SEQ ID NO: 1168 - DNA, 39 bp, Artificial Sequence
  Target Specific Oligonucleotide
gccaccagtctagtgccaactagactcagaaatctcacg

SEQ ID NO: 1169 - DNA, 40 bp, Artificial Sequence
  Target Specific Oligonucleotide
caactaccccggacaatttgatagactcagaaatctcacg

SEQ ID NO: 1170 - DNA, 41 bp, Artificial Sequence
  Target Specific Oligonucleotide
aggacttaacatgggctatgcctagactcagaaatctcacg

SEQ ID NO: 1171 - DNA, 41 bp, Artificial Sequence
  Target Specific Oligonucleotide
ctctgcgaacgaagtgtttttgtagactcagaaatctcacg

SEQ ID NO: 1172 - DNA, 43 bp, Artificial Sequence
  Target Specific Oligonucleotide
tgcagcatattaaggtgttgattttagactcagaaatctcacg

SEQ ID NO: 1173 - DNA, 44 bp, Artificial Sequence
  Target Specific Oligonucleotide
aagtaactaaacccatagactgaaatagactcagaaatctcacg

SEQ ID NO: 1174 - DNA, 40 bp, Artificial Sequence
  Target Specific Oligonucleotide
ctgccagggcttaacataaggtagactcagaaatctcacg

SEQ ID NO: 1175 - DNA, 42 bp, Artificial Sequence
  Target Specific Oligonucleotide
ataacatttgatagggccactcctagactcagaaatctcacg

SEQ ID NO: 1176 - DNA, 41 bp, Artificial Sequence
  Target Specific Oligonucleotide
ctgaagataatcgcagacaccctagacgtctcccgtatttt

SEQ ID NO: 1177 - DNA, 41 bp, Artificial Sequence
  Target Specific Oligonucleotide
tgactgaacacgtgttctgttctagacgtctcccgtatttt

SEQ ID NO: 1178 - DNA, 39 bp, Artificial Sequence
  Target Specific Oligonucleotide
tttcagccatacccggatggtagacgtctcccgtatttt

SEQ ID NO: 1179 - DNA, 41 bp, Artificial Sequence
  Target Specific Oligonucleotide
tgagaagagtaaccgctgaatatagacgtctcccgtatttt

SEQ ID NO: 1180 - DNA, 38 bp, Artificial Sequence
  Target Specific Oligonucleotide
acactgcaattccgccgtctagacgtctcccgtatttt

SEQ ID NO: 1181 - DNA, 38 bp, Artificial Sequence
  Target Specific Oligonucleotide
ttgtcgtggccggtactgatagacgtctcccgtatttt

SEQ ID NO: 1182 - DNA, 39 bp, Artificial Sequence
  Target Specific Oligonucleotide
ggcaggcgacactaccttcatagacgtctcccgtatttt

SEQ ID NO: 1183 - DNA, 40 bp, Artificial Sequence
  Target Specific Oligonucleotide
ctgggccagtattgaccactgtagacgtctcccgtatttt

SEQ ID NO: 1184 - DNA, 40 bp, Artificial Sequence
  Target Specific Oligonucleotide
tctcagatcagtgcggagaaatagacgtctcccgtatttt

SEQ ID NO: 1185 - DNA, 39 bp, Artificial Sequence
  Target Specific Oligonucleotide
gcccacaaatatccgtctgctagacgtctcccgtatttt

SEQ ID NO: 1186 - DNA, 36 bp, Artificial Sequence
  Target Specific Oligonucleotide
ccatgttcgcgcggcgatagacgtctcccgtatttt

SEQ ID NO: 1187 - DNA, 42 bp, Artificial Sequence
  Target Specific Oligonucleotide
atgaaaggtcgctagagaagtcctagacgtctcccgtatttt

SEQ ID NO: 1188 - DNA, 40 bp, Artificial Sequence
  Target Specific Oligonucleotide
aatcacaaacccggacttggatagacgtctcccgtatttt

SEQ ID NO: 1189 - DNA, 40 bp, Artificial Sequence
  Target Specific Oligonucleotide
actagcctgtcacgtttgtcttagacgtctcccgtatttt

SEQ ID NO: 1190 - DNA, 41 bp, Artificial Sequence
  Target Specific Oligonucleotide
atctttacaatgcggtcggtcctagacgtctcccgtatttt

SEQ ID NO: 1191 - DNA, 41 bp, Artificial Sequence
  Target Specific Oligonucleotide
tgtcctggtatgatcccaacactagacgtctcccgtatttt

SEQ ID NO: 1192 - DNA, 41 bp, Artificial Sequence
  Target Specific Oligonucleotide
ctgatcatttcgcatcacagtgtagacgtctcccgtatttt

SEQ ID NO: 1193 - DNA, 41 bp, Artificial Sequence
  Target Specific Oligonucleotide
ctttccttacgtgtgtcacacatagacgtctcccgtatttt

SEQ ID NO: 1194 - DNA, 42 bp, Artificial Sequence
  Target Specific Oligonucleotide
tagtttgatcgcctccttaaacctagacgtctcccgtatttt

SEQ ID NO: 1195 - DNA, 42 bp, Artificial Sequence
  Target Specific Oligonucleotide
aaatccctaggatagcacctttgtagacgtctcccgtatttt

SEQ ID NO: 1196 - DNA, 42 bp, Artificial Sequence
  Target Specific Oligonucleotide
cttacacttacgatgagcagatgtagacgtctcccgtatttt

SEQ ID NO: 1197 - DNA, 43 bp, Artificial Sequence
  Target Specific Oligonucleotide
catttgctacctagcatatgagagtagacgtctcccgtatttt

SEQ ID NO: 1198 - DNA, 41 bp, Artificial Sequence
  Target Specific Oligonucleotide
acagatgtcaaacggtcacatttagacgtctcccgtatttt

SEQ ID NO: 1199 - DNA, 43 bp, Artificial Sequence
  Target Specific Oligonucleotide
ttctaagatacgtcactccaattctagacgtctcccgtatttt

SEQ ID NO: 1200 - DNA, 42 bp, Artificial Sequence
  Target Specific Oligonucleotide
acagtccgtcgattctttacaatagactctataaatcagcgt

SEQ ID NO: 1201 - DNA, 41 bp, Artificial Sequence
  Target Specific Oligonucleotide
tctccacgtaaatcccgatcttagactctataaatcagcgt

SEQ ID NO: 1202 - DNA, 40 bp, Artificial Sequence
  Target Specific Oligonucleotide
cacgtgctgaaccaaccacttagactctataaatcagcgt

SEQ ID NO: 1203 - DNA, 39 bp, Artificial Sequence
  Target Specific Oligonucleotide
tcttgccgaagttggccgttagactctataaatcagcgt

SEQ ID NO: 1204 - DNA, 41 bp, Artificial Sequence
  Target Specific Oligonucleotide
agcagcagttggatctacagttagactctataaatcagcgt

SEQ ID NO: 1205 - DNA, 43 bp, Artificial Sequence
  Target Specific Oligonucleotide
gtctagcagtaaacctgtaaacctagactctataaatcagcgt

SEQ ID NO: 1206 - DNA, 42 bp, Artificial Sequence
  Target Specific Oligonucleotide
gtgcattattggacgaacatcatagactctataaatcagcgt

SEQ ID NO: 1207 - DNA, 45 bp, Artificial Sequence
  Target Specific Oligonucleotide
gctgttgttaatggtctataatccatagactctataaatcagcgt

SEQ ID NO: 1208 - DNA, 45 bp, Artificial Sequence
  Target Specific Oligonucleotide
cacacatatcctatgttcatcaatatagactctataaatcagcgt

SEQ ID NO: 1209 - DNA, 43 bp, Artificial Sequence
  Target Specific Oligonucleotide
ttgagtggtcgttttcttacacatagactctataaatcagcgt

SEQ ID NO: 1210 - DNA, 45 bp, Artificial Sequence
  Target Specific Oligonucleotide
cataatcaaaacgaaatgtttggtttagactctataaatcagcgt

SEQ ID NO: 1211 - DNA, 41 bp, Artificial Sequence
  Target Specific Oligonucleotide
tttgtagcatcaacgtcctggtagactctataaatcagcgt

SEQ ID NO: 1212 - DNA, 41 bp, Artificial Sequence
  Target Specific Oligonucleotide
caacacatttgacctcccgtttagactctataaatcagcgt

SEQ ID NO: 1213 - DNA, 43 bp, Artificial Sequence
  Target Specific Oligonucleotide
ctaaccaccgaatagattcctggtagactctataaatcagcgt

SEQ ID NO: 1214 - DNA, 41 bp, Artificial Sequence
  Target Specific Oligonucleotide
gcagatgtttgaccggatgtttagactctataaatcagcgt

SEQ ID NO: 1215 - DNA, 42 bp, Artificial Sequence
  Target Specific Oligonucleotide
tgcttgatgtattcggccatcgtagactctataaatcagcgt

SEQ ID NO: 1216 - DNA, 42 bp, Artificial Sequence
  Target Specific Oligonucleotide
tgagtttacttgcacggaaaggtagactctataaatcagcgt

SEQ ID NO: 1217 - DNA, 42 bp, Artificial Sequence
  Target Specific Oligonucleotide
cactttatcccgcagttcagtttagactctataaatcagcgt

SEQ ID NO: 1218 - DNA, 41 bp, Artificial Sequence
  Target Specific Oligonucleotide
gcaaagcatgtagccattccctagactctataaatcagcgt

SEQ ID NO: 1219 - DNA, 43 bp, Artificial Sequence
  Target Specific Oligonucleotide
aaagacatctcgagctgctaatgtagactctataaatcagcgt

SEQ ID NO: 1220 - DNA, 43 bp, Artificial Sequence
  Target Specific Oligonucleotide
gtcaccacccatagtatgagttttagactctataaatcagcgt

SEQ ID NO: 1221 - DNA, 43 bp, Artificial Sequence
  Target Specific Oligonucleotide
tttctacctaaggctctgatgcatagactctataaatcagcgt

SEQ ID NO: 1222 - DNA, 41 bp, Artificial Sequence
  Target Specific Oligonucleotide
tagagctgcacggaaagattttagactctataaatcagcgt

SEQ ID NO: 1223 - DNA, 43 bp, Artificial Sequence
  Target Specific Oligonucleotide
gtgtagcatatgaatcgacatcatagactctataaatcagcgt

SEQ ID NO: 1224 - DNA, 43 bp, Artificial Sequence
  Target Specific Oligonucleotide
tagccttgtatggcagaatcgtagacctaaattactgtgaatg

SEQ ID NO: 1225 - DNA, 45 bp, Artificial Sequence
  Target Specific Oligonucleotide
tgaattgccctgggtataactcctagacctaaattactgtgaatg

SEQ ID NO: 1226 - DNA, 42 bp, Artificial Sequence
  Target Specific Oligonucleotide
ttcctgttgcaacgtgagggtagacctaaattactgtgaatg

SEQ ID NO: 1227 - DNA, 46 bp, Artificial Sequence
  Target Specific Oligonucleotide
atgaaagacacgatcaaaattgaatagacctaaattactgtgaatg

SEQ ID NO: 1228 - DNA, 43 bp, Artificial Sequence
  Target Specific Oligonucleotide
gtcagcacgaagtgagttcaatagacctaaattactgtgaatg

SEQ ID NO: 1229 - DNA, 44 bp, Artificial Sequence
  Target Specific Oligonucleotide
cagtttgcagcgtagaatctcttagacctaaattactgtgaatg

SEQ ID NO: 1230 - DNA, 45 bp, Artificial Sequence
  Target Specific Oligonucleotide
gtgcagataatacgtgtctttgctagacctaaattactgtgaatg

SEQ ID NO: 1231 - DNA, 45 bp, Artificial Sequence
  Target Specific Oligonucleotide
tccaatgagtatgacaagtccattagacctaaattactgtgaatg

SEQ ID NO: 1232 - DNA, 45 bp, Artificial Sequence
  Target Specific Oligonucleotide
tgtaagaaagctcggtcttcaaatagacctaaattactgtgaatg

SEQ ID NO: 1233 - DNA, 46 bp, Artificial Sequence
  Target Specific Oligonucleotide
ccatgtaagatacacgtaagagaatagacctaaattactgtgaatg

SEQ ID NO: 1234 - DNA, 46 bp, Artificial Sequence
  Target Specific Oligonucleotide
gactcaagagcatttcgtaattgttagacctaaattactgtgaatg

SEQ ID NO: 1235 - DNA, 46 bp, Artificial Sequence
  Target Specific Oligonucleotide
atgtcattagtcacctcaagttcttagacctaaattactgtgaatg

SEQ ID NO: 1236 - DNA, 39 bp, Artificial Sequence
  Target Specific Oligonucleotide
ccgcactcgcacgcagatagacctaaattactgtgaatg

SEQ ID NO: 1237 - DNA, 47 bp, Artificial Sequence
  Target Specific Oligonucleotide
tgttaacagtatcgtgaatatcacttagacctaaattactgtgaatg

SEQ ID NO: 1238 - DNA, 45 bp, Artificial Sequence
  Target Specific Oligonucleotide
actttgccatctataagggaggttagacctaaattactgtgaatg

SEQ ID NO: 1239 - DNA, 48 bp, Artificial Sequence
  Target Specific Oligonucleotide
cttgctatctcgataatttatgaaactagacctaaattactgtgaatg

SEQ ID NO: 1240 - DNA, 48 bp, Artificial Sequence
  Target Specific Oligonucleotide
ttaatgtatcaagagtgttactgaaatagacctaaattactgtgaatg

SEQ ID NO: 1241 - DNA, 46 bp, Artificial Sequence
  Target Specific Oligonucleotide
tcaaatgggataccttaacagatctagacctaaattactgtgaatg

SEQ ID NO: 1242 - DNA, 50 bp, Artificial Sequence
  Target Specific Oligonucleotide
ataaataagcttcgatttatattacagctagacctaaattactgtgaatg

SEQ ID NO: 1243 - DNA, 45 bp, Artificial Sequence
  Target Specific Oligonucleotide
cttgtgtggtaggattaacctggtagacctaaattactgtgaatg

SEQ ID NO: 1244 - DNA, 43 bp, Artificial Sequence
  Target Specific Oligonucleotide
gttggtcaactcgagcatctttagacctaaattactgtgaatg

SEQ ID NO: 1245 - DNA, 44 bp, Artificial Sequence
  Target Specific Oligonucleotide
tttggtttgtaggcctgtagcttagacctaaattactgtgaatg

SEQ ID NO: 1246 - DNA, 45 bp, Artificial Sequence
  Target Specific Oligonucleotide
cattaggcagtaactcttgttcctagacctaaattactgtgaatg

SEQ ID NO: 1247 - DNA, 45 bp, Artificial Sequence
  Target Specific Oligonucleotide
cttctcagatacgcttcttcttatagacctaaattactgtgaatg

SEQ ID NO: 1248 - DNA, 31 bp, Artificial Sequence
  Decoding Oligonucleotide
ggctaatttattaccttctagctataacgat

SEQ ID NO: 1249 - DNA, 31 bp, Artificial Sequence
  Decoding Oligonucleotide
accttaagaatggaattctagctataacgat

SEQ ID NO: 1250 - DNA, 30 bp, Artificial Sequence
  Decoding Oligonucleotide
acttaacttgagtggtctagctataacgat

SEQ ID NO: 1251 - DNA, 29 bp, Artificial Sequence
  Decoding Oligonucleotide
ggctaatttattacctcggtctagatgga

SEQ ID NO: 1252 - DNA, 29 bp, Artificial Sequence
  Decoding Oligonucleotide
accttaagaatggaatcggtctagatgga

SEQ ID NO: 1253 - DNA, 28 bp, Artificial Sequence
  Decoding Oligonucleotide
acttaacttgagtggcggtctagatgga

SEQ ID NO: 1254 - DNA, 32 bp, Artificial Sequence
  Decoding Oligonucleotide
ggctaatttattacctgttaactccgtagtaa

SEQ ID NO: 1255 - DNA, 32 bp, Artificial Sequence
  Decoding Oligonucleotide
accttaagaatggaatgttaactccgtagtaa

SEQ ID NO: 1256 - DNA, 31 bp, Artificial Sequence
  Decoding Oligonucleotide
acttaacttgagtgggttaactccgtagtaa

SEQ ID NO: 1257 - DNA, 31 bp, Artificial Sequence
  Decoding Oligonucleotide
ggctaatttattacctcgctatatctactga

SEQ ID NO: 1258 - DNA, 31 bp, Artificial Sequence
  Decoding Oligonucleotide
accttaagaatggaatcgctatatctactga

SEQ ID NO: 1259 - DNA, 30 bp, Artificial Sequence
  Decoding Oligonucleotide
acttaacttgagtggcgctatatctactga

SEQ ID NO: 1260 - DNA, 30 bp, Artificial Sequence
  Decoding Oligonucleotide
ggctaatttattacctgtttccgtttcgaa

SEQ ID NO: 1261 - DNA, 30 bp, Artificial Sequence
  Decoding Oligonucleotide
accttaagaatggaatgtttccgtttcgaa

SEQ ID NO: 1262 - DNA, 29 bp, Artificial Sequence
  Decoding Oligonucleotide
acttaacttgagtgggtttccgtttcgaa

SEQ ID NO: 1263 - DNA, 30 bp, Artificial Sequence
  Decoding Oligonucleotide
ggctaatttattaccttagcgtttcaaaag

SEQ ID NO: 1264 - DNA, 30 bp, Artificial Sequence
  Decoding Oligonucleotide
accttaagaatggaattagcgtttcaaaag

SEQ ID NO: 1265 - DNA, 29 bp, Artificial Sequence
  Decoding Oligonucleotide
acttaacttgagtggtagcgtttcaaaag

SEQ ID NO: 1266 - DNA, 28 bp, Artificial Sequence
  Decoding Oligonucleotide
ggctaatttattaccttgatccgacgag

SEQ ID NO: 1267 - DNA, 28 bp, Artificial Sequence
  Decoding Oligonucleotide
accttaagaatggaattgatccgacgag

SEQ ID NO: 1268 - DNA, 27 bp, Artificial Sequence
  Decoding Oligonucleotide
acttaacttgagtggtgatccgacgag

SEQ ID NO: 1269 - DNA, 31 bp, Artificial Sequence
  Decoding Oligonucleotide
ggctaatttattacctaatctacatgtctga

SEQ ID NO: 1270 - DNA, 31 bp, Artificial Sequence
  Decoding Oligonucleotide
accttaagaatggaataatctacatgtctga

SEQ ID NO: 1271 - DNA, 30 bp, Artificial Sequence
  Decoding Oligonucleotide
acttaacttgagtggaatctacatgtctga

SEQ ID NO: 1272 - DNA, 30 bp, Artificial Sequence
  Decoding Oligonucleotide
ggctaatttattacctggacgttgaataca

SEQ ID NO: 1273 - DNA, 30 bp, Artificial Sequence
  Decoding Oligonucleotide
accttaagaatggaatggacgttgaataca

SEQ ID NO: 1274 - DNA, 29 bp, Artificial Sequence
  Decoding Oligonucleotide
acttaacttgagtggggacgttgaataca

SEQ ID NO: 1275 - DNA, 31 bp, Artificial Sequence
  Decoding Oligonucleotide
ggctaatttattaccttaaagtgacaattca

SEQ ID NO: 1276 - DNA, 31 bp, Artificial Sequence
  Decoding Oligonucleotide
accttaagaatggaattaaagtgacaattca

SEQ ID NO: 1277 - DNA, 30 bp, Artificial Sequence
  Decoding Oligonucleotide
acttaacttgagtggtaaagtgacaattca

SEQ ID NO: 1278 - DNA, 32 bp, Artificial Sequence
  Decoding Oligonucleotide
ggctaatttattacctgattcccataattagg

SEQ ID NO: 1279 - DNA, 32 bp, Artificial Sequence
  Decoding Oligonucleotide
accttaagaatggaatgattcccataattagg

SEQ ID NO: 1280 - DNA, 31 bp, Artificial Sequence
  Decoding Oligonucleotide
acttaacttgagtgggattcccataattagg

SEQ ID NO: 1281 - DNA, 33 bp, Artificial Sequence
  Decoding Oligonucleotide
ggctaatttattacctcgtcgtataaatctatc

SEQ ID NO: 1282 - DNA, 33 bp, Artificial Sequence
  Decoding Oligonucleotide
accttaagaatggaatcgtcgtataaatctatc

SEQ ID NO: 1283 - DNA, 32 bp, Artificial Sequence
  Decoding Oligonucleotide
acttaacttgagtggcgtcgtataaatctatc

SEQ ID NO: 1284 - DNA, 34 bp, Artificial Sequence
  Decoding Oligonucleotide
ggctaatttattacctgatgtctatcctttataa

SEQ ID NO: 1285 - DNA, 34 bp, Artificial Sequence
  Decoding Oligonucleotide
accttaagaatggaatgatgtctatcctttataa

SEQ ID NO: 1286 - DNA, 33 bp, Artificial Sequence
  Decoding Oligonucleotide
acttaacttgagtgggatgtctatcctttataa

SEQ ID NO: 1287 - DNA, 30 bp, Artificial Sequence
  Decoding Oligonucleotide
ggctaatttattaccttttccacgataacc

SEQ ID NO: 1288 - DNA, 30 bp, Artificial Sequence
  Decoding Oligonucleotide
accttaagaatggaattttccacgataacc

SEQ ID NO: 1289 - DNA, 29 bp, Artificial Sequence
  Decoding Oligonucleotide
acttaacttgagtggtttccacgataacc

SEQ ID NO: 1290 - DNA, 31 bp, Artificial Sequence
  Decoding Oligonucleotide
ggctaatttattaccttagtaccgaataagg

SEQ ID NO: 1291 - DNA, 31 bp, Artificial Sequence
  Decoding Oligonucleotide
accttaagaatggaattagtaccgaataagg

SEQ ID NO: 1292 - DNA, 30 bp, Artificial Sequence
  Decoding Oligonucleotide
acttaacttgagtggtagtaccgaataagg

SEQ ID NO: 1293 - DNA, 32 bp, Artificial Sequence
  Decoding Oligonucleotide
ggctaatttattaccttaaagttgtaaaatcc

SEQ ID NO: 1294 - DNA, 32 bp, Artificial Sequence
  Decoding Oligonucleotide
accttaagaatggaattaaagttgtaaaatcc

SEQ ID NO: 1295 - DNA, 31 bp, Artificial Sequence
  Decoding Oligonucleotide
acttaacttgagtggtaaagttgtaaaatcc

SEQ ID NO: 1296 - DNA, 32 bp, Artificial Sequence
  Decoding Oligonucleotide
ggctaatttattacctggataagtcggtaatc

SEQ ID NO: 1297 - DNA, 32 bp, Artificial Sequence
  Decoding Oligonucleotide
accttaagaatggaatggataagtcggtaatc

SEQ ID NO: 1298 - DNA, 31 bp, Artificial Sequence
  Decoding Oligonucleotide
acttaacttgagtggggataagtcggtaatc

SEQ ID NO: 1299 - DNA, 33 bp, Artificial Sequence
  Decoding Oligonucleotide
ggctaatttattacctggtatttcactctaatt

SEQ ID NO: 1300 - DNA, 33 bp, Artificial Sequence
  Decoding Oligonucleotide
accttaagaatggaatggtatttcactctaatt

SEQ ID NO: 1301 - DNA, 32 bp, Artificial Sequence
  Decoding Oligonucleotide
acttaacttgagtggggtatttcactctaatt

SEQ ID NO: 1302 - DNA, 32 bp, Artificial Sequence
  Decoding Oligonucleotide
ggctaatttattacctagctagataaagtacc

SEQ ID NO: 1303 - DNA, 32 bp, Artificial Sequence
  Decoding Oligonucleotide
accttaagaatggaatagctagataaagtacc

SEQ ID NO: 1304 - DNA, 31 bp, Artificial Sequence
  Decoding Oligonucleotide
acttaacttgagtggagctagataaagtacc

SEQ ID NO: 1305 - DNA, 32 bp, Artificial Sequence
  Decoding Oligonucleotide
ggctaatttattacctccgaaactacgattat

SEQ ID NO: 1306 - DNA, 32 bp, Artificial Sequence
  Decoding Oligonucleotide
accttaagaatggaatccgaaactacgattat

SEQ ID NO: 1307 - DNA, 31 bp, Artificial Sequence
  Decoding Oligonucleotide
acttaacttgagtggccgaaactacgattat

SEQ ID NO: 1308 - DNA, 32 bp, Artificial Sequence
  Decoding Oligonucleotide
ggctaatttattacctcatcgaatttatgtta

SEQ ID NO: 1309 - DNA, 32 bp, Artificial Sequence
  Decoding Oligonucleotide
accttaagaatggaatcatcgaatttatgtta

SEQ ID NO: 1310 - DNA, 31 bp, Artificial Sequence
  Decoding Oligonucleotide
acttaacttgagtggcatcgaatttatgtta

SEQ ID NO: 1311 - DNA, 30 bp, Artificial Sequence
  Decoding Oligonucleotide
ggctaatttattacctccgtaattctaccg

SEQ ID NO: 1312 - DNA, 30 bp, Artificial Sequence
  Decoding Oligonucleotide
accttaagaatggaatccgtaattctaccg

SEQ ID NO: 1313 - DNA, 29 bp, Artificial Sequence
  Decoding Oligonucleotide
acttaacttgagtggccgtaattctaccg

SEQ ID NO: 1314 - DNA, 29 bp, Artificial Sequence
  Decoding Oligonucleotide
ggctaatttattacctgggtaatcgtcac

SEQ ID NO: 1315 - DNA, 29 bp, Artificial Sequence
  Decoding Oligonucleotide
accttaagaatggaatgggtaatcgtcac

SEQ ID NO: 1316 - DNA, 28 bp, Artificial Sequence
  Decoding Oligonucleotide
acttaacttgagtgggggtaatcgtcac

SEQ ID NO: 1317 - DNA, 31 bp, Artificial Sequence
  Decoding Oligonucleotide
ggctaatttattacctagtgttatgagaagc

SEQ ID NO: 1318 - DNA, 31 bp, Artificial Sequence
  Decoding Oligonucleotide
accttaagaatggaatagtgttatgagaagc

SEQ ID NO: 1319 - DNA, 30 bp, Artificial Sequence
  Decoding Oligonucleotide
acttaacttgagtggagtgttatgagaagc

SEQ ID NO: 1320 - DNA, 31 bp, Artificial Sequence
  Decoding Oligonucleotide
ggctaatttattacctctgcaatagaagaga

SEQ ID NO: 1321 - DNA, 31 bp, Artificial Sequence
  Decoding Oligonucleotide
accttaagaatggaatctgcaatagaagaga

SEQ ID NO: 1322 - DNA, 30 bp, Artificial Sequence
  Decoding Oligonucleotide
acttaacttgagtggctgcaatagaagaga

SEQ ID NO: 1323 - DNA, 30 bp, Artificial Sequence
  Decoding Oligonucleotide
ggctaatttattacctgatttttcagcgac

SEQ ID NO: 1324 - DNA, 30 bp, Artificial Sequence
  Decoding Oligonucleotide
accttaagaatggaatgatttttcagcgac

SEQ ID NO: 1325 - DNA, 29 bp, Artificial Sequence
  Decoding Oligonucleotide
acttaacttgagtgggatttttcagcgac

SEQ ID NO: 1326 - DNA, 32 bp, Artificial Sequence
  Decoding Oligonucleotide
ggctaatttattacctcgtaatatgaagatca

SEQ ID NO: 1327 - DNA, 32 bp, Artificial Sequence
  Decoding Oligonucleotide
accttaagaatggaatcgtaatatgaagatca

SEQ ID NO: 1328 - DNA, 31 bp, Artificial Sequence
  Decoding Oligonucleotide
acttaacttgagtggcgtaatatgaagatca

SEQ ID NO: 1329 - DNA, 32 bp, Artificial Sequence
  Decoding Oligonucleotide
ggctaatttattacctgaattggaatattcgt

SEQ ID NO: 1330 - DNA, 32 bp, Artificial Sequence
  Decoding Oligonucleotide
accttaagaatggaatgaattggaatattcgt

SEQ ID NO: 1331 - DNA, 31 bp, Artificial Sequence
  Decoding Oligonucleotide
acttaacttgagtgggaattggaatattcgt

SEQ ID NO: 1332 - DNA, 33 bp, Artificial Sequence
  Decoding Oligonucleotide
ggctaatttattacctggtattaatcttgaaga

SEQ ID NO: 1333 - DNA, 33 bp, Artificial Sequence
  Decoding Oligonucleotide
accttaagaatggaatggtattaatcttgaaga

SEQ ID NO: 1334 - DNA, 32 bp, Artificial Sequence
  Decoding Oligonucleotide
acttaacttgagtggggtattaatcttgaaga

SEQ ID NO: 1335 - DNA, 29 bp, Artificial Sequence
  Decoding Oligonucleotide
ggctaatttattacctacgatgtattgcc

SEQ ID NO: 1336 - DNA, 29 bp, Artificial Sequence
  Decoding Oligonucleotide
accttaagaatggaatacgatgtattgcc

SEQ ID NO: 1337 - DNA, 28 bp, Artificial Sequence
  Decoding Oligonucleotide
acttaacttgagtggacgatgtattgcc

SEQ ID NO: 1338 - DNA, 32 bp, Artificial Sequence
  Decoding Oligonucleotide
ggctaatttattacctcatatacaaacggtac

SEQ ID NO: 1339 - DNA, 32 bp, Artificial Sequence
  Decoding Oligonucleotide
accttaagaatggaatcatatacaaacggtac

SEQ ID NO: 1340 - DNA, 31 bp, Artificial Sequence
  Decoding Oligonucleotide
acttaacttgagtggcatatacaaacggtac

SEQ ID NO: 1341 - DNA, 31 bp, Artificial Sequence
  Decoding Oligonucleotide
ggctaatttattacctttccgatcttaactt

SEQ ID NO: 1342 - DNA, 31 bp, Artificial Sequence
  Decoding Oligonucleotide
accttaagaatggaatttccgatcttaactt

SEQ ID NO: 1343 - DNA, 30 bp, Artificial Sequence
  Decoding Oligonucleotide
acttaacttgagtggttccgatcttaactt

SEQ ID NO: 1344 - DNA, 31 bp, Artificial Sequence
  Decoding Oligonucleotide
ggctaatttattacctgtgttaattcgcatc

SEQ ID NO: 1345 - DNA, 31 bp, Artificial Sequence
  Decoding Oligonucleotide
accttaagaatggaatgtgttaattcgcatc

SEQ ID NO: 1346 - DNA, 30 bp, Artificial Sequence
  Decoding Oligonucleotide
acttaacttgagtgggtgttaattcgcatc

SEQ ID NO: 1347 - DNA, 30 bp, Artificial Sequence
  Decoding Oligonucleotide
ggctaatttattacctcgtttaattgggct

SEQ ID NO: 1348 - DNA, 30 bp, Artificial Sequence
  Decoding Oligonucleotide
accttaagaatggaatcgtttaattgggct

SEQ ID NO: 1349 - DNA, 29 bp, Artificial Sequence
  Decoding Oligonucleotide
acttaacttgagtggcgtttaattgggct

SEQ ID NO: 1350 - DNA, 31 bp, Artificial Sequence
  Decoding Oligonucleotide
ggctaatttattacctcaacacttataacgg

SEQ ID NO: 1351 - DNA, 31 bp, Artificial Sequence
  Decoding Oligonucleotide
accttaagaatggaatcaacacttataacgg

SEQ ID NO: 1352 - DNA, 30 bp, Artificial Sequence
  Decoding Oligonucleotide
acttaacttgagtggcaacacttataacgg

SEQ ID NO: 1353 - DNA, 34 bp, Artificial Sequence
  Decoding Oligonucleotide
ggctaatttattacctggaaataagtattatacc

SEQ ID NO: 1354 - DNA, 34 bp, Artificial Sequence
  Decoding Oligonucleotide
accttaagaatggaatggaaataagtattatacc

SEQ ID NO: 1355 - DNA, 33 bp, Artificial Sequence
  Decoding Oligonucleotide
acttaacttgagtggggaaataagtattatacc

SEQ ID NO: 1356 - DNA, 29 bp, Artificial Sequence
  Decoding Oligonucleotide
ggctaatttattacctcttaacgacggtt

SEQ ID NO: 1357 - DNA, 29 bp, Artificial Sequence
  Decoding Oligonucleotide
accttaagaatggaatcttaacgacggtt

SEQ ID NO: 1358 - DNA, 28 bp, Artificial Sequence
  Decoding Oligonucleotide
acttaacttgagtggcttaacgacggtt

SEQ ID NO: 1359 - DNA, 33 bp, Artificial Sequence
  Decoding Oligonucleotide
ggctaatttattacctgctcgttagaatttatt

SEQ ID NO: 1360 - DNA, 33 bp, Artificial Sequence
  Decoding Oligonucleotide
accttaagaatggaatgctcgttagaatttatt

SEQ ID NO: 1361 - DNA, 32 bp, Artificial Sequence
  Decoding Oligonucleotide
acttaacttgagtgggctcgttagaatttatt

SEQ ID NO: 1362 - DNA, 32 bp, Artificial Sequence
  Decoding Oligonucleotide
ggctaatttattaccttctttatcatcattga

SEQ ID NO: 1363 - DNA, 32 bp, Artificial Sequence
  Decoding Oligonucleotide
accttaagaatggaattctttatcatcattga

SEQ ID NO: 1364 - DNA, 31 bp, Artificial Sequence
  Decoding Oligonucleotide
acttaacttgagtggtctttatcatcattga

SEQ ID NO: 1365 - DNA, 31 bp, Artificial Sequence
  Decoding Oligonucleotide
ggctaatttattacctcgttcgcaatttatc

SEQ ID NO: 1366 - DNA, 31 bp, Artificial Sequence
  Decoding Oligonucleotide
accttaagaatggaatcgttcgcaatttatc

SEQ ID NO: 1367 - DNA, 30 bp, Artificial Sequence
  Decoding Oligonucleotide
acttaacttgagtggcgttcgcaatttatc

SEQ ID NO: 1368 - DNA, 31 bp, Artificial Sequence
  Decoding Oligonucleotide
ggctaatttattacctcgatcttttgttgta

SEQ ID NO: 1369 - DNA, 31 bp, Artificial Sequence
  Decoding Oligonucleotide
accttaagaatggaatcgatcttttgttgta

SEQ ID NO: 1370 - DNA, 30 bp, Artificial Sequence
  Decoding Oligonucleotide
acttaacttgagtggcgatcttttgttgta

SEQ ID NO: 1371 - DNA, 32 bp, Artificial Sequence
  Decoding Oligonucleotide
ggctaatttattacctaccactcaatataaga

SEQ ID NO: 1372 - DNA, 32 bp, Artificial Sequence
  Decoding Oligonucleotide
accttaagaatggaataccactcaatataaga

SEQ ID NO: 1373 - DNA, 31 bp, Artificial Sequence
  Decoding Oligonucleotide
acttaacttgagtggaccactcaatataaga

SEQ ID NO: 1374 - DNA, 30 bp, Artificial Sequence
  Decoding Oligonucleotide
ggctaatttattacctccgtaaattcggtc

SEQ ID NO: 1375 - DNA, 30 bp, Artificial Sequence
  Decoding Oligonucleotide
accttaagaatggaatccgtaaattcggtc

SEQ ID NO: 1376 - DNA, 29 bp, Artificial Sequence
  Decoding Oligonucleotide
acttaacttgagtggccgtaaattcggtc

SEQ ID NO: 1377 - DNA, 30 bp, Artificial Sequence
  Decoding Oligonucleotide
ggctaatttattacctcgtgagatttctga

SEQ ID NO: 1378 - DNA, 30 bp, Artificial Sequence
  Decoding Oligonucleotide
accttaagaatggaatcgtgagatttctga

SEQ ID NO: 1379 - DNA, 29 bp, Artificial Sequence
  Decoding Oligonucleotide
acttaacttgagtggcgtgagatttctga

SEQ ID NO: 1380 - DNA, 31 bp, Artificial Sequence
  Decoding Oligonucleotide
ggctaatttattacctcgaagcatagaatag

SEQ ID NO: 1381 - DNA, 31 bp, Artificial Sequence
  Decoding Oligonucleotide
accttaagaatggaatcgaagcatagaatag

SEQ ID NO: 1382 - DNA, 30 bp, Artificial Sequence
  Decoding Oligonucleotide
acttaacttgagtggcgaagcatagaatag

SEQ ID NO: 1383 - DNA, 30 bp, Artificial Sequence
  Decoding Oligonucleotide
ggctaatttattacctaaaatacgggagac

SEQ ID NO: 1384 - DNA, 30 bp, Artificial Sequence
  Decoding Oligonucleotide
accttaagaatggaataaaatacgggagac

SEQ ID NO: 1385 - DNA, 29 bp, Artificial Sequence
  Decoding Oligonucleotide
acttaacttgagtggaaaatacgggagac

SEQ ID NO: 1386 - DNA, 31 bp, Artificial Sequence
  Decoding Oligonucleotide
ggctaatttattacctcgcatggtataacat

SEQ ID NO: 1387 - DNA, 31 bp, Artificial Sequence
  Decoding Oligonucleotide
accttaagaatggaatcgcatggtataacat

SEQ ID NO: 1388 - DNA, 30 bp, Artificial Sequence
  Decoding Oligonucleotide
acttaacttgagtggcgcatggtataacat

SEQ ID NO: 1389 - DNA, 31 bp, Artificial Sequence
  Decoding Oligonucleotide
ggctaatttattacctacgctgatttataga

SEQ ID NO: 1390 - DNA, 31 bp, Artificial Sequence
  Decoding Oligonucleotide
accttaagaatggaatacgctgatttataga

SEQ ID NO: 1391 - DNA, 30 bp, Artificial Sequence
  Decoding Oligonucleotide
acttaacttgagtggacgctgatttataga

SEQ ID NO: 1392 - DNA, 33 bp, Artificial Sequence
  Decoding Oligonucleotide
ggctaatttattacctcgtagggtaaatagatt

SEQ ID NO: 1393 - DNA, 33 bp, Artificial Sequence
  Decoding Oligonucleotide
accttaagaatggaatcgtagggtaaatagatt

SEQ ID NO: 1394 - DNA, 32 bp, Artificial Sequence
  Decoding Oligonucleotide
acttaacttgagtggcgtagggtaaatagatt

SEQ ID NO: 1395 - DNA, 33 bp, Artificial Sequence
  Decoding Oligonucleotide
ggctaatttattacctcattcacagtaatttag

SEQ ID NO: 1396 - DNA, 33 bp, Artificial Sequence
  Decoding Oligonucleotide
accttaagaatggaatcattcacagtaatttag

SEQ ID NO: 1397 - DNA, 32 bp, Artificial Sequence
  Decoding Oligonucleotide
acttaacttgagtggcattcacagtaatttag

SEQ ID NO: 1398 - DNA, 15 bp, Artificial Sequence
  Signal Oligonucleotide
ccactcaagttaagt

SEQ ID NO: 1399 - DNA, 16 bp, Artificial Sequence
  Signal Oligonucleotide
attccattcttaaggt

SEQ ID NO: 1400 - DNA, 16 bp, Artificial Sequence
  Signal Oligonucleotide
aggtaataaattagcc

(19) United States
(12) Patent Application Publication
(10) Pub. No.: US 2024/0279736 A1
(43) Pub. Date: Aug. 22, 2024

(54) MULTIPLEX METHOD FOR DETECTING DIFFERENT ANALYTES AND DIFFERENT SUBGROUPS/VARIATIONS OF AN ANALYTE IN A SAMPLE

(71) Applicant: Resolve BioSciences GmbH, Monheim am Rhein (DE)

(72) Inventors: Andreas Geipel, Mettmann (DE); Frank Reinecke, Eaaen (DE); Christian Korfhage, Langenfeld (DE)

(73) Assignee: Resolve BioSciences GmbH, Monheim am Rhein (DE)

(21) Appl. No.: 18/001,930

(22) PCT Filed: Jun. 18, 2021
(86) PCT No.: PCT/EP2021/066668

§ 371 Date: Apr. 19, 2023

Related U.S. Application Data
(63) Continuation of application No. PCT/EP2020/067010, filed on Jun. 18, 2020.
(60) Provisional application No. 63/127,910, filed on Dec. 18, 2020.
(60) Provisional application No. 63/129,942, filed on Dec. 23, 2020.

(51) Int. Cl. C12Q 1/6876; C12Q 1/682

(52) U.S. Cl. CPC C12Q 1/6876; C12Q 1/682; C12Q 2600/16

(57) ABSTRACT

The technology provided herein relates to multiplex methods and kits for detecting different analytes and different subgroups/variations of an analyte in a sample, as well as in vitro methods for screening, identifying and/or testing a substance and/or drug and in vitro methods for diagnosis of a disease, and an optical multiplexing system.

### CROSS-REFERENCE TO RELATED APPLICATIONS

This document is a US National Phase entry of PCT Application No PCT/EP2021/066668, filed Jun. 18, 2021, and published Dec. 23, 2021 as PCT Publication No WO2021/255263, and claims priority to PCT Application No PCT/EP2020/067010, filed Jun. 18, 2020, and to U.S. Prov. Ser. No. 63/127,910, filed Dec. 18, 2020, and to U.S. Prov. Ser. No. 63/129,942, filed Dec. 23, 2020, the contents of each of which are hereby incorporated by reference in their entirety.

### SEQUENCE LISTING

This document contains a sequence listing in computer readable format entitled Resolve. 015NP sequence listing.txt, created Dec. 23, 2021, having a size of about 275 kb. The computer readable form is hereby incorporated by reference in its entirety.

### FIELD OF THE DISCLOSURE

The technology provided herein relates to multiplex methods and kits for detecting different analytes and different subgroups/variations of an analyte in a sample in parallel by sequential signal-encoding of said analytes, as well as in vitro methods for screening, identifying and/or testing a substance and/or drug and in vitro methods for diagnosis of a disease, and an optical multiplexing system.

### BACKGROUND

The analysis and detection of small quantities of analytes in biological and non-biological samples has become a routine practice in the clinical and analytical environment. Numerous analytical methods have been established for this purpose. Some of them use encoding techniques assigning a particular readable code to a specific first analyte which differs from a code assigned to a specific second analyte. One of the prior art techniques in this field is the so-called ‘single molecule fluorescence in situ hybridization’ (smFISH) essentially developed to detect mRNA molecules in a sample. In Lubeck et al. (2014), Single-cell in situ RNA profiling by sequential hybridization, Nat. Methods11(4), p. 360-361, the mRNAs of interest are detected via specific directly labeled probe sets. After one round of hybridization and detection, the set of mRNA specific probes is eluted from the mRNAs and the same set of probes with other (or the same) fluorescent labels is used in the next round of hybridization and imaging to generate gene specific color-code schemes over several rounds. The technology needs several differently tagged probe sets per transcript and needs to denature these probe sets after every detection round.

A further development of this technology does not use directly labeled probe sets. Instead, the oligonucleotides of the probe sets provide nucleic acid sequences that serve as initiator for hybridization chain reactions (HCR), a technology that enables signal amplification; see Shah et al. (2016), In situ transcription profiling of single cells reveals spatial organization of cells in the mouse hippocampus, Neuron 92(2), p. 342-357.

Another technique referred to as ‘multiplexed error robust fluorescence in situ hybridization’ (merFISH) is described by Chen et al. (2015), RNA imaging. Spatially resolved, highly multiplexed RNA profiling in single cells, Science 348(6233):aaa6090. There, the mRNAs of interest are detected via specific probe sets that provide additional sequence elements for the subsequent specific hybridization of fluorescently labeled oligonucleotides. Each probe set provides four different sequence elements out of a total of 16 sequence elements. After hybridization of the specific probe sets to the mRNAs of interest, the so-called readout hybridizations are performed. In each readout hybridization one out of the 16 fluorescently labeled oligonucleotides complementary to one of the sequence elements is hybridized. All readout oligonucleotides use the same fluorescent color. After imaging, the fluorescent signals are destroyed via illumination and the next round of readout hybridization takes place without a denaturing step. As a result, a binary code is generated for each mRNA species. A unique signal signature of 4 signals in 16 rounds is created using only a single hybridization round for binding of specific probe sets to the mRNAs of interest, followed by 16 rounds of hybridization of readout oligonucleotides labeled by a single fluorescence color.

A further development of this technology improves the throughput by using two different fluorescent colors, eliminating the signals via disulfide cleavage between the readout-oligonucleotides and the fluorescent label and an alternative hybridization buffer; see Moffitt et al. (2016), High-throughput single-cell gene-expression profiling with multiplexed error-robust fluorescence in situ hybridization, Proc. Natl. Acad. Sci. USA. 113(39), p. 11046-11051.

A technology referred to as ‘intron seqFISH’ is described in Shah et al. (2018), Dynamics and spatial genomics of the nascent transcriptome by intron seqFISH, Cell 117(2), p. 363-376. There, the mRNAs of interest are detected via specific probe sets that provide additional sequence elements for the subsequent specific hybridization of fluorescently labeled oligonucleotides. Each probe set provides one out of 12 possible sequence elements (representing the 12 ‘pseudocolors’ used) per color-coding round. Each color-coding round consists of four serial hybridizations. In each of these serial hybridizations, three readout probes, each labeled with a different fluorophore, are hybridized to the corresponding elements of the mRNA-specific probe sets. After imaging, the readout probes are stripped off by a 55% formamide buffer and the next hybridization follows. After 5 color-coding rounds with 4 serial hybridizations each, the color-codes are completed.

EP 0 611 828 discloses the use of a bridging element to recruit a signal generating element to probes that specifically bind to an analyte. A more specific statement describes the detection of nucleic acids via specific probes that recruit a bridging nucleic acid molecule. This bridging nucleic acids eventually recruit signal generating nucleic acids. This document also describes the use of a bridging element with more than one binding site for the signal generating element for signal amplification like branched DNA.

Player et al. (2001), Single-copy gene detection using branched DNA (bDNA) in situ hybridization, J. Histochem. Cytochem. 49(5), p. 603-611, describe a method where the nucleic acids of interest are detected via specific probe sets providing an additional sequence element. In a second step, a preamplifier oligonucleotide is hybridized to this sequence element. This preamplifier oligonucleotide comprises multiple binding sites for amplifier oligonucleotides that are hybridized in a subsequent step. These amplifier oligonucleotides provide multiple sequence elements for the labeled oligonucleotides. This way a branched oligonucleotide tree is build up that leads to an amplification of the signal.

A further development of this method referred to as is described by Wang et al. (2012), RNAscope: a novel in situ RNA analysis platform for formalin-fixed, paraffin-embedded tissues, J. Mol. Diagn. 14(1), p. 22-29, which uses another design of the mRNA-specific probes. Here two of the mRNA-specific oligonucleotides have to hybridize in close proximity to provide a sequence that can recruit the preamplifier oligonucleotide. This way the specificity of the method is increased by reducing the number of false positive signals.

Choi et al. (2010), Programmable in situ amplification for multiplexed imaging of mRNA expression, Nat. Biotechnol. 28(11), p. 1208-1212, disclose a method known as ‘HCR-hybridization chain reaction’. The mRNAs of interest are detected via specific probe sets that provide an additional sequence element. The additional sequence element is an initiator sequence to start the hybridization chain reaction. Basically, the hybridization chain reaction is based on metastable oligonucleotide hairpins that self-assemble into polymers after a first hairpin is opened via the initiator sequence.

A further development of the technology uses so called split initiator probes that have to hybridize in close proximity to form the initiator sequence for HCR, similarly to the RNAscope technology, this reduces the number of false positive signals; see Choi et al. (2018), Third-generation in situ hybridization chain reaction: multiplexed, quantitative, sensitive, versatile, robust. Development 145(12).

Mateo et al. (2019), Visualizing DNA folding and RNA in embryos at single-cell resolution, Nature Vol, 568, p. 49ff., disclose a method called ‘optical reconstruction of chromatin structure (ORCA). This method is intended to make the chromosome line visible.

EP 2 992 115 B1 describes a method of sequential single molecule hybridization and provides technologies for detecting and/or quantifying nucleic acids in cells, tissues, organs or organisms through sequential barcoding.

The methods known in the art, however, have numerous disadvantages. In particular, they are inflexible, expensive, complex, time consuming and quite often provide non-accurate results. In particular, the encoding capacities of the existing methods are low and do not meet the requirements of modern molecular biology and medicine.

Against this background, it is an object underlying the present disclosure to provide a method by means of which the disadvantages of the prior art methods can be reduced or even avoided.

### SUMMARY OF THE DISCLOSURE

The present disclosure pertains to novel multiplex method for detecting different analytes and different subgroups/variations of an analyte in a sample in parallel by sequential signal-encoding of said analytes and variations.

In particular, the present disclosure pertains multiplex method for detecting different analytes and different subgroups/variations of an analyte in a sample comprising:
(A) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element (S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
contacting the sample with at least two different sets of analyte-specific probes for at least one analyte and a variation thereof,
wherein the analyte-specific probes comprised in these different sets interacting with the same analyte, but specifically interact with different sub-structures of the same analyte,
wherein the analyte-specific probes of the first set of analyte-specific probes interacts with a sub-structure which is comprised in all variations of an analyte,
wherein the analyte-specific probes of the second set of analyte-specific probes (subgroup-specific probes) interacts with a sub-structure which is comprised only in a specific variation of the analyte,
wherein the analyte-specific probes of the first set of analyte-specific probes comprise the same identifier element (T) comprising a nucleotide sequence which is unique to the analyte to be encoded (unique identifier sequence), and
wherein the analyte-specific probes of the second set of analyte-specific probes comprise the same identifier element (T) comprising a nucleotide sequence which is unique to the analyte to be encoded (unique identifier sequence),
wherein the identifier elements (T) of the analyte-specific probes of the first set of analyte-specific probes and the identifier elements (T) of the analyte-specific probes of the second set of analyte-specific probes are different for binding different decoding oligonucleotides and/or non-signal decoding oligonucleotides.
(B) contacting the sample with at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(C) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.
(D) Detecting the signal caused by the signal element;
(E) selectively removing the decoding oligonucleotides and signal oligonucleotides from the sample, thereby essentially maintaining the specific binding of the analyte-specific probes to the analytes to be encoded;
(F) Performing at least three (3) further cycles comprising steps B) to E) to generate an encoding scheme with a code word per analyte,
(G) Performing at least one (1) further cycle comprising steps B) to E) to identify the subgroup-specific probes, wherein in particular the cycle may stop with step (D).

Furthermore, the present disclosure pertains to the use of improved decoding-oligonucleotides to increase the efficiency of the encoding scheme. The so called “multi-decoders” allows the recruiting of more than just one signal oligonucleotide and therefore can generate new signal types by utilizing the combination of two or more different signal-oligonucleotides without decreasing the brightness of the signals.

In a further aspect, embodiments of the disclosure in particular to a multiplex method for detecting different analytes in a sample by sequential signal-encoding of said analytes, comprising:
(A) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
   (aa) a binding element (S) that specifically interacts with one of the different analytes to be encoded, and
   (bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(B) contacting the sample with at least one set of decoding oligonucleotides per analyte, wherein in each set of decoding oligonucleotides for an individual analyte each decoding oligonucleotide comprises:
   (aa) an identifier connector element (t) comprising a nucleotide sequence which is essentially complementary to at least a section of the unique identifier sequence of the identifier element (T) of the corresponding analyte-specific probe set, and
   (bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(C) contacting the sample with at least a set of signal oligonucleotides, each signal oligonucleotide comprising:
   (aa) a translator connector element (C) comprising a nucleotide sequence which is essentially complementary to at least a section of the nucleotide sequence of a translator element (c) comprised in a decoding oligonucleotide, and
   (bb) a signal element.
(D) Detecting the signal caused by the signal element;
(E) selectively removing the decoding oligonucleotides and signal oligonucleotides from the sample, thereby essentially maintaining the specific binding of the analyte-specific probes to the analytes to be encoded;
(F) Performing at least three (3) further cycles comprising steps B) to E) to generate an encoding scheme with a code word per analyte, wherein in particular the last cycle may stop with step (D).

In a further aspect, embodiments of this disclosure relate to kits for multiplex analyte encoding, comprising
(A) at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element (S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(B) at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

In a further aspect, embodiments of this disclosure relate to in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure.

In a further aspect, embodiments of this disclosure provide in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the multiplex method according to the present disclosure.

In a further aspect, some embodiments of this disclosure relate to optical multiplexing systems suitable for the method according to the present disclosure, comprising at least:
at least one reaction vessel for containing the kits or part of the kits according to the present disclosure;
a detection unit comprising a microscope, in particular a fluorescence microscope
a camera
a liquid handling device.

In a further aspect, some embodiments of this disclosure relates to a kit for multiplex analyte encoding, comprising
(A) at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element (S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(B) at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element,
wherein the kit comprises at least two different sets of analyte-specific probes for an analyte,
wherein the analyte-specific probes comprised in these different sets interacting with the same analyte, but specifically interact with different sub-structures of the same analyte,
wherein the analyte-specific probes of the first set of analyte-specific probes interacts with a sub-structure which is comprised in all variations of an analyte,
wherein the analyte-specific probes of the second set of analyte-specific probes (subgroup-specific probes) interacts with a sub-structure which is comprised only in a specific variation of the analyte,
wherein the analyte-specific probes of the first set of analyte-specific probes comprise the same identifier element (T) comprising a nucleotide sequence which is unique to the analyte to be encoded (unique identifier sequence), and
wherein the analyte-specific probes of the second set of analyte-specific probes comprise the same identifier element (T) comprising a nucleotide sequence which is unique to the analyte to be encoded (unique identifier sequence),
wherein the identifier elements (T) of the analyte-specific probes of the first set of analyte-specific probes and the identifier elements (T) of the analyte-specific probes of the second set of analyte-specific probes are different.

Further, some embodiments pertain to kits for multiplex analyte encoding, comprising
(A) optionally at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element (S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(B) at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

In a further aspect, some embodiments provide in vitro methods for screening, identifying and/or testing a substance and/or drug comprising:
(a) contacting a test sample comprising a sample with a substance and/or drug
(b) detecting different analytes in a sample by sequential signal-encoding of said analytes with a method according to the present disclosure.

In a further aspect, embodiments of the disclosure extend the multiplex method for detecting different analytes (described in the first aspect) by targeting subgroups of targets in a sample. Sequential signal-encoding of one set of probes if performed as described and at least one additional set of probes is added to discriminate target subgroups.

Decoding of the main analyte (multiple rounds) is performed as described (A to I of aspect one). To identify subgroups of said analytes, additional signals are generated and analyzed in combination with the main analyte's encode. The method comprises:
(A1) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
   (aa) a binding element (S) that specifically interacts with one of the different analytes to be encoded, and
   (bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and,
(A2) contacting a subgroup of at least one analyte with a set of at least five (5) subgroup-specific probes which differ from the analyte-specific probes of another set of analyte-specific probes in the nucleotide sequence of the identifier element (T),
(B) contacting the sample with at least one set of decoding oligonucleotides per analyte, wherein in each set of decoding oligonucleotides for an individual analyte each decoding oligonucleotide comprises:
   (aa) an identifier connector element (t) comprising a nucleotide sequence which is essentially complementary to at least a section of the unique identifier sequence of the identifier element (T) of the corresponding analyte-specific probe set, and
   (bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(C) contacting the sample with at least a set of signal oligonucleotides, each signal oligonucleotide comprising:
   (aa) a translator connector element (C) comprising a nucleotide sequence which is essentially complementary to at least a section of the nucleotide sequence of a translator element (c) comprised in a decoding oligonucleotide, and
   (bb) a signal element.
(G) Detecting the signal caused by the signal element;
(H) selectively removing the decoding oligonucleotides and signal oligonucleotides from the sample, thereby essentially maintaining the specific binding of the analyte-specific probes to the analytes to be encoded;
(I) Performing at least three (3) further cycles comprising steps B) to E) to generate an encoding scheme with a code word per analyte.
(J) Performing at least one (1) further cycle comprising steps B) to E) to identify the subgroup-specific probes contacted in step A2), wherein in particular the last cycle may stop with step (D).

According to the present disclosure, unique tags (identifier) are used per target (e.g. mRNA of one single gene) or for a target group. Groups can be formed to be indicative for a certain identity, process, biological function or disease (examples: cell type, inflammation, signal processing, cancer).

Surprisingly, the methods and kits according to the present disclosure lead to the reduction of complexity. Many different probes with different binding sequences share the same (one per target) unique tag. These tags have reduced the sequence complexity (to one per target) and also have predetermined constant properties (e.g. thermodynamic stability).

Advantages of the methods and kits according to the present disclosure are:
a) Full flexibility of the process to determine the identity of the tag, e.g. use more or less signals and/or rounds, varying numbers of fluorophores, number of total signals per tag→lower numbers of targets (e.g. 20) can be identified with high confidence in less rounds (e.g. 4) than a large number of targets (e.g. 100, these need 8 rounds for the same level of confidence), even if in both cases the exact same unique tags are used.
b) All unique tags are used (recycled) in many consecutive rounds of hybridization and all primary probes contribute (provide information about their identity) in every round of identification.
c) As all tags share the same predefined properties (e.g. thermodynamic stability which allows for selective denaturing).
d)

In some advantageous embodiments, the unique tags are design as follow:
No cross-hybridization between all oligonucleotides of the process (probes, decoders, readout), so that all tag sequences are usable together (compatible)
No cross-hybridization between connector elements (bridges) of different unique tags
Stability of hybridization of the unique tags should be in a narrow range: as stable as possible (fast hybridization, i.e. short cycle times) but significantly different (in this case less stable) than the primary probe (for differential denaturation, without removing primary probes)

Therefore, the present description pertains in particular to the usage of a set of labeled and unlabeled nucleic acid sequences for specific quantitative and/or spatial detection of different analytes in parallel via specific hybridization. The technology allows the discrimination of more different analytes than different detection signals are available. The discrimination is realized via sequential signal-coding of the analytes achieved by several cycles of specific hybridization, detection of signals and selective elution of the hybridized nucleic acid sequences. In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds. The utilization of decoding-oligonucleotides leads to a sequential signal-coding technology that is e.g. more flexible, cheaper, simpler, faster and/or more accurate than other methods.

Examples for the use of the kits and method according to the present disclosure comprising subgroup-specific probes are:

### 1.) Fusion-Transcript Detection in Cancer Research

Gene fusion events that generate a chimeric protein are causative for several cancer types, accounting for approximately 20% of tumors overall (Mitelman 2007). Detection of RNA fusions has facilitated the molecular characterization and diagnosis of various tumors (reviewed by Neckles 2020). The recent approval molecules that target oncogenic fusion transcripts for degradation suggests that these are promising therapeutic targets. However, the inter- and intra-tumoral diversity of oncogenic fusion transcripts needs to be understood in more detail, ideally on the cellular level or even with subcellular resolution.
Mitelman, F., Johansson, B., & Mertens, F. (2007): The impact of translocations and gene fusions on cancer causation. Nature Reviews. Cancer, 7(4), 233-245.
Neckles, C, Sundara Rajan, S, Caplen, NJ. (2020): Fusion transcripts: Unexploited vulnerabilities in cancer? WIREs RNA; 11:e1562. https://doi.org/10.1002/wrna.1562

### 2.) RNA Subgroups (Alternative Splicing)

RNA splicing is a fundamental process of gene expression and alternative splicing plays an important role in transcriptome complexity, cell-type differentiation, and organism development. The detection of splicing products is important because aberrant splicing can lead to numerous diseases, including cancer and neurodegeneration. Splicing variability between individual cells is primarily responsible for gene expression heterogeneity. Investigations of RNA splicing variants on a single cell level will help to decipher regulatory circuits, and to classify and understand cell types and subtypes (Walks 2011). Single-molecule FISH (smFISH) was applied to detect RNA splicing variants before. Vargas (2011) was able to detect unspliced pre-mRNA, spliced introns, and spliced mRNA are detected simultaneously in a single cell, but this does not allow any multiplexing.
T. Maniatis, B. Tasic. (2002): Alternative pre-mRNA splicing and proteome expansion in metazoans. Nature, 418, pp. 236-243
Z. Waks, A. M. Klein, P. A. Silver. (2011): Cell-to-cell variability of alternative RNA splicing. Mol. Syst. Biol., 7, p. 506
D. Y. Vargas, K. Shah, M. Batish, M. Levandoski, S. Sinha, S. A. Marras, P. Schedl, S. Tyagi. (2011): Single-molecule imaging of transcriptionally coupled and uncoupled splicing. Cell, 147, pp. 1054-1065

### 3.) Viral Transcript Length

Many virus genomes harbor multiple promotors which can lead to mRNA species of various lengths, some of which have some parts in common. Detecting the exact length and composition is crucial in understanding the current phase of viral infection. For example, the HBV genome serves as the template for synthesis of multiple genomic and sub-genomic viral mRNA transcripts: Four viral promoters, Core, Pre S1, Pre S2, and X, and two enhancers, enhancer I and enhancer II, control the transcription of HBV (Zheng 2004). The quantification of each of these sub-genomic mRNA transcripts is key to understand the phase of infection and replication status.
Zheng, Y., Li, J. & Ou, J. (2004): Regulation of Hepatitis B Virus Core Promoter by Transcription Factors HNF1 and HNF4 and the Viral X Protein Regulation of Hepatitis B Virus Core Promoter by Transcription Factors HNF1 and HNF4 and the Viral X Protein. 78, 6908-6914

Before the disclosure is described in detail, it is to be understood that this disclosure is not limited to the particular component parts of the steps of the methods described. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1: Embodiment where the analyte is a nucleic acid and the probe set comprises oligonucleotides specifically binding to the analyte. The probes comprise a unique identifier sequence allowing hybridization of decoding oligonucleotides.

FIG. 2: Embodiment where the analyte is a protein and the probe set comprises proteins (here: antibodies) specifically binding to the analyte. The probes comprise a unique identifier sequence allowing hybridization of decoding oligonucleotides.

FIG. 3: Flowchart of the method according to the disclosure.

FIG. 4: Alternative options for the application of decoding and signal oligonucleotides.

FIG. 5: Example for signal encoding of three different nucleic acid sequences by two different signal types and three detection rounds; in this example, the encoding scheme includes error detection.

FIG. 6: Number of generated code words (logarithmic scale) against number of detection cycles.

FIG. 7: Calculated total efficiency of a 5-round encoding scheme based on single step efficiencies.

FIG. 8: Comparison of relative transcript abundances between different experiments.

FIG. 9: Correlation of relative transcript abundances between different experiments.

FIG. 10: Comparison of intercellular distribution of signals.

FIG. 11: Comparison of intracellular distribution of signals.

FIG. 12: Distribution pattern of different cell cycle dependent transcripts.

FIG. 13: Detection of multiple targets using a 8 round code with 2 labels (A and B) and no label (−). The targets 1, 2, 3, 4, 5, 20, and n are represented. The rounds 1, 2, 3, and 8 of the coding scheme are represented.

FIG. 14: Detection of multiple targets can be performed by an encoding scheme using a detectable marker. The ending scheme may comprise also the “0” as a marker. That means that at a specific position the transcript is not detected. Consequently, the encoding scheme may be represented by the following constructs using only two gene specific probes:
1) With detectable label F: detectable during imaging
2) With detectable label F and quencher Q: not detectable during imaging
3) With quencher Q: not detectable during imaging
4) Without label F: not detectable during imaging
5) Without signalling oligonucleotide: not detectable during imaging
6) With a decoder oligonucleotide that cannot recruit a signalling oligonucleotide
7) Without decoder oligonucleotide: not detectable during imaging

FIG. 15: Detection of subgroups of different targets (Round 0) using additional, subgroup-specific probe sets. The procedure comprises contacting the analyte-specific probe sets in common (shared) parts and, in addition, contacting subgroup-specific probe sets to subgroup-determining (exclusive) parts (see Round 1; description, A2). Detection of the analytes using the probe sets bound to the shared parts (Round 1 through 4), using the decoding schema also described. Round 5: In at least one dedicated round, only the subgroup-specific probe sets are detected. The presence of the exclusive part of the target is then combined with the results from the previous rounds, allowing to discriminate subgroup 1′ within group 1 and subgroup 2′ within group 2.

FIG. 16: Possible structures of a multi-decoder. The numbers depict the examples. (A) is the unique identifier sequence, (a) is the corresponding sequence of the decoding oligonucleotide or multi-decoder and (c1) to (c3) are different sequence elements, that specifically bind to different signal oligonucleotides. Examples 2 to 5 show different versions of multi decoders. The order of the different sequence elements as well as the number of signal oligonucleotide binding elements is not fixed. Example 1 shows a normal decoding oligonucleotide since there is only one signal oligonucleotide binding element (c1).

FIG. 17: Example for signal encoding of three different nucleic acid sequences by using multi-decoders and two different signal oligonucleotides creating three different signal types and three detection rounds. In this example, the encoding scheme includes error detection and correction.

FIG. 18: Number of generated codewords (logarithmic scale) against number of detection cycles. The number of codewords for merFISH does not exponentially increase with the number of detection cycles but gets less effective with each added round. In contrast, the number of codewords for intronSeqFISH, the method of the present disclosure without using multi-decoders, the method with multi-decoders increases exponentially. The slope of the curve for the method using multi-decoders is much higher than that of the prior invention, leading to more than 20 000 000 times more code words usable after 20 rounds of detection.

### DETAILED DESCRIPTION OF THE DISCLOSURE

Disclosed herein are novel multiplex methods and kits for detecting different analytes and different subgroups/variations of an analyte in a sample.

The present disclosure describes the usage of a set of labeled and unlabeled nucleic acid sequences for specific quantitative and/or spatial detection of different analytes in parallel via specific hybridization. The technology allows the discrimination of more different analytes than different detection signals are available. The discrimination may be realized via sequential signal-coding of the analytes achieved by several cycles of specific hybridization, detection of signals and selective elution of the hybridized nucleic acid sequences.

In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds.

The utilization of decoding-oligonucleotides leads to a sequential signal-coding technology that is more flexible, cheaper, simpler, faster and/or more accurate than other methods.

### A. Definitions

According to the present disclosure an “analyte” is the subject to be specifically detected as being present or absent in a sample and, in case of its presence, to encode it. It can be any kind of entity, including a protein, polypeptide, protein or a nucleic acid molecule (e.g. RNA, PNA or DNA) of interest. The analyte provides at least one site for specific binding with analyte-specific probes. Sometimes herein the term “analyte” is replaced by “target”. An “analyte” according to the disclosure incudes a complex of subjects, e.g. at least two individual nucleic acid, protein or peptides molecules. In an embodiment of the disclosure an “analyte” excludes a chromosome. In another embodiment of the disclosure an “analyte” excludes DNA. The term “analyte” according to the present disclosure may include a group of different variations/embodiments of the same analyte e.g. splice variants of an analyte, variations comprising different introns and/or exons, sequences comprising an UTR and/or sequences having different length. In particular, the basic sequence and the variations having a sequence identity of at least 50, %, at least 75%, at least 80%, at least 85%, at least 90%, or at least 95%.

In some embodiments, an analyte may be a “coding sequence”, “encoding sequence”, “structural nucleotide sequence” or “structural nucleic acid molecule” which refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

A “sample” as referred to herein is a composition in liquid or solid form suspected of comprising the analytes to be encoded. In particular, the sample is a biological sample, preferably comprising biological tissue, further preferably comprising biological cells and/or extracts and/or part of cells. For example, the cell is a prokaryotic cells or a eukaryotic cell, in particular a mammalian cell, in particular a human cell. In some embodiments, the biological tissue, biological cells, extracts and/or part of cells are fixed. In particular, the analytes are fixed in a permeabilized sample, such as a cell-containing sample.

As used in the present disclosure, “cell”, “cell line”, and “cell culture” can be used interchangeably and all such designations include progeny. Thus, the words “transformants” or “transformed cells” include the primary subject cell and cultures derived therefrom without regard for the number of transfers. It is also understood that all progeny may not be precisely identical in DNA content, due to deliberate or inadvertent mutations. Mutant progeny that have the same functionality as screened for in the originally transformed cell are included.

An “encoding scheme” may describe a set of code words that are associated with the analytes to be detected. Each code word refers to one of the analytes and can be distinguished from all other code words. A code word hereby is a sequence of signs provided by the detection cycles of the method. A sign within a code word is a detectable signal or the absence of a signal. A code word does not need to comprise of all different signals used in the method. The number of signs in a code word is defined by the number of detection cycles.

An “oligonucleotide” as used herein, refers to s short nucleic acid molecule, such as DNA, PNA, LNA or RNA. The length of the oligonucleotides is within the range 4-200 nucleotides (nt), preferably 6-80 nt, more preferably 8-60 nt, more preferably 10-50 nt, more preferably 12 to 35 depending on the number of consecutive sequence elements. The nucleic acid molecule can be fully or partially single-stranded. The oligonucleotides may be linear or may comprise hairpin or loop structures. The oligonucleotides may comprise modifications such as biotin, labeling moieties, blocking moieties, or other modifications.

The “analyte-specific probe” consists of at least two elements, namely the so-called binding element (S) which specifically interacts with one of the analytes, and a so-called identifier element (T) comprising the ‘unique identifier sequence’. The binding element (S) may be a nucleic acid such as a hybridization sequence or an aptamer, or a peptidic structure such as an antibody.

In particular, in some embodiments the binding element (S) comprises moieties which are affinity moieties from affinity substances or affinity substances in their entirety selected from the group consisting of antibodies, antibody fragments, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and their derivatives, mutants or combinations thereof. In further advantageous embodiments, the antibody fragment is a Fab, an scFv; a single domain, or a fragment thereof, a bis scFv, Fab2, Fab3, minibody, maxibody, diabody, triabody, tetrabody or tandab, in particular a single-chain variable fragment (scFv).

The “unique identifier sequence” as comprised by the analyte-specific probe is unique in its sequence compared to other unique identifiers. “Unique” in this context means that it specifically identifies only one analyte, such as Cyclin A, Cyclin D, Cyclin E etc., or, alternatively, it specifically identifies only a group of analytes, independently whether the group of analytes comprises a gene family or not. Therefore, the analyte or a group of analytes to be encoded by this unique identifier can be distinguished from all other analytes or groups of analytes that are to be encoded based on the unique identifier sequence of the identifier element (T). Or, in other words, there is only one ‘unique identifier sequence’ for a particular analyte or a group of analytes, but not more than one, i.e. not even two. Due to the uniqueness of the unique identifier sequence the identifier element (T) hybridizes to exactly one type of decoding oligonucleotides. The length of the unique identifier sequence is within the range 8-60 nt, preferably 12-40 nt, more preferably 14-20 nt, depending on the number of analytes encoded in parallel and the stability of interaction needed. A unique identifier may be a sequence element of the analyte-specific probe, attached directly or by a linker, a covalent bond or high affinity binding modes, e.g. antibody-antigen interaction, streptavidin-biotin interaction etc. It is understood that the term “analyte specific probe” includes a plurality of probes which may differ in their binding elements (S) in a way that each probe binds to the same analyte but possibly to different parts thereof, for instance to different (e.g. neighboring) or overlapping sections of the nucleotide sequence comprised by the nucleic acid molecule to be encoded. However, each of the plurality of the probes comprises the same identifier element (T).

A “decoding oligonucleotide” consists of at least two sequence elements. One sequence element that can specifically bind to a unique identifier sequence, referred to as an “identifier connector element” (t) or “first connector element” (t), and a second sequence element specifically binding to a signal oligonucleotide, referred to as “translator element” (c). The length of the sequence elements is within the range 8-60 nt, preferably 12-40 nt, more preferably 14-20 nt, de-pending on the number of analytes to be encoded in parallel, the stability of interaction needed and the number of different signal oligonucleotides used. The length of the two sequence elements may or may not be the same.

In some advantageous embodiments, the decoding oligonucleotide in the kits and/or methods of the present disclosure may be a “multi-decoder”. A “multi-decoder” is a decoding oligonucleotide that consists of at least three sequence elements. One sequence element (the identifier connector element (t)) can specifically bind to a unique identifier sequence (identifier element (T)) and at least two other sequence elements (translator elements (c)) specifically bind different signal oligonucleotides (each of these sequence elements specifically binds a signal oligonucleotide that differs to all other signal oligonucleotides recruited by other elements of the multi-decoder). The length of the sequence elements is within the range 8-60 nt, preferably 12-40 nt, more preferably 14-20 nt, depending on the number of analytes detected in parallel, the stability needed and the number of different signal oligonucleotides used. The length of the sequence elements may or may not be the same.

Therefore, in some advantageous embodiments, the decoding oligonucleotide is a multi-decoder comprising
an identifier connector element (t) comprising a nucleotide sequence which is essentially complementary to at least a section of the unique identifier sequence of the identifier element (T) of the corresponding analyte-specific probe set, and
at least two translator elements (c) comprising each a nucleotide sequence allowing a specific hybridization of a different signal oligonucleotide.

Therefor, the first translator element binds a different signal oligonucleotide as the second translator element. In particular, the signal oligonucleotides differ in the signal element comprised in the signal oligonucleotide, e.g. in the kind of the fluorophore.

A “signal oligonucleotide” as used herein comprises at least two elements, a so-called “translator connector element” (C) or “second connector element” (C) having a nucleotide sequence specifically hybridizable to at least a section of the nucleotide sequence of the translator element (c) of the decoding oligonucleotide, and a “signal element” which provides a detectable signal. This element can either actively generate a detectable signal or provide such a signal via manipulation, e.g. fluorescent excitation. Typical signal elements are, for example, enzymes that catalyze a detectable reaction, fluorophores, radioactive elements or dyes.

A “set” refers to a plurality of moieties or subjects, e.g. analyte-specific probes or decoding oligonucleotides, whether the individual members of said plurality are identical or different from each other. In an analyte specific probe set, the analyte specific probes are identical in the identifier element (T) but may comprise a different binding element (S) for specifically interacting with the same analyte but for specifically interacting with different sub-structures of the same analyte to be encoded.

“Selective denaturation” may be the process of eliminating bound decoding oligonucleotides and signal oligonucleotides with highest efficiency while at the same time the target specific probes have to stay hybridized with the highest efficiency. The total efficiency of these two combined events may to be at least 0.22 for two detection cycles, 0.37 for three detection cycles, 0.47 for four detection cycles, 0.55 for five detection cycles, 0.61 for six detection cycles, 0.65 for seven detection cycles, 0.69 for eight detection cycles, 0.72 for nine detection cycles and 0.74 for 10 detection cycles, 0.76 for 11 detection cycles and 0.78 for 12 detection cycles.

In an embodiment of the disclosure a single set refers to a plurality of oligonucleotides

An “analyte specific probe set” refers to a plurality of moieties or sub-jects, e.g. analyte-specific probes that are different from each other and bind to independent regions of the analyte. A single analyte specific probe set is further characterized at least by the same unique identifier.

A “subgroup specific probe set” may comprise the same characteristics as the “analyte specific probe set”, but differs in terms of encoding and decoding information. The “subgroup specific probe sets” are only used to add information (mostly presence/absence) to a code that is already encoded by a “analyte specific probe set”.

A “subgroup” or also called “variation” of an analyte refers to an embodiment of an analyte, wherein the variation comprises a common or “shared” element (e.g. identical nucleic acid sequence) comprised in all embodiments (or variations) of the same analyte and at least an additional element discriminating the analyte(target)-subgroups/variations among each other and/or from the basic analyte.

In some embodiments, a subgroup/variation of at least one analyte with a set of at least five (5) is contacted with subgroup-specific probes which differ from the analyte-specific probes of another set of analyte-specific probes in the nucleotide sequence of the identifier element (T).

A “decoding oligonucleotide set” refers to a plurality of decoding oligonucleotides specific for a certain unique identifier needed to realize the encoding independent of the length of the code word. Each and all of the decoding oligonucleotides included in a “decoding oligonucleotide set” bind to the same unique identifier element (T) of the analyte-specific probe.

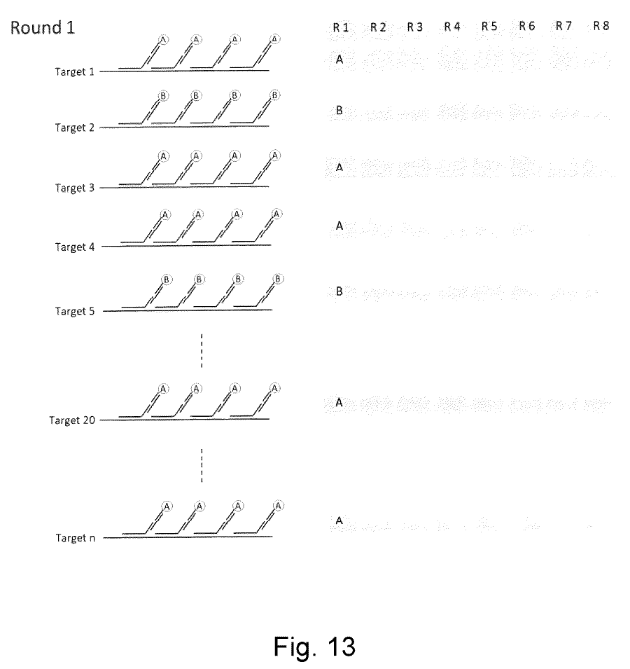

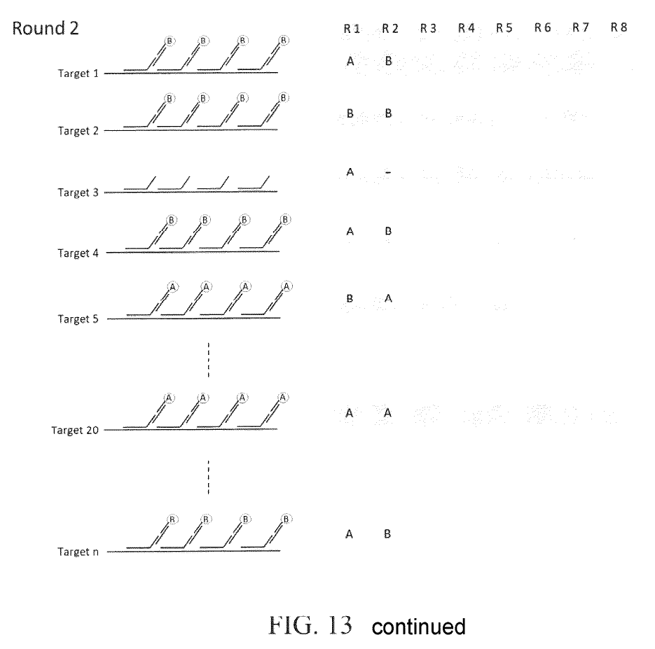

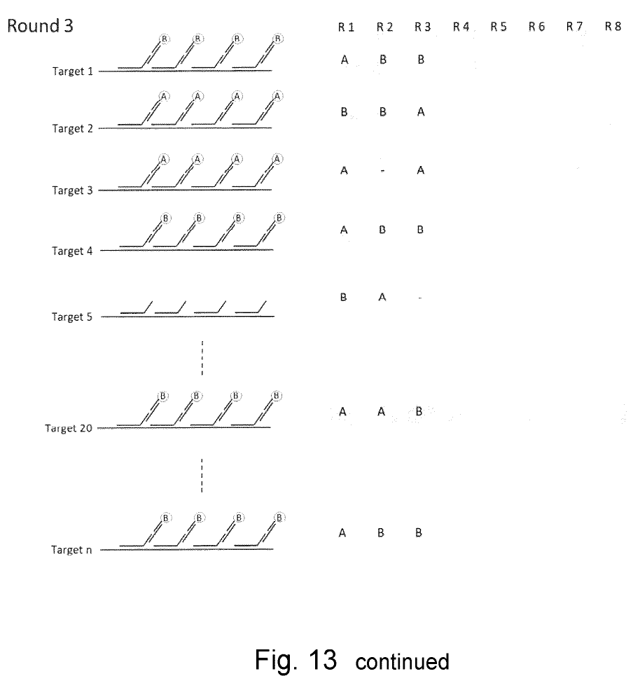

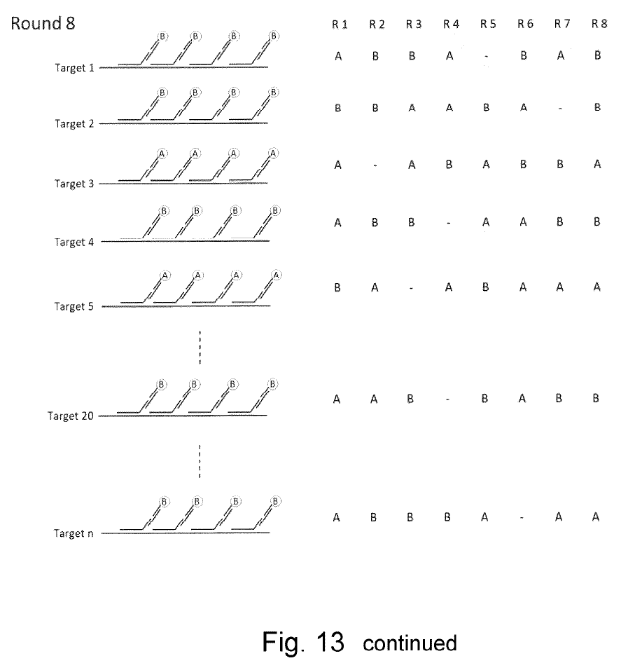

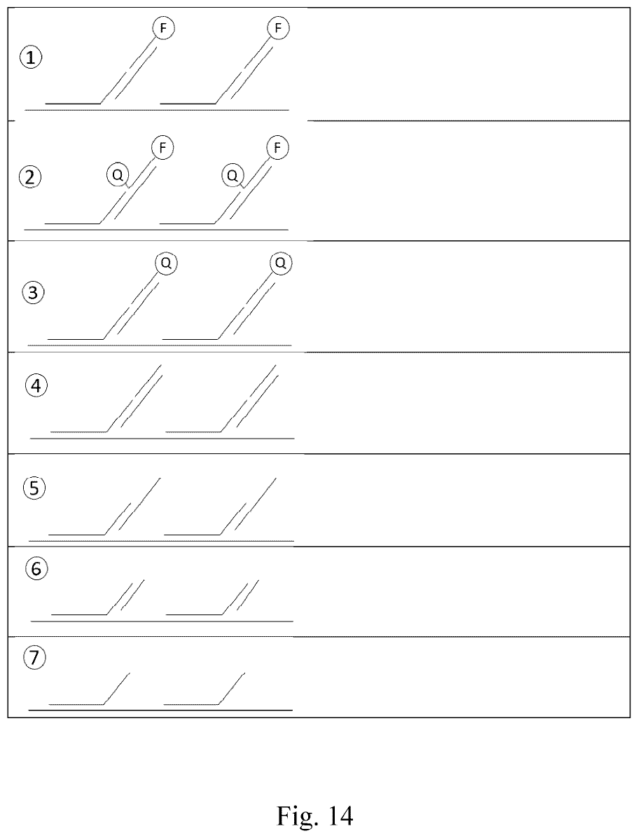

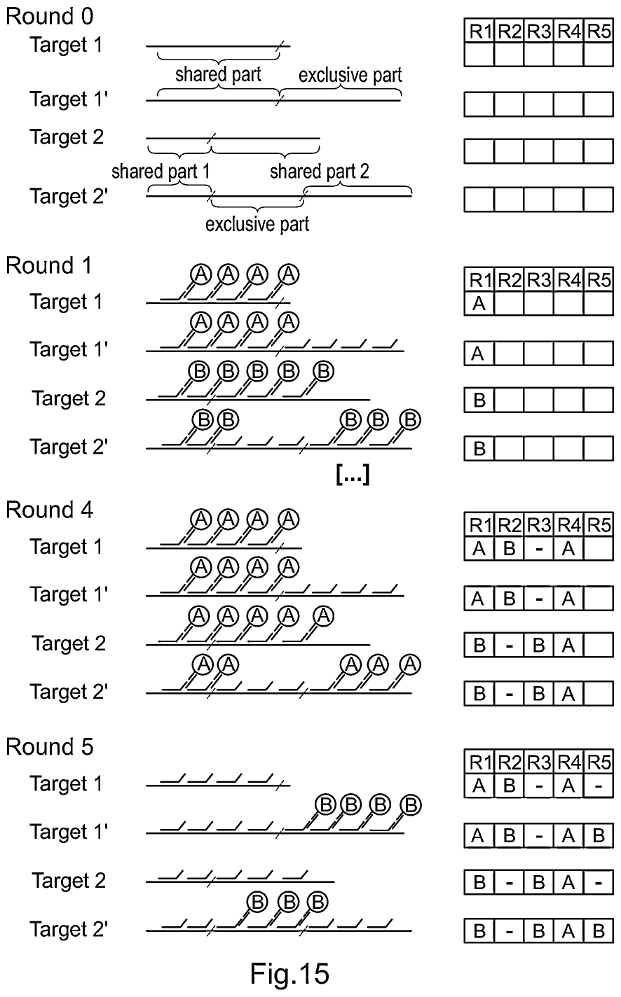

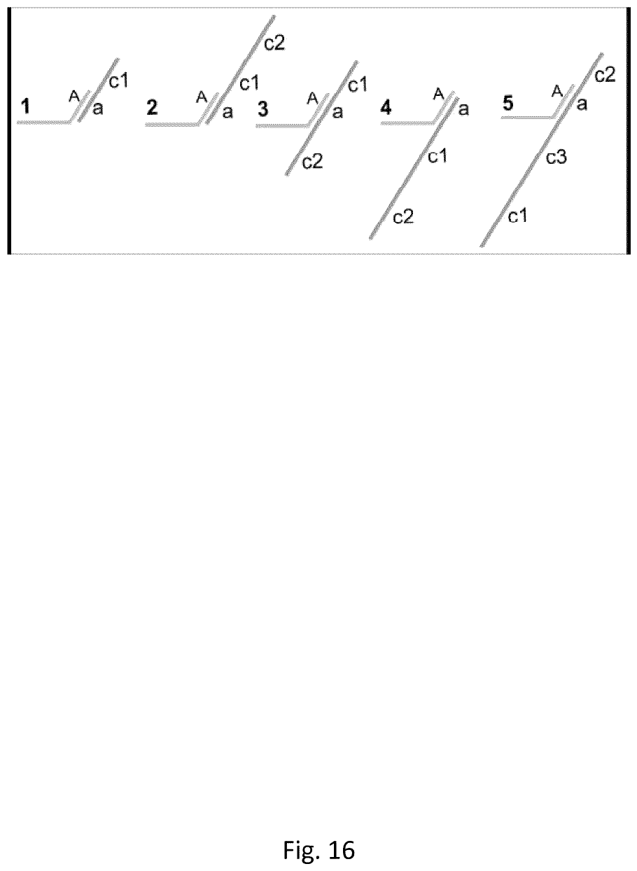

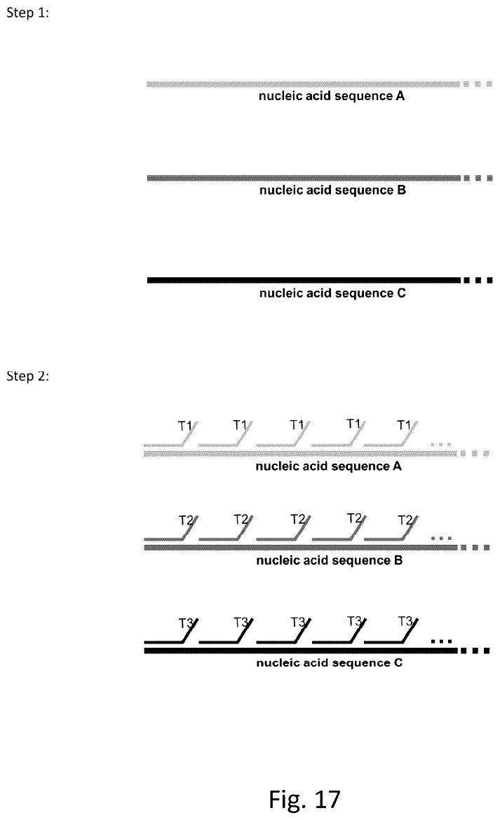

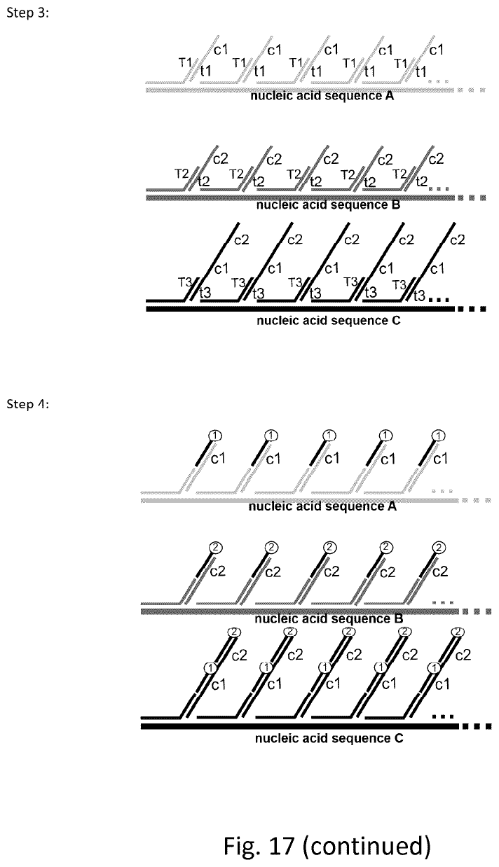

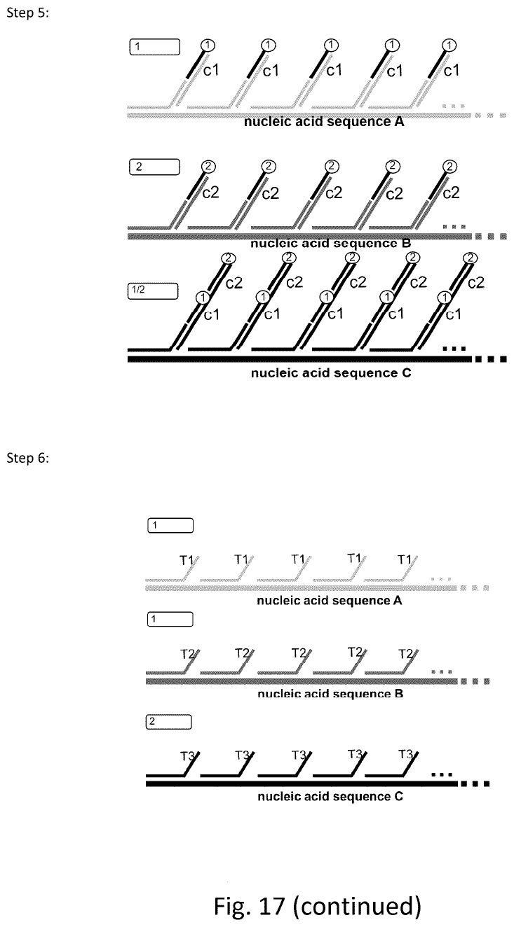

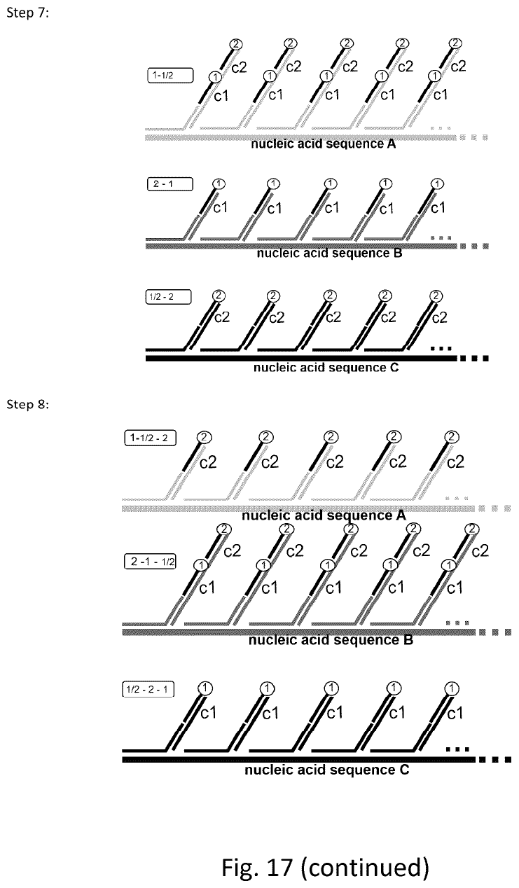

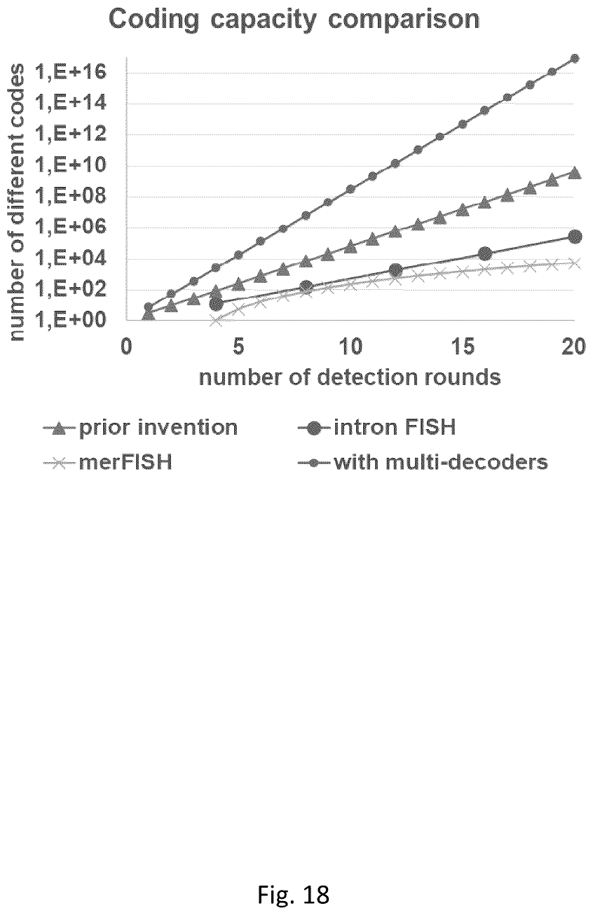

In certain embodiments, this pattern of binding or hybridization of the decoding oligonucleotides may be converted into a “codeword.” For example, the codewords could be also “101” and “110” for an analyte, where a value of 1 represents binding and a value of 0 represents no binding. The codewords may also have longer lengths in other embodiments (see FIG. 13). A codeword can be directly related to a specific unique identifier sequence of a analyte-specific probe. Accordingly, different analyte-specific probe may match certain codewords, which can then be used to identify the different analytes of the analyte-specific probe based on the binding patterns of the decoding oligonucleotide. However, if no binding is evident, then the codeword would be “000” in this example.

The values in each codeword can also be assigned in different fashions in some embodiments. For example, a value of 0 could represent binding while a value of 1 represents no binding. Similarly, a value of 1 could represent binding of a secondary nucleic acid probe with one type of signaling entity while a value of 0 could represent binding of a secondary nucleic acid probe with another type of distinguishable signaling entity. These signaling entities could be distinguished, for example, via different colors of fluorescence. In some cases, values in codewords need not be confined to 0 and 1. The values could also be drawn from larger alphabets, such as ternary (e.g., 0, 1, and 2) or quaternary (e.g., 0, 1, 2, and 3) systems. Each different value could, for example, be represented by a different distinguishable signaling entity, including (in some cases) one value that may be represented by the absence of signal.

The codewords for each analyte may be assigned sequentially, or may be assigned at random. For instance, a first analyte may be assigned to 101, while a second nucleic acid target may be assigned to 110. In addition, in some embodiments, the codewords may be assigned using an error-detection system or an error-correcting system, such as a Hamming system, a Golay code, or an extended Hamming system (or a SECDED system, i.e., single error correction, double error detection). Generally speaking, such systems can be used to identify where errors have occurred, and in some cases, such systems can also be used to correct the errors and determine what the correct codeword should have been. For example, a codeword such as 001 may be detected as invalid and corrected using such a system to 101, e.g., if 001 is not previously assigned to a different target sequence. A variety of different error-correcting codes can be used, many of which have previously been developed for use within the computer industry; however, such error-correcting systems have not typically been used within biological systems. Additional examples of such error-correcting codes are discussed in more detail below.

“Essentially complementary” means, when referring to two nucleotide sequences, that both sequences can specifically hybridize to each other under stringent conditions, thereby forming a hybrid nucleic acid molecule with a sense and an antisense strand connected to each other via hydrogen bonds (Watson-and-Crick base pairs). “Essentially complementary” includes not only perfect base-pairing along the entire strands, i.e. perfect complementary sequences but also imperfect complementary sequences which, however, still have the capability to hybridize to each other under stringent conditions. Among experts it is well accepted that an “essentially complementary” sequence has at least 88% sequence identity to a fully or perfectly complementary sequence.

“Percent sequence identity” or “percent identity” in turn means that a sequence is compared to a claimed or described sequence after alignment of the sequence to be compared (the “Compared Sequence”) with the described or claimed sequence (the “Reference Sequence”). The percent identity is then determined according to the following formula: percent identity=100 [1−(C/R)]
wherein C is the number of differences between the Reference Sequence and the Compared Sequence over the length of alignment between the Reference Sequence and the Compared Sequence, wherein
(i) each base or amino acid in the Reference Sequence that does not have a corresponding aligned base or amino acid in the Compared Sequence and
(ii) each gap in the Reference Sequence and
(iii) each aligned base or amino acid in the Reference Sequence that is different from an aligned base or amino acid in the Compared Sequence, constitutes a difference and (iiii) the alignment has to start at position 1 of the aligned sequences;
and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the herein above calculated percent identity is less than the specified percent identity.

In the “incubation” steps as understood herein the respective moieties or subjects such as probes or oligonucleotide, are brought into contact with each other under conditions well known to the skilled person allowing a specific binding or hybridization reaction, e.g. pH, temperature, salt conditions etc. Such steps may therefore, be preferably carried out in a liquid environment such as a buffer system which is well known in the art.

The “removing” steps according to the disclosure may include the washing away of the moieties or subjects to be removed such as the probes or oligonucleotides by certain conditions, e.g. pH, temperature, salt conditions etc., as known in the art.

It is understood that in an embodiment of the method according to the present disclosure a plurality of analytes can be encoded in parallel. This requires the use of different sets of analyte-specific probes in step (1). The analyte-specific probes of a particular set differ from the analyte-specific probes of another set. This means that the analyte-specific probes of set 1 bind to analyte 1, the analyte-specific probes of set 2 bind to analyte 2, the analyte-specific probes of set 3 bind to analyte 3, etc. In this embodiment also the use of different sets of decoding oligonucleotides is required in the methods according to the present disclosure.

The decoding oligonucleotides of a particular set differ from the decoding oligonucleotides of another set. This means, the decoding oligonucleotides of set 1 bind to the analyte-specific probes of above set 1 of analyte-specific probes, the decoding oligonucleotides of set 2 bind to the analyte-specific probes of above set 2 of analyte-specific probes, the decoding oligonucleotides of set 3 bind to the analyte-specific probes of above set 3 of analyte-specific probes, etc.

In this embodiment where a plurality of analytes is to be encoded in parallel the different sets of analyte-specific probes may be provided as a premixture of different sets of analyte-specific probes and/or the different sets of decoding oligonucleotides may be provided as a premixture of different sets of decoding oligonucleotides. Each mixture may be contained in a single vial. Alternatively, the different sets of analyte-specific probes and/or the different sets of decoding oligonucleotides may be provided in steps singularly.

A “kit” is a combination of individual elements useful for carrying out the use and/or method of the disclosure, wherein the elements are optimized for use together in the methods. The kits may also contain additional reagents, chemicals, buffers, reaction vials etc. which may be useful for carrying out the method according to the disclosure. Such kits unify all essential elements required to work the method according to the disclosure, thus minimizing the risk of errors. Therefore, such kits also allow semi-skilled laboratory staff to perform the method according to the present disclosure.

The term “quencher” or “quencher dye” or “quencher molecule” refers to a dye or an equivalent molecule, such as nucleoside guanosine (G) or 2′-deoxyguanosine (dG), which is capable of reducing the fluorescence of a fluorescent reporter dye or donor dye. A quencher dye may be a fluorescent dye or non-fluorescent dye. When the quencher is a fluorescent dye, its fluorescence wavelength is typically substantially different from that of the reporter dye and the quencher fluorescence is usually not monitored during an assay. Some embodiments of the present disclosure disclose signal oligonucleotides comprising a quencher and/or a quencher in combination with a signal element (see FIG. 14), and therefore the signal oligonucleotides is not detectable during imaging.

In an embodiment of the disclosure the sample is a biological sample, preferably comprising biological tissue, further preferably comprising biological cells. A biological sample may be derived from an organ, organoids, cell cultures, stem cells, cell suspensions, primary cells, samples infected by viruses, bacteria or fungi, eukaryotic or prokaryotic samples, smears, disease samples, a tissue section.

The method is particularly qualified to encode, identify, detect, count or quantify analytes or single analytes molecules in a biological sample, i.e. such as a sample which contains nucleic acids or proteins as said analytes. It is understood that the biological sample may be in a form as it is in its natural environment (i.e. liquid, semi-liquid, solid etc.), or processed, e.g. as a dried film on the surface of a device which may be re-liquefied before the method is carried out.

In another embodiment of the disclosure prior to step (2) the biological tissue and/or biological cells are fixed. For example, in some embodiments, the cell and/or the tissue is fixed prior to introducing the probes, e.g., to preserve the positions of the analytes like nucleic acids within the cell. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE).

This measure has the advantage that the analytes to be encoded, e.g. the nuclei acids or proteins, are immobilized and cannot escape. In doing so, the analytes then prepared for a better detection or encoding by the method according to the disclosure.

In yet a further embodiment within the set of analyte-specific probes the individual analyte-specific probes comprise binding elements (S1, S2, S3, S4, S5) which specifically interact with different sub-structures of one of the analytes to be encoded.

By this measure the method becomes even more robust and reliable because the signal intensity obtained at the end of the method or a cycle, respectively, is increased. It is understood, that the individual probes of a set while binding to the same analyte differ in their binding position or binding site at or on the analyte. The binding elements S1, S2, S3, S4, S5 etc. of the first, second, third fourth, fifth etc. analyte-specific probes therefore bind to or at a different position which, however, may or may not overlap.

In an advantageous embodiment, the present disclosure pertains to kit for multiplex analyte encoding, comprising:
(A) at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes, and—optionally—also at least a subgroup specific probe set to discriminate targets (analytes/variations) with shared and exclusive parts of an analyte, which specifically interact with different sub-structures of the same analyte, each analyte-specific probe comprising
(aa) a binding element (S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(B) at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

A multiplex method or assay allow the simultaneously measurement of multiple analytes according to the present disclosure it may be used to determine the presence or absence of a plurality of predetermined (known) analytes like nucleic acid target sequences in a sample. An analyte may be “predetermined” in that its sequence is known to design a probe that binds to the that target.

In some advantageous embodiments according to the present disclosure at least 20, in particular at least 25, in particular at least 30 different analytes are detected and/or quantified in a sample in parallel. For example, there may be at least 5, at least 10, at least 20, at least 50, at least 75, at least 100, at least 300, at least 1,000, at least 3,000, at least 10,000, or at least 30,000 distinguishable analyte-specific probes that are applied to a sample, e.g., simultaneously or sequentially.

In some advantageous embodiments for the multiplexing twenty (20) or more different sets of analyte-specific probes for encoding of at least 20 different analytes or more are required, in particular more than 50, more than 100 or more than 200. In the multiplexing methods of the present disclosure, in particular at least 20 different groups of analytes (e.g. mRNA molecules) i.e. tags are targeted.

In an advantageous embodiment, the kit comprises at least two different sets of analyte-specific probes per analyte,
wherein the analyte-specific probes comprised in these different sets interacting with the same analyte, but specifically interact with different sub-structures of the same analyte,
wherein the analyte-specific probes of the first set of analyte-specific probes interacts with a sub-structure which is comprised in all variations of an analyte,
wherein the analyte-specific probes of the second set of analyte-specific probes interacts with a sub-structure which is comprised only in a specific variation of the analyte (subgroup specific probe set),
wherein the analyte-specific probes of the first set of analyte-specific probes comprise the same identifier element (T) comprising a nucleotide sequence which is unique to the analyte to be encoded (unique identifier sequence), and
wherein the analyte-specific probes of the second set of analyte-specific probes comprise the same identifier element (T) comprising a nucleotide sequence which is unique to the analyte to be encoded (unique identifier sequence),
wherein the identifier elements (T) of the analyte-specific probes of the first set of analyte-specific probes and the identifier elements (T) of the analyte-specific probes of the second set of analyte-specific probes are different for binding different decoding oligonucleotides and/or non-signal decoding oligonucleotides.

In some advantageous embodiments, at least 4 rounds to collect information for identification of the analyte are carried out, wherein multiple readout increases the accuracy of identification and avoids false positives. The unique tag can be identified by various techniques, including hybridization, e.g. with labeled probes, directly or indirectly or by sequencing (by synthesis, ligation). In particular, the identity of the tag can be encoded with one single signal (binary code), two or more signals, wherein the signal can be a fluorescent label (e.g. attached to an oligonucleotide).

In some advantageous embodiments according to the present disclosure, the kit does not comprise sets of analyte-specific probes as defined under item A).

Preferably, if the analyte in the kits or methods according to the present disclosure is a nucleic acid, each set of analyte-specific probes comprises at least five (5) analyte-specific probes, in particular at least ten (10) analyte-specific probes, in particular at least fifteen (15) analyte-specific probes, in particular at least twenty (20) analyte-specific probes which specifically interact with different sub-structures of the same analyte. Nucleic acid analyte includes specific DNA molecules, e.g. genomic DNA, nuclear DNA, mitochondrial DNA, viral DNA, bacterial DNA, extra- or intracellular DNA etc., and specific mRNA molecules, e.g. hnRNA, miRNA, viral RNA, bacterial RNA, extra- or intracellular RNA, etc.

Preferably, if the analyte in the kits or methods according to the present disclosure is a peptide, a polypeptide or a protein, each set of analyte-specific probes comprises at least two (2) analyte-specific probes, in particular at least three (3) analyte-specific probes, in particular at least four (4) analyte-specific probes which specifically interact with different sub-structures of the same analyte.

In some advantageous embodiments according to the present disclosure the kit comprises at least two different sets of signal oligonucleotides, wherein the signal oligonucleotides in each set comprise a different signal element and comprise a different connector element (C).

In particular, the kit may comprise at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide.

In some embodiments the kit comprises at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets for at least one analyte differ in the translator element (c) comprising a nucleotide sequence allowing a specific hybridization of a signal oligonucleotide.

In some advantageous embodiments, the number of different sets of decoding oligonucleotides per analyte comprising different translator elements (c) corresponds to the number of different sets of signal oligonucleotides comprising different connector elements (C). However, the decoding oligonucleotides in a particular set of decoding oligonucleotides may interacts with identical identifier elements (T) which are unique to a particular analyte. In particular, all sets of decoding oligonucleotides for the different analytes may comprise the same type(s) of translator element(s) (c).

In another aspect, the present disclosure is generally directed to a methods including acts of exposing a sample to a plurality of analyte-specific probes; for each of the analyte-specific probes, determining binding of the analyte-specific probes within the sample; creating codewords based on the binding of the analyte-specific probes, the decoding oligonucleotides and the signal oligonucleotides; and for at least some of the codewords, matching the codeword to a valid codeword. In certain embodiments, this pattern of binding or hybridization of the analyte-specific probes, the decoding oligonucleotides and the signal oligonucleotides may be converted into a “codeword.” For example, for instance, the codewords may be “101” and “110” for a first analyte and a second analyte, respectively, where a value of 1 represents binding and a value of 0 represents no binding of decoding oligonucleotides and/or the binding of signal oligonucleotides without and/or quenched signal element. The analyte in the detection round/cycle is therefore not detectable during imaging.

To create such a zero (0) in a codeword for an individual analyte the kit may comprise:
(D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T),
   wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

To create such a zero (0) in a codeword for an individual analyte the kit may comprise:
(D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T),
   wherein each non-signal decoding oligonucleotide comprises an identifier connector element (t) comprising a nucleotide sequence which is essentially complementary to at least a section of a unique identifier sequence, and comprise a translator element that does not interact/bind to a signal oligonucleotide due to an instabil binding sequence and/or due to the translator element is to short (c) comprising a nucleotide sequence allowing a specific hybridization of a signal oligonucleotide.

In some advantageous embodiments, the kit comprises:
(D) at least two (2) different sets of non-signal decoding oligonucleotides for binding to at least two different identifier elements (T) of analyte-specific probes, each set of non-signal decoding oligonucleotides interacting with a different identifier element (T),
   wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments, the different sets of non-signal decoding oligonucleotides may be comprised in a pre-mixture of different sets of non-signal decoding oligonucleotides or exist separately.

Furthermore, in some advantageous embodiments the kit may comprises:
(E) a set of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

In some advantageous embodiments, the kit comprises:
(E) at least two sets of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

In some advantageous embodiments, the different sets of non-signal oligonucleotides may be comprised in a pre-mixture of different sets of non-signal oligonucleotides or exist separately.

Further, in some embodiments the decoding oligonucleotides in a particular set of decoding oligonucleotides interacts with identical identifier elements (T) which are unique to a particular analyte.

In some advantageous embodiments, the different sets of decoding oligonucleotides may be comprised in a pre-mixture of different sets of decoding oligonucleotides or exist separately. In some advantageous embodiments, the different sets of analyte-specific probes may be comprised in a pre-mixture of different sets of analyte-specific probes or exist separately. In some advantageous embodiments, the different sets of signal oligonucleotides may be comprised in a pre-mixture of different sets of signal oligonucleotides or exist separately.

In some advantageous embodiments, a mixture of decoding oligonucleotides and/or multi-decoders is provided that specifically hybridize to the unique identifier sequences of the probe sets. In some embodiments, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide. In some embodiments multi-decoders comprising at least three sequence elements are used, a first element that is complementary to the unique identifier sequences of the corresponding probe set and at least to additional sequence elements (translator elements) that provide sequences for the specific hybridization of at least two different signal oligonucleotides. The translator elements define the type of signals that are recruited to the multi-decoder. Different possible structures of a multi-decoder can be seen in FIG. 17. Since a multi-decoder does recruit a full signal oligonucleotide per translator element, the brightness of the signals in each channel is not lower than the brightness of signals with decoding oligonucleotides.

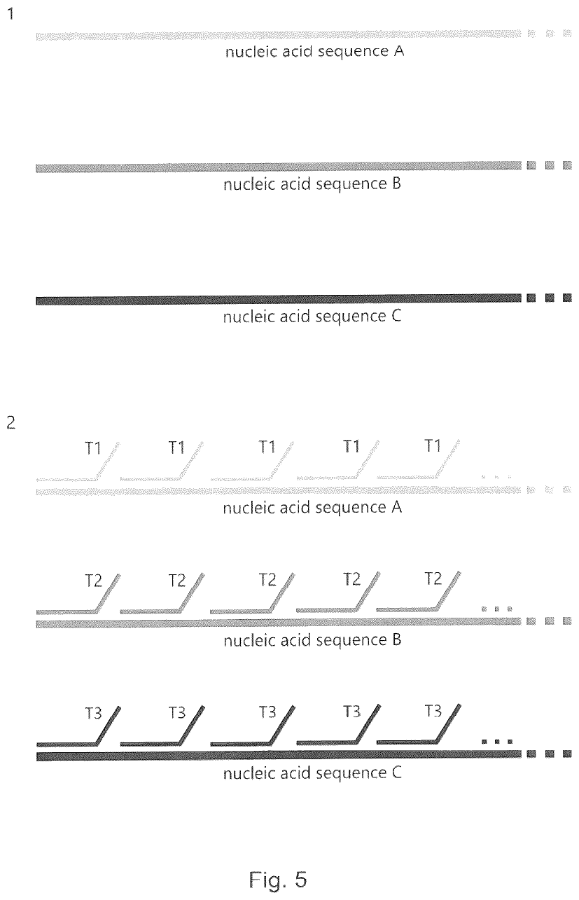

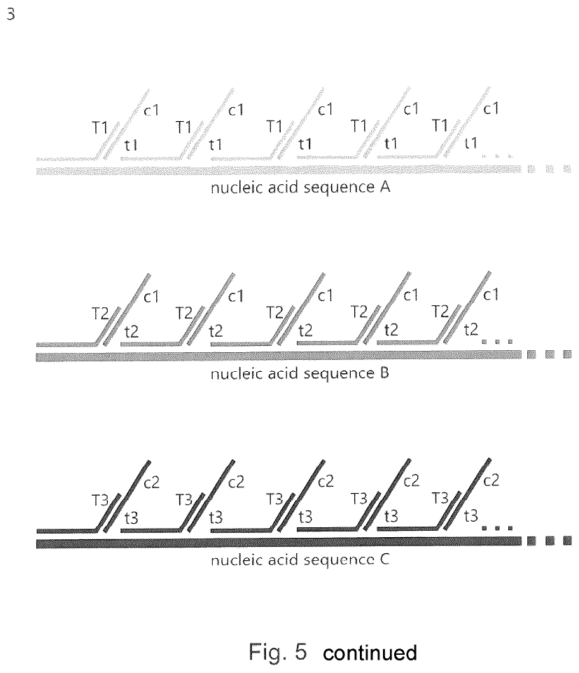

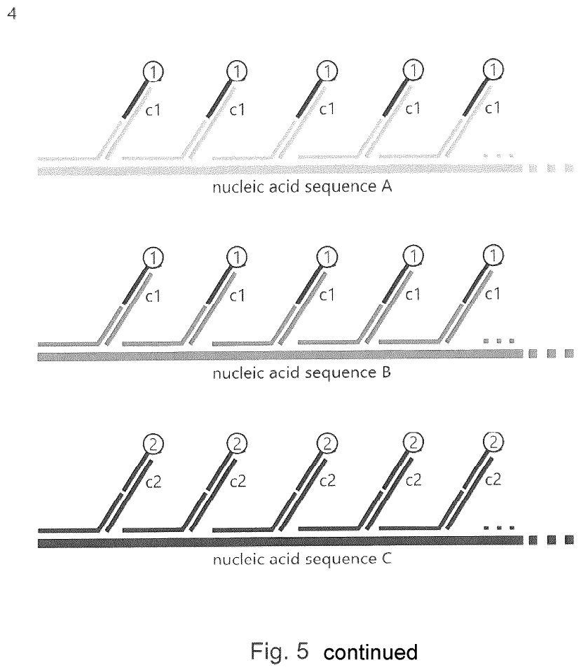

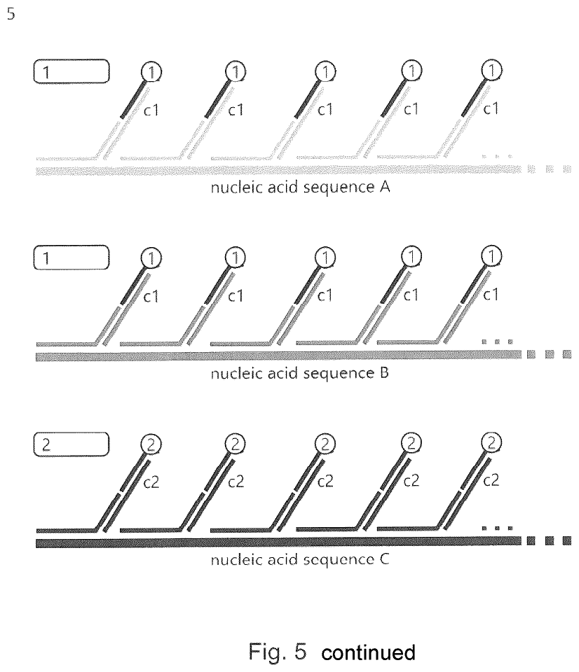

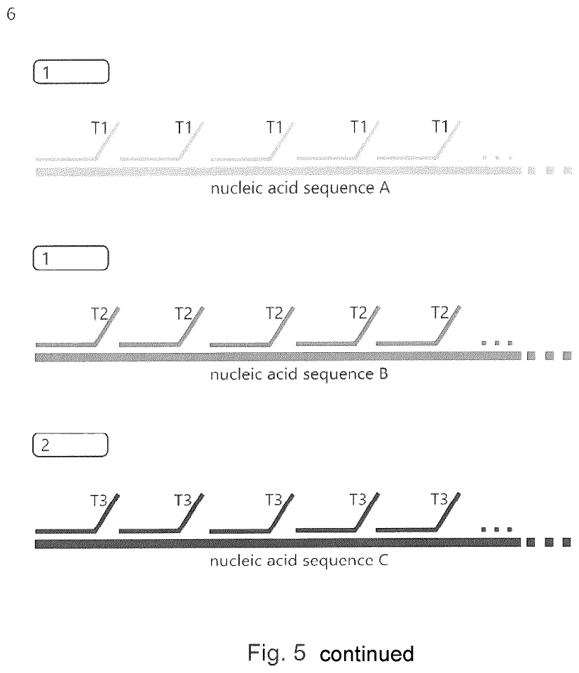

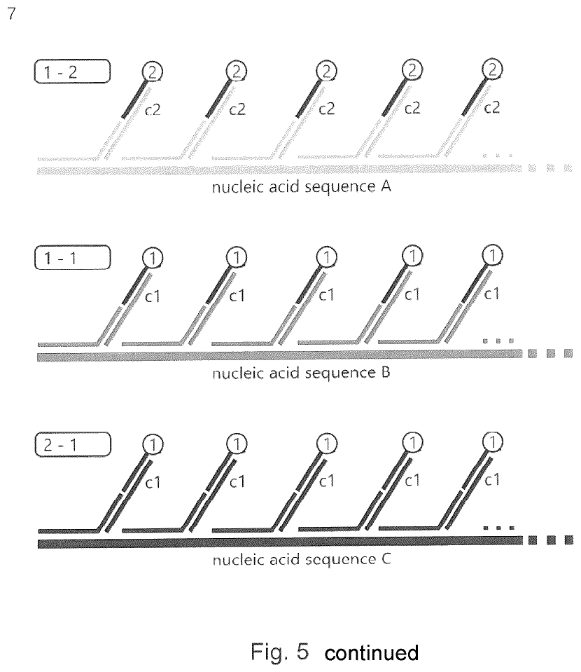

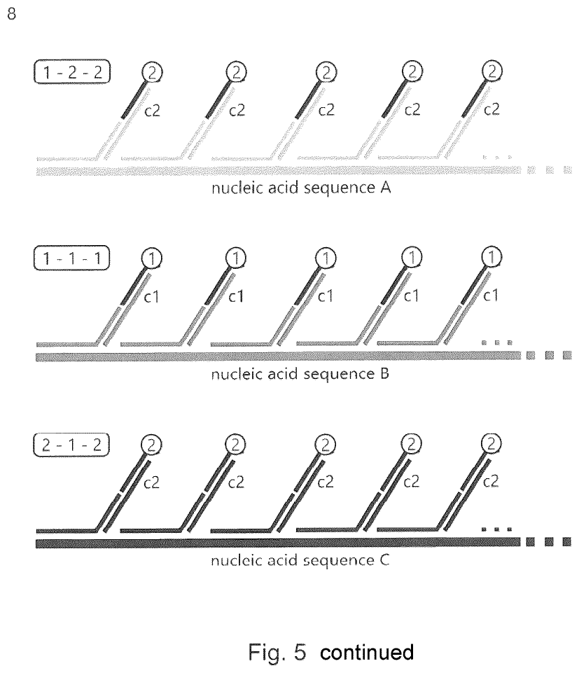

The usage of multi-decoders increases further the efficiency of the encoding scheme. FIG. 17 shows a possible encoding scheme using multi-decoders based upon the same conditions used for the examples with the decoding oligonucleotide with two sequence elements. One can clearly see that the multi-decoder based encoding scheme can create a higher hamming distance, with the same number of rounds and the same number of different signal oligonucleotides used in the example of FIG. 5.

As mentioned above the analyte to be encoded may be a nucleic acid, preferably DNA, PNA or RNA, in particular mRNA, a peptide, polypeptide, a protein, and/or mixtures thereof.

In some advantageous embodiments, the binding element (S) comprises an amino acid sequence allowing a specific binding to the analyte to be encoded. The binding element (S) may comprise moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof.

In some advantageous embodiments, the binding element (S) may comprise or is an antibody or an antibody fragment selected from the group consisting of Fab, scFv; single domain, or a fragment thereof, bis scFv, F(ab)2, F(ab)3, minibody, diabody, triabody, tetrabody and tandab.

The present disclosure pertains in particular to a multiplex method for detecting different analytes in a sample by sequential signal-encoding of said analytes, comprising the steps of:
(A) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
   (aa) a binding element (S) that specifically interacts with one of the different analytes to be encoded, and
   (bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(B) contacting the sample with at least one set of decoding oligonucleotides per analyte, wherein in each set of decoding oligonucleotides for an individual analyte each decoding oligonucleotide comprises:
   (aa) an identifier connector element (t) comprising a nucleotide sequence which is essentially complementary to at least a section of the unique identifier sequence of the identifier element (T) of the corresponding analyte-specific probe set, and
   (bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(C) contacting the sample with at least a set of signal oligonucleotides, each signal oligonucleotide comprising:
   (aa) a translator connector element (C) comprising a nucleotide sequence which is essentially complementary to at least a section of the nucleotide sequence of a translator element (c) comprised in a decoding oligonucleotide, and
   (bb) a signal element.
(D) Detecting the signal caused by the signal element;
(E) selectively removing the decoding oligonucleotides and signal oligonucleotides from the sample, thereby essentially maintaining the specific binding of the analyte-specific probes to the analytes to be encoded;
(F) Performing at least three (3) further cycles comprising steps B) to E) to generate an encoding scheme with a code word per analyte, wherein in particular the last cycle may stop with step (D).

As mentioned above, the method according to the present disclosure comprises selectively removing the decoding oligonucleotides and signal oligonucleotides from the sample, thereby essentially maintaining the specific binding of the analyte-specific probes to the analyte to be encoded. In particular all steps are performed sequentially. However some steps may be performed simultaneously, in particular the contacting steps A) to C), in particular B) and C).

By this measure the requirements for another round/cycle of binding further decoding oligonucleotides to the same analyte-specific probes are established, thus finally resulting in a code or encoding scheme comprising more than one signal. This step is realized by applying conditions and factors well known to the skilled person, e.g. pH, temperature, salt conditions, oligonucleotide concentration, polymers etc.

In another embodiment of the present disclosure, the method may comprise repeating steps (B)-(E) at least three times to generate an encoding scheme. With this measure a code of four signals in case of four cycles/rounds which are carried out by the user, where ‘n’ is an integer representing the number of rounds. The encoding capacity of the method according to the disclosure is herewith increased depending on the nature of the analyte and the needs of the operator. In an embodiment of the disclosure said encoding scheme is predetermined and allocated to the analyte to be encoded.

However, this measure enables a precise experimental set-up by providing the appropriate sequential order of the employed decoding and signal oligonucleotides and, therefore, allows the correct allocation of a specific analyte to a respective encoding scheme. The decoding oligonucleotides which are used in repeated steps (B)-(D2) may comprise a translator element (c2) which is identical with the translator element (c1) of the decoding oligonucleotides used in previous steps (B)-(E). In another embodiment of the disclosure decoding oligonucleotides are used in repeated steps (B)-(E) comprising a translator element (c2) which differs from the translator element (c1) of the decoding oligonucleotides used in previous steps (B)-(E). It is understood that the decoding elements may or may not be changed from round to round, i.e. in the second round (B)-(E) comprising the translator element c2, in the third round (B)-(E) comprising the translator element c3, in the fourth round (B)-(E) comprising the translator element c4 etc., wherein ‘n’ is an integer representing the number of rounds.

The signal oligonucleotides which are used in repeated steps (B)-(E) may comprise a signal element which is identical with the signal element of the decoding oligonucleotides used in previous steps (B)-(E). In a further embodiment of the disclosure signal oligonucleotides are used in repeated steps (B)-(E) comprising a signal element which differs from the signal element of the decoding oligonucleotides used in previous steps (B)-(E). In some embodiments no-signal oligonucleotides and/or no-signal decoding oligonucleotides for an individual analyte are used, resulting to the value 0 in the codeword for this cycle/position. In some embodiments in a repeated cycle no decoding oligonucleotides for an individual analyte is contacted with the sample resulting also to the value 0 in the codeword for this cycle/position.

By this measure each round the same or a different signal is provided resulting in an encoding scheme characterized by a signal sequence consisting of numerous different signals. This measure allows the creation of a unique code or code word which differs from all other code words of the encoding scheme. In another embodiment of the disclosure, the binding element (S) of the analyte-specific probe comprises a nucleic acid comprising a nucleotide sequence allowing a specific binding to the analyte to be encoded, preferably a specific hybridization to the analyte to be encoded.

In another embodiment of the disclosure, subgroups of the same type of analyte (variations) can be detected performing step G) to detect the presence or absence of an exclusive element.

In an advantageous embodiment, the sample is contacted with at least two different sets of analyte-specific probes per analyte,
wherein the analyte-specific probes comprised in these different sets interacting with the same analyte, but specifically interact with different sub-structures of the same analyte,
wherein the analyte-specific probes of the first set of analyte-specific probes interacts with a sub-structure which is comprised in all variations of an analyte,
wherein the analyte-specific probes of the second set of analyte-specific probes (subgroup-specific probe set) interacts with a sub-structure which is comprised only in a specific variation of the analyte,
wherein the analyte-specific probes of the first set of analyte-specific probes comprise the same identifier element (T) comprising a nucleotide sequence which is unique to the analyte to be encoded (unique identifier sequence), and
wherein the analyte-specific probes of the second set of analyte-specific probes comprise the same identifier element (T) comprising a nucleotide sequence which is unique to the analyte to be encoded (unique identifier sequence),
wherein the identifier elements (T) of the analyte-specific probes of the first set of analyte-specific probes and the identifier elements (T) of the analyte-specific probes of the second set of analyte-specific probes are different for binding different decoding oligonucleotides and/or non-signal decoding oligonucleotides.

In some advantageous embodiments, all steps are automated, in particular wherein steps B) to F) are automated, in particular by using a robotic system and/or an optical multiplexing system according to the present disclosure. In some examples, the steps may be performed in a fluidic system.

As mentioned above, with the methods according to the present disclosure an encoding scheme with a code word per analyte is generated. Therefore, each analyte may be associated with a specific code word, wherein said code word comprise a number of positions, and wherein each position corresponds to one cycle resulting in a plurality of distinguishable encoding schemes with the plurality of code words. In particular, said encoding scheme may be predetermined and allocated to the analyte to be encoded.

In some advantageous embodiments, the code words obtained for the individual analytes in the performed cycles comprise the detected signals and additionally at least one element corresponding to no detected signal like 0, 1 or 0, 1, 2 etc. (see also FIG. 13 and FIG. 14). In particular, no signal is detected for at least one analyte within at least one cycle if using the a non-signal probe according to FIG. 14, Nr. 2 to 4, or a non-signal decoding oligonucleotide as shown in FIG. 14 Nr. 5, or if in one cycle no decoding oligonucleotide is contacted with the corresponding identifier sequence comprised on analyte-specific probe interacting with the corresponding analyte in the sample. In this cycle the position has the value zero (0).

In some advantageous embodiments, at least for one individual analyte a position of the code word is zero (0). In particular, the code word zero (0) is generated by using no decoding oligonucleotides having an identifier connector element (t) comprising a nucleotide sequence which is essentially complementary to at least a section of the unique identifier sequence of the identifier element (T) of a corresponding analyte-specific probe for an individual analyte. As mentioned above, in some embodiments, if at least for one individual analyte a position of the code word is zero (0) in this cycle no corresponding decoding oligonucleotides having an identifier connector element (t) comprising a nucleotide sequence which is essentially complementary to at least a section of the unique identifier sequence of the identifier element (T) of a corresponding analyte-specific probe for an individual analyte are used.

Furthermore, in some advantageous embodiments the sample is contacted with at least two different sets of signal oligonucleotides, wherein the signal oligonucleotides in each set comprise a different signal element and comprise a different connector element (C).

In more particular embodiments, the sample is contacted with at least two different sets of decoding oligonucleotides per analyte,
wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and
wherein the decoding oligonucleotides of the different sets per analyte differ in the translator element (c) comprising a nucleotide sequence allowing a specific hybridization of a signal oligonucleotide.

In more particular embodiments, the sample is contacted with at least two different sets of decoding oligonucleotides per analyte,
wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and
wherein the decoding oligonucleotides of the different sets per analyte differ in the translator element (c) comprising a nucleotide sequence allowing a specific hybridization of a signal oligonucleotide;
wherein only one set of decoding oligonucleotides per analyte is used per cycle, and/or wherein different sets of decoding oligonucleotides are used in different cycles in combination with the corresponding set of signal oligonucleotides in the same cycle, in particular
wherein a set of decoding oligonucleotides and/or non-signal decoding oligonucleotides is reserved for optional detection of subgroups of analytes.

In some advantageous embodiments, the number of different sets of decoding oligonucleotides per analyte comprising different translator elements (c) corresponds to the number of different sets of signal oligonucleotides comprising different connector elements (C). All sets of decoding oligonucleotides for the different analytes may comprise the same type(s) of translator element(s) (c).

In some advantageous embodiments of the method according to the present disclosure, the sample is contacted with at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

As mentioned above, the sample may be contacted with at least two (2) different sets of non-signal decoding oligonucleotides for binding to at least two different identifier elements (T) of analyte-specific probes, each set of non-signal decoding oligonucleotides interacting with a different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments of the method according to the present disclosure, the different sets of non-signal decoding oligonucleotides may be comprised in a pre-mixture of different sets of non-signal decoding oligonucleotides or exist separately.

Furthermore, in some advantageous embodiments of the method according to the present disclosure, the sample is contacted with a set of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

In further embodiments, the sample may be contacted with:
at least two sets of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

As mentioned above, the different sets of non-signal oligonucleotides may be comprised in a pre-mixture of different sets of non-signal oligonucleotides or exist separately.

In further embodiments, the decoding oligonucleotides in a particular set of decoding oligonucleotides interacts with identical identifier elements (T) which are unique to a particular analyte.

As mentioned above, the different sets of decoding oligonucleotides may be comprised in a pre-mixture of different sets of decoding oligonucleotides or exist separately as well as the different sets of analyte-specific probes may be comprised in a pre-mixture of different sets of analyte-specific probes or exist separately as well the different sets of signal oligonucleotides may be comprised in a pre-mixture of different sets of signal oligonucleotides or exist separately.

In some advantageous embodiments of the method according to the present disclosure, the binding element (S) comprise a nucleic acid comprising a nucleotide sequence allowing a specific binding to the analyte to be encoded, preferably a specific hybridization to the analyte to be encoded.

In some advantageous embodiments of the method according to the present disclosure, after step A) and before step B) the non-bound analyte-specific probes may be removed, in particular by washing, further after step B) and before step C) the non-bound decoding oligonucleotides may be emoved, in particular by washing further, after step C) and before step D) the non-bound signal oligonucleotides may be removed, in particular by washing.

In some advantageous embodiments of the method according to the present disclosure, the analyte specific probes may be incubated with the sample, thereby allowing a specific binding of the analyte specific probes to the analytes to be encoded, further the decoding oligonucleotides may be incubated with the sample, thereby allowing a specific hybridization of the decoding oligonucleotides to identifier elements (T) of the respective analyte-specific probes, further the signal oligonucleotides may be incubated with the sample, thereby allowing a specific hybridization of the signal oligonucleotides to translator elements (T) of the respective decoding oligonucleotides.

As mentioned above, the analyte to be encoded may be a nucleic acid, preferably DNA, PNA, RNA, in particular mRNA, a peptide, polypeptide, a protein or combinations thereof. Therefore, the binding element (S) may comprise an amino acid sequence allowing a specific binding to the analyte to be encoded. Examples for a binding element (S) are moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof. In particular, the binding element (S) is an antibody or an antibody fragment selected from the group consisting of Fab, scFv; single domain, or a fragment thereof, bis scFv, Fab2, Fab3, minibody, diabody, triabody, tetrabody and tandab.

By this measure the method is further developed to such an extent that the encoded analytes can be detected by any means which is adapted to visualize the signal element. Examples of detectable physical features include e.g. light, chemical reactions, molecular mass, radioactivity, etc.

In some advantageous embodiments, the signal caused by the signal element, therefore in particular the binding of the signal oligonucleotides to the decoding oligonucleotides, interacting with the corresponding analyte probes, bound to the respective analyte is determined by:
(a) Imaging at least a portion of the sample; and/or
(b) Using an optical imaging technique; and/or
(c) Using a fluorescence imaging technique; and/or
(d) Multi-color fluorescence imaging technique; and/or
(e) Super-resolution fluorescence imaging technique.

The kits and method according to the present disclosure may be used ideally for in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases.

Further, the kits and method according to the present disclosure may be used also ideally for in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment.

Further, the kits and method according to the present disclosure may be used also ideally for in vitro methods for screening, identifying and/or testing a substance and/or drug comprising:
(a) contacting a test sample comprising a sample with a substance and/or drug
(b) detecting different analytes in a sample by sequential signal-encoding of said analytes with a method according to the present disclosure.

An optical multiplexing system suitable for the method according to the present disclosure, comprising at least:
a reaction vessel for containing the kits or part of the kits according to the present disclosure;
a detection unit comprising a microscope, in particular a fluorescence microscope;
a camera;
a liquid handling device.

In some embodiments, optical multiplexing system may comprises further a heat and cooling device and/or a robotic system.

Some examples of suitable construction techniques or materials that may be adapted for use in connection with the present disclosure may be described in, e.g., commonly-assigned U.S. Pat. No. 6,734,401 titled “ENHANCED SAMPLE PROCESSING DEVICES SYSTEMS AND METHODS” (Bedingham et al.) and U.S. Patent Application Publication No. US 2002/0064885 titled “SAMPLE PROCESSING DEVICES.” Other useable device constructions may be found in, e.g., U.S. Provisional Patent Application Ser. No. 60/214,508 filed on Jun. 28, 2000 and entitled “THERMAL PROCESSING DEVICES AND METHODS”; U.S. Provisional Patent Application Ser. No. 60/214,642 filed on Jun. 28, 2000 and entitled “SAMPLE PROCESSING DEVICES, SYSTEMS AND METHODS”; U.S. Provisional Patent Application Ser. No. 60/237,072 filed on Oct. 2, 2000 and entitled “SAMPLE PROCESSING DEVICES, SYSTEMS AND METHODS”; U.S. Provisional Patent Application Ser. No. 60/260,063 filed on Jan. 6, 2001 and titled “SAMPLE PROCESSING DEVICES, SYSTEMS AND METHODS”; U.S. Provisional Patent Application Ser. No. 60/284,637 filed on Apr. 18, 2001 and titled “ENHANCED SAMPLE PROCESSING DEVICES, SYSTEMS AND METHODS”; and U.S. Patent Application Publication No. US 2002/0048533 titled “SAMPLE PROCESSING DEVICES AND CARRIERS.” Other potential device constructions may be found in, e.g., U.S. Pat. No. 6,627,159 titled “CENTRIFUGAL FILLING OF SAMPLE PROCESSING DEVICES” (Bedingham et al.).

The optical multiplexing system according to the present disclosure may comprise a plurality of process chambers (e.g. reaction vessel) each for holding a respective sample and one or more sets of probes like sets of analyte specific probes, sets of decoding oligonucleotides/non-signal oligonucleotides and/or sets of signal oligonucleotides/non-signal-oligonucleotides. For example, the process chambers may comprised in a rotatable disk or in a movable well-plate like a 96-well plate; a motor to rotate said disk or to move the well-plate, wherein in particular the motor may be part of a robotic system.

The optical multiplexing system according to the present disclosure may comprise further at least one or a plurality of optical modules, in particular wherein the system comprises a housing having at least one or a plurality of locations adapted to receive the optical modules, wherein each of the plurality of optical module(s) are removable from the locations of the housing.

In some advantageous embodiments, the optical multiplexing system according to the present disclosure may comprise a detector, and in particular a fiber optic bundle coupled to the plurality of optical modules to convey the fluorescent light from the multiple optical modules to the detector.

In particular, the optical module(s) includes an optical channel each optical module having a light source selected for exciting a different one of the dyes and a lens to capture fluorescent light emitted, said optical module(s) being optically configured to interrogate the fluorescent dyes at different wavelengths.

In a further embodiment, the system may include a microfluidic cartridge (also referred to herein as a microfluidic device) having at least one flow-through channel. The optical multiplexing system includes a fluorescence imaging system. Further features of the system may be a temperature measurement and/or a control system. In some embodiments, the system comprises a pressure measurement and control system for applying variable pneumatic pressures, e.g to the microfluidic cartridge. The optical multiplexing system may comprise a storage device for holding multiple reagents, such as a well-plate. Further, the optical multiplexing system may comprise a liquid handling system, in particular comprising e.g. at least one robotic pipettor for aspirating, mixing, and dispensing reagent mixtures e.g. to the microfluidic cartridge and/or to the reaction vessel(s). Furthermore, the system may comprise means for data storage, processing, and output; and in particular a system controller to coordinate the various devices and functions.

In some embodiments, the method according to the present disclosure encodes a nucleic acid analyte, such as an mRNA, e.g. such an mRNA coding for a particular protein.

In some advantageous embodiments, the method described herein is used for specific detection of many different analytes in parallel. The technology allows to distinguish a higher number of analytes than different signals are available. The process includes at least four consecutive rounds of specific binding, signal detection and selective denaturation (if a next round is required), eventually producing a signal code. To decouple the dependency between the analyte specific binding and the oligonucleotides providing the detectable signal, a so called “decoding”-oligonucleotide is introduced. The decoding oligonucleotide transcribes the information of the analyte specific probe set to the signal oligonucleotides.

In a specific embodiment the method may comprise the steps of: 1. providing one or more analyte specific probe sets, the set of analyte specific probes consist of one or more different probes, each differing in the binding moiety that specifically interacts with the analyte, all probes of a single probe set are tethered to a sequence element (unique identifier), that is unique to a single probe set and allows the specific hybridization of a decoding oligonucleotide, 2. specific binding of the probe sets to their target binding sites of the analyte, 3. eliminating non-bound probes (e.g. by a wash step), 4. providing a mixture of decoding oligonucleotides that specifically hybridize to the unique identifier sequences of the probe sets, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide, 5. specific hybridization of the decoding oligonucleotides to the unique identifier sequences provided by the bound probe sets, 6. eliminating non-bound decoding oligonucleotides (e.g. by washing step), 7. providing a mixture of signal oligonucleotides, comprising of a signal that can be detected and a nucleic acid sequence that specifically hybridizes to the translator element of one of the decoding oligonucleotides used in the former hybridization step, 8. specific hybridization of the signal oligonucleotides, 9. eliminating non-bound signal oligonucleotides, 10. detection of the signals, 11. selective release of decoding oligonucleotides and signal oligonucleotides while the binding of specific probe sets to the analyte is almost or completely unaffected, 12. eliminating released decoding oligonucleotide and signal oligonucleotides (e.g. by a washing step) while the binding of specific probes sets to the analytes is almost or completely unaffected, repeating the steps 4 to 12 at least three times until the detection of a sufficient number of signals to generate an encoding scheme for each different analyte of interest.

It is to be understood that the before-mentioned features and those to be mentioned in the following cannot only be used in the combination indicated in the respective case, but also in other combinations or in an isolated manner without departing from the scope of the disclosure.

The disclosure is now further explained by means of embodiments resulting in additional features, characteristics and advantages of the disclosure. The embodiments are of pure illustrative nature and do not limit the scope or range of the disclosure. The features mentioned in the specific embodiments are general features of the disclosure which are not only applicable in the specific embodiment but also in an isolated manner in the context of any embodiment of the disclosure.

The method disclosed herein is used for specific detection of many different analytes in parallel. The technology allows distinguishing a higher number of analytes than different signals are available. The process preferably includes at least two consecutive rounds of specific binding, signal detection and selective denaturation (if a next round is required), eventually producing a signal code. To decouple the dependency between the analyte specific binding and the oligonucleotides providing the detectable signal, a so called “decoding” oligonucleotide is introduced. The decoding oligonucleotide transcribes the information of the analyte specific probe set to the signal oligonucleotides.

### METHODS AND EXAMPLES

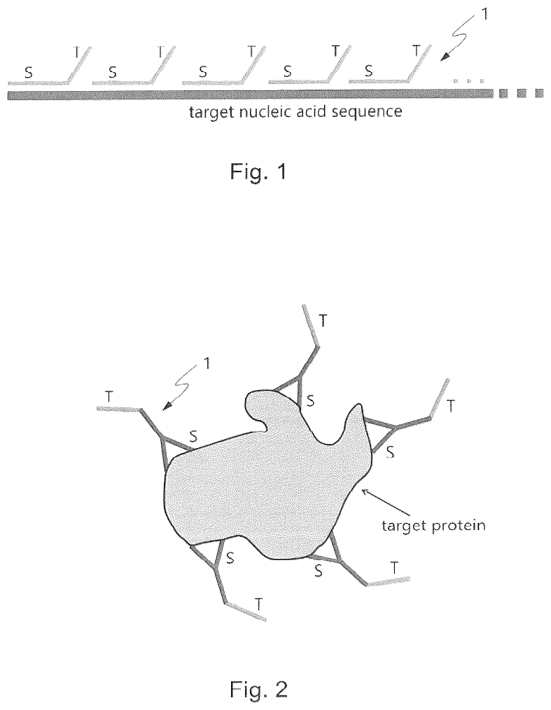

In an application variant, the analyte or target is nucleic acid, e.g. DNA or RNA, and the probe set comprises oligonucleotides that are partially or completely complementary to the whole sequence or a subsequence of the nucleic acid sequence to be detected (FIG. 1). The nucleic acid sequence specific oligonucleotide probe sets comprising analyte-specific probes (1) including a binding element (S) that specifically hybridizes to the target nucleic acid sequence to be detected, and an identifier element (T) comprising a nucleotide sequence which is unique to said set of analyte-specific probes (unique identifier sequence).

In an advantageous embodiment of the present disclosure, the analyte/target is a nucleic acid, e.g. RNA, and two probe sets comprising oligonucleotides that are partially or completely complementary to distinctive regions of a single nucleic acid sequence target (FIG. 15). The nucleic acid sequence specific oligonucleotide probe sets comprising two sets of analyte-specific probes each (1 and 1′, 2 and 2′), both including a binding element (S) that specifically hybridizes to the target nucleic acid sequence to be detected, and each with a different identifier element (T) comprising a nucleotide sequence which is unique to said set of analyte-specific probes (unique identifier sequence). The two sets are used for decoding (1,2) of the analyte and detection of presence/absence of exclusive elements that differentiate subgroups of analytes (1′ and 2′).

In a further application variant, the analyte or target is a protein and the probe set comprises one or more proteins, e.g. antibodies (FIG. 2). The protein specific probe set comprising analyte-specific probes (1) including a binding element (T) such as the (hyper-)variable region of an antibody, that specifically interacts with the target protein to be detected, and the identifier element (T).

In a further application variant, at least one analyte is a nucleic acid and at least a second analyte is a protein and at least the first probe set binds to the nucleic acid sequence and at least the second probe set binds specifically to the protein analyte. Other combinations are possible as well.

An Embodiments of the general method present may be:
Step 1: Applying the at least 20 analyte- or target-specific probe sets. The target nucleic acid sequence is incubated with a probe set consisting of oligonucleotides with sequences complementary to the target nucleic acid. In this example, a probe set of 5 different probes is shown, each comprising a sequence element complementary to an individual subsequence of the target nucleic acid sequence (S1 to S5). In this example, the regions do not overlap. Each of the oligonucleotides targeting the same nucleic acid sequence comprises the identifier element or unique identifier sequence (T), respectively.
Step 2: Hybridization of the probe set. The probe set is hybridized to the target nucleic acid sequence under conditions allowing a specific hybridization. After the incubation, the probes are hybridized to their corresponding target sequences and provide the identifier element (T) for the next steps.
Step 3: Eliminating non-bound probes. After hybridization, the unbound oligonucleotides are eliminated, e.g. by washing steps.
Step 4: Applying the decoding oligonucleotides. The decoding oligonucleotides consisting of at least two sequence elements (t) and (c) are applied. While sequence element (t) is complementary to the unique identifier sequence (T), the sequence element (c) provides a region for the subsequent hybridization of signal oligonucleotides (translator element).
Step 5: Hybridization of decoding oligonucleotides. The decoding oligonucleotides are hybridized with the unique identifier sequences of the probes (T) via their complementary first sequence elements (t). After incubation, the decoding oligonucleotides provide the translator sequence element (c) for a subsequent hybridization step.
Step 6: Eliminating the excess of decoding oligonucleotides. After hybridization, the unbound decoding oligonucleotides are eliminated, e.g. by washing steps.
Step 7: Applying the signal oligonucleotide. The signal oligonucleotides are applied. The signal oligonucleotides comprise at least one second connector element (C) that is essentially complementary to the translator sequence element (c) and at least one signal element that provides a detectable signal (F).
Step 8: Hybridization of the signal oligonucleotides. The signal oligonucleotides are hybridized via the complementary sequence connector element (C) to the translator element (c) of decoding oligonucleotide. After incubation, the signal oligonucleotides are hybridized to their corresponding decoding oligonucleotides and provide a signal (F) that can be detected.
Step 9: Eliminating the excess of signal oligonucleotides. After hybridization, the unbound signal oligonucleotides are eliminated, e.g. by washing steps.
Step 10: Signal detection. The signals provided by the signal oligonucleotides are detected.

The following steps (steps 11 and 12) are unnecessary for the last detection round.
Step 11: Selective denaturation. The hybridization between the unique identifier sequence (T) and the first sequence element (t) of the decoding oligonucleotides is dissolved. The destabilization can be achieved via different mechanisms well known to the trained person like for example: increased temperature, denaturing agents, etc. The target- or analyte-specific probes are not affected by this step.
Step 12: Eliminating the denatured decoding oligonucleotides. The denatured decoding oligonucleotides and signal oligonucleotides are eliminated (e.g. by washing steps) leaving the specific probe sets with free unique identifier sequences, reusable in a next round of hybridization and detection (steps 4 to 10). This detection cycle (steps 4 to 12) is repeated at least four times until the planed encoding scheme is completed.

In some advantageous embodiments, in a Step 13 an additional cycle of steps 4 to 10 is performed to read subgroup/variation specific signals.

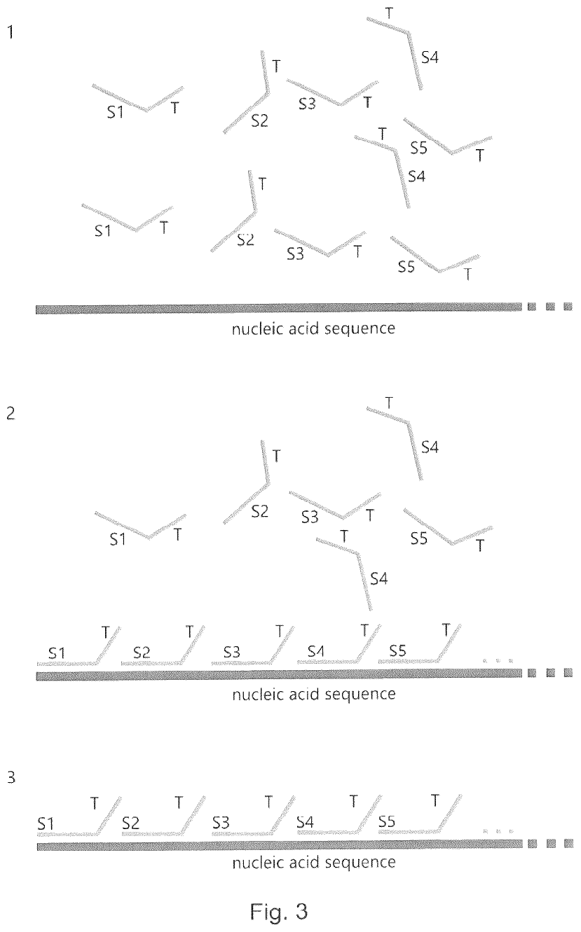

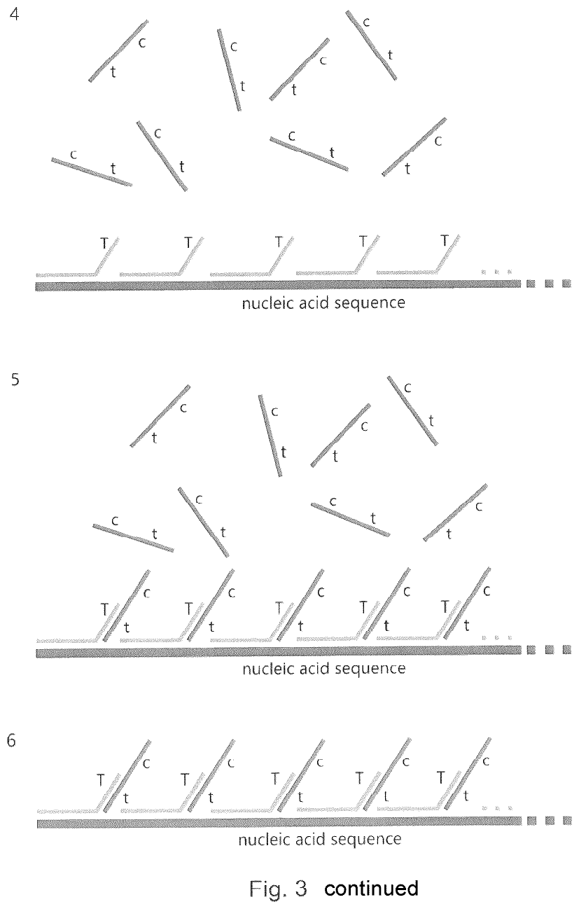

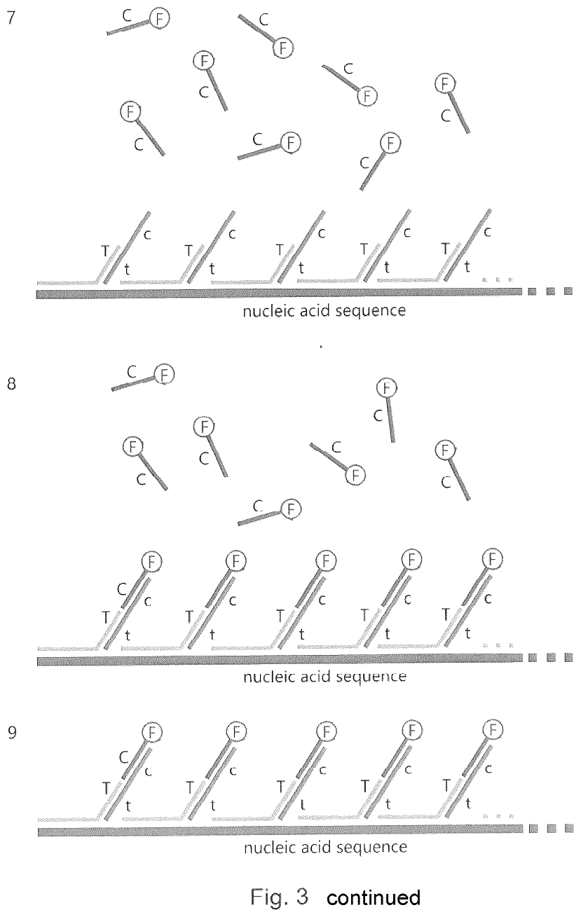

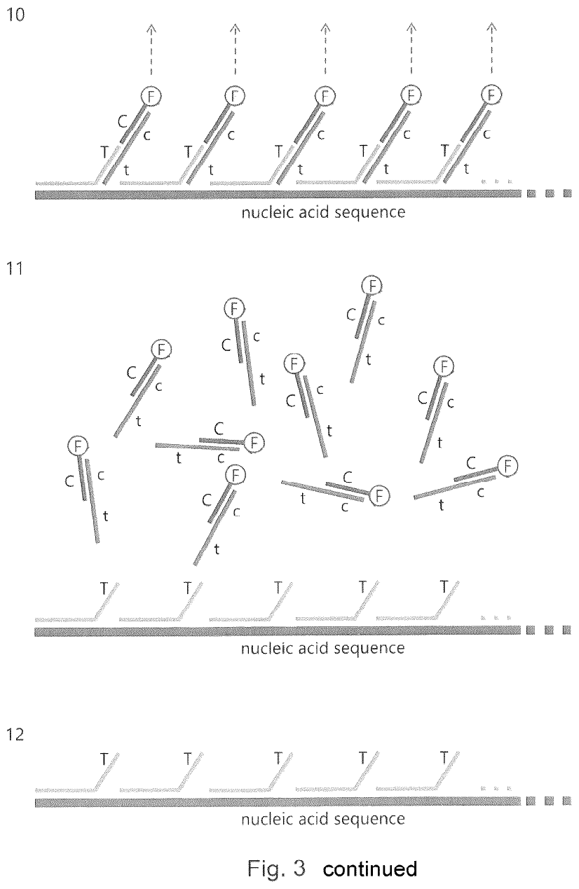

Another Embodiment of the general method of the present disclosure using multi-decoders may be (FIG. 16):
Step 1: Target nucleic acids: In this example three different target nucleic acids (A), (B) and (C) have to be detected and differentiated by using only two different types of signal oligonucleotides. Before starting the experiment, a certain encoding scheme is set. In this example, the three different nucleic acid sequences are encoded by three rounds of detection with three different signal types (1), (2) and (1/2) and a resulting hamming distance of 3 to allow for error detection. The planed code words are:
sequence A: (1)-(1)-(2)
sequence B: (2)-(2)-(1/2)
sequence C: (1/2)-(1/2)-(1)
Step 2: Hybridization of the probe sets: For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (A) is labeled with (T1), sequence (B) with (T2) and sequence (C) with (T3). The illustration in FIG. 16 summarizes Steps 1 to 3 of FIG. 3.
Step 3: Hybridization of the decoding oligonucleotides and multi-decoders: For each unique identifier present, a certain decoding oligonucleotide or multi-decoder is applied specifically hybridizing to the corresponding unique identifier sequence by its first sequence element (here (t1) to (T1), (t2) to (T2) and (t3) to (T3)). Each of the decoding oligonucleotides or multi-decoders provides a translator or two translator elements that define the signals that will be generated after hybridization of signal oligonucleotides. Here nucleic acid sequence (A) is labeled with (c1), (B) is labeled with (c2) and (C) is labeled with both translator elements (c1) and (c2) resulting in the signal (1/2). The illustration in FIG. 16 summarizes steps 4 to 6 of FIG. 3.
Step 4: Hybridization of signal oligonucleotides: For each type of translator element, a signal oligonucleotide with a certain signal, differentiable from signals of other signal oligonucleotides, is applied. This signal oligonucleotide can specifically hybridize to the corresponding translator element. The illustration in FIG. 16 summarizes steps 7 to 9 of FIG. 3
Step 5: Signal detection for the encoding scheme: The different signals are detected. Note that in this example the nucleic acids (A), (B) and (C) can already be distinguished after the first round of detection. This is in contrast to the step 5 of FIG. 5 explained by the additional signal type (1/2) that can be realized due to multi-decoders. Although nucleic acid sequences can already be distinguished, the additional rounds contribute to the planned hamming distance of 3. The illustration in FIG. 16 corresponds to step 10 of FIG. 3.
Step 6: Selective denaturation: The decoding (and signal) oligonucleotides and/or multi-decoders of all nucleic acid sequences to be detected are selectively denatured and eliminated as described in steps 11 and 12 of FIG. 3. Afterwards the unique identifier sequences of the different probe sets can be used for the next round of hybridization and detection.
Step 7: Second round of detection: A next round of hybridization and detection is done as described in steps 3 to 5. Note that in this new round the mix of different decoding oligonucleotides and multi-decoders is changed. For example, decoding oligonucleotide of nucleic acid sequence (A) used in the first round comprised of sequence elements (t1) and (c1) while the new multi decoder of round 2 comprises of the sequence elements (t1), (c1) and (c2). Note that now a hamming distance of 2 is already given after 2 rounds, which is the final result of the example in FIG. 3 after 3 rounds.
Step 8: Third round of detection: Again, a new combination of decoding oligonucleotides and/or multi-decoders is used leading to new signal combinations. After signal detection, the resulting code words for the three different nucleic acid sequences are not only unique and therefore distinguishable but comprise a hamming distance of 3 to other code words. Due to the hamming distance, an error in the detection of the signals (signal exchange) would not result in a valid code word and therefore could be detected and because of hamming distance 3 also corrected, in contrast to the encoding scheme of FIG. 3. This way three different nucleic acids can be distinguished in three detection rounds with two different signals, allowing an error detection and correction.

Note that in every round of detection, the type of signal provided by a certain unique identifier is controlled by the use of a certain decoding oligonucleotide. As a result, the sequence of decoding oligonucleotides applied in the detection cycles transcribes the binding specificity of the probe set into a unique signal sequence.

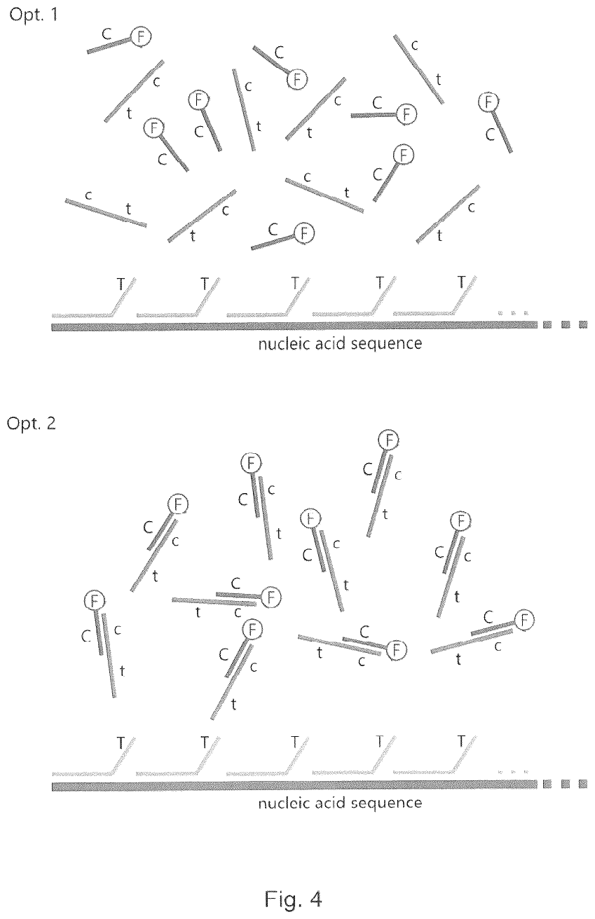

The steps of decoding oligonucleotide hybridization (steps 4 to 6) and signal oligonucleotide hybridization (steps 7 to 9) can also be combined in two alternative ways as shown in FIG. 4.

Opt. 1: Simultaneous hybridization. Instead of the steps 4 to 9 of FIG. 3, specific hybridization of decoding oligonucleotides and signal oligonucleotides can also be done simultaneously leading to the same result as shown in step 9 of FIG. 3, after eliminating the excess decoding- and signal oligonucleotides.

Opt. 2: Preincubation. Additionally to option 1 of FIG. 3, decoding- and signal oligonucleotides can be preincubated in a separate reaction before being applied to the target nucleic acid with the already bound specific probe set.

### 1. Example for Signal Encoding of Three Different Nucleic Acid Sequences by Two Different Signal Types and Three Detection Rounds

FIG. 3 shows the general concept of generation and detection of specific signals mediated by decoding oligonucleotides. It does not show the general concept of encoding that can be achieved by this procedure. To illustrate the use of the process shown in FIG. 3 for the generation of an encoding scheme, FIG. 5 shows a general example for a multiple round encoding experiment with three different nucleic acid sequences. In this example, the encoding scheme includes error detection.
Step 1: Target nucleic acids. In this example three different target nucleic acids (A), (B) and (C) have to be detected and differentiated by using only two different types of signal. Before starting the experiment, a certain encoding scheme is set. In this example, the three different nucleic acid sequences are encoded by three rounds of detection with two different signals (1) and (2) and a resulting hamming distance of 2 to allow for error detection. The planed code words are:
sequence A: (1)-(2)-(2);
sequence B: (1)-(1)-(1);
sequence C: (2)-(1)-(2).
Step 2: Hybridization of the probe sets. For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (T) is labeled with (T1), sequence (B) with (T2) and sequence (C) with (T3). The illustration summarizes steps 1 to 3 of FIG. 3.
Step 3: Hybridization of the decoding oligonucleotides. For each unique identifier present, a certain decoding oligonucleotide is applied specifically hybridizing to the corresponding unique identifier sequence by its first sequence element (here (t1) to (T1), (t2) to (T2) and (t3) to (T3)). Each of the decoding oligonucleotides provides a translator element that defines the signal that will be generated after hybridization of signal oligonucleotides. Here nucleic acid sequences (A) and (B) are labeled with the translator element (c1) and sequence (C) is labeled with (c2). The illustration summarizes steps 4 to 6 of FIG. 3.
Step 4: Hybridization of signal oligonucleotides. For each type of translator element, a signal oligonucleotide with a certain signal (2), differentiable from signals of other signal oligonucleotides, is applied. This signal oligonucleotide can specifically hybridize to the corresponding translator element. The illustration summarizes steps 7 to 9 of FIG. 3.
Step 5: Signal detection for the encoding scheme. The different signals are detected. Note that in this example the nucleic acid sequence (C) can be distinguished from the other sequences by the unique signal (2) it provides, while sequences (A) and (B) provide the same kind of signal (1) and cannot be distinguished after the first cycle of detection. This is due to the fact, that the number of different nucleic acid sequences to be detected exceeds the number of different signals available. The illustration corresponds to step 10 of FIG. 3.
Step 6: Selective denaturation. The decoding (and signal) oligonucleotides of all nucleic acid sequences to be detected are selectively denatured and eliminated as described in steps 11 and 12 of FIG. 3. Afterwards the unique identifier sequences of the different probe sets can be used for the next round of hybridization and detection.
Step 7: Second round of detection. A next round of hybridization and detection is done as described in steps 3 to 5. Note that in this new round the mix of different decoding oligonucleotides is changed. For example, decoding oligonucleotide of nucleic acid sequence (A) used in the first round comprised of sequence elements (t1) and (c1) while the new decoding oligonucleotide comprises of the sequence elements (t1) and (c2). Note that now all three sequences can clearly be distinguished due to the unique combination of first and second round signals.
Step 8: Third round of detection. Again, a new combination of decoding oligonucleotides is used leading to new signal combinations. After signal detection, the resulting code words for the three different nucleic acid sequences are not only unique and therefore distinguishable but comprise a hamming distance of 2 to other code words. Due to the hamming distance, an error in the detection of the signals (signal exchange) would not result in a valid code word and therefore could be detected. By this way three different nucleic acids can be distinguished in three detection rounds with two different signals, allowing error detection.

### 2. Advantages Over Prior Art Technologies

### Coding Strategy

Compared to state-of-the-art methods, one particular advantage of the method according to the disclosure is the use of decoding oligonucleotides breaking the dependencies between the target specific probes and the signal oligonucleotides.

Without decoupling target specific probes and signal generation, two different signals can only be generated for a certain target if using two different molecular tags. Each of these molecular tags can only be used once. Multiple readouts of the same molecular tag do not increase the information about the target. In order to create an encoding scheme, a change of the target specific probe set after each round is required (SeqFISH) or multiple molecular tags must be present on the same probe set (like merFISH, intronSeqFISH).

Following the method according to the disclosure, different signals are achieved by using different decoding oligonucleotides reusing the same unique identifier (molecular tag) and a small number of different, mostly cost-intensive signal oligonucleotides. This leads to several advantages in contrast to the other methods.
(1) The encoding scheme is not defined by the target specific probe set as it is the case for all other methods of prior art. Here the encoding scheme is transcribed by the decoding oligonucleotides. This leads to a much higher flexibility concerning the number of rounds and the freedom in signal choice for the codewords. Looking on the methods of prior art (e.g. merFISH or intronSeqFISH), the encoding scheme (number, type and sequence of detectable signals) for all target sequences is predefined by the presence of the different tag sequences on the specific probe sets (4 of 16 different tags per probe set in the case of merFISH and 5 of 60 different tags in the case of intron FISH). In order to produce a sufficient number of different tags per probe set, the methods use rather complex oligonucleotide designs with several tags present on one target specific oligonucleotide. In order to change the encoding scheme for a certain target nucleic acid, the specific probe set has to be replaced. The method according to the disclosure describes the use of a single unique tag sequence (unique identifier) per analyte, because it can be reused in every detection round to produce a new information. The encoding scheme is defined by the order of decoding oligonucleotides that are used in the detection rounds. Therefore, the encoding scheme is not predefined by the specific probes (or the unique tag sequence) but can be adjusted to different needs, even during the experiment. This is achieved by simply changing the decoding oligonucleotides used in the detection rounds or adding additional detection rounds.
(2) The number of different signal oligonucleotides must match the number of different tag sequences with methods of prior art (16 in the case of merFISH and 60 in the case of intronSeqFISH). Using the method according to the disclosure, the number of different signal oligonucleotides matches the number of different signals used. Due to this, the number of signal oligonucleotides stays constant for the method described here and never exceeds the number of different signals but increases with the complexity of the encoding scheme in the methods of prior art (more detection rounds more different signal oligonucleotides needed). As a result, the method described here leads to a much lower complexity (unintended interactions of signal oligonucleotides with environment or with each other) and dramatically reduces the cost of the assay since the major cost factor are the signal oligonucleotides.
(3) In the methods of prior art, the number of different signals generated by a target specific probe set is restricted by the number of different tag sequences the probe set can provide. Since each additional tag sequence increases the total size of the target specific probe, there is a limitation to the number of different tags a single probe can provide. This limitation is given by the size dependent increase of several problems (unintended inter- and intramolecular interactions, costs, diffusion rate, stability, errors during synthesis etc.). Additionally, there is a limitation of the total number of target specific probes that can be applied to a certain analyte. In case of nucleic acids, this limitation is given by the length of the target sequence and the proportion of suitable binding sites. These factors lead to severe limitations in the number of different signals a probe set can provide (4 signals in the case of merFISH and 5 signals in the case of intronSeqFISH). This limitation substantially affects the number of different code words that can be produced with a certain number of detection rounds. In the approach of the disclosure only one tag is needed and can be freely reused in every detection round. This leads to a low oligonucleotide complexity/length and at the same time to the maximum encoding efficiency possible (number of colors^{number of rounds}). The vast differences of coding capacity of our method compared to the other methods is shown in FIGS. 1 and 5. Due to this in approach of the disclosure a much lower number of detection rounds is needed to produce the same amount of information. A lower number of detection rounds is connected to lower cost, lower experimental time, lower complexity, higher stability and success rate, lower amount of data to be collected and analyzed and a higher accuracy of the results.

### Coding Capacity

All three methods compared in the Table 1 below use specific probe sets that are not denatured between different rounds of detection. For intronSeqFISH there are four detection rounds needed to produce the pseudo colors of one coding round, therefore data is only given for rounds 4, 8, 12, 16 and 20. The merFISH-method uses a constant number of 4 signals, therefore the data starts with the smallest number of rounds possible. After 8 detection rounds our method exceeds the maximum coding capacity reached with 20 rounds of merFISH (depicted with one asterisk) and after 12 rounds of detection the maximum coding capacity of intron FISH is exceeded (depicted with two asterisks). For the method according to the disclosure usage of 3 different signals is assumed (as is with intronSeqFISH).

TABLE 1
Comparison of coding capacity

| | CODING CAPACITY | | |
|---|---|---|---|
| | Method of the | | |
| NUMBER OF | present | intron | |
| ROUNDS: | disclosure | FISH | merFISH |
| 1 | 3 | — | — |
| 2 | 9 | — | — |
| 3 | 27 | — | — |
| 4 | 81 | 12 | 1 |
| 5 | 243 | — | 5 |
| 6 | 729 | — | 15 |
| 7 | 2187 | — | 35 |
| 8* | 6561 | 144 | 70 |
| 9 | 19683 | — | 126 |
| 10 | 59049 | — | 210 |
| 11 | 177147 | — | 330 |
| 12** | 531441 | 1728 | 495 |
| 13 | 1594323 | — | 715 |
| 14 | 4782969 | — | 1001 |
| 15 | 14348907 | — | 1365 |
| 16 | 43046721 | 20736 | 1820 |
| 17 | 129140163 | — | 2380 |
| 18 | 387420489 | — | 3060 |
| 19 | 1162261467 | — | 3876 |
| 20 | 3486784401 | 248832 | 4845 |

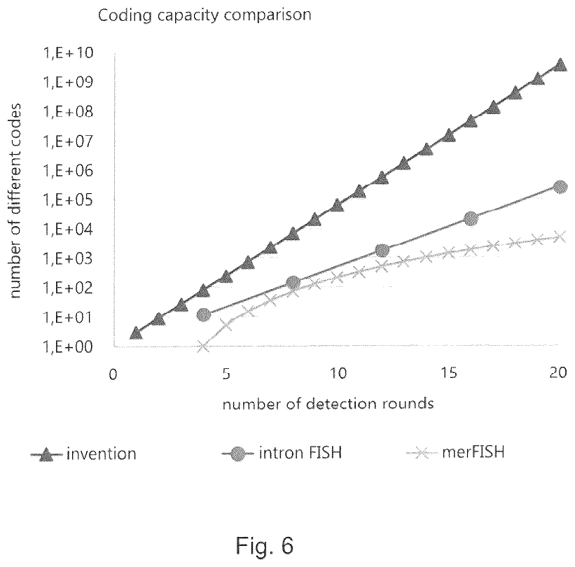

As shown in FIG. 6 the number of codewords for merFISH does not exponentially increase with the number of detection cycles but gets less effective with each added round. In contrast, the number of codewords for intronSeqFISH in the method according to the disclosure increases exponentially. The slope of the curve for the proposed method is much higher than that of intron FISH, leading to more than 10,000 times more code words usable after 20 rounds of detection.

Note that this maximum efficiency of coding capacity is also reached in case of seqFISH, where specific probes are denatured after every detection round and a new probe set is specifically hybridized to the target sequence for each detection round. However, this method has major downsides to technologies using only one specific hybridization for their encoding scheme (all other methods):
(1) For the efficient denaturation of the specific probes, rather crude conditions must be used (high temperatures, high concentrations of denaturing agent, long incubation times) leading to much higher probability for the loss or the damage of the analyte.
(2) For each round of detection an own probe set has to be used for every target nucleic acid sequence. Therefore, the number of specific probes needed for the experiment scales with the number of different signals needed for the encoding scheme. This dramatically increases the complexity and the cost of the assay.
(3) Because the hybridization efficiency of every target nucleic acid molecule is subject to some probabilistic effects, the fluctuations of signal intensity between the different detection rounds is much higher than in methods using only one specific hybridization event, reducing the proportion of complete codes.
(4) The time needed for the specific hybridization is much longer than for the hybridization of signal oligonucleotides or decoding oligonucleotides (as can be seen in the method parts of the intronSeqFISH, merFISH and seqFISH publications), which dramatically increases the time needed to complete an experiment.

Due to these reasons all other methods use a single specific hybridization event and accept the major downside of lower code complexity and therefore the need of more detection rounds and a higher oligonucleotide design complexity.

The method according to the disclosure combines the advantages of seqFISH (mainly complete freedom concerning the encoding scheme) with all advantages of methods using only one specific hybridization event while eliminating the major problems of such methods.

Note that the high numbers of code words produced after 20 rounds can also be used to introduce higher hamming distances (differences) between different codewords, allowing error detection of 1, 2 or even more errors and even error corrections. Therefore, even very high coding capacities are still practically relevant.

For the detection of subgroups/variations within a group of analytes/targets that share certain parts (e.g. mRNA splice variants), two separate code words might be used. However, signals will be generated at the very same physical location, so that a mixed readout will be the results for this naïve approach. Using an additional round to add information to an already established code circumvents this issue.

As mentioned above, the usage of multi-decoders further increases the coding capacity of the encoding scheme. Instead of being limited to the having exactly the same number of different signal types as different signal oligonucleotides and corresponding translator elements, the use of multi-decoders increases the signal types that can be used to: (N×(N+1))/2 (with N being the number of different signal oligonucleotides used). For the code used in table 1 with 3 different signal oligonucleotides this would mean the following 7 different signal types could be used: (S1), (S2), (S3), (S1/S2), (S1/S3), (S2/S3), (S1/S2/S3). The effect to the coding efficiency can be seen in Table 1b and FIG. 18.

| | CODING CAPACITY | | | |
|---|---|---|---|---|
| | Method | Method of the | | |
| | of the | present disclosure | | |
| NUMBER OF | present | with multi- | intron | |
| ROUNDS: | disclosure | decoders | FISH | merFISH |
| 1 | 3 | 7 | — | — |
| 2 | 9 | 49 | — | — |
| 3 | 27 | 343 | — | — |
| 4 | 81 | 2401 | 12 | 1 |
| 5* | 243 | 16807 | — | 5 |
| 6 | 729 | 117649 | — | 15 |
| 7** | 2187 | 823543 | — | 35 |
| 8 | 6561 | 5764801 | 144 | 70 |
| 9 | 19683 | 40353607 | — | 126 |
| 10 | 59049 | 282475249 | — | 210 |
| 11 | 177147 | 1977326743 | — | 330 |
| 12*** | 531441 | 1,3841 × 10\hat{ }10 | 1728 | 495 |
| 13 | 1594323 | 9,6889 × 10\hat{ }10 | — | 715 |
| 14 | 4782969 | 6,7822 × 10\hat{ }11 | — | 1001 |
| 15 | 14348907 | 4,7476 × 10\hat{ }12 | — | 1365 |
| 16 | 43046721 | 3,3233 × 10\hat{ }13 | 20736 | 1820 |
| 17 | 129140163 | 2,3263 × 10\hat{ }14 | — | 2380 |
| 18 | 387420489 | 1,6284 × 10\hat{ }15 | — | 3060 |
| 19 | 1162261467 | 1,1399 × 10\hat{ }16 | — | 3876 |
| 20 | 3486784401 | 7,9792 × 10\hat{ }16 | 248832 | 4845 |

Table 1b shows the coding capacity of the four methods.

All four methods compared in table 1b use specific probe sets that are not denatured between different rounds of detection. For intronSeqFISH there are four detection rounds needed to produce the pseudo colors of one coding round, therefore data is only given for rounds 4, 8, 12, 16 and 20. The merFISH-method uses a constant number of 4 signals, therefore the data starts with the smallest number of rounds possible. After 4 detection rounds the method with multi-decoders as described here exceeds the maximum coding capacity reached with 20 rounds of merFISH (depicted with one asterisk), after 7 rounds of detection the maximum coding capacity of intron FISH is exceeded (depicted with two asterisks) and after 12 rounds of detection the maximum coding capacity of the method of the present disclosure is exceeded (depicted with three asterisks). The usage of 3 different signal oligonucleotides is assumed (as is with intronSeqFISH).

### 3. Selective Denaturation, Oligonucleotide Assembly and Reuse of Unique Identifiers are Surprisingly Efficient

A key factor of the method according to the disclosure is the consecutive process of decoding oligonucleotide binding, signal oligonucleotide binding, signal detection and selective denaturation. In order to generate an encoding scheme, this process has to be repeated several times (depending on the length of the code word). Because the same unique identifier is reused in every detection cycle, all events from the first to the last detection cycle are depending on each other. Additionally, the selective denaturation depends on two different events: While the decoding oligonucleotide has to be dissolved from the unique identifier with highest efficiency, specific probes have to stay hybridized with highest efficiency.

Due to this the efficiency E of the whole encoding process can be described by the following equation:

$$\begin{matrix}E = {B}_{s p} \times {( {B}_{d e} \times {B}_{s i} \times {E}_{d e} \times {S}_{s p} )}^{n} \\ E = total efficiency \\ {B}_{sp} = binding of specific probes \\ {B}_{de} = binding of decoding oligonucleotides \\ {B}_{si} = binding of signal oligonucleotides \\ {E}_{de} = elimination of decoding oligonucleotides \\ {S}_{sp} = stability of specific probes during elimantion process \\ n = number of detection cycles\end{matrix}$$

Based on this equation the efficiency of each single step can be estimated for a given total efficiency of the method. The calculation is hereby based on the assumption, that each process has the same efficiency. The total efficiency describes the portion of successfully decodable signals of the total signals present.

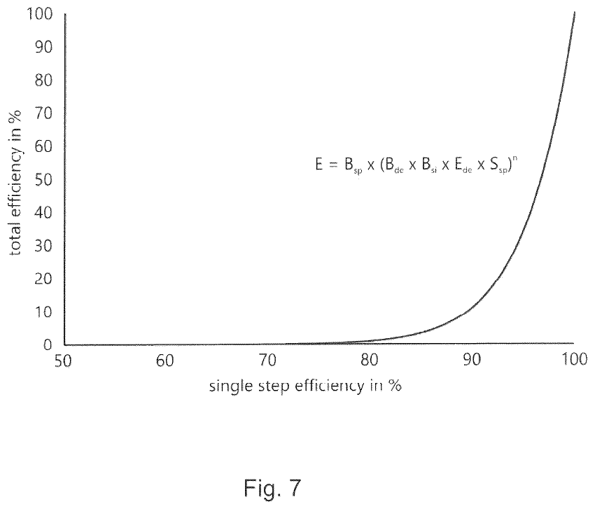

The total efficiency of the method is dependent on the efficiency of each single step of the different factors described by the equation. Under the assumption of an equally distributed efficiency the total efficiency can be plotted against the single step efficiency as shown in FIG. 7. As can be seen, a practically relevant total efficiency for an encoding scheme with 5 detection cycles can only be achieved with single step efficiencies clearly above 90%. For example, to achieve a total efficiency of 50% an average efficiency within each single step of 97.8% is needed. These calculations are even based on the assumption of a 100% signal detection and analysis efficiency. Due to broad DNA melting curves of oligonucleotides of a variety of sequences, the inventors assumed prior to experiments that the selective denaturation would work less efficient for denaturation of decoding oligonucleotides and that sequence specific binding probes are not stable enough. In contrast to this assumption, we found a surprising effectiveness of all steps and a high stability of sequence specific probes during selective denaturation.

Experimentally, the inventors achieved a total decoding efficiency of about 30% to 65% based on 5 detection cycles. A calculation of the efficiency of each single step (Bsp, Bde, Bsi, Ede, Ssp) by the formula given above revealed an average efficiency of about 94.4% to 98%. These high efficiencies are very surprising and cannot easily be anticipated by a well-trained person in this field.

### 4. Experimental Data

### Background

The experiment shows the specific detection of 10 to 50 different mRNAs species in parallel with single molecule resolution. It is based on 5 detection cycles, 3 different fluorescent signals and an encoding scheme without signal gaps and a hamming distance of 2 (error detection). The experiment proofs the enablement and functionality of the method according to the disclosure.

Oligonucleotides and their Sequences

All oligonucleotide sequences used in the experiment (target specific probes, decoding oligonucleotides, signal oligonucleotides) are listed in the sequence listing of the appendix. The signal oligonucleotide R:ST05*O_Atto594 was ordered from biomers.net GmbH. All other oligonucleotides were ordered from Integrated DNA Technologies. Oligonucleotides were dissolved in water. The stock solutions (100 μM) were stored at −20° C.

### Experimental Overview

The 50 different target specific probe sets are divided into 5 groups. The name of the transcript to be detected and the name of the target specific probe set are the same (transcript variant names of www.ensemble.org). The term “new” indicates a revised probe design. All oligonucleotide sequences of the probe sets can be found in the sequence listing. The table lists the unique identifier name of the probe set as well as the names of the decoding oligonucleotides used in the different detection cycles. The resulting code shows the sequence of fluorescent signals generated during the 5 detection cycles (G(reen)=Alexa Fluor 488, O(range)=Atto 594, Y(ellow)=Alexa Fluor 546).

TABLE 2
Experimental overview

| target | unique | Decoding oligonucleotides in detection cycle: | | | | | resulting |
|---|---|---|---|---|---|---|---|
| transcript | identifier | 1 | 2 | 3 | 4 | 5 | code |

Group 1

| | | | | | | | |
|---|---|---|---|---|---|---|---|
| DDX5-201 | ST21 | ST21-ST07 | ST21-ST05 | ST21-ST07 | ST21-ST05 | ST21-ST06 | GOGOY |
| RAD17-208 | ST02 | ST02-ST06 | ST02-ST07 | ST02-ST06 | ST02-ST06 | ST02-ST07 | YGYYG |
| SPOCK1-202 | ST03 | ST03-ST06 | ST03-ST06 | ST03-ST07 | ST03-ST05 | ST03-ST05 | YYGOO |
| FBXO32-203 | ST04 | ST04-ST07 | ST04-ST06 | ST04-ST06 | ST04-ST06 | ST04-ST05 | GYYYO |
| THRAP3-203 | ST14 | ST14-ST07 | ST14-ST05 | ST14-ST05 | ST14-ST07 | ST14-ST05 | GOOGO |
| GART-203 | ST11 | ST11-ST06 | ST11-ST07 | ST11-ST06 | ST11-ST05 | ST11-ST05 | YGYOO |
| KAT2A-201 | ST13 | ST13-ST06 | ST13-ST06 | ST13-ST07 | ST13-ST06 | ST13-ST07 | YYGYG |
| HPRT1-201 | ST12 | ST12-ST06 | ST12-ST07 | ST12-ST07 | ST12-ST06 | ST12-ST06 | YGGYY |
| CCNA2-201 | ST22 | ST22-ST05 | ST22-ST07 | ST22-ST06 | ST22-ST06 | ST22-ST05 | OGYYO |
| NKRF-201 | ST23 | ST23-ST05 | ST23-ST06 | ST23-ST07 | ST23-ST06 | ST23-ST05 | OYGYO |

Group 2

| | | | | | | | |
|---|---|---|---|---|---|---|---|
| CCNE1-201-new | NT01 | NT01-ST07 | NT01-ST07 | NT01-ST06 | NT01-ST06 | NT01-ST06 | GGYYY |
| COG5-201 | NT03 | NT03-ST06 | NT03-ST06 | NT03-ST05 | NT03-ST05 | NT03-ST06 | YYOOY |
| FBN1-201 | NT04 | NT04-ST05 | NT04-ST07 | NT04-ST06 | NT04-ST07 | NT04-ST07 | OGYGG |
| DYNC1H1-201 | NT05 | NT05-ST07 | NT05-ST06 | NT05-ST07 | NT05-ST06 | NT05-ST06 | GYGYY |
| CKAP5-202 | NT06 | NT06-ST05 | NT06-ST06 | NT06-ST07 | NT06-ST05 | NT06-ST06 | OYGOY |
| KRAS-202 | NT07 | NT07-ST06 | NT07-ST05 | NT07-ST06 | NT07-ST06 | NT07-ST05 | YOYYO |
| EGFR-207 | NT08 | NT08-ST07 | NT08-ST06 | NT08-ST05 | NT08-ST05 | NT08-ST05 | GYOOO |
| TP53-205 | NT09 | NT09-ST06 | NT09-ST05 | NT09-ST06 | NT09-ST07 | NT09-ST07 | YOYGG |
| NF1-204 | XT01 | XT01-ST06 | XT01-ST06 | XT01-ST07 | XT01-ST07 | XT01-ST06 | YYGGY |
| NF2-204 | XT02 | XT02-ST07 | XT02-ST06 | XT02-ST05 | XT02-ST06 | XT02-ST07 | GYOYG |

Group 3

| | | | | | | | |
|---|---|---|---|---|---|---|---|
| ACO2-201 | XT03 | XT03-ST06 | XT03-ST06 | XT03-ST06 | XT03-ST07 | XT03-ST05 | YYYGO |
| AKT1-211 | XT04 | XT04-ST07 | XT04-ST06 | XT04-ST07 | XT04-ST07 | XT04-ST05 | GYGGO |
| LYPLAL1-202 | XT05 | XT05-ST07 | XT05-ST05 | XT05-ST06 | XT05-ST05 | XT05-ST05 | GOYOO |
| PKD2-201 | XT06 | XT06-ST06 | XT06-ST07 | XT06-ST05 | XT06-ST05 | XT06-ST07 | YGOOG |
| ENG-204 | XT09 | XT09-ST05 | XT09-ST05 | XT09-ST06 | XT09-ST06 | XT09-ST06 | OOYYY |
| FANCE-201 | XT10 | XT10-ST05 | XT10-ST07 | XT10-ST06 | XT10-ST05 | XT10-ST06 | OGYOY |
| MET-201 | XT12 | XT12-ST05 | XT12-ST06 | XT12-ST05 | XT12-ST06 | XT12-ST06 | OYOYY |
| NOTCH2-201 | XT13 | XT13-ST05 | XT13-ST05 | XT13-ST06 | XT13-ST07 | XT13-ST05 | OOYGO |
| SPOP-206 | XT14 | XT14-ST05 | XT14-ST07 | XT14-ST05 | XT14-ST07 | XT14-ST06 | OGOGY |
| ABL1-202 | XT16 | XT16-ST05 | XT16-ST06 | XT16-ST06 | XT16-ST05 | XT16-ST05 | OYYOO |

Group 4

| | | | | | | | |
|---|---|---|---|---|---|---|---|
| ATP11C-202 | XT17 | XT17-ST07 | XT17-ST06 | XT17-ST06 | XT17-ST05 | XT17-ST06 | GYYOY |
| BCR-202 | XT18 | XT18-ST05 | XT18-ST06 | XT18-ST06 | XT18-ST07 | XT18-ST06 | OYYGY |
| CAV1-205 | XT19 | XT19-ST07 | XT19-ST05 | XT19-ST06 | XT19-ST07 | XT19-ST06 | GOYGY |
| CDK2-201 | XT20 | XT20-ST05 | XT20-ST05 | XT20-ST07 | XT20-ST07 | XT20-ST06 | OOGGY |
| DCAF1-202 | XT201 | XT201-ST06 | XT201-ST06 | XT201-ST05 | XT201-ST07 | XT201-ST07 | YYOGG |
| FHOD1-201 | XT202 | XT202-ST05 | XT202-ST07 | XT202-ST07 | XT202-ST05 | XT202-ST07 | OGGOG |
| GMDS-202 | XT203 | XT203-ST07 | XT203-ST05 | XT203-ST07 | XT203-ST06 | XT203-ST05 | GOGYO |
| IFNAR1-201 | XT204 | XT204-ST06 | XT204-ST07 | XT204-ST05 | XT204-ST07 | XT204-ST05 | YGOGO |
| NSMF-203 | XT206 | XT206-ST07 | XT206-ST06 | XT206-ST07 | XT206-ST05 | XT206-ST07 | GYGOG |
| POLA2-201 | XT208 | XT208-ST06 | XT208-ST06 | XT208-ST06 | XT208-ST05 | XT208-ST07 | YYYOG |

Group 5

| | | | | | | | |
|---|---|---|---|---|---|---|---|
| BRCA1-210new | NT10 | NT10-ST07 | NT10-ST05 | NT10-ST06 | NT10-ST06 | NT10-ST07 | GOYYG |
| JAK1-201new | XT11 | XT11-ST05 | XT11-ST05 | XT11-ST07 | XT11-ST06 | XT11-ST07 | OOGYG |
| STRAP-202 | XT207 | XT207-ST07 | XT207-ST07 | XT207-ST06 | XT207-ST05 | XT207-ST07 | GGYOG |
| SERPINB5-201 | XT209 | XT209-ST07 | XT209-ST05 | XT209-ST05 | XT209-ST05 | XT209-ST07 | GOOOG |
| SETX-201 | XT210 | XT210-ST06 | XT210-ST07 | XT210-ST06 | XT210-ST07 | XT210-ST06 | YGYGY |
| WDFY1-201 | XT212 | XT212-ST05 | XT212-ST07 | XT212-ST05 | XT212-ST06 | XT212-ST07 | OGOYG |
| TACC1-201 | XT213 | XT213-ST05 | XT213-ST07 | XT213-ST07 | XT213-ST07 | XT213-ST05 | OGGGO |
| KIF2A-203 | XT214 | XT214-ST07 | XT214-ST07 | XT214-ST06 | XT214-ST07 | XT214-ST05 | GGYGO |
| CDT1-201 | XT215 | XT215-ST07 | XT215-ST07 | XT215-ST07 | XT215-ST05 | XT215-ST05 | GGGOO |
| CENPE-202 | XT216 | XT216-ST07 | XT216-ST06 | XT216-ST05 | XT216-ST07 | XT216-ST06 | GYOGY |

### Variations of the Experiment

Some variations of the experiment have been performed. Experiments 1 to 4 mainly differ in the number of transcripts detected in parallel. The groups listed as target specific probe sets refer to table 6. Experiments 5 to 8 are single round, single target controls for comparison with the decoded signals.

TABLE 3
Variations of the experiment

| | | Nr. of | Imaging |
|---|---|---|---|
| | Target specific | detection | with |
| Experiment | probe sets used | cycles | trolox |
| 1. 50 transcripts_T+ | Groups 1 to 5 of table 6 | 5 | + |
| 2. 50 transcripts_T− | Groups 1 to 5 of table 6 | 5 | − |
| 3. 30 transcripts_T+ | Groups 2 to 4 of table 6 | 5 | + |
| 4. 10 transcripts_T+ | Group 1 of table 6 | 5 | + |
| 5. DDX5 | DDX5-ST21 | 1 | − |
| 6. RAD17 | RAD17-ST02 | 1 | − |
| 7. SPOCK1 | SPOCK1-ST03 | 1 | − |
| 8. THRAP3 | THRAP3-ST14 | 1 | − |

### Experimental Details

### A. Seeding and Cultivation of Cells

Hela cells were grown in Hela cell culture medium to nearly 100% confluency. The Hela cell culture medium comprises DMEM (Thermo Fisher, Cat.: 31885) with 10% FCS (Biochrom, Cat.: S0415), 1% Penicillin-Streptomycin (Sigma-Adrich, Cat.: P0781) and 1% MEM Non-Essential Amino Acids Solution (Thermo Fisher, Cat.: 11140035). After aspiration of cell culture medium, cells were trypsinized by incubation with trypsin EDTA solution (Sigma-Aldrich, Cat.: T3924) for 5 min at 37° C. after a washing step with PBS (1,424 g/l Na₂HPO₄*2H₂O, 0.276 g/l, NaH₂PO₄*2H₂O, 8.19 g/l NaCl in water, pH 7.4). Cells were then seeded on the wells of a μ-Slide 8 Well ibidiTreat (Ibidi, Cat.: 80826). The number of cells per well was adjusted to reach about 50% confluency after adhesion of the cells. Cells were incubated over night with 200 μl HeLa cell culture medium per well.

### B. Fixation of Cells

After aspiration of cell culture medium and two washing steps with 200 μl 37° C. warm PBS per well, cells were fixed with 200 μl precooled methanol (−20° C., Roth, Cat.: 0082.1) for 10 min at −20° C.

C. Counterstaining with Sudan Black

Methanol was aspirated and 150 μl of 0.2% Sudan Black-solution diluted in 70% ethanol were added to each well. Wells were incubated for 5 min in the dark at room temperature. After incubation cells were washed three times with 400 μl 70% ethanol per well to eliminate the excess of Sudan Black-solution.

### D. Hybridization of Analyte/Target-Specific Probes

Before hybridization, cells were equilibrated with 200 μl sm-wash-buffer. The sm-wash-buffer comprises 30 mM Na₃Citrate, 300 mM NaCl, pH7, 10% formamide (Roth, Cat.: P040.1) and 5 mM Ribonucleoside Vanadyl Complex (NEB, Cat.: S1402S). For each target-specific probe set 1 μl of a 100 μM oligonucleotide stock solution was added to the mixture. The oligonucleotide stock solution comprises equimolar amounts of all target specific oligonucleotides of the corresponding target specific probe set. The total volume of the mixture was adjusted to 100 μl with water and mixed with 100 μl of a 2× concentrated hybridization buffer solution. The 2× concentrated hybridization buffer comprises 120 mM Na₃Citrate, 1200 mM NaCl, pH7, 20% formamide and 20 mM Ribonucleoside Vanadyl Complex. The resulting 200 μl hybridization mixture was added to the corresponding well and incubated at 37° C. for 2 h. Afterwards cells were washed three times with 200 μl per well for 10 min with target probe wash buffer at 37° C. The target probe wash buffer comprises 30 mM Na₃Citrate, 300 mM NaCl, pH7, 20% formamide and 5 mM Ribonucleoside Vanadyl Complex.

### E. Hybridization of Decoding Oligonucleotides

Before hybridization, cells were equilibrated with 200 μl sm-wash-buffer. For each decoding oligonucleotide 1.5 μl of a 5 μM stock solution were added to the mixture. The total volume of the mixture was adjusted to 75 μl with water and mixed with 75 μl of a 2× concentrated hybridization buffer solution. The resulting 150 μl decoding oligonucleotide hybridization mixture was added to the corresponding well and incubated at room temperature for 45 min. Afterwards cells were washed three times with 200 μl per well for 2 min with sm-wash-buffer at room temperature.

### F. Hybridization of Signal Oligonucleotides

Before hybridization, cells were equilibrated with 200 μl sm-wash-buffer. The signal oligonucleotide hybridization mixture was the same for all rounds of experiments 1 to 4 and comprised 0.3 μM of each signal oligonucleotide (see table A3) in 1× concentrated hybridization buffer solution. In each round 150 μl of this solution were added per well and incubated at room temperature for 45 min. The procedure was the same for experiments 5 to 8 with the exception that the final concentration of each signal oligonucleotide was 0.15 μM. Afterwards cells were washed three times with 200 μl per well for 2 min with sm-wash-buffer at room temperature.

### G. Fluorescence and White Light Imaging

Cells were washed once with 200 μl of imaging buffer per well at room temperature. In experiments without Trolox (see table 7, last column) imaging buffer comprises 30 mM Na₃Citrate, 300 mM NaCl, pH7 and 5 mM Ribonucleoside Vanadyl Complex. In experiments with Trolox, imaging buffer additionally contains 10% VectaCell Trolox Antifade Reagent (Vector laboratories, Cat.: CB-1000), resulting in a final Trolox concentration of 10 mM.

A Zeiss Axiovert 200M microscope with a 63×immersion oil objective (Zeiss, apochromat) with numerical aperture of 1.4, a pco.edge 4.2 CMOS camera (PCO AG) and an LED-light source (Zeiss, colibri 7) was used for imaging of the regions. Filter sets and LED-wavelengths were adjusted to the different optima of the fluorophores used. Illumination times per image were 1000 ms for Alexa Fluor 546 and Atto 594 and 400 ms for Alexa Fluor 488.

In each experiment, three regions were randomly chosen for imaging. For each region, a z-stack of 32 images was detected with a z-step size of 350 nm. Additionally, one white light image was taken from the regions. In experiments with more than one detection cycle, the regions of the first detection round were found back and imaged in every subsequent round.

### H. Selective Denaturation

For selective denaturation, every well was incubated with 200 μl of sm-wash-buffer at 42° C. for 6 min. This procedure was repeated six times.

Steps (E) to (H) were repeated 5 times in experiments 1 to 4. Step (H) was omitted for the 5th detection cycle.

### I. Analysis

Based on custom ImageJ-plugins a semi-automated analysis of the raw data was performed to distinguish the specific fluorescent signals from the background. The resulting 3D-point clouds of all three fluorescent channels were combined in silico with a custom VBA script. The resulting combined 3D-point clouds of the 5 detection cycles were aligned to each other on the basis of a VBA script. The resulting alignments revealed the code words for each unique signal detected. Successfully decoded signals were used for quantitative and spatial analysis of the experiments based on custom VBA-scripts and ImageJ-plugins.

### Results

### 1. Absolute Numbers of Decoded Signals

The absolute numbers of successfully decoded signals for all transcripts are listed for each region of each experiment in the following Table 4. In summary, the sum of correct codes depicts the total number of decoded signals that were assigned to transcripts detectable in the corresponding experiment, while the sum of incorrect codes it the total number of decoded signals not detectable in the corresponding experiment. The total number of signals comprises successfully decoded as well as unsuccessfully decoded signals.

TABLE 4
Absolute numbers of decoded signals

| | Experiment 1: region: | | | Experiment 2: region: | | | Experiment 3: region: | | | Experiment 4: region: | | |
|---|---|---|---|---|---|---|---|---|---|---|---|---|
| transcript name: | 1 | 2 | 3 | 1 | 2 | 3 | 1 | 2 | 3 | 1 | 2 | 3 |

Group 1

| | | | | | | | | | | | | |
|---|---|---|---|---|---|---|---|---|---|---|---|---|
| DDX5-201 | 1214 | 1136 | 927 | 1144 | 1509 | 1176 | 2964 | 2034 | 2141 | 8 | 2 | 2 |
| RAD17-208 | 40 | 126 | 50 | 26 | 55 | 62 | 22 | 30 | 33 | 7 | 9 | 10 |
| SPOCK1-202 | 581 | 655 | 301 | 483 | 875 | 149 | 1349 | 621 | 539 | 2 | 10 | 8 |
| FBXO32-203 | 153 | 175 | 68 | 113 | 106 | 78 | 301 | 160 | 269 | 5 | 13 | 9 |
| THRAP3-203 | 1079 | 2180 | 1035 | 810 | 1179 | 1047 | 2318 | 1609 | 1609 | 6 | 8 | 16 |
| GART-203 | 422 | 397 | 346 | 350 | 333 | 202 | 766 | 658 | 569 | 5 | 3 | 2 |
| KAT2A-201 | 141 | 310 | 166 | 174 | 307 | 186 | 382 | 315 | 340 | 5 | 1 | 3 |
| HPRT1-201 | 63 | 79 | 34 | 44 | 116 | 71 | 85 | 88 | 112 | 1 | 0 | 6 |
| CCNA2-201 | 91 | 248 | 205 | 95 | 134 | 138 | 241 | 151 | 238 | 10 | 20 | 9 |
| NKRF-201 | 162 | 318 | 153 | 101 | 99 | 135 | 313 | 254 | 209 | 7 | 5 | 12 |

Group 2

| | | | | | | | | | | | | |
|---|---|---|---|---|---|---|---|---|---|---|---|---|
| CCNE1-201-new | 75 | 180 | 38 | 57 | 110 | 97 | 0 | 0 | 1 | 122 | 104 | 120 |
| COG5-201 | 57 | 21 | 38 | 26 | 45 | 23 | 0 | 0 | 1 | 56 | 86 | 60 |
| FBN1-201 | 202 | 1338 | 499 | 456 | 513 | 571 | 0 | 1 | 3 | 450 | 778 | 895 |
| DYNC1H1-201 | 554 | 892 | 398 | 664 | 1026 | 666 | 4 | 3 | 7 | 823 | 1148 | 1248 |
| CKAP5-202 | 43 | 23 | 77 | 51 | 98 | 74 | 1 | 2 | 1 | 94 | 190 | 212 |
| KRAS-202 | 331 | 417 | 355 | 302 | 333 | 230 | 0 | 0 | 1 | 279 | 637 | 371 |
| EGFR-207 | 527 | 252 | 372 | 293 | 519 | 322 | 0 | 1 | 0 | 411 | 719 | 818 |
| TP53-205 | 116 | 324 | 194 | 138 | 198 | 169 | 0 | 1 | 4 | 182 | 280 | 181 |
| NF1-204 | 381 | 676 | 320 | 347 | 522 | 416 | 3 | 2 | 0 | 507 | 642 | 659 |
| NF2-204 | 434 | 638 | 361 | 336 | 468 | 401 | 0 | 0 | 2 | 508 | 523 | 636 |

Group 3

| | | | | | | | | | | | | |
|---|---|---|---|---|---|---|---|---|---|---|---|---|
| ACO2-201 | 456 | 759 | 444 | 333 | 367 | 345 | 0 | 0 | 0 | 556 | 681 | 681 |
| AKT1-211 | 351 | 710 | 301 | 230 | 437 | 297 | 0 | 0 | 3 | 558 | 614 | 690 |
| LYPLAL1-202 | 65 | 62 | 51 | 33 | 58 | 51 | 7 | 6 | 8 | 28 | 34 | 42 |
| PKD2-201 | 72 | 194 | 124 | 95 | 129 | 69 | 1 | 0 | 0 | 122 | 164 | 169 |
| ENG-204 | 472 | 890 | 458 | 446 | 494 | 558 | 0 | 2 | 0 | 1145 | 799 | 1119 |
| FANCE-201 | 24 | 82 | 61 | 56 | 67 | 56 | 0 | 0 | 2 | 119 | 131 | 153 |
| MET-201 | 268 | 744 | 333 | 426 | 275 | 462 | 0 | 0 | 0 | 790 | 662 | 782 |
| NOTCH2-201 | 344 | 823 | 404 | 172 | 256 | 208 | 4 | 0 | 3 | 402 | 779 | 772 |
| SPOP-206 | 43 | 377 | 139 | 68 | 117 | 100 | 0 | 0 | 0 | 215 | 289 | 300 |
| ABL1-202 | 224 | 72 | 218 | 107 | 122 | 153 | 4 | 1 | 1 | 302 | 393 | 480 |

Group 4

| | | | | | | | | | | | | |
|---|---|---|---|---|---|---|---|---|---|---|---|---|
| ATP11C-202 | 170 | 116 | 121 | 86 | 165 | 128 | 2 | 1 | 1 | 130 | 206 | 234 |
| BCR-202 | 180 | 401 | 185 | 177 | 149 | 169 | 1 | 0 | 0 | 321 | 372 | 485 |
| CAV1-205 | 728 | 777 | 644 | 328 | 653 | 567 | 2 | 0 | 5 | 613 | 997 | 852 |
| CDK2-201 | 306 | 937 | 367 | 297 | 385 | 358 | 4 | 1 | 3 | 568 | 742 | 888 |
| DCAF1-202 | 119 | 292 | 187 | 59 | 67 | 65 | 0 | 1 | 0 | 108 | 171 | 131 |
| FHOD1-201 | 60 | 233 | 143 | 132 | 185 | 157 | 1 | 0 | 1 | 194 | 294 | 300 |
| GMDS-202 | 67 | 124 | 49 | 56 | 80 | 63 | 7 | 2 | 3 | 59 | 73 | 114 |
| IFNAR1-201 | 81 | 221 | 135 | 99 | 104 | 55 | 1 | 0 | 1 | 159 | 266 | 238 |
| NSMF-203 | 448 | 583 | 386 | 293 | 453 | 331 | 8 | 4 | 11 | 525 | 608 | 616 |
| POLA2-201 | 74 | 111 | 62 | 45 | 72 | 67 | 2 | 4 | 2 | 41 | 75 | 57 |

Group 5

| | | | | | | | | | | | | |
|---|---|---|---|---|---|---|---|---|---|---|---|---|
| BRCA1-210new | 230 | 704 | 248 | 324 | 439 | 374 | 2 | 1 | 0 | 2 | 3 | 3 |
| JAK1-201new | 157 | 554 | 223 | 185 | 259 | 263 | 0 | 1 | 0 | 5 | 4 | 2 |
| STRAP-202 | 42 | 75 | 31 | 18 | 52 | 40 | 3 | 1 | 0 | 6 | 4 | 4 |
| SERPINB5-201 | 324 | 598 | 343 | 286 | 344 | 364 | 6 | 13 | 9 | 0 | 1 | 2 |
| SETX-201 | 212 | 540 | 254 | 291 | 439 | 336 | 2 | 2 | 2 | 12 | 9 | 5 |
| WDFY1-201 | 43 | 516 | 218 | 176 | 206 | 147 | 0 | 0 | 0 | 4 | 2 | 8 |
| TACC1-201 | 69 | 373 | 131 | 80 | 156 | 118 | 1 | 0 | 0 | 21 | 29 | 32 |
| KIF2A-203 | 442 | 879 | 445 | 298 | 519 | 430 | 5 | 1 | 2 | 6 | 11 | 9 |
| CDT1-201 | 31 | 27 | 33 | 19 | 61 | 28 | 15 | 8 | 9 | 0 | 8 | 1 |
| CENPE-202 | 192 | 246 | 215 | 94 | 262 | 225 | 0 | 1 | 0 | 8 | 9 | 9 |

Summary

| | | | | | | | | | | | | |
|---|---|---|---|---|---|---|---|---|---|---|---|---|
| sum of correct | 12960 | 23405 | 12890 | 11319 | 15917 | 12797 | 8741 | 5920 | 6059 | 10387 | 13457 | 14303 |
| codes: | | | | | | | | | | | | |
| sum of incorrect | 0 | 0 | 0 | 0 | 0 | 0 | 86 | 60 | 86 | 120 | 151 | 152 |
| codes: | | | | | | | | | | | | |
| total number of | 42959 | 72157 | 32185 | 32037 | 58793 | 35470 | 13549 | 9182 | 10109 | 26701 | 32966 | 35451 |
| signals: | | | | | | | | | | | | |
| % successfully | 30.2 | 32.4 | 40.0 | 35.3 | 27.1 | 36.1 | 64.5 | 64.5 | 59.9 | 38.9 | 40.8 | 40.3 |
| decoded: | | | | | | | | | | | | |

Table 4 shows a very low number of incorrectly decoded signals compared to the number of correctly decoded signals. The absolute values for decoded signals of a certain transcript are very similar between different regions of one experiment. The fraction of the total number of signals that can be successfully decoded is between 27.1% and 64.5%. This fraction depends on the number of transcripts and/or the total number of signals present in the respective region/experiment.

### Conclusion

The method according to the disclosure produces a low amount of incorrectly assigned code words and can therefore be considered specific. The fraction of successfully decodable signals is very high, even with very high numbers of signals per region and very high numbers of transcripts detected in parallel. The high fraction of assignable signals and the high specificity make the method practically useful.

### Comparison of Relative Transcript Abundancies Between Different Experiments

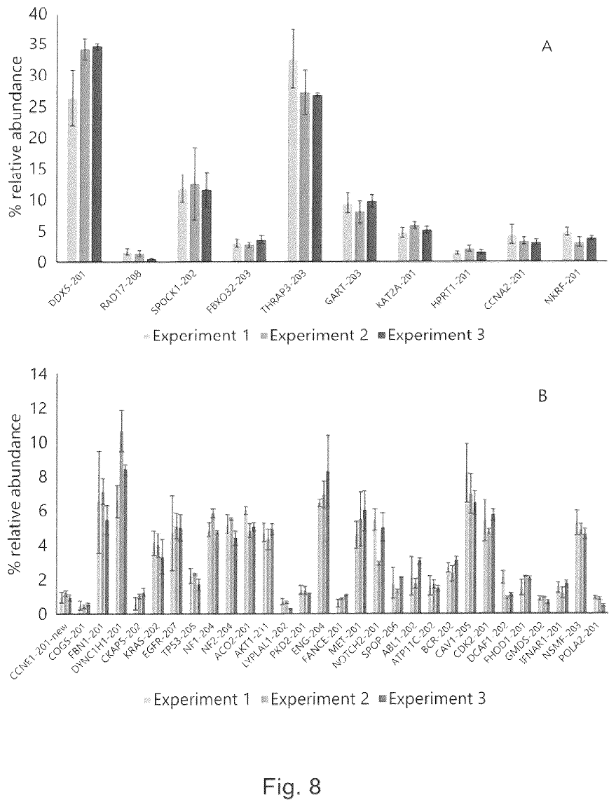

As shown in FIG. 8 for both comparisons (A and B) the overlap of detected transcripts between the experiments is used for the analysis. Each bar represents the mean abundance of all three regions of an experiment. The standard deviation between these regions is also indicated.

### Correlation of Relative Transcript Abundancies Between Different Experiments

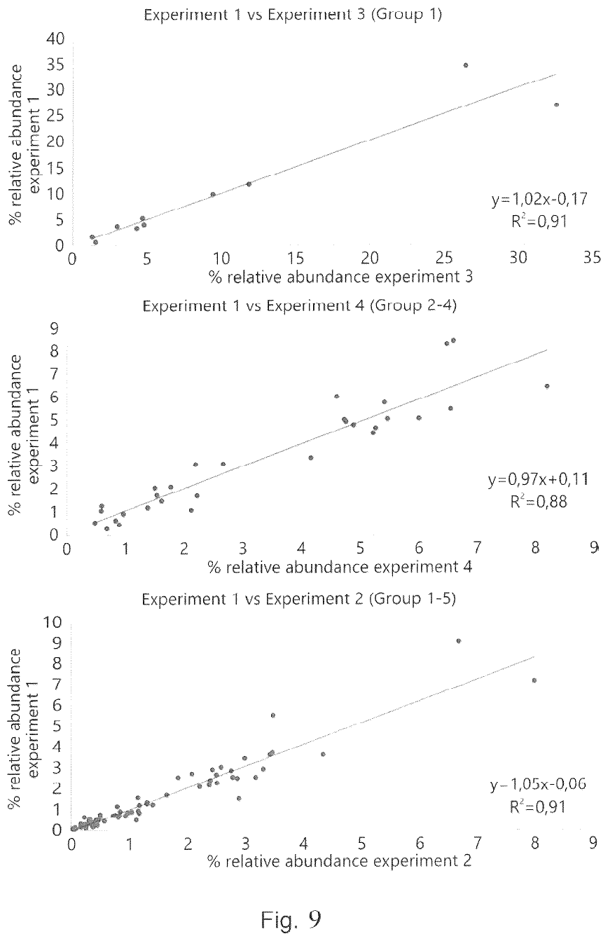

As can be seen in FIG. 9 the mean relative abundances of transcripts from experiment 1 are correlated to the abundances of the overlapping transcripts of experiment 3, 4 and 2. The correlation coefficient as well as the formula for the linear regression are indicated for each correlation.

FIG. 8 shows low standard deviations, indicating low variations of relative abundances between different regions of one experiment. The differences of relative abundances between transcripts from different experiments are also very low. This is the case for the comparison of transcripts from group 1 (FIG. 8A), that were detected in experiments 1, 2 and 3. It is also the case for the comparison of the transcripts from groups 2, 3 and 4 that were overlapping between experiments 1, 2 and 4. The very high correlation of these abundances can also be seen in FIG. 9. The abundances of transcripts from experiment 1 correlate very well with the abundances of the other multi round experiments. The correlation factors are between 0.88 and 0.91, while the slope of the linear regressions is between 0.97 and 1.05.

### Conclusion

The relative abundancies of transcripts correlate very well between different regions of one experiment but also between different experiments. This can be clearly seen by the comparisons of FIGS. 3 and 4. The main difference between the experiments is the number of different targets and hence the total number of signals detected. Therefore, the number of transcripts detected as well as the number and density of signals does not interfere with the ability of the method to accurately quantify the number of transcripts. The very good correlations further support the specificity and stability of the method, even with very high numbers of signals.

### Comparison of Intercellular Distribution of Signals

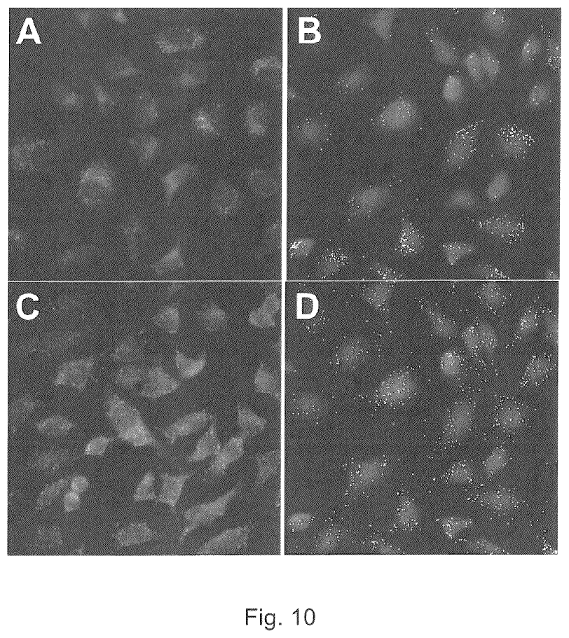

In FIG. 10 the maximum projections of image stacks are shown. A: region 1 of experiment 7 (single round, single transcript experiment detecting SPOCK1), B: 2D-projection of all selected signals from experiment 1, region 1 assigned to SPOCK1, C: region 1 of experiment 8 (single round, single transcript experiment detecting THRAP3), D: 2D-projection of all selected signals from experiment 1, region 1 assigned to THRAP3.

### Comparison of Intracellular Distribution of Signals

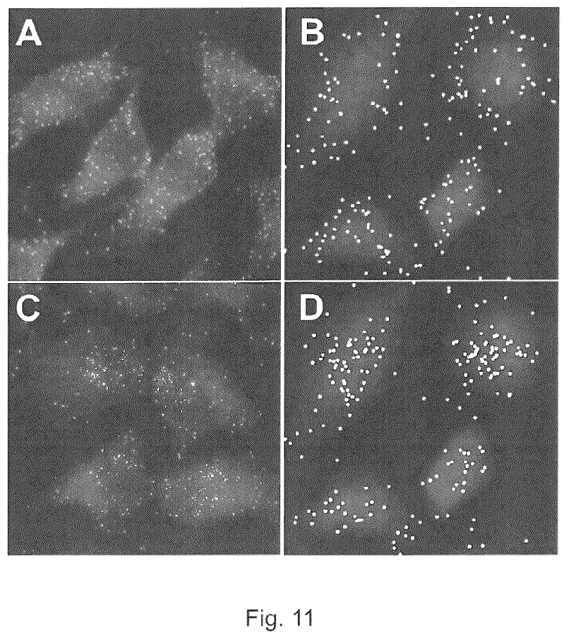

In FIG. 11 the maximum projections of image stacks are shown. Magnified sub regions of the corresponding regions are shown. A: region 1 of experiment 8 (single round, single transcript experiment detecting THRAP3), B: 2D-projection of selected signals from experiment 1, region 1 assigned to THRAP3, C: region 1 of experiment 5 (single round, single transcript experiment detecting DDX5), D: 2D-projection of all selected signals from experiment 1, region 1 assigned to DDX5.

FIG. 10 shows huge differences of intercellular distributions between different transcripts. SPOCK1 seems to be highly abundant in some cells but nearly absent in other cells (FIG. 10A). THRAP3 shows a more uniform distribution over all cells of a region (FIG. 10C). These spatial distribution patterns can also clearly be observed with the point clouds assigned to the corresponding transcripts from experiment 1 (FIGS. 10B and D).

FIG. 11 shows huge differences of intracellular distributions between different transcripts. THRAP3 can be mainly observed in the periphery (cytoplasm) of the cells (FIG. 11A), while DDX5 shows a higher abundance in the center (nucleus) of the cells (FIG. 11C). These intracellular distributions can also be observed with the point clouds of experiment 1 assigned to THRAP3 and DDX5 (FIGS. 11B and D).

### Conclusion

Next to the reliability of quantification, the point clouds of multi round experiments also show the same intracellular and intercellular distribution patterns of transcripts. This is clearly proven by the direct comparison of the assigned point clouds with signals from single round experiments detecting only one characteristic mRNA-species.

### Distribution Pattern of Different Cell Cycle Dependent Transcripts

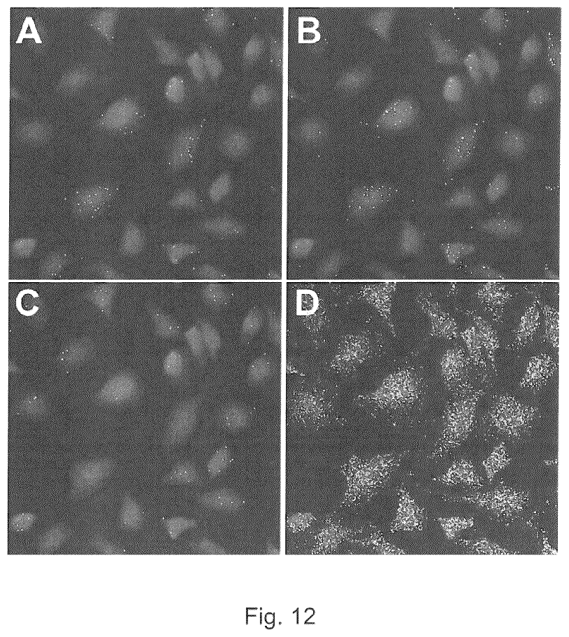

All images of FIG. 12 show region 1 of experiment 1. In each image, a point cloud is shown, that is assigned to a certain transcript, A: CCNA2, B: CENPE, C: CCNE1, D: all transcripts. FIG. 12 shows the transcripts of three different cell cycle dependent proteins. CENPE (FIG. 12B) is also known as Centromere protein E and accumulates during G2 phase. It is proposed to be responsible for spindle elongation and for chromosome movement. It is not present during interphase. CCNA2 (FIG. 12A) is also known as Cyclin A2. It regulates the cell cycle progression by interacting with CDK1 during transition from G2 to M-phase. Interestingly there is an obvious colocalization of both mRNA-species. They are mainly present in the three central cells of region 1. CCNE1 (FIG. 12C) is also known as Cyclin E1. This cyclin interacts with CDK2 and is responsible for the transition from G1 to S-phase. FIG. 12 shows clearly, that the transcripts of this gene are not present in the three central cells, but quite equally distributed over the other cells. It therefore shows an anti-localization to the other two transcripts. The data for the corresponding point-clouds are derived from a point cloud with a very high number of points and a very high point density (FIG. 12D gives an impression).

### Conclusion

The three decoded point clouds of cell cycle dependent proteins shown in FIG. 12, show distribution patterns that can be explained by their corresponding function. These data strongly suggest, that the method of the present disclosure reliably produces biological relevant data, even with a low number of signals per cell (FIG. 12C) and with very high signal densities (FIG. 12D).

### SEQUENCE LISTING

In the accompanying sequence listing SEQ ID Nos. 1-1247 refer to nucleotide sequences of exemplary target-specific oligonucleotides. The oligonucleotides listed consist of a target specific binding site (5′-end) a spacer/linker sequence (gtaac or tagac) and the unique identifier sequence, which is the same for all oligonucleotides of one probe set.

In the accompanying sequence listing SEQ ID Nos. 1248-1397 refer to nucleotide sequences of exemplary decoding oligonucleotides.

In the accompanying sequence listing SEQ ID Nos. 1398-1400 refer to the nucleotide sequences of exemplary signal oligonucleotides. For each signal oligonucleotide the corresponding fluorophore is present twice. One fluorophore is covalently linked to the 5′-end and one fluorophore is covalently linked to the 3′-end. SEQ ID No. 1398 comprises at its 5′ terminus “5Alex488N”, and at its 3′ terminus “3AlexF488N”. SEQ ID No. 1399 comprises at its 5′ terminus “5Alex546”, and at its 3′ terminus 3Alex546N. SEQ ID No. 1400 comprises at its 5′ terminus and at its 3′ terminus “Atto594”.

We claim:

1. A multiplex method for detecting different analytes in a sample, the method comprising:
(i) contacting the sample with at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element (S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
contacting the sample with at least two different sets of analyte-specific probes for at least one analyte and a variation thereof,
wherein the analyte-specific probes comprised in these different sets interacting with the same analyte, but specifically interact with different sub-structures of the same analyte,
wherein the analyte-specific probes of the first set of analyte-specific probes interacts with a sub-structure which is comprised in all variations of an analyte,
wherein the analyte-specific probes of the second set of analyte-specific probes (subgroup-specific probes) interacts with a sub-structure which is comprised only in a specific variation of the analyte,
wherein the analyte-specific probes of the first set of analyte-specific probes comprise the same identifier element (T) comprising a nucleotide sequence which is unique to the analyte to be encoded (unique identifier sequence), and
wherein the analyte-specific probes of the second set of analyte-specific probes comprise the same identifier element (T) comprising a nucleotide sequence which is unique to the analyte to be encoded (unique identifier sequence),
wherein the identifier elements (T) of the analyte-specific probes of the first set of analyte-specific probes and the identifier elements (T) of the analyte-specific probes of the second set of analyte-specific probes are different for binding different decoding oligonucleotides and/or non-signal decoding oligonucleotides.
(ii) contacting the sample with at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(iii) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element;
(iv) detecting the signal caused by the signal element;
(v) selectively removing the decoding oligonucleotides and signal oligonucleotides from the sample, thereby essentially maintaining the specific binding of the analyte-specific probes to the analytes to be encoded; and
(vi) performing at least three (3) further cycles comprising steps (ii) to
(v) to generate an encoding scheme with a code word per analyte.

2. The method according to claim 1, wherein the set of analyte-specific probes comprises at least five (5) subgroup-specific probes which specifically interact with different sub-structures of the same variation of an analyte.

3. The method according to claim 1, comprising at least one (1) further cycle comprising steps (ii) to (v) to identify the subgroup-specific probes, wherein in particular the cycle may stop with step (iv).

4.-5. (canceled)

6. The method according to claim 1, wherein all steps are automated.

7. (canceled)

8. The method according to claim 1, wherein each analyte is associated with a specific code word, wherein said code word comprises a number of positions, and wherein each position corresponds to one cycle resulting in a plurality of distinguishable encoding schemes with the plurality of code words.

9. (canceled)

10. The method according to claim 1 wherein the code words obtained for the individual analytes in the performed cycles comprise the detected signals and additionally at least one element corresponding to no detected signal.

11.-12. (canceled)

13. The method according to claim 1, wherein the code word zero (0) is generated by using no decoding oligonucleotides having an identifier connector element (t) comprising a nucleotide sequence which is essentially complementary to at least a section of the unique identifier sequence of the identifier element (T) of a corresponding analyte-specific probe for an individual analyte.

14. The method according to claim 1, wherein if at least for one individual analyte a position of the code word is zero (0) in this cycle no corresponding decoding oligonucleotides having an identifier connector element (t) comprising a nucleotide sequence which is essentially complementary to at least a section of the unique identifier sequence of the identifier element (T) of a corresponding analyte-specific probe for an individual analyte are used.

15. The method according to claim 1, wherein the sample is contacted with at least two different sets of signal oligonucleotides, wherein the signal oligonucleotides in each set comprise a different signal element and comprise a different connector element (C).

16.-19. (canceled)

20. The method according to claim 1, wherein
the sample is contacted with:
at least two (2) different sets of non-signal decoding oligonucleotides for binding to at least two different identifier elements (T) of analyte-specific probes, each set of non-signal decoding oligonucleotides interacting with a different identifier element (T)
wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

21.-22. (canceled)

23. The method according to claim 1, wherein
the sample is contacted with
a set of non-signal oligonucleotides, each non-signal oligonucleotide comprising:
(aa) a translator connector element (C) comprising a nucleotide sequence which is essentially complementary to at least a section of the nucleotide sequence of the translator element (c), and
(bb) a quencher (Q), a signal element and a quencher (Q), or does not comprise a signal element.

24.-43. (canceled)

44. The method according to claim 1, wherein the binding element (S) comprises moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof.

45. The method according to claim 1, wherein the binding element (S) is an antibody or an antibody fragment selected from the group consisting of Fab, scFv; single domain, or a fragment thereof, bis scFv, Fab2, Fab3, minibody, diabody, triabody, tetrabody and tandab.

46. The method according to claim 1, wherein the signal caused by the signal element, therefore in particular the binding of the signal oligonucleotides to the decoding oligonucleotides, interacting with the corresponding analyte probes, bound to the respective analyte is determined by at least one approach selected from the list consisting of
Imaging at least a portion of the sample;
Using an optical imaging technique;
Using a fluorescence imaging technique;
Multi-color fluorescence imaging technique; and
Super-resolution fluorescence imaging technique.

47. The method according to claim 1, wherein the decoding oligonucleotides in at least one set of decoding oligonucleotides are multi-decoders comprising
(aa) an identifier connector element (t) comprising a nucleotide sequence which is essentially complementary to at least a section of the unique identifier sequence of the identifier element (T) of the corresponding analyte-specific probe set, and
(bb) at least two translator elements (c), wherein the translator elements comprising different nucleotide sequences allowing a specific hybridization of a different signal oligonucleotide.

48. The method according to claim 47, wherein the different signal oligonucleotides comprise a different signal element and comprise a different connector element (C).

49. A kit for multiplex analyte encoding, comprising
(i) at least twenty (20) different sets of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising
(aa) a binding element (S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(ii) at least one set of decoding oligonucleotides per analyte, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(iii) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

50. The kit according to claim 49, wherein the kit comprises at least two different sets of analyte-specific probes for an analyte,
wherein the analyte-specific probes comprised in these different sets interacting with the same analyte, but specifically interact with different sub-structures of the same analyte,
wherein the analyte-specific probes of the first set of analyte-specific probes interacts with a sub-structure which is comprised in all variations of an analyte,
wherein the analyte-specific probes of the second set of analyte-specific probes (subgroup-specific probes) interacts with a sub-structure which is comprised only in a specific variation of the analyte,
wherein the analyte-specific probes of the first set of analyte-specific probes comprise the same identifier element (T) comprising a nucleotide sequence which is unique to the analyte to be encoded (unique identifier sequence), and
wherein the analyte-specific probes of the second set of analyte-specific probes comprise the same identifier element (T) comprising a nucleotide sequence which is unique to the analyte to be encoded (unique identifier sequence),
wherein the identifier elements (T) of the analyte-specific probes of the first set of analyte-specific probes and the identifier elements (T) of the analyte-specific probes of the second set of analyte-specific probes are different.

51. The kit according to claim 50, wherein the kit comprises at least five (5) sets of subgroup-specific probes that differ from the analyte-specific probes of another set of analyte-specific probes in the nucleotide sequence of the identifier element (T).

52.-54. (canceled)

55. The kit according to claim 49, wherein the kit comprises at least two different sets of signal oligonucleotides, wherein the signal oligonucleotides in each set comprise a different signal element and comprise a different connector element (C).

56.-132. (canceled)
